(19) United States
(12) Patent Application Publication
(10) Pub. No.: US 2021/0040205 A1
(43) Pub. Date: Feb. 11, 2021

(54) ANTIBODIES TARGETING CD32B AND METHODS OF USE THEREOF

(71) Applicant: NOVARTIS AG, Basel (CH)

(72) Inventors: Sunyoung JANG, Cambridge, MA (US); Haihui LU, Winchester, MA (US); Matthew John MEYER, Framingham, MA (US); Ryan MOLONY, Cambridge, MA (US); Karrie WONG, Somerville, MA (US)

(21) Appl. No.: 16/758,748

(22) PCT Filed: Oct. 23, 2018
(86) PCT No.: PCT/IB2018/001339

§ 371 Date: Apr. 23, 2020

Related U.S. Application Data
(60) Provisional application No. 62/576,970, filed on Oct. 25, 2017.

(51) Int. Cl. C07K 16/28; A61K 47/68; A61K 45/06

(52) U.S. Cl. CPC C07K 16/283; A61K 47/6849; C07K 16/2896; C07K 2317/76; C07K 2317/41; C07K 2317/732; C07K 2317/24; A61K 45/06

(57) ABSTRACT

The present disclosure relates to anti-CD32b antibody molecules which selectively bind human CD32b. Also provided herein are compositions comprising anti-CD32b antibody molecules in combination with other compounds, and methods of using the combinations to treat subject with cancer.

### SEQUENCE LISTING

The instant application contains a Sequence Listing which has been submitted electronically in ASCII format and is hereby incorporated by reference in its entirety. Said ASCII copy, created on Oct. 22, 2018, is named PAT057911-WO-PCT_SL.txt and is 373,110 bytes in size.

### FIELD OF THE DISCLOSURE

The present disclosure relates to methods of using antibodies and antigen-binding fragments thereof which bind human CD32b, and compositions thereof.

### BACKGROUND

Fc gamma receptors (FcγR) bind IgG and they are expressed by many immune cells, enabling them to serve as the link between innate and humoral immunity. Activatory FcγR contain immune-receptor tyrosine-based activating motifs (ITAMs) either directly in their intracellular portion or in the cytoplasmic domain of associated signaling units such as the homodimeric common γ chain. These ITAM motifs become phosphorylated when the receptors are cross-linked by antigen-antibody complexes. Activatory FcγR contain or are associated with immune-receptor tyrosine-based activating motifs (ITAMs) which become phosphorylated when the receptors are cross-linked by antigen-antibody complexes. Upon activation, these receptors mediate immune responses including phagocytosis and antibody dependent cellular cytotoxicity (ADCC) (Nimmerjahn and Ravetch, Nature Rev. Immunol. 2008: 8(1) 34-47). CD32b is the sole inhibitory FcγR and contains an intracellular immune-receptor tyrosine-based inhibitory mofit (ITIM). CD32b is expressed by immune cells including dendritic cells and macrophages (Nimmerjahn and Ravetch, Nature Rev. Immunol. 2008: 8(1) 34-47) and is the only FcγR expressed on B cells (Amigorena et al., Eur. J. Immunol. 1989:19(8) 1379-1385). Activation of CD32b and ITIM phosphorylation results in inhibition of activatory FcγR functions (Smith and Clatworthy, Nat. Rev. Immunol. 2010: (5) 328-343) or, when cross-linked to the B cell receptor, reduced B cell function (Horton et al., J. Immunol. 2011: 186(7):4223-4233). Consistent with its inhibitory role, therapeutic antibodies with Fc dependent activity/ADCC mode of action have a more robust anti-tumor response in CD32b knockout mice than in WT mice (Clynes et al., Nat. Med. 2000: 6(4):443-6). Additionally, polymorphisms that impair CD32b function are associated with development of autoimmunity (Floto et al., Nat. Med. 2005: 11(10) 1056-1058).

CD32b is expressed as two splice variants, CD32b1 and CD32b2, which have similar extracellular domains but different intracellular domains that dictate their propensity for internalization. The full length variant, CD32b1 (UniProtKB P31944-1), is expressed on lymphoid cells and has an intracellular signal sequence that prevents internalization. CD32b2 (UniProtKB P31944-2), which is expressed on myloid cells, lacks this signal sequence and is therefore more susceptible to internalization (Brooks et al., J. Exp. Med. 1989: 170(4) 1369-1385).

In addition to being expressed throughout B cell maturation, CD32b is found highly expressed on the malignant counter parts of these cells. Specifically, CD32b is found expressed on B cell lymphomas including CLL, NHL, multiple myeloma, and CD32b has been proposed as a therapeutic target for these indications (e.g. Rankin et al., Blood 2006: 108(7) 2384-2391) and others including systemic light-chain amyloidosis (Zhou et al., Blood 2008: 111(7) 3403-3406).

Expression of CD32b on tumor cells has been shown to correlate with reduced clinical benefit from rituximab containing treatment regimens (Lim et al., Blood 2011: 118(9) 2530-2540). Furthermore, CD32b expression was found to be increased in a B cell leukemia model upon developing resistance to alemtuzumab in vivo and knockdown of CD32b re-sensitized the leukemic cells to alemtuzumab mediated ADCC activity (Pallasch et al., Cell 2014: 156(3) 590-602). Taken together, these data support a role for CD32b as a mechanism of resistance to antibodies with Fc dependent (e.g. ADCC mediated) anti-tumor activity. This mechanism is not well understood but several hypotheses exist. Lim et al. (Blood 2011: 118(9) 2530-2540) and Vaughan et al. (Blood 2014: 123(5) 669-677) demonstrated with lymphoma cells that CD32b binds the Fc of CD20 bound rituximab causing the tripartite complex to internalize and ultimately resulting in reduced CD20 bound rituximab coating the lymphoma cell surface. It has also been proposed that CD32b on lymphoma cells engage the Fc region of, for example, CD20 bound rituximab in cis effectively masking the rituximab Fc. The anticipated consequence of the rituximab Fc masking is a reduced opportunity to engage the activatory FcγR on effector cells in trans (Vaughan et al. Blood 2014: 123(5) 669-677). Evidence that FcγR can function in this manner has been demonstrated during herpes simplex virus infection, where a virally encoded FcγR engages the Fc region of antibodies bound to viral antigens expressed by the infected cell thereby protecting it from antibody-dependent cellular cytotoxicity (Van Vliet et al., Immunology 1992: 77(1) 109-115). In both mechanisms outlined above, CD32b effectively reduces the interactions between a therapeutic mAb Fc, e.g. rituximab, and activatory FcγR on effector cells resulting in a diminished immune response/ADCC activity.

### SUMMARY

The disclosure provides, at least in part, methods and compositions comprising an anti-CD32b antibody molecule described herein, e.g., in Tables 1, 2 or 3, optionally, in combination with a second therapeutic agent, e.g., one or more therapeutic agents, e.g., 1, 2, 3, 4 or more therapeutic agents described herein. In some embodiments, the second therapeutic agent is chosen from one or more of: (i) an antibody that binds a cell surface antigen, e.g., a surface antigen on an immune cell or a cancer or a tumor cell; (ii) an immunomodulatory compound or an immune-based therapy, e.g., one or more of a cytokine, an activator of a costimulatory molecule or an agonist of a costimulatory moleculeor an inhibitor of an inhibitory molecule (e.g., an inhibitor of a checkpoint inhibitor), as described herein; (iii) an anti-cancer therapy, e.g., one or more of a targeted anti-cancer therapy (e.g., an antibody molecule), or a cytotoxic agent (e.g., a chemotherapy, an oncolytic drug, or a small molecule inhibitor) as described herein.

In other embodiments, the anti-CD32b antibody molecule is administered in combination with an antibody that binds a cell surface antigen, e.g., a surface antigen on an immune cell or a cancer or a tumor cell, further in combination with one or both of: an immunomodulatory compound or an immune-based therapy, e.g., one or more of a cytokine, an activator of a costimulatory molecule, or an inhibitor of an inhibitory molecule (e.g., an inhibitor of a checkpoint inhibitor), as described herein; or an anti-cancer therapy, e.g., one or more of a targeted anti-cancer therapy (e.g., an antibody molecule), or a cytotoxic agent (e.g., a chemotherapy, an oncolytic drug, or a small molecule inhibitor) as described herein.

In some embodiments, the anti-CD32b antibodies disclosed herein, selectively bind human CD32b over human CD32a. In one embodiment, the anti-CD32b antibody or antigen-binding fragment thereof disclosed in the present application inhibits binding of human CD32b to immunoglobulin Fc domains. For example, the anti-CD32b antibody or antigen-binding fragment thereof can inhibit binding of human CD32b to an immunoglobulin Fc domain from a second therapeutic antibody or other Fc domain-containing molecule. The compositions, combinations and methods described herein can provide a beneficial effect, e.g., in the treatment of a cancer, such as an enhanced anti-cancer effect, reduced toxicity and/or reduced side effects. For example, the anti-CD32b antibody molecule, the second therapeutic agent, or both, can be administered at a lower dosage than would be required to achieve the same therapeutic effect compared to a monotherapy dose. Thus, compositions and methods for treating proliferative disorders, including cancer, using the aforesaid anti-CD32b antibodies and combination therapies are disclosed.

Accordingly, in one aspect, the disclosure provides a method of treating (e.g., inhibiting, reducing, or ameliorating) a CD32b related condition or disorder, e.g., a proliferative condition or disorder (e.g., a cancer) in a subject.

In one embodiment, this application discloses a method of treating a cancer in a subject, comprising administering to the subject an anti-CD32b antibody molecule, in combination with one or more second therapeutic agents, wherein the second therapeutic agent is chosen from one or more of:

(i) an antibody that binds a cell surface antigen on a cancer cell, tumor cell, or an immune cell;

(ii) an immunomodulatory compound; or

(iii) an anti-cancer therapy,

wherein the anti-CD32b antibody molecule is chosen from an antibody disclosed in Table 1, 2, or 3; thereby treating the cancer.

In another embodiment, this application discloses use of an anti-CD32b antibody molecule in combination with one or more second therapeutic agents to treat cancer in a subject, wherein the second therapeutic agent is chosen from one or more of:

(i) an antibody that binds a cell surface antigen on a cancer cell, tumor cell, or an immune cell;

(ii) an immunomodulatory compound; or

(iii) an anti-cancer therapy,

wherein the anti-CD32b antibody molecule is chosen from an antibody disclosed in Table 1, 2, or 3; thereby treating the cancer.

In another embodiment, this application discloses a composition comprising an anti-CD32b antibody molecule in combination with one or more second therapeutic agents, for use in treating a cancer in a subject, wherein the second agent is chosen from one or more of:

(i) an antibody that binds a cell surface antigen on a cancer cell, tumor cell, or an immune cell;

(ii) an immunomodulatory compound; or

(iii) an anti-cancer therapy,

wherein the anti-CD32b antibody molecule is chosen from an antibody disclosed in Table 1, 2, or 3.

In another embodiment, this application discloses use of anti-CD32b antibody molecule in the manufacture of a medicament for use in combination with

(i) an antibody that binds a cell surface antigen on a cancer cell, tumor cell, or an immune cell;

(ii) an immunomodulatory compound; or

(iii) an anti-cancer therapy,

to treat cancer in a subject, wherein the anti-CD32b antibody molecule is chosen from an antibody disclosed in Table 1, 2, or 3.

In some embodiments, the anti-CD32b antibody molecule is selected from an antibody comprising:

a heavy chain variable region comprising the amino acid sequence of SEQ ID NO: 1, or an amino acid sequence at least 95% identical thereto, and a light chain variable region comprising the amino acid sequence of SEQ ID NO: 2, or an amino acid sequence at least 95% identical thereto;

a heavy chain variable region comprising the amino acid sequence of SEQ ID NO: 9, or an amino acid sequence at least 95% identical thereto, and a light chain variable region comprising the amino acid sequence of SEQ ID NO: 10, or an amino acid sequence at least 95% identical thereto;

a heavy chain variable region comprising the amino acid sequence of SEQ ID NO: 17, or an amino acid sequence at least 95% identical thereto, and a light chain variable region comprising the amino acid sequence of SEQ ID NO: 18, or an amino acid sequence at least 95% identical thereto;

a heavy chain variable region comprising the amino acid sequence of SEQ ID NO: 25, or an amino acid sequence at least 95% identical thereto, and a light chain variable region comprising the amino acid sequence of SEQ ID NO: 26, or an amino acid sequence at least 95% identical thereto;

a heavy chain variable region comprising the amino acid sequence of SEQ ID NO: 33, or an amino acid sequence at least 95% identical thereto, and a light chain variable region comprising the amino acid sequence of SEQ ID NO: 34, or an amino acid sequence at least 95% identical thereto;

a heavy chain variable region comprising the amino acid sequence of SEQ ID NO: 41, or an amino acid sequence at least 95% identical thereto, and a light chain variable region comprising the amino acid sequence of SEQ ID NO: 42, or an amino acid sequence at least 95% identical thereto;

a heavy chain variable region comprising the amino acid sequence of SEQ ID NO: 49, or an amino acid sequence at least 95% identical thereto, and a light chain variable region comprising the amino acid sequence of SEQ ID NO: 50, or an amino acid sequence at least 95% identical thereto;

a heavy chain variable region comprising the amino acid sequence of SEQ ID NO: 57, or an amino acid sequence at least 95% identical thereto, and a light chain variable region comprising the amino acid sequence of SEQ ID NO: 58, or an amino acid sequence at least 95% identical thereto;

a heavy chain variable region comprising the amino acid sequence of SEQ ID NO: 65, or an amino acid sequence at least 95% identical thereto, and a light chain variable region comprising the amino acid sequence of SEQ ID NO: 66, or an amino acid sequence at least 95% identical thereto;

a heavy chain variable region comprising the amino acid sequence of SEQ ID NO: 73, or an amino acid sequence at least 95% identical thereto, and a light chain variable region comprising the amino acid sequence of SEQ ID NO: 74, or an amino acid sequence at least 95% identical thereto;

a heavy chain variable region comprising the amino acid sequence of SEQ ID NO: 81, or an amino acid sequence at least 95% identical thereto, and a light chain variable region comprising the amino acid sequence of SEQ ID NO: 82, or an amino acid sequence at least 95% identical thereto;

a heavy chain variable region comprising the amino acid sequence of SEQ ID NO: 89, or an amino acid sequence at least 95% identical thereto, and a light chain variable region comprising the amino acid sequence of SEQ ID NO: 90, or an amino acid sequence at least 95% identical thereto;

a heavy chain variable region comprising the amino acid sequence of SEQ ID NO: 97, or an amino acid sequence at least 95% identical thereto, and a light chain variable region comprising the amino acid sequence of SEQ ID NO: 98, or an amino acid sequence at least 95% identical thereto;

a heavy chain variable region comprising the amino acid sequence of SEQ ID NO: 105, or an amino acid sequence at least 95% identical thereto, and a light chain variable region comprising the amino acid sequence of SEQ ID NO: 106, or an amino acid sequence at least 95% identical thereto;

a heavy chain variable region comprising the amino acid sequence of SEQ ID NO: 113, or an amino acid sequence at least 95% identical thereto, and a light chain variable region comprising the amino acid sequence of SEQ ID NO: 114, or an amino acid sequence at least 95% identical thereto;

a heavy chain variable region comprising the amino acid sequence of SEQ ID NO: 121, or an amino acid sequence at least 95% identical thereto, and a light chain variable region comprising the amino acid sequence of SEQ ID NO: 122, or an amino acid sequence at least 95% identical thereto;

a heavy chain variable region comprising the amino acid sequence of SEQ ID NO: 129, or an amino acid sequence at least 95% identical thereto, and a light chain variable region comprising the amino acid sequence of SEQ ID NO: 130, or an amino acid sequence at least 95% identical thereto;

a heavy chain variable region comprising the amino acid sequence of SEQ ID NO: 201, or an amino acid sequence at least 95% identical thereto, and a light chain variable region comprising the amino acid sequence of SEQ ID NO: 205, or an amino acid sequence at least 95% identical thereto;

a heavy chain variable region comprising the amino acid sequence of SEQ ID NO: 209, or an amino acid sequence at least 95% identical thereto, and a light chain variable region comprising the amino acid sequence of SEQ ID NO: 213, or an amino acid sequence at least 95% identical thereto;

a heavy chain variable region comprising the amino acid sequence of SEQ ID NO: 217, or an amino acid sequence at least 95% identical thereto, and a light chain variable region comprising the amino acid sequence of SEQ ID NO: 221, or an amino acid sequence at least 95% identical thereto;

a heavy chain variable region comprising the amino acid sequence of SEQ ID NO: 225, or an amino acid sequence at least 95% identical thereto, and a light chain variable region comprising the amino acid sequence of SEQ ID NO: 229, or an amino acid sequence at least 95% identical thereto;

a heavy chain variable region comprising the amino acid sequence of SEQ ID NO: 233, or an amino acid sequence at least 95% identical thereto, and a light chain variable region comprising the amino acid sequence of SEQ ID NO: 237, or an amino acid sequence at least 95% identical thereto;

a heavy chain variable region comprising the amino acid sequence of SEQ ID NO: 241, or an amino acid sequence at least 95% identical thereto, and a light chain variable region comprising the amino acid sequence of SEQ ID NO: 213, or an amino acid sequence at least 95% identical thereto;

a heavy chain variable region comprising the amino acid sequence of SEQ ID NO: 245, or an amino acid sequence at least 95% identical thereto, and a light chain variable region comprising the amino acid sequence of SEQ ID NO: 249, or an amino acid sequence at least 95% identical thereto;

a heavy chain variable region comprising the amino acid sequence of SEQ ID NO: 253, or an amino acid sequence at least 95% identical thereto, and a light chain variable region comprising the amino acid sequence of SEQ ID NO: 257, or an amino acid sequence at least 95% identical thereto;

a heavy chain variable region comprising the amino acid sequence of SEQ ID NO: 261, or an amino acid sequence at least 95% identical thereto, and a light chain variable region comprising the amino acid sequence of SEQ ID NO: 265, or an amino acid sequence at least 95% identical thereto;

a heavy chain variable region comprising the amino acid sequence of SEQ ID NO: 269, or an amino acid sequence at least 95% identical thereto, and a light chain variable region comprising the amino acid sequence of SEQ ID NO: 273, or an amino acid sequence at least 95% identical thereto;

a heavy chain variable region comprising the amino acid sequence of SEQ ID NO: 300, or an amino acid sequence at least 95% identical thereto, and a light chain variable region comprising the amino acid sequence of SEQ ID NO: 301, or an amino acid sequence at least 95% identical thereto;

a heavy chain variable region comprising the amino acid sequence of SEQ ID NO: 313, or an amino acid sequence at least 95% identical thereto, and a light chain variable region comprising the amino acid sequence of SEQ ID NO: 310, or an amino acid sequence at least 95% identical thereto;

a heavy chain variable region comprising the amino acid sequence of SEQ ID NO: 313, or an amino acid sequence at least 95% identical thereto, and a light chain variable region comprising the amino acid sequence of SEQ ID NO: 311, or an amino acid sequence at least 95% identical thereto;

a heavy chain variable region comprising the amino acid sequence of SEQ ID NO: 313, or an amino acid sequence at least 95% identical thereto, and a light chain variable region comprising the amino acid sequence of SEQ ID NO: 312, or an amino acid sequence at least 95% identical thereto;

a heavy chain variable region comprising the amino acid sequence of SEQ ID NO: 317, or an amino acid sequence at least 95% identical thereto, and a light chain variable region comprising the amino acid sequence of SEQ ID NO: 321, or an amino acid sequence at least 95% identical thereto;

a heavy chain variable region comprising the amino acid sequence of SEQ ID NO: 323, or an amino acid sequence at least 95% identical thereto, and a light chain variable region comprising the amino acid sequence of SEQ ID NO: 322, or an amino acid sequence at least 95% identical thereto;

a heavy chain variable region of an antibody produced by hybridoma clone ID5 having ATCC accession number PTA-5958, or an amino acid sequence at least 95% identical thereto, and a light chain variable region of an antibody produced by hybridoma clone ID5 having ATCC accession number PTA-5958, or an amino acid sequence at least 95% identical thereto;

a heavy chain variable region of an antibody produced by hybridoma clone 2E1 having ATCC accession number PTA-5961, or an amino acid sequence at least 95% identical thereto, and a light chain variable region of an antibody produced by hybridoma clone 2E1 having ATCC accession number PTA-5961, or an amino acid sequence at least 95% identical thereto;

a heavy chain variable region of an antibody produced by hybridoma clone 2H9 having ATCC accession number PTA-5962, or an amino acid sequence at least 95% identical thereto, and a light chain variable region of an antibody produced by hybridoma clone 2H9 having ATCC accession number PTA-5962, or an amino acid sequence at least 95% identical thereto;

a heavy chain variable region of an antibody produced by clone 2D11 having ATCC accession number PTA-5960, or an amino acid sequence at least 95% identical thereto, and a light chain variable region of an antibody produced by hybridoma clone 2D11 having ATCC accession number PTA-5960, or an amino acid sequence at least 95% identical thereto; or

a heavy chain variable region of an antibody produced by clone 1F2 having ATCC accession number PTA-5959, or an amino acid sequence at least 95% identical thereto, and a light chain variable region of an antibody produced by hybridoma clone 1F2 having ATCC accession number PTA-5959, or an amino acid sequence at least 95% identical thereto.

In some embodiments of the methods, uses, or compositions disclosed herein, the second therapeutic agent comprises one or more antibodies that bind a cell surface antigen. In some embodiments, the cell surface antigen is co-expressed with CD32b. In further embodiments, the cell surface antigen is co-expressed with CD32b on B cells. In certain embodiments, the cell surface antigen is chosen from CD20, CD38, CD52, CS1/SLAMF7, CD56, CD138, KiR, CD19, CD40, Thy-1, Ly-6, CD49, Fas, Cd95, APO-1, EGFR, HER2, CXCR4, HLA molecules, GM1, CD22, CD23, CD80, CD74, or DRD. In other embodiments, the cell surface antigen is chosen from CD20, CD38, CS1/SLAMF7 or CD52. In yet other embodiments, the cell surface antigen is CD38. In some embodiments, the second therapeutic agent comprises an antibody selected from elotuzumab, ofatumumab, obinutuzumab, daratumumab, or alemtuzumab.

In some embodiments of the methods, uses, or compositions disclosed herein, the immunomodulatory compound is selected from a cytokine, an agonist of a costimulatory molecule, or an inhibitor of an inhibitory compound. In some embodiments, the immunomodulatory compound is a cytokine chosen from one or more of IL-15, IL-2, IL-6, IL-7, IL-9, IL-12, IL-18, IL-21, IL-23, or IL-27. In other embodiments, the immunomodulatory compound is an agonist of a costimulatory molecule selected from OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, CD83 ligand, or STING. In yet other embodiments, the immunomodulatory compound is an inhibitor of an inhibitory molecule selected from PD-1, PD-L1, PD-L2, CTLA-4, TIM-3, LAG-3, CEACAM-1, CEACAM-3, CEACAM-5, VISTA, BTLA, TIGIT, LAIR1, CD160, 2B4, TGF beta, or IDO.

In some embodiments of the methods, uses, or compositions disclosed herein, the anti-cancer therapy is selected from a targeted anti-cancer therapy, a cytotoxic agent, or a chemotherapeutic agent. In some embodiments, the anti-cancer therapy comprises a targeted anti-cancer therapy is selected from ofatumumab, romidepsin, brentuximab, obinutuzumab, elotuzumab, daratumumab, or alemtuzumab. In some embodiments, the cytotoxic agent is chosen from ibrutinib, belinostat, romidepsin, brentuximab vedotin, pralatrexate, pentostatin, dexamethasone, idelalisib, ixazomib, liposomal doxyrubicin, pomalidomide, panobinostat, thalidomide, or lenalidomide.

In some embodiments of the methods, uses, or compositions disclosed herein, the anti-CD32b antibody molecule, is administered in an amount sufficient to result in one or more of:

decreased B cell inhibition,

increased B cell activation;

enhanced immune cell-mediated ADCC, e.g., macrophage- or NK cell-mediated ADCC;

enhanced macrophage-mediated ADCP; or enhanced DC activity, e.g., DC maturation, antigen presentation and T cell priming.

In some embodiments, the methods, uses, or compositions disclosed herein are used to treat a subject with a CD32b-related condition or disorder.

In some embodiments, the methods, uses, or compositions disclosed herein are used to treat a subject has a condition or disorder chosen from: B cell malignancies, Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma, follicular lymphoma, systemic light chain amyloidosis, acute myeloid leukemia (AML), myelodysplasia, myelodysplastic syndrome, myelofibrosis, myeloproliferative neoplasms, acute lymphoid leukemia (ALL), hairy cell leukemia, prolymphocytic leukemia, chronic myeloid leukemia (CML), or blastic plasmacytoid dendritic cell neoplasm. In other embodiments, the subject has a solid cancer chosen from one or more of: pancreatic (e.g., pancreatic adenocarcinoma or pancreatic ductal adenocarcinoma), breast, colorectal, colon, lung (e.g., small or non-small cell lung cancer), skin, ovarian, prostate, cervix, gastrointestinal (e.g., carcinoid or stromal), stomach, head and neck, kidney, or liver cancer, or a metastatic lesion thereof.

Other features, objects, and advantages of the disclosure will be apparent from the description and drawings, and from the claims.

### DETAILED DESCRIPTION

The present disclosure provides anti-CD32b antibody molecules that specifically bind to human CD32b protein, and pharmaceutical compositions, production methods, and methods of use of such antibodies and compositions.

### Definitions

Unless defined otherwise, all technical and scientific terms used herein have the same meaning as commonly understood by those of ordinary skill in the art to which this disclosure pertains. All publications, patent applications, patents, and other references mentioned herein are incorporated by reference in their entirety.

The term “CD32A” or “CD32a”, as used herein, means human CD32a protein, also referred to as human FCγ Receptor 2A or FCγR2A or FCGR2a or FCGR2A. There are two variants known as H131 and R131 (when referenced without the signal sequence) or H167 and R167 (when referenced with the signal sequence). The amino acid sequence of the H167 variant is deposited under accession number UniProtKB P12318 and set forth below:

(SEQ ID NO: 677)
MTMETQMSQNVCPRNLWLLQPLTVLLLLASADSQAAAPPKAVLKLEPPWI
NVLQEDSVTLTCQGARSPESDSIQWFHNGNLIPTHTQPSYRFKANNNDSG
EYTCQTGQTSLSDPVHLTVLSEWLVLQTPHLEFQEGETIMLRCHSWKDKP
LVKVTFFQNGKSQKFSHLDPTFSIPQANHSHSGDYHCTGNIGYTLFSSKP
VTITVQVPSMGSSSPMGIIVAVVIATAVAAIVAAVVALIYCRKKRISANS
TDPVKAAQFEPPGRQMIAIRKRQLEETNNDYETADGGYMTLNPRAPTDDD
KNIYLTLPPNDHVNSNN.

“CD32B” or “CD32b”, as used herein, means human CD32b protein, also referred to as human FCγ Receptor 2B or FCγR2B or FCGR2b or FCGR2B. The amino acid sequence for CD32b variant 1 is deposited under accession number UniProtKB P31994-land set forth below:

(SEQ ID NO: 678)
MGILSFLPVLATESDWADCKSPQPWGHMLLWTAVLFLAPVAGTPAAPPKA
VLKLEPQWINVLQEDSVTLTCRGTHSPESDSIQWFHNGNLIPTHTQPSYR
FKANNNDSGEYTCQTGQTSLSDPVHLTVLSEWLVLQTPHLEFQEGETIVL
RCHSWKDKPLVKVTFFQNGKSKKFSRSDPNFSIPQANHSHSGDYHCTGNI
GYTLYSSKPVTITVQAPSSSPMGIIVAVVTGIAVAAIVAAVVALIYCRKK
RISALPGYPECREMGETLPEKPANPTNPDEADKVGAENTITYSLLMHPDA
LEEPDDQNRI.

The amino acid sequence for CD32b variant 2 is deposited under accession number UniProtKB P31994-2 and set forth below:

(SEQ ID NO: 679)
MGILSFLPVLATESDWADCKSPQPWGHMLLWTAVLFLAPVAGTPAAPPKA
VLKLEPQWINVLQEDSVTLTCRGTHSPESDSIQWFHNGNLIPTHTQPSYR
FKANNNDSGEYTCQTGQTSLSDPVHLTVLSEWLVLQTPHLEFQEGETIVL
RCHSWKDKPLVKVTFFQNGKSKKFSRSDPNFSIPQANHSHSGDYHCTGNI
GYTLYSSKPVTITVQAPSSSPMGIIVAVVTGIAVAAIVAAVVALIYCRKK
RISANPTNPDEADKVGAENTITYSLLMHPDALEEPDDQNRI.

As described herein, an antibody or antigen-binding fragment thereof which binds to CD32b binds to human CD32b protein. As used herein “huCD32b” refers to human CD32b protein or a fragment thereof.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda.

A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The terms “antigen-binding fragment”, “antigen-binding fragment thereof,” “antigen binding portion” of an antibody, and the like, as used herein, refer to one or more fragments of an intact antibody that retain the ability to specifically bind to a given antigen (e.g., CD32b). Antigen binding functions of an antibody can be performed by fragments of an intact antibody. Examples of binding fragments encompassed within the term “antigen binding portion” of an antibody include a Fab fragment, a monovalent fragment consisting of the VL, VH, CL and CH1 domains; a F (ab)2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989 Nature 341:544-546), which consists of a VH domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies.

Antibody molecules, e.g., antigen binding portions, can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding portions can be incorporated into single chain molecules comprising a pair of tandem Fv segments (VH-CH1-VH-CH1) which, together with complementary light chain polypeptides, form a pair of antigen binding regions (Zapata et al., 1995 Protein Eng. 8 (10):1057-1062; and U.S. Pat. No. 5,641,870).

As used herein, the term “Affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “Avidity” refers to an informative measure of the overall stability or strength of the antibody-antigen complex. It is controlled by three major factors: antibody epitope affinity; the valency of both the antigen and antibody; and the structural arrangement of the interacting parts. Ultimately these factors define the specificity of the antibody, that is, the likelihood that the particular antibody is binding to a precise antigen epitope.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

The term “binding specificity” as used herein refers to the ability of an individual antibody combining site to react with one antigenic determinant and not with a different antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

Specific binding between two entities means a binding with an equilibrium constant (KA or KA) of at least 1×10⁷ M⁻¹, 10⁸ M⁻¹, 10⁹ M⁻¹, 10¹⁰ M⁻¹, 10¹¹ M⁻¹, 10¹² M⁻¹, 10¹³ M⁻¹, or 10¹⁴M⁻¹. The phrase “specifically (or selectively) binds” to an antigen (e.g., CD32b-binding antibody) refers to a binding reaction that is determinative of the presence of a cognate antigen (e.g., a human CD32b protein) in a heterogeneous population of proteins and other biologics. A CD32b-binding antibody of the disclosure binds to CD32b with a greater affinity than it does to a non-specific antigen (e.g., CD32a). The phrases “an antibody recognizing an antigen” and “an antibody specific for an antigen” are used interchangeably herein with the term “an antibody which binds specifically to an antigen”.

The term “chimeric antibody” (or antigen-binding fragment thereof) is an antibody molecule (or antigen-binding fragment thereof) in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In one embodiment, the term “conservative sequence modifications” are used to refer to amino acid modifications that do not significantly affect or alter the binding characteristics of the antibody containing the amino acid sequence.

The term “blocks” as used herein refers to stopping or preventing an interaction or a process, e.g., stopping ligand-dependent or ligand-independent signaling.

The term “recognize” as used herein refers to an antibody antigen-binding fragment thereof that finds and interacts (e.g., binds) with its conformational epitope.

The terms “cross-block”, “cross-blocked”, “cross-blocking”, “compete”, “cross compete” and related terms are used interchangeably herein to mean the ability of an antibody or other binding agent to interfere with the binding of other antibodies or binding agents to CD32b in a standard competitive binding assay.

The ability or extent to which an antibody or other binding agent is able to interfere with the binding of another antibody or binding molecule to CD32b, and therefore whether it can be said to cross-block according to the disclosure, can be determined using standard competition binding assays. One suitable assay involves the use of the Biacore technology (e.g. by using the BIAcore 3000 instrument (Biacore, Uppsala, Sweden)), which can measure the extent of interactions using surface plasmon resonance technology. Another assay for measuring cross-blocking uses an ELISA-based approach. Although the techniques are expected to produce substantially similar results, measurement by the Biacore technique is considered definitive.

The term “neutralizes” means that an antibody, upon binding to its target, reduces the activity, level or stability of the target; e.g., a CD32b antibody, upon binding to CD32b neutralizes CD32b by at least partially reducing an activity, level or stability of CD32b, such as its role in engaging Fc portions of antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “epitope” includes any protein determinant capable of specific binding to an immunoglobulin or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational.”

The term “linear epitope” refers to an epitope with all of the points of interaction between the protein and the interacting molecule (such as an antibody) occurring linearally along the primary amino acid sequence of the protein (continuous).

As used herein, the term “high affinity” for an IgG antibody refers to an antibody having a KD of 10-8 M or less, 10-9 M or less, or 10-10 M, or 10-11 M or less for a target antigen, e.g., CD32b. However, “high affinity” binding can vary for other antibody isotypes. For example, “high affinity” binding for an IgM isotype refers to an antibody having a KD of 10-7 M or less, or 10-8 M or less.

The term “human antibody” (or antigen-binding fragment thereof), as used herein, is intended to include antibodies (and antigen-binding fragments thereof) having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies and antigen-binding fragments thereof of the disclosure may include amino acid residues not encoded by human sequences (e.g., mutations introduced by random or site-specific mutagenesis in vitro or by somatic mutation in vivo).

The phrases “monoclonal antibody” or “monoclonal antibody composition” (or antigen-binding fragment thereof) as used herein refers to polypeptides, including antibodies, antibody fragments, bispecific antibodies, etc. that have substantially identical to amino acid sequence or are derived from the same genetic source. This term also includes preparations of antibody molecules of single molecular composition. A monoclonal antibody composition displays a single binding specificity and affinity for a particular epitope.

The term “human monoclonal antibody” (or antigen-binding fragment thereof) refers to antibodies (and antigen-binding fragments thereof) displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The phrase “recombinant human antibody” (or antigen-binding fragment thereof), as used herein, includes all human antibodies (and antigen-binding fragments thereof) that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In one embodiment, such recombinant human antibodies can be subjected to in vitro mutagenesis (or, when an animal transgenic for human Ig sequences is used, in vivo somatic mutagenesis) and thus the amino acid sequences of the VH and

VL regions of the recombinant antibodies are sequences that, while derived from and related to human germline VH and VL sequences, may not naturally exist within the human antibody germline repertoire in vivo.

A “humanized” antibody (or antigen-binding fragment thereof), as used herein, is an antibody (or antigen-binding fragment thereof) that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun, 28:489-498, 1991; and Padlan, Molec. Immun, 31:169-217, 1994. Other examples of human engineering technology include, but is not limited to Xoma technology disclosed in U.S. Pat. No. 5,766,886.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, 96%, 97%, 98% or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length. Optionally, the identity exists over a region that is at least 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, John Wiley & Sons, Inc. (ringbou ed., 2003)).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (N) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated antibody” (or antigen-binding fragment thereof), as used herein, refers to an antibody (or antigen-binding fragment thereof) that is substantially free of other antibodies having different antigenic specificities (e.g., an isolated antibody that specifically binds CD32b is substantially free of antibodies that specifically bind antigens other than CD32b). Moreover, an isolated antibody may be substantially free of other cellular material and/or chemicals.

The term “isotype” refers to the antibody class (e.g., IgM, IgE, IgG such as IgG1 or IgG4) that is provided by the heavy chain constant region genes. Isotype also includes modified versions of one of these classes, where modifications have been made to after the Fc function, for example, to enhance or reduce effector functions or binding to Fc receptors.

The term “Kassoc”, “Ka” or “Kon”, as used herein, is intended to refer to the association rate of a particular antibody-antigen interaction, whereas the term “Kdis”, “Kd,” or “Koff”, as used herein, is intended to refer to the dissociation rate of a particular antibody-antigen interaction. In one embodiment, the term “KD”, as used herein, is intended to refer to the dissociation constant, which is obtained from the ratio of Kd to Ka (i.e. Kd/Ka) and is expressed as a molar concentration (M). KD values for antibodies can be determined using methods well established in the art. A method for determining the KD of an antibody is by using surface plasmon resonance, or using a biosensor system such as a Biacore® system. Where the dissociation constant is less than about 10-9 M, solution equilibrium kinetic exclusion KD measurement (MSD-SET) is a preferred method for determining the KD of an antibody (see, e.g., Friquet, B., Chaffotte, A. F., Djavadi-Ohaniance, L., and Goldberg, M. E. (1985). Measurements of the true affinity constant in solution of antigen-antibody complexes by enzyme-linked immunosorbent assay J Immnunol Meth 77, 305-319; herein incorporated by reference).

The term “IC50,” as used herein, refers to the concentration of an antibody or an antigen-binding fragment thereof, which induces an inhibitory response, either in an in vitro or an in vivo assay, which is 50% of the maximal response, i.e., halfway between the maximal response and the baseline.

The terms “monoclonal antibody” (or antigen-binding fragment thereof) or “monoclonal antibody (or antigen-binding fragment thereof) composition” as used herein refer to a preparation of an antibody molecule (or antigen-binding fragment thereof) of single molecular composition. A monoclonal antibody composition displays a single binding specificity and affinity for a particular epitope.

The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonisation and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “recombinant human antibody” (or antigen-binding fragment thereof), as used herein, includes all human antibodies (and antigen-binding fragments thereof) that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In one embodiment, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treat,” “treated,” “treating,” and “treatment,” includes the administration of compositions or antibodies to alleviate the symptoms or arrest or inhibit further development of the disease, condition, or disorder. Treatment may be therapeutic suppression or alleviation of symptoms after the manifestation of the disease. Treatment can be measured by the therapeutic measures described herein. The methods of “treatment” of the present disclosure include administration of a CD32b antibody or antigen binding fragment thereof to a subject in order to cure, reduce the severity of, or ameliorate one or more symptoms of a disease or condition, in order to prolong the health or survival of a subject beyond that expected in the absence of such treatment. For example, “treatment” includes the alleviation of a disease symptom in a subject by at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or more.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

### Cd32B Antibodies and Antigen-Binding Fragments Thereof

The present disclosure provides antibodies and antigen-binding fragments thereof that specifically bind to human CD32b.

In one embodiment, the present disclosure provides anti-CD32b antibody molecules that bind with a higher affinity for human CD32b protein, than to human CD32a protein. Selectivity for CD32b over CD32a is desired to ensure selective binding to CD32b positive B-cell malignancies and B-cells while lacking binding to CD32a positive immune cells, including monocytes and neutrophils.

Antibodies of the disclosure include, but are not limited to, the human and humanized monoclonal antibodies isolated as described herein, including in Tables 1, 2 or 3. Examples of such anti-human CD32b antibodies are antibodies 6G11, 5C04, 5C05, 5D07, 5E12, 5G08, 5H06, 6A09, 6B01, 6C11, 6C12, 6D01, 6G03, 7C07, 4B02, 6G08, 6H08, 016, 020, 022, 024, 026, 028, 034, 038, 053, 063, 2B6, 3H7, 8B5, 1D5, 2E1, 2H9, 2D11, and 1F2. Additional exemplary anti-CD32b antibodies are disclosed in International Applications WO 2012/022985, WO 2015/173384, WO 2009/083009, WO 2004/016750, WO 2005/115452, WO 2005/110474, WO 2006/066078, and WO 2008/019199, the entire contents of all of which are hereby incorporated by reference in their entirety.

The present disclosure provides antibodies that specifically bind CD32b (e.g., human CD32b protein), said antibodies comprising a VH domain listed in Tables 1, 2 or 3. The present disclosure also provides antibodies that specifically bind to CD32b protein, said antibodies comprising a VH CDR having an amino acid sequence of any one of the VH CDRs listed in Tables 1, 2 or 3. In particular, the disclosure provides antibodies that specifically bind to CD32b protein, said antibodies comprising (or alternatively, consisting of) one, two, three, four, five or more VH CDRs having an amino acid sequence of any of the VH CDRs listed in Tables 1, 2 or 3.

The disclosure also provides antibodies and antigen-binding fragments thereof that specifically bind to CD32b, said antibodies or antigen-binding fragments thereof comprising (or alternatively, consisting of) a VH amino acid sequence listed in Tables 1, 2 or 3, wherein no more than about 10 amino acids in a framework sequence (for example, a sequence which is not a CDR) have been mutated (wherein a mutation is, as various non-limiting examples, an addition, substitution or deletion). The disclosure also provides antibodies and antigen-binding fragments thereof that specifically bind to CD32b, said antibodies or antigen-binding fragments thereof comprising (or alternatively, consisting of) a VH amino acid sequence listed in Tables 1, 2 or 3, wherein no more than 10 amino acids in a framework sequence (for example, a sequence which is not a CDR) have been mutated (wherein a mutation is, as various non-limiting examples, an addition, substitution or deletion).

The disclosure also provides antibodies and antigen-binding fragments thereof that specifically bind to CD32b, said antibodies or antigen-binding fragments thereof comprising (or alternatively, consisting of) a VH amino acid sequence listed in Tables 1, 2 or 3, wherein no more than about 20 amino acids in a framework sequence (for example, a sequence which is not a CDR) have been mutated (wherein a mutation is, as various non-limiting examples, an addition, substitution or deletion). The disclosure also provides antibodies and antigen-binding fragments thereof that specifically bind to CD32b, said antibodies or antigen-binding fragments thereof comprising (or alternatively, consisting of) a VH amino acid sequence listed in Tables 1, 2 or 3, wherein no more than 20 amino acids in a framework sequence (for example, a sequence which is not a CDR) have been mutated (wherein a mutation is, as various non-limiting examples, an addition, substitution or deletion).

The present disclosure provides anti-CD32b antibody molecules that specifically bind to CD32b protein, said antibodies or antigen-binding fragments thereof comprising a VL domain listed in Tables 1, 2 or 3. The present disclosure also provides anti-CD32b antibody molecules that specifically bind to CD32b protein, said antibodies or antigen-binding fragments thereof comprising a VL CDR having an amino acid sequence of any one of the VL CDRs listed in Tables 1, 2 or 3. In particular, the disclosure provides anti-CD32b antibody molecules that specifically bind to CD32b protein, said antibodies or antigen-binding fragments thereof comprising (or alternatively, consisting of) one, two, three or more VL CDRs having an amino acid sequence of any of the VL CDRs listed in Tables 1, 2 or 3.

The disclosure also provides anti-CD32b antibody molecules that specifically bind to CD32b, said anti-CD32b antibody molecules comprising (or alternatively, consisting of) a VL amino acid sequence listed in Tables 1, 2 or 3, wherein no more than about 10 amino acids in a framework sequence (for example, a sequence which is not a CDR) have been mutated (wherein a mutation is, as various non-limiting examples, an addition, substitution or deletion). The disclosure also anti-CD32b antibody molecules that specifically bind to CD32b, said anti-CD32b antibody molecules comprising (or alternatively, consisting of) a VL amino acid sequence listed in Tables 1, 2 or 3, wherein no more than 10 amino acids in a framework sequence (for example, a sequence which is not a CDR) have been mutated (wherein a mutation is, as various non-limiting examples, an addition, substitution or deletion).

The disclosure also provides anti-CD32b antibody molecules that specifically bind to CD32b, said anti-CD32b antibody molecules comprising (or alternatively, consisting of) a VL amino acid sequence listed in Tables 1, 2 or 3, wherein no more than about 20 amino acids in a framework sequence (for example, a sequence which is not a CDR) have been mutated (wherein a mutation is, as various non-limiting examples, an addition, substitution or deletion). The disclosure also provides anti-CD32b antibody molecules that specifically bind to CD32b, said anti-CD32b antibody molecules comprising (or alternatively, consisting of) a VL amino acid sequence listed in Tables 1, 2 or 3, wherein no more than 20 amino acids in a framework sequence (for example, a sequence which is not a CDR) have been mutated (wherein a mutation is, as various non-limiting examples, an addition, substitution or deletion).

Other anti-CD32b antibody molecules of the disclosure include amino acids that have been mutated, yet have at least 60, 70, 80, 90, 91, 92, 93, 94, 95, 96, 97, 98 or 99 percent identity in the CDR regions with the CDR regions depicted in the sequences described in Tables 1, 2 or 3. In one aspect, other anti-CD32b antibody molecules of the disclosure includes mutant amino acid sequences wherein no more than 1, 2, 3, 4 or 5 amino acids have been mutated in the CDR regions when compared with the CDR regions depicted in the sequence described in Tables 1, 2 or 3.

Throughout the text of this application, should there be a discrepancy between the text of the specification (e.g., Tables 1, 2 or 3) and the sequence listing, the text of the specification shall prevail.

TABLE 1
Exemplary anti-CD32b antibodies as disclosed
in WO 2012/022985 and WO 2015/173384

| | | |
|---|---|---|
| SEQ | Sequence | |
| ID | descrip- | |
| NO | tion | Sequence |

Clone 10: 6G11

| | | |
|---|---|---|
| 1 | VH | EVQLLESGGGLVQPGGSLRLSCAASGFTFSSY |
| | | GMHWVRQAPGKGLEWMAVISYDGSNKYYADSV |
| | | KGRFTISRDNSKNTLYLQMNSLRAEDTAVYYC |
| | | ARELYDAFDIWGQGTLVTVSS |
| 2 | VL | QSVLTQPPSASGTPGQRVTISCTGSSSNIGAG |
| | | YDVHWYQQLPGTAPKLLIYADDHRPSGVPDRF |
| | | SGSKSGTSASLAISGLRSEDEADYYCASWDDS |
| | | QRAVIFGGGTKLTVLG |
| 3 | HCDR1 | SYGMH |
| 4 | HCDR2 | VISYDGSNKYYADSVKG |
| 5 | HCDR3 | ELYDAFDI |
| 6 | LCDR1 | TGSSSNIGAGYDVH |
| 7 | LCDR2 | ADDHRPS |
| 8 | LCDR3 | ASWDDSQRAVI |

Clone 1: 5C04

| | | |
|---|---|---|
| 9 | VH | EVQLLESGGGLVQPGGSLRLSCAASGFTFSNY |
| | | GMHWVRQAPGKGLEWVAVISYDGSNKYYADSV |
| | | KGRFTISRDNSKNTLYLQMNSLRAEDTAVYYC |
| | | AREWRDAFDIWGQGTLVTVSS |
| 10 | VL | QSVLTQPPSASGTPGQRVTISCTGSSSNIGAG |
| | | YDVHWYQQLPGTAPKLLIYSDNQRPSGVPDRF |
| | | SGSKSGTSASLAISGLRSEDEADYYCAAWDDS |
| | | LSGSWVFGGGTKLTVLG |
| 11 | HCDR1 | NYGMH |
| 12 | HCDR2 | VISYDGSNKYYADSVKG |
| 13 | HCDR3 | WRDAFDI |
| 14 | LCDR1 | TGSSSNIGAGYDVH |
| 15 | LCDR2 | SDNQRPS |
| 16 | LCDR3 | AAWDDSLSGSWV |

Clone 2: 5D07

| | | |
|---|---|---|
| 17 | VH | EVQLLESGGGLVQPGGSLRLSCAASGFTFSTY |
| | | GMHWVRQAPGKGLEWVAVIAYDGSKKDYADSV |
| | | KGRFTISRDNSKNTLYLQMNSLRAEDTAVYYC |
| | | AREYRDAFDIWGQGTLVTVSS |
| 18 | VL | QSVLTQPPSASGTPGQRVTISCTGSSSNIGAG |
| | | YDVHWYQQLPGTAPKLLIYGNSNRPSGVPDRF |
| | | SGSKSGTTASLAISGLRSEDEADYYCAAWDDS |
| | | VSGWMFGGGTKLTVLG |
| 19 | HCDR1 | TYGMH |
| 20 | HCDR2 | VIAYDGSKKDYADSVKG |
| 21 | HCDR3 | EYRDAFDI |
| 22 | LCDR1 | TGSSSNIGAGYDVH |
| 23 | LCDR2 | GNSNRPS |
| 24 | LCDR3 | AAWDDSVSGWM |

Clone 3: 5G08

| | | |
|---|---|---|
| 25 | VH | EVQLLESGGGLVQPGGSLRLSCAASGFTFNNY |
| | | GMHWVRQAPGKGLEWVAVISYDGSNRYYADSV |
| | | KGRFTMSRDNSKNTLYLQMNSLRAEDTAVYYC |
| | | ARDRWNGMDVWGQGTLVTVSS |
| 26 | VL | QSVLTQPPSASGTPGQRVTISCSGSSSNIGAG |
| | | YDVHWYQQLPGTAPKLLIYANNQRPSGVPDRF |
| | | SGSKSGTSASLAISGLRSEDEADYYCAAWDDS |
| | | LNGPWVFGGGTKLTVLG |
| 27 | HCDR1 | NYGMH |
| 28 | HCDR2 | VISYDGSNRYYADSVKG |
| 29 | HCDR3 | DRWNGMDV |
| 30 | LCDR1 | SGSSSNIGAGYDVH |
| 31 | LCDR2 | ANNQRPS |
| 32 | LCDR3 | AAWDDSLNGPWV |

Clone 4: 5H06

| | | |
|---|---|---|
| 33 | VH | EVQLLESGGGLVQPGGSLRLSCAASGFTFSSY |
| | | GMHWVRQAPGKGLEWVAVISYDGSDTAYADSV |
| | | KGRFTISRDNSKNTLYLQMNSLRAEDTAVYYC |
| | | ARDHSVIGAFDIWGQGTLVTVSS |
| 34 | VL | QSVLTQPPSASGTPGQRVTISCSGSSSNIGSN |
| | | TVNWYQQLPGTAPKLLIYDNNKRPSGVPDRFS |
| | | GSKSGTSASLAISGLRSEDEADYYCSSYAGSN |
| | | NVVFGGGTKLTVLG |
| 35 | HCDR1 | SYGMH |
| 36 | HCDR2 | VISYDGSDTAYADSVKG |
| 37 | HCDR3 | DHSVIGAFDI |
| 38 | LCDR1 | SGSSSNIGSNTVN |
| 39 | LCDR2 | DNNKRPS |
| 40 | LCDR3 | SSYAGSNNVV |

Clone 5: 6B01

| | | |
|---|---|---|
| 41 | VH | EVQLLESGGGLVQPGGSLRLSCAASGFTFSNY |
| | | GMHWVRQAPGKGLEWVAVISYDGSNKYYADSV |
| | | KGRFTISRDNSKNTLYLQMNSLRAEDTAVYYC |
| | | ARDQLGEAFDIWGQGTLVTVSS |
| 42 | VL | QSVLTQPPSASGTPGQRVTISCTGSSSNIGAG |
| | | YDVHWYQQLPGTAPKLLIYDNNKRPSGVPDRF |
| | | SGSKSGTSASLAISGLRSEDEADYYCATWDDS |
| | | LSGPVFGGGTKLTVLG |
| 43 | HCDR1 | NYGMH |
| 44 | HCDR2 | VISYDGSNKYYADSVKG |
| 45 | HCDR3 | DQLGEAFDI |
| 46 | LCDR1 | TGSSSNIGAGYDVH |
| 47 | LCDR2 | DNNKRPS |
| 48 | LCDR3 | ATWDDSLSGPV |

Clone 6: 6C11

| | | |
|---|---|---|
| 49 | VH | EVQLLESGGGLVQPGGSLRLSCAASGFTFDDY |
| | | GMSWVRQAPGKGLEWVSAISGSGSSTYYADSV |
| | | KGRFTISRDNSKNTLYLQMNSLRAEDTAVYYC |
| | | AGGDIDYFDYWGQGTLVTVSS |
| 50 | VL | QSVLTQPPSASGTPGQRVTISCTGSSSNFGAG |
| | | YDVHWYQQLPGTAPKLLIYENNKRPSGVPDRF |
| | | SGSKSGTSASLAISGLRSEDEADYYCAAWDDS |
| | | LNGPVFGGGTKLTVLG |
| 51 | HCDR1 | DYGMS |
| 52 | HCDR2 | AISGSGSSTYYADSVKG |
| 53 | HCDR3 | GDIDYFDY |
| 54 | LCDR1 | TGSSSNFGAGYDVH |
| 55 | LCDR2 | ENNKRPS |
| 56 | LCDR3 | AAWDDSLNGPV |

Clone 7: 6C12

| | | |
|---|---|---|
| 57 | VH | EVQLLESGGGLVQPGGSLRLSCAASGFTFSSY |
| | | GMHWVRQAPGKGLEWVAVISYDGSNKYYADSV |
| | | KGRFTISRDNSKNTLYLQMNSLRAEDTAVYYC |
| | | ARERRDAFDIWGQGTLVTVSS |
| 58 | VL | QSVLTQPPSASGTPGQRVTISCTGSSSNIGAG |
| | | YDVHWYQQLPGTAPKLLIYSDNQRPSGVPDRF |
| | | SGSKSGTSASLAISGLRSEDEADYYCATWDSD |
| | | TPVFGGGTKLTVLG |
| 59 | HCDR1 | SYGMH |
| 60 | HCDR2 | VISYDGSNKYYADSVKG |
| 61 | HCDR3 | ERRDAFDI |
| 62 | LCDR1 | TGSSSNIGAGYDVH |
| 63 | LCDR2 | SDNQRPS |
| 64 | LCDR3 | ATWDSDTPV |

Clone 8: 6D01

| | | |
|---|---|---|
| 65 | VH | EVQLLESGGGLVQPGGSLRLSCAASGFTFSSY |
| | | GMHWVRQAPGKGLEWVAVISYDGSNKYYADSV |
| | | KGRFTISRDNSKNTLYLQMNSLRAEDTAMYYC |
| | | ARDHSAAGYFDYWGQGTLVTVSS |
| 66 | VL | QSVLTQPPSASGTPGQRVTISCSGSSSNIGSN |
| | | TVNWYQQLPGTAPKLLIYGNSIRPSGGPDRFS |
| | | GSKSGTSASLAISGLRSEDEADYYCASWDDSL |
| | | SSPVFGGGTKLTVLG |
| 67 | HCDR1 | SYGMH |
| 68 | HCDR2 | VISYDGSNKYYADSVKG |
| 69 | HCDR3 | DHSAAGYFDY |
| 70 | LCDR1 | SGSSSNIGSNTVN |
| 71 | LCDR2 | GNSIRPS |
| 72 | LCDR3 | ASWDDSLSSPV |

Clone 9: 6G03

| | | |
|---|---|---|
| 73 | VH | EVQLLESGGGLVQPGGSLRLSCAASGFTFGSY |
| | | GMHWVRQAPGKGLEWVSGISWDSAIIDYAGSV |
| | | KGRFTISRDNSKNTLYLQMNSLRAEDTAVYYC |
| | | AKDEAAAGAFDIWGQGTLVTVSS |
| 74 | VL | QSVLTQPPSASGTPGQRVTISCTGSSSNIGAG |
| | | YDVHWYQQLPGTAPKLLIYGNTDRPSGVPDRF |
| | | SGSKSGTSASLAISGLRSEDEADYYCAAWDDS |
| | | LSGPVVFGGGTKLTVLG |
| 75 | HCDR1 | SYGMH |
| 76 | HCDR2 | GISWDSAIIDYAGSVKG |
| 77 | HCDR3 | DEAAAGAFDI |
| 78 | LCDR1 | TGSSSNIGAGYDVH |
| 79 | LCDR2 | GNTDRPS |
| 80 | LCDR3 | AAWDDSLSGPVV |

Clone 11: 7C07

| | | |
|---|---|---|
| 81 | VH | EVQLLESGGGLVQPGGSLRLSCAASGFTFSSY |
| | | GMHWVRQAPGKGLEWVAVISYDGSNKYYADSV |
| | | KGRFTISRDNSQNTLYLQMNSLRAEDTAVYYC |
| | | AREFGYIILDYWGQGTLVTVSS |
| 82 | VL | QSVLTQPPSASGTPGQRVTISCSGSSSNIGSN |
| | | TVNWYQQLPGTAPKLLIYRDYERPSGVPDRFS |
| | | GSKSGTSASLAISGLRSEDEADYYCMAWDDSL |
| | | SGVVFGGGTKLTVLG |
| 83 | HCDR1 | SYGMH |
| 84 | HCDR2 | VISYDGSNKYYADSVKG |
| 85 | HCDR3 | EFGYIILDY |
| 86 | LCDR1 | SGSSSNIGSNTVN |
| 87 | LCDR2 | RDYERPS |
| 88 | LCDR3 | MAWDDSLSGVV |

Clone 12: 4B02

| | | |
|---|---|---|
| 89 | VH | EVQLLESGGGLVQPGGSLRLSCAASGFTFSNH |
| | | GMHWVRQAPGKGLEWVAVISYDGTNKYYADSV |
| | | RGRFTISRDNSKNTLYLQMNSLRAEDTAVYYC |
| | | ARETWDAFDVWGQGTLVTVSS |
| 90 | VL | QSVLTQPPSASGTPGQRVTISCSGSSSNIGSN |
| | | NANWYQQLPGTAPKLLIYDNNKRPSGVPDRFS |
| | | GSKSGTSASLAISGLRSEDEADYYCQAWDSST |
| | | VVFGGGTKLTVLG |
| 91 | HCDR1 | NHGMH |
| 92 | HCDR2 | VISYDGTNKYYADSVRG |
| 93 | HCDR3 | ETWDAFDV |
| 94 | LCDR1 | SGSSSNIGSNNAN |
| 95 | LCDR2 | DNNKRPS |
| 96 | LCDR3 | QAWDSSTVV |

Clone 13: 6G08

| | | |
|---|---|---|
| 97 | VH | EVQLLESGGGLVQPGGSLRLSCAASGFTLSSY |
| | | GISWVRQAPGKGLEWVSGISGSGGNTYYADSV |
| | | KGRFTISRDNSKNTLYLQMNSLRAEDTAVYYC |
| | | ASSVGAYANDAFDIWGQGTLVTVSS |
| 98 | VL | QSVLTQPPSASGTPGQRVTISCTGSSSNIGAG |
| | | YDVHWYQQLPGTAPKLLIYGDTNRPSGVPDRF |
| | | SGSKSGTSASLAISGLRSEDEADYYCAAWDDS |
| | | LNGPVFGGGTKLTVLG |
| 99 | HCDR1 | SYGIS |
| 100 | HCDR2 | GISGSGGNTYYADSVKG |
| 101 | HCDR3 | SVGAYANDAFDI |
| 102 | LCDR1 | TGSSSNIGAGYDVH |
| 103 | LCDR2 | GDTNRPS |
| 104 | LCDR3 | AAWDDSLNGPV |

Clone 5C05

| | | |
|---|---|---|
| 105 | VH | EVQLLESGGGLVQPGGSLRLSCAASGFTFSTY |
| | | GMHWVRQAPGKGLEWVAVISYDGSNKYYADSV |
| | | KGRFTISRDNSKNTLYLQMNSLRAEDTAVYYC |
| | | ARENFDAFDVWGQGTLVTVSS |
| 106 | VL | QSVLTQPPSASGTPGQRVTISCTGSSSNIGAG |
| | | YDVHWYQQLPGTAPKLLIYSNSQRPSGVPDRF |
| | | SGSKSGTSASLAISGLRSEDEADYYCAAWDDS |
| | | LNGQVVFGGGTKLTVLG |
| 107 | HCDR1 | TYGMH |
| 108 | HCDR2 | VISYDGSNKYYADSVKG |
| 109 | HCDR3 | ENFDAFDV |
| 110 | LCDR1 | TGSSSNIGAGYDVH |
| 111 | LCDR2 | SNSQRPS |
| 112 | LCDR3 | AAWDDSLNGQVV |

Clone 5E12

| | | |
|---|---|---|
| 113 | VH | EVQLLESGGGLVQPGGSLRLSCAASGFTFSSY |
| | | GMHWVRQAPGKGLEWVAVISYDGINKDYADSM |
| | | KGRFTISRDNSKNTLYLQMNSLRAEDTAVYYC |
| | | ARERKDAFDIWGQGTLVTVSS |
| 114 | VL | QSVLTQPPSASGTPGQRVTISCTGSSSNIGAG |
| | | YDVHWYQQLPGTAPKLLIYSNNQRPSGVPDRF |
| | | SGSKSGTSASLAISGLRSEDEADYYCATWDDS |
| | | LNGLVFGGGTKLTVLG |
| 115 | HCDR1 | SYGMH |
| 116 | HCDR2 | VISYDGINKDYADSMKG |
| 117 | HCDR3 | ERKDAFDI |
| 118 | LCDR1 | TGSSSNIGAGYDVH |
| 119 | LCDR2 | SNNQRPS |
| 120 | LCDR3 | ATWDDSLNGLV |

Clone: 6A09

| | | |
|---|---|---|
| 121 | VH | EVQLLESGGGLVQPGGSLRLSCAASGFTFSSY |
| | | GMHWVRQAPGKGLEWVAVTSYDGNTKYYANSV |
| | | KGRFTISRDNSKNTLYLQMNSLRAEDTAVYYC |
| | | AREDCGGDCFDYWGQGTLVTVSS |
| 122 | VL | QSVLTQPPSASGTPGQRVTISCTGSSSNIGAG |
| | | YDVHWYQQLPGTAPKLLIYGNSNRPSGVPDRF |
| | | SGSKSGTSASLAISGLRSEDEADYYCAAWDDS |
| | | LNEGVFGGGTKLTVLG |
| 123 | HCDR1 | SYGMH |
| 124 | HCDR2 | VTSYDGNTKYYANSVKG |
| 125 | HCDR3 | EDCGGDCFDY |
| 126 | LCDR1 | TGSSSNIGAGYDVH |
| 127 | LCDR2 | GNSNRPS |
| 128 | LCDR3 | AAWDDSLNEGV |

Clone: 6H08

| | | |
|---|---|---|
| 129 | VH | EVQLLESGGGLVQPGGSLRLSCAASGFTFNNY |
| | | GMHWVRQAPGKGLEWVAVISYDGSNKYYADSV |
| | | KGRFTISKDNSKNTLYLQMNSLRAEDTAVYYC |
| | | AREYKDAFDIWGQGTLVTVSS |
| 130 | VL | QSVLTQPPSASGTPGQRVTISCTGSSSNIGSN |
| | | TVNWYQQLPGTAPKLLIYDNNKRPSGVPDRFS |
| | | GSKSGTSASLAISGLRSEDEA |
| | | DYYCQAWGTGIRVFGGGTKLTVLG |
| 131 | HCDR1 | NYGMH |
| 132 | HCDR2 | VISYDGSNKYYADSVKG |
| 133 | HCDR3 | EYKDAFDI |
| 134 | LCDR1 | TGSSSNIGSNTVN |
| 135 | LCDR2 | DNNKRPS |
| 136 | LCDR3 | QAWGTGIRV |

TABLE 2
Exemplary anti-CD32b antibodies as disclosed
in WO 2009/083009

| | | |
|---|---|---|
| | Se- | |
| | quence | |
| SEQ | de- | |
| ID | scrip- | |
| NO | tion | Sequence |

Antibody 016

| | | |
|---|---|---|
| 201 | VH | QVQLVESGGGVVQPGRSLRLSCAASGFTFSSYGIHWVR |
| | | QAPGKGLEWVAVIGYDGSDKNYADSVKGRFTIFRDNSK |
| | | NTLYLQMNSLRAEDTAVYYCARDQLGDAFDIWGQGTMV |
| | | TVSS |
| 202 | HCDR1 | SYGIH |
| 203 | HCDR2 | VIGYDGSDKNYADSVKG |
| 204 | HCDR3 | DQLGDAFDI |
| 205 | VL | EIVLTQSPATLSLSPGERATLSCKASQSVSSSLAWYQQ |
| | | KPGQAPRLLIYDASNRATGIPARFSGSGSGTDFTLTIS |
| | | SLEPEDFAVYYCQQRSNWPPYTFGQGTKLEIKR |
| 206 | LCDR1 | KASQSVSSSLA |
| 207 | LCDR2 | DASNRAT |
| 208 | LCDR3 | QQRSNWPPYT |

Antibody 020

| | | |
|---|---|---|
| 209 | VH | QVQLVQSGGEVKKPGASVKVSCKTSGYTFTSYGISWVR |
| | | QAPGQGLEWMGWISAYNGNTKYAQKLQGRLTMTTDTST |
| | | TTAYMELRSLRSDDTAVYYCARDSAAHGMDVWGQGTTV |
| | | TVSS |
| 210 | HCDR1 | SYGIS |
| 211 | HCDR2 | WISAYNGNTKYAQKLQG |
| 212 | HCDR3 | DSAAHGMDV |
| 213 | VL | DIQMTQSPSSLSASVGDRVTITCRASQGISSWLAWYQQ |
| | | KPEKAPKSLIYAASSLQSGVPSRFRGSGSGTDFTLTIS |
| | | SLQPEDFATYYCQQYNSYPYTFGQGTKLEIKR |
| 214 | LCDR1 | RASQGISSWLA |
| 215 | LCDR2 | AASSLQS |
| 216 | LCDR3 | QQYNSYPYT |

Antibody 022

| | | |
|---|---|---|
| 217 | VH | QVQLVQSGAEVKKPGASVKVSCKASGYTFTSYGLSWVR |
| | | QAPGQGLEWMGWISPYNGNTHYAQKLQGRVTMTTDTST |
| | | STADMDLRSLRSDDTAVYYCARASAAHGMDVWGQGTTV |
| | | TVSS |
| 218 | HCDR1 | SYGLS |
| 219 | HCDR2 | WISPYNGNTHYAQKLQG |
| 220 | HCDR3 | ASAAHGMDV |
| 221 | VL | DIQMTQSPSSLSASVGDRVTITCRASQGISSWLAWYQQ |
| | | KPEKAPKSLIYAASSLQSGVPSRFSGSRSGTDFTLTIS |
| | | SLQPEDFATYYCQQYNSYPYTFGQGTKLEIKR |
| 222 | LCDR1 | RASQGISSWLA |
| 223 | LCDR2 | AASSLQS |
| 224 | LCDR3 | QQYNSYPYT |

Antibody 024

| | | |
|---|---|---|
| 225 | VH | QVQLVQSGAEVKKPGASVKVSCKASGYTFTSYGLSWVR |
| | | QAPGQGLEWMGWISPYNGNTHYAQKLQGRVTMTTDTST |
| | | STAYMDLRSLRSDDTAVYYCARDSAAHGMDVWGQGTTV |
| | | TVSS |
| 226 | HCDR1 | SYGLS |
| 227 | HCDR2 | WISPYNGNTHYAQKLQG |
| 228 | HCDR3 | DSAAHGMDV |
| 229 | VL | DIQMTQSPSSLSASVGDRVTITCRASQGISSWLAWYQQ |
| | | KPEKAPKSLIYAASSLQSGVPSRFSGSRSGTDFTLTIS |
| | | SLQPEDFATYYCQQYNSYPYTFGQGTKLEIKR |
| 230 | LCDR1 | RASQGISSWLA |
| 231 | LCDR2 | AASSLQS |
| 232 | LCDR3 | QQYNSYPYT |

Antibody 026

| | | |
|---|---|---|
| 233 | VH | QVQLVQSGAEVKKPGASVKVSCKASGYTFTSYGLSWVR |
| | | QAPGQGLEWMGWISAYNGNTNYAQKLQGRVTMTTDTST |
| | | STAYMDLRSLRSDDTAVYYCARDSAAHGMDVWGQGTTV |
| | | TVSS |
| 234 | HCDR1 | SYGLS |
| 235 | HCDR2 | WISAYNGNTNYAQKLQG |
| 236 | HCDR3 | DSAAHGMDV |
| 237 | VL | DIQMTQSPSSLSASVGDRVTITCRASQGISSWLAWYQQ |
| | | KPEKAPKSLIYAASSLQSGVPSRFSGSRSGTDFTLTIS |
| | | SLQPEDFATYYCQQYNSYPYTFGQGTKLEIKR |
| 238 | LCDR1 | RASQGISSWLA |
| 239 | LCDR2 | AASSLQS |
| 240 | LCDR3 | QQYNSYPYT |

Antibody 028

| | | |
|---|---|---|
| 241 | VH | QVQVVQSGAEVKKPGASVKVSCKTSGYTFTSYGISWVR |
| | | QAPGQGLEWMGWISAYNGNTKYAQKLQGRLTMTTDTST |
| | | TTAYMELRSLRSDDTAVYYCARDSAAHGMDVWGQGTTV |
| | | SVSS |
| 242 | HCDR1 | SYGIS |
| 243 | HCDR2 | WISAYNGNTKYAQKLQG |
| 244 | HCDR3 | DSAAHGMDV |
| 213 | VL | DIQMTQSPSSLSASVGDRVTITCRASQGISSWLAWYQQ |
| | | KPEKAPKSLIYAASSLQSGVPSRFRGSGSGTDFTLTIS |
| | | SLQPEDFATYYCQQYNSYPYTFGQGTKLEIKR |
| 214 | LCDR1 | RASQGISSWLA |
| 215 | LCDR2 | AASSLQS |
| 216 | LCDR3 | QQYNSYPYT |

Antibody 034

| | | |
|---|---|---|
| 245 | VH | EVQLLESGGGLVQPGGSLRLSCAASGFTFSNFVMSWVR |
| | | QAPGKGLEWVSGISGSGGNTDHADSVKGRFTISRDNSK |
| | | NTVYLQMNSLRAEDTAVYYCAKDSGGLFDYWGLGTLVT |
| | | VSS |
| 246 | HCDR1 | NFVMS |
| 247 | HCDR2 | GISGSGGNTDHADSVKG |
| 248 | HCDR3 | DSGGLFDY |
| 249 | VL | EIVLTQSPATLSLSPGERATLSCRASQSVSSYLAWYQQ |
| | | KPGQAPRLLIYDASNRATGIPARFSGSGSRTDFTLTIS |
| | | SLEPEDFAVYYCQQRSNWPHLTFGGGTKVEIKR |
| 250 | LCDR1 | RASQSVSSYLA |
| 251 | LCDR2 | DASNRAT |
| 252 | LCDR3 | QQRSNWPHLT |

Antibody 038

| | | |
|---|---|---|
| 253 | VH | QVQLVESGGGVVQPGRSLRLSCAASGFTFSTYGMHWVR |
| | | QAPGKGLEWVAVISHDGSDKYYADSVKGRFTISRDNSK |
| | | NTLYLQMNSLRAEDTAVYYCARDQSIIETFDYWGQGTL |
| | | VTVSS |
| 254 | HCDR1 | TYGMH |
| 255 | HCDR2 | VISHDGSDKYYADSVKG |
| 256 | HCDR3 | DQSIIETFDY |
| 257 | VL | EIVLTQSPATLSLSPGERATLSCRASQSVSSYLAWYQQ |
| | | KPGQAPRLLIYDASNRATGIPARFSGSGSGTDFTLTIS |
| | | SLEPEDFAVYYCQQRSNWGFTFGPGTKVDIKR |
| 258 | LCDR1 | RASQSVSSYLA |
| 259 | LCDR2 | DASNRAT |
| 260 | LCDR3 | QQRSNWGFT |

Antibody 053

| | | |
|---|---|---|
| 261 | VH | QVQLVESGGGVVQPGRSLRLSCAVSGFTFRSYGMHWVR |
| | | QAPGKGLEWVAVIWYDGSIKYYADSVKGRFTISRDNSK |
| | | NTLYLQMNSLRAEDTAVYFCAREGGRDAFDIWGQGTMV |
| | | TVSS |
| 262 | HCDR1 | SYGMH |
| 263 | HCDR2 | VIWYDGSIKYYADSVKG |
| 264 | HCDR3 | EGGRDAFDI |
| 265 | VL | AVQLTQSPSSLSASVGDRVTITCRASQGISSALAWYQQ |
| | | KPGKAPKLLIYDASSLESGVPSRFSGSGSGTDFTLTIS |
| | | SLQPEDFATYCCQQFNSYPHTFGGGTKVEIKR |
| 266 | LCDR1 | RASQGISSALA |
| 267 | LCDR2 | DASSLES |
| 268 | LCDR3 | QQFNSYPHT |

Antibody 063

| | | |
|---|---|---|
| 269 | VH | EVQLLESGGGLVQPGGSLRLSCAASGFTFSSYAMSWVR |
| | | QAPGKGLEWVSAISDSGGSTYYADSVKGRFTISRDNSK |
| | | NTLYLQMNSLRAEDTAAYYCAKEIAVALFDYWGQGTLV |
| | | TVSS |
| 270 | HCDR1 | SYAMS |
| 271 | HCDR2 | AISDSGGSTYYADSVKG |
| 272 | HCDR3 | EIAVALFDY |
| 273 | VL | EIVLTQSPATLSLSPGERATLSCRASQSVSSYLAWYQQ |
| | | KPGQAPRLLIYDASNRATGIPARFSGSGSGTDFTLTIS |
| | | SLEPEDFAVYYCQQRSSWPPYTFGQGTKLEIKR |
| 274 | LCDR1 | RASQSVSSYLA |
| 275 | LCDR2 | DASNRAT |
| 276 | LCDR3 | QQRSSWPPYT |

TABLE 3
Exemplary anti-CD32b antibody sequences as dis-
closed in WO 2004/016750, WO 2005/115452, WO
2005/110474, WO 2006/066078, and WO 2008/019199

| | | |
|---|---|---|
| | Se- | |
| | quence | |
| SEQ | de- | |
| ID | scrip- | |
| NO | tion | Sequence |

Clone 2B6

| | | |
|---|---|---|
| 300 | VH | QVQLQQPVTELVRPGASVMLSCKASDYPFTNYWIHW |
| | | VKQRPGQGLEWIGVIDPSDTYPNYNKKFKGKATLTV |
| | | VVSSSTAYMQLSSLTSDDSAVYYCARNGDSDYYSGM |
| | | DYWGQGTSVTVSS |
| 301 | VL | DILLTQSPAILSVSPGERVSFSCRTSQSIGTNIHWY |
| | | QQRTNGFPRLLIKNVSESISGIPSRFSGSGSGTDFI |
| | | LSINSVESEDIADYYCQQSNTWPFTFGGGTKLEIK |
| 302 | HCDR1 | NYWIH |
| 303 | HCDR2 | VIDPSDTYPNYNKKFKG |
| 304 | HCDR3 | NGDSDYYSGMDY |
| 305 | LCDR1 | RTSQSIGTNIH |
| 306 | LCDR2 | NVSESIS |
| 307 | LCDR2 | YVSESIS |
| 308 | LCDR2 | YASESIS |
| 309 | LCDR3 | QQSNTWPFT |
| 310 | Human- | EIVLTQSPDFQSVTPKEKVTITCRTSQSIGTNIHWY |
| | ized | QQKPDQSPKLLIKNVSESISGVPSRFSGSGSGTDFT |
| | 2B6 | LTINSLEAEDAATYYCQQSNTWPFTFGGGTKVEIK |
| | VL-1 | |
| 311 | Human- | EIVLTQSPDFQSVTPKEKVTITCRTSQSIGTNIHWY |
| | ized | QQKPDQSPKLLIKYVSESISGVPSRFSGSGSGTDFT |
| | 2B6 | LTINSLEAEDAATYYCQQSNTWPFTFGGGTKVEIK |
| | VL-2 | |
| 312 | Human- | EIVLTQSPDFQSVTPKEKVTITCRTSQSIGTNIHWY |
| | ized | QQKPDQSPKLLIKYASESISGVPSRFSGSGSGTDFT |
| | 2B6 | LTINSLEAEDAATYCQQSNTWPFTFGGGTKVEIK |
| | VL-3 | |
| 313 | Human- | QVQLVQSGAEVKKPGASVKVSCKASGYTFTNYWIHW |
| | ized | VRQAPGQGLEWMGVIDPSDTYPNYNKKFKGRVTMTT |
| | 2B6 | DTSTSTAYMELRSLRSDDTAVYYCARNGDSDYYSGM |
| | VH | DYWGQGTTVTVSS |

Clone 3H7

| | | |
|---|---|---|
| 314 | HCDR1 | DAWMD |
| 315 | HCDR2 | EIRNKANNLATYYAESVKG |
| 316 | HCDR3 | YSPFAY |
| 317 | VH | EVKFEESGGGLVQPGGSMKLSCAASGFTFSDAWMDW |
| | | VRQGPEKGLEWVAEIRNKANNLATYYAESVKGRFTI |
| | | PRDDSKSSVYLHMNSLRAEDTGIYYCYSPFAYWGQG |
| | | TLVTVSA |
| 318 | LCDR1 | RASQEISGYLS |
| 319 | LCDR2 | AASTLDS |
| 320 | LCDR3 | LQYVSYPYT |
| 321 | VL | DIQMTQSPSSLSASLGERVSLTCRASQEISGYLSWL |
| | | QQKPDGTIRRLIYAASTLDSGVPKRFSGSWSGSDYS |
| | | LTISSLESEDFADYYCLQYVSYPYTFGGGTKLEIK |

Clone: 8B5

| | | |
|---|---|---|
| 322 | VL | DIQMTQSPSSLLAALGERVSLTCRASQEISGYLSWL |
| | | QQKPDGTIKRLIYAASTLDSGVPKRFSGSESGSDYS |
| | | LTISSLESEDFADYYCLQYFSYPLTFGAGTKLELK |
| 323 | VH | EVKLEESGGGLVQPGGSMKLSCEASGFTFSDAWMDW |
| | | VRQSPEKGLEWVAEIRNKAKNHATYYAESVIGRFTI |
| | | SRDDSKSSVYLQMNSLRAEDTGIYYCGALGLDYWGQ |
| | | GTTLTVSS |
| 324 | LCDR1 | RASQEISGYLS |
| 325 | LCDR2 | AASTLDS |
| 326 | LCDR3 | LQYFSYPLT |
| 327 | HCDR1 | DAWMD |
| 328 | HCDR2 | EIRNKAKNHATYYAESVIG |
| 329 | HCDR3 | GALGLDY |

Clone: 1D5, ATCC accession number PTA-5958
Clone: 2E1, ATCC accession number PTA-5961
Clone: 2H9, ATCC accession number PTA-5962
Clone: 2D11, ATCC accession number PTA-5960
Clone: 1F2, ATCC accession number PTA-5959

Other anti-CD32b antibody molecules include those wherein the amino acids or nucleic acids encoding the amino acids have been mutated, yet have at least 60, 70, 80, 90 or 95 percent identity to the sequences described in Tables 1, 2, or 3. In one embodiment, it includes mutant amino acid sequences wherein no more than 1, 2, 3, 4 or 5 amino acids have been mutated in the variable regions when compared with the variable regions depicted in the sequence described in Tables 1, 2 or 3, while retaining substantially the same therapeutic activity.

In some embodiments, the anti-CD32b antibody molecule includes a VH and/or VL, or CDR sequence substantially identical to any of the aforesaid amino acid sequences. In an embodiment, the anti-CD32b antibody molecule comprises: a light chain variable region comprising an amino acid sequence having at least one, two or three modifications (e.g., substitutions) but not more than 30, 20 or 10 modifications (e.g., substitutions) of an amino acid sequence of any of the aforesaid light chain variable regions, or a sequence with at least 95, 96, 97, 98, 99% identity with any of the aforesaid light chain variable amino acid sequence; and/or a heavy chain variable region comprising an amino acid sequence having at least one, two or three modifications (e.g., substitutions) but not more than 30, 20 or 10 modifications (e.g., substitutions) of an amino acid sequence of any of the aforesaid light chain variable regions, or a sequence with 95-99% identity to an amino acid of any of the aforesaid light chain variable regions.

In one embodiment, the anti-CD32b antibody molecule comprises one, two or more (e.g., all three) light chain complementarity determining region 1 (LCDR1), light chain complementarity determining region 2 (LCDR2), and light chain complementarity determining region 3 (LCDR3) of an anti-CD32b antibody molecule described in Table 1, 2 or 3, and one, two or more (e.g., all three) heavy chain complementarity determining region 1 (HCDR1), heavy chain complementarity determining region 2 (HCDR2), and heavy chain complementarity determining region 3 (HCDR3) of an anti-CD32b antibody molecule described in Table 1, 2 or 3, e.g., an anti-CD32b antibody molecule comprising one, two or more, e.g., all three, LCDRs and one or more, e.g., all three, HCDRs.

In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, 2, or 3.

In another embodiment, the anti-CD32b antibody molecule comprises one, two or more (e.g., all three) light chain complementarity determining region 1 (LCDR1), light chain complementarity determining region 2 (LCDR2), and light chain complementarity determining region 3 (LCDR3) of an anti-CD32b antibody molecule produced by any one of hybridoma clones 1D5, 2E1, 2H9, 2D11, or 1F2 having ATCC Accession numbers, PTA-5958, PTA-5961, PTA-5962, PTA-5960, and PTA-5959, respectively, and one, two or more (e.g., all three) heavy chain complementarity determining region 1 (HCDR1), heavy chain complementarity determining region 2 (HCDR2), and heavy chain complementarity determining region 3 (HCDR3) of an anti-CD32b antibody molecule produced by any one of hybridoma clones 1D5, 2E1, 2H9, 2D11, or 1F2 having ATCC Accession numbers, PTA-5958, PTA-5961, PTA-5962, PTA-5960, and PTA-5959, respectively.

In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence of a monoclonal anti-CD32b antibody produced by any one of hybridoma clones 1D5, 2E1, 2H9, 2D11, or 1F2 having ATCC Accession numbers, PTA-5958, PTA-5961, PTA-5962, PTA-5960, and PTA-5959, respectively.

In another specific embodiment, the anti-CD32b antibody molecule binds human CD32b and comprises the HCDR1, HCDR2, and HCDR3 sequences of SEQ ID NOs: 3, 4, and 5, respectively, and the LCDR1, LCDR2, and LCDR3 sequences of SEQ ID NOs: 6, 7, and 8, respectively.

In another specific embodiment, the anti-CD32b antibody molecule binds human CD32b and comprises the HCDR1, HCDR2, and HCDR3 sequences of SEQ ID NOs: 11, 12, and 13, respectively, and the LCDR1, LCDR2, and LCDR3 sequences of SEQ ID NOs: 14, 15 and 16, respectively.

In another specific embodiment, the anti-CD32b antibody molecule binds human CD32b and comprises the HCDR1, HCDR2, and HCDR3 sequences of SEQ ID NOs: 19, 20 and 21, respectively, and the LCDR1, LCDR2, and LCDR3 sequences of SEQ ID NOs: 22, 23 and 24 respectively.

In another specific embodiment, the anti-CD32b antibody molecule binds human CD32b and comprises the HCDR1, HCDR2, and HCDR3 sequences of SEQ ID NOs: 27, 28 and 29, respectively, and the LCDR1, LCDR2, and LCDR3 sequences of SEQ ID NOs: 30, 31 and 32 respectively.

In another specific embodiment, the anti-CD32b antibody molecule binds human CD32b and comprises the HCDR1, HCDR2, and HCDR3 sequences of SEQ ID NOs: 35, 36 and 37, respectively, and the LCDR1, LCDR2, and LCDR3 sequences of SEQ ID NOs: 38, 39 and 40 respectively.

In another specific embodiment, the anti-CD32b antibody molecule binds human CD32b and comprises the HCDR1, HCDR2, and HCDR3 sequences of SEQ ID NOs: 43, 44 and 45 respectively, and the LCDR1, LCDR2, and LCDR3 sequences of SEQ ID NOs: 46, 47 and 48, respectively.

In another specific embodiment, the anti-CD32b antibody molecule binds human CD32b and comprises the HCDR1, HCDR2, and HCDR3 sequences of SEQ ID NOs: 51, 52 and 53, respectively, and the LCDR1, LCDR2, and LCDR3 sequences of SEQ ID NOs: 54, 55 and 56, respectively.

In another specific embodiment, the anti-CD32b antibody molecule binds human CD32b and comprises the HCDR1, HCDR2, and HCDR3 sequences of SEQ ID NOs: 59, 60 and 61 respectively, and the LCDR1, LCDR2, and LCDR3 sequences of SEQ ID NOs: 62, 63 and 64, respectively.

In another specific embodiment, the anti-CD32b antibody molecule binds human CD32b and comprises the HCDR1, HCDR2, and HCDR3 sequences of SEQ ID NOs: 67, 68 and 69, respectively, and the LCDR1, LCDR2, and LCDR3 sequences of SEQ ID NOs: 70, 71, and 72, respectively.

In another specific embodiment, the anti-CD32b antibody molecule binds human CD32b and comprises the HCDR1, HCDR2, and HCDR3 sequences of SEQ ID NOs: 75, 76 and 77, respectively, and the LCDR1, LCDR2, and LCDR3 sequences of SEQ ID NOs: 78, 79 and 80, respectively.

In another specific embodiment, the anti-CD32b antibody molecule binds human CD32b and comprises the HCDR1, HCDR2, and HCDR3 sequences of SEQ ID NOs: 83, 84 and 85, respectively, and the LCDR1, LCDR2, and LCDR3 sequences of SEQ ID NOs: 86, 87 and 88, respectively.

In another specific embodiment, the anti-CD32b antibody molecule binds human CD32b and comprises the HCDR1, HCDR2, and HCDR3 sequences of SEQ ID NOs: 91, 92 and 93, respectively, and the LCDR1, LCDR2, and LCDR3 sequences of SEQ ID NOs: 94, 95 and 96, respectively.

In another specific embodiment, the anti-CD32b antibody binds human CD32b and comprises the HCDR1, HCDR2, and HCDR3 sequences of SEQ ID NOs: 99, 100 and 101, respectively, and the LCDR1, LCDR2, and LCDR3 sequences of SEQ ID NOs: 102, 103 and 104, respectively.

In another specific embodiment, the anti-CD32b antibody binds human CD32b and comprises the HCDR1, HCDR2, and HCDR3 sequences of SEQ ID NOs: 107, 108 and 109, respectively, and the LCDR1, LCDR2, and LCDR3 sequences of SEQ ID NOs: 110, 111 and 112, respectively.

In another specific embodiment, the anti-CD32b antibody binds human CD32b and comprises the HCDR1, HCDR2, and HCDR3 sequences of SEQ ID NOs: 115, 116 and 117, respectively, and the LCDR1, LCDR2, and LCDR3 sequences of SEQ ID NOs: 118, 119 and 120, respectively.

In another specific embodiment, the anti-CD32b antibody binds human CD32b and comprises the HCDR1, HCDR2, and HCDR3 sequences of SEQ ID NOs: 123, 124 and 125, respectively, and the LCDR1, LCDR2, and LCDR3 sequences of SEQ ID NOs: 126, 127, and 128, respectively.

In another specific embodiment, the anti-CD32b antibody binds human CD32b and comprises the HCDR1, HCDR2, and HCDR3 sequences of SEQ ID NOs: 131, 132 and 133, respectively, and the LCDR1, LCDR2, and LCDR3 sequences of SEQ ID NOs: 134, 135 and 136, respectively.

In another specific embodiment, the anti-CD32b antibody binds human CD32b and comprises the HCDR1, HCDR2, and HCDR3 sequences of SEQ ID NOs: 202, 203 and 204 respectively, and the LCDR1, LCDR2, and LCDR3 sequences of SEQ ID NOs: 206, 207 and 208, respectively.

In another specific embodiment, the anti-CD32b antibody binds human CD32b and comprises the HCDR1, HCDR2, and HCDR3 sequences of SEQ ID NOs: 210, 211 and 212, respectively, and the LCDR1, LCDR2, and LCDR3 sequences of SEQ ID NOs: 214, 215 and 216, respectively.

In another specific embodiment, the anti-CD32b antibody binds human CD32b and comprises the HCDR1, HCDR2, and HCDR3 sequences of SEQ ID NOs: 218, 219 and 220, respectively, and the LCDR1, LCDR2, and LCDR3 sequences of SEQ ID NOs: 222, 223 and 224, respectively.

In another specific embodiment, the anti-CD32b antibody binds human CD32b and comprises the HCDR1, HCDR2, and HCDR3 sequences of SEQ ID NOs: 226, 227 and 228, respectively, and the LCDR1, LCDR2, and LCDR3 sequences of SEQ ID NOs: 230, 231 and 232, respectively. In another specific embodiment, the anti-CD32b antibody binds human CD32b and comprises the HCDR1, HCDR2, and HCDR3 sequences of SEQ ID NOs: 234, 235 and 236 respectively, and the LCDR1, LCDR2, and LCDR3 sequences of SEQ ID NOs: 238, 239 and 240, respectively.

In another specific embodiment, the anti-CD32b antibody binds human CD32b and comprises the HCDR1, HCDR2, and HCDR3 sequences of SEQ ID NOs: 242, 243 and 244, respectively, and the LCDR1, LCDR2, and LCDR3 sequences of SEQ ID NOs: 214, 215 and 216, respectively.

In another specific embodiment, the anti-CD32b antibody binds human CD32b and comprises the HCDR1, HCDR2, and HCDR3 sequences of SEQ ID NOs: 246, 247 and 248, respectively, and the LCDR1, LCDR2, and LCDR3 sequences of SEQ ID NOs: 250, 251 and 252, respectively.

In another specific embodiment, the anti-CD32b antibody binds human CD32b and comprises the HCDR1, HCDR2, and HCDR3 sequences of SEQ ID NOs: 254, 255 and 256, respectively, and the LCDR1, LCDR2, and LCDR3 sequences of SEQ ID NOs: 258, 259 and 260, respectively.

In another specific embodiment, the anti-CD32b antibody binds human CD32b and comprises the HCDR1, HCDR2, and HCDR3 sequences of SEQ ID NOs: 262, 263 and 264, respectively, and the LCDR1, LCDR2, and LCDR3 sequences of SEQ ID NOs: 266, 267 and 268, respectively.

In another specific embodiment, the anti-CD32b antibody binds human CD32b and comprises the HCDR1, HCDR2, and HCDR3 sequences of SEQ ID NOs: 270, 271 and 272, respectively, and the LCDR1, LCDR2, and LCDR3 sequences of SEQ ID NOs: 274, 275 and 276, respectively.

In another specific embodiment, the anti-CD32b antibody binds human CD32b and comprises the HCDR1, HCDR2, and HCDR3 sequences of SEQ ID NOs: 302, 303 and 304, respectively, and the LCDR1, LCDR2, and LCDR3 sequences of SEQ ID NOs: 305, 306 and 309, respectively.

In another specific embodiment, the anti-CD32b antibody binds human CD32b and comprises the HCDR1, HCDR2, and HCDR3 sequences of SEQ ID NOs: 314, 315 and 316, respectively, and the LCDR1, LCDR2, and LCDR3 sequences of SEQ ID NOs: 318, 319 and 320, respectively.

In another specific embodiment, the anti-CD32b antibody binds human CD32b and comprises the HCDR1, HCDR2, and HCDR3 sequences of SEQ ID NOs: 327, 328 and 329, respectively, and the LCDR1, LCDR2, and LCDR3 sequences of SEQ ID NOs: 324, 325 and 326, respectively.

In another specific embodiment, the anti-CD32B antibody binds human CD32b and comprises the HCDR1, HCDR2, and HCDR3 amino acid sequences, and LCDR1, LCDR2, and LCDR3 amino acid sequences of a monoclonal antibody produced by hybridoma clone 1D5 having ATCC accession number PTA-5958.

In another specific embodiment, the anti-CD32B antibody binds human CD32b and comprises the HCDR1, HCDR2, and HCDR3 amino acid sequences, and LCDR1, LCDR2, and LCDR3 amino acid sequences of a monoclonal antibody produced by hybridoma clone 2E1 having ATCC accession number PTA-5961.

In another specific embodiment, the anti-CD32B antibody binds human CD32b and comprises the HCDR1, HCDR2, and HCDR3 amino acid sequences, and LCDR1, LCDR2, and LCDR3 amino acid sequences of a monoclonal antibody produced by hybridoma clone 2H9 having ATCC accession number PTA-5962.

In another specific embodiment, the anti-CD32B antibody binds human CD32b and comprises the HCDR1, HCDR2, and HCDR3 amino acid sequences, and LCDR1, LCDR2, and LCDR3 amino acid sequences of a monoclonal antibody produced by hybridoma clone 2D11 having ATCC accession number PTA-5960.

In another specific embodiment, the anti-CD32B antibody binds human CD32b and comprises the HCDR1, HCDR2, and HCDR3 amino acid sequences, and LCDR1, LCDR2, and LCDR3 amino acid sequences of a monoclonal antibody produced by hybridoma clone 1F2 having ATCC accession number PTA-5959.

In an embodiment, the anti-CD32b antibody molecule comprises: a light chain variable region comprising an amino acid sequence of any of the light chain variable regions disclosed in Tables 1, 2 or 3, or a sequence with at least 95, 96, 97, 98, 99% identity with any of the light chain variable amino acid sequences disclosed in Tables 1, 2 or 3; and/or a heavy chain variable region comprising an amino acid sequence of any of the heavy chain variable regions disclosed in Tables 1, 2 or 3, or a sequence with at least 95, 96, 97, 98, 99% identity with any of the heavy chain variable amino acid sequences disclosed in Tables 1, 2 or 3.

In an embodiment, the anti-CD32b antibody molecule comprises: a light chain variable region comprising an amino acid sequence of a light chain variable region of a monoclonal antibody produced by any one of hybridoma clones 1D5, 2E1, 2H9, 2D11, or 1F2 having ATCC Accession numbers, PTA-5958, PTA-5961, PTA-5962, PTA-5960, and PTA-5959, respectively, or a sequence with at least 95, 96, 97, 98, 99% identity with any of the light chain variable amino acid sequences of said clones; and/or a heavy chain variable region comprising an amino acid sequence of a heavy chain variable region of a monoclonal antibody produced by any one of hybridoma clones 1D5, 2E1, 2H9, 2D11, or 1F2 having ATCC Accession numbers, PTA-5958, PTA-5961, PTA-5962, PTA-5960, and PTA-5959, respectively, or a sequence with at least 95, 96, 97, 98, 99% identity with any of the heavy chain variable amino acid sequences of said clones.

In another specific embodiment, the anti-CD32b antibody binds human CD32b and comprises the VH amino acid sequence of SEQ ID NO: 1 and the VL amino acid sequence of SEQ ID NO: 2.

In another specific embodiment, the anti-CD32b antibody binds human CD32b and comprises the VH amino acid sequence of SEQ ID NO: 9 and the VL amino acid sequence of SEQ ID NO: 10.

In another specific embodiment, the anti-CD32b antibody binds human CD32b and comprises the VH amino acid sequence of SEQ ID NO: 17 and the VL amino acid sequence of SEQ ID NO: 18.

In another specific embodiment, the anti-CD32b antibody binds human CD32b and comprises the VH amino acid sequence of SEQ ID NO: 25 and the VL amino acid sequence of SEQ ID NO: 26.

In another specific embodiment, the anti-CD32b antibody binds human CD32b and comprises the VH amino acid sequence of SEQ ID NO: 33 and the VL amino acid sequence of SEQ ID NO: 34.

In another specific embodiment, the anti-CD32b antibody binds human CD32b and comprises the VH amino acid sequence of SEQ ID NO: 41 and the VL amino acid sequence of SEQ ID NO: 42.

In another specific embodiment, the anti-CD32b antibody binds human CD32b and comprises the VH amino acid sequence of SEQ ID NO: 49 and the VL amino acid sequence of SEQ ID NO: 50.

In another specific embodiment, the anti-CD32b antibody binds human CD32b and comprises the VH amino acid sequence of SEQ ID NO: 57 and the VL amino acid sequence of SEQ ID NO: 58.

In another specific embodiment, the anti-CD32b antibody binds human CD32b and comprises the VH amino acid sequence of SEQ ID NO: 65 and the VL amino acid sequence of SEQ ID NO: 66.

In another specific embodiment, the anti-CD32b antibody binds human CD32b and comprises the VH amino acid sequence of SEQ ID NO: 73 and the VL amino acid sequence of SEQ ID NO: 74.

In another specific embodiment, the anti-CD32b antibody binds human CD32b and comprises the VH amino acid sequence of SEQ ID NO: 81 and the VL amino acid sequence of SEQ ID NO: 82.

In another specific embodiment, the anti-CD32b antibody binds human CD32b and comprises the VH amino acid sequence of SEQ ID NO: 89 and the VL amino acid sequence of SEQ ID NO: 90.

In another specific embodiment, the anti-CD32b antibody binds human CD32b and comprises the VH amino acid sequence of SEQ ID NO: 97 and the VL amino acid sequence of SEQ ID NO: 98.

In another specific embodiment, the anti-CD32b antibody binds human CD32b and comprises the VH amino acid sequence of SEQ ID NO: 105 and the VL amino acid sequence of SEQ ID NO: 106.

In another specific embodiment, the anti-CD32b antibody binds human CD32b and comprises the VH amino acid sequence of SEQ ID NO: 113 and the VL amino acid sequence of SEQ ID NO: 114.

In another specific embodiment, the anti-CD32b antibody binds human CD32b and comprises the VH amino acid sequence of SEQ ID NO: 121 and the VL amino acid sequence of SEQ ID NO: 122.

In another specific embodiment, the anti-CD32b antibody binds human CD32b and comprises the VH amino acid sequence of SEQ ID NO: 129 and the VL amino acid sequence of SEQ ID NO: 130.

In another specific embodiment, the anti-CD32b antibody binds human CD32b and comprises the VH amino acid sequence of SEQ ID NO: 201 and the VL amino acid sequence of SEQ ID NO: 205.

In another specific embodiment, the anti-CD32b antibody binds human CD32b and comprises the VH amino acid sequence of SEQ ID NO: 209 and the VL amino acid sequence of SEQ ID NO: 213.

In another specific embodiment, the anti-CD32b antibody binds human CD32b and comprises the VH amino acid sequence of SEQ ID NO: 217 and the VL amino acid sequence of SEQ ID NO: 221.

In another specific embodiment, the anti-CD32b antibody binds human CD32b and comprises the VH amino acid sequence of SEQ ID NO: 225 and the VL amino acid sequence of SEQ ID NO: 229.

In another specific embodiment, the anti-CD32b antibody binds human CD32b and comprises the VH amino acid sequence of SEQ ID NO: 233 and the VL amino acid sequence of SEQ ID NO: 237.

In another specific embodiment, the anti-CD32b antibody binds human CD32b and comprises the VH amino acid sequence of SEQ ID NO: 241 and the VL amino acid sequence of SEQ ID NO: 213.

In another specific embodiment, the anti-CD32b antibody binds human CD32b and comprises the VH amino acid sequence of SEQ ID NO: 245 and the VL amino acid sequence of SEQ ID NO: 249.

In another specific embodiment, the anti-CD32b antibody binds human CD32b and comprises the VH amino acid sequence of SEQ ID NO: 253 and the VL amino acid sequence of SEQ ID NO: 257.

In another specific embodiment, the anti-CD32b antibody binds human CD32b and comprises the VH amino acid sequence of SEQ ID NO: 261 and the VL amino acid sequence of SEQ ID NO: 265.

In another specific embodiment, the anti-CD32b antibody binds human CD32b and comprises the VH amino acid sequence of SEQ ID NO: 269 and the VL amino acid sequence of SEQ ID NO:

273.

In another specific embodiment, the anti-CD32b antibody binds human CD32b and comprises the VH amino acid sequence of SEQ ID NO: 300 and the VL amino acid sequence of SEQ ID NO: 301.

In another specific embodiment, the anti-CD32b antibody binds human CD32b and comprises the VH amino acid sequence of SEQ ID NO: 313 and the VL amino acid sequence of SEQ ID NO: 310.

In another specific embodiment, the anti-CD32b antibody binds human CD32b and comprises the VH amino acid sequence of SEQ ID NO: 313 and the VL amino acid sequence of SEQ ID NO: 311.

In another specific embodiment, the anti-CD32b antibody binds human CD32b and comprises the VH amino acid sequence of SEQ ID NO: 313 and the VL amino acid sequence of SEQ ID NO: 312.

In another specific embodiment, the anti-CD32b antibody binds human CD32b and comprises the VH amino acid sequence of SEQ ID NO: 317 and the VL amino acid sequence of SEQ ID NO: 321.

In another specific embodiment, the anti-CD32b antibody binds human CD32b and comprises the VH amino acid sequence of SEQ ID NO: 323 and the VL amino acid sequence of SEQ ID NO: 322.

In another specific embodiment, the anti-CD32b antibody binds human CD32b and comprises the VH amino acid sequence and VL amino acid sequence of a monoclonal antibody produced by hybridoma clone 1D5 having ATCC accession number PTA-5958.

In another specific embodiment, the anti-CD32b antibody binds human CD32b and comprises the VH amino acid sequence and VL amino acid sequence of a monoclonal antibody produced by hybridoma clone 2E1 having ATCC accession number PTA-5961.

In another specific embodiment, the anti-CD32b antibody binds human CD32b and comprises the VH amino acid sequence and VL amino acid sequence of a monoclonal antibody produced by hybridoma clone 2H9 having ATCC accession number PTA-5962.

In another specific embodiment, the anti-CD32b antibody binds human CD32b and comprises the VH amino acid sequence and VL amino acid sequence of a monoclonal antibody produced by hybridoma clone 2D11 having ATCC accession number PTA-5960.

In another specific embodiment, the anti-CD32b antibody binds human CD32b and comprises the VH amino acid sequence and VL amino acid sequence of a monoclonal antibody produced by hybridoma clone 1F2 having ATCC accession number PTA-5959.

Since each of these antibodies can bind to CD32b, the VH, VL, full length light chain, and full length heavy chain sequences (amino acid sequences and the nucleotide sequences encoding the amino acid sequences) can be “mixed and matched” to create other CD32b-binding antibodies and antigen-binding fragments thereof of the disclosure. Such “mixed and matched” CD32b-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence.

In another aspect, the present disclosure provides CD32b-binding antibodies that comprise the heavy chain and light chain CDR1s, CDR2s and CDR3s as described in Tables 1, 2 or 3, or combinations thereof. The CDR regions are delineated using the Kabat system (Kabat et al. 1991 Sequences of Proteins of Immunological Interest, Fifth Edition, U.S. Department of Health and Human Services, NIH Publication No. 91-3242), or using the Chothia system (Chothia et al. 1987 J. Mol. Biol. 196: 901-917; and Al-Lazikani et al. 1997 J. Mol. Biol. 273: 927-948). Other methods for delineating the CDR regions may alternatively be used. For example, the CDR definitions of both Kabat and Chothia may be combined such that, the CDRs may comprise some or all of the amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Given that each of these antibodies can bind to CD32b and that antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and match, although each antibody must contain a VH CDR1, 2 and 3 and a VL CDR1, 2 and 3 to create other CD32b-binding binding molecules of the disclosure. Such “mixed and matched” CD32b-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence (s). Likewise, when VL CDR sequences are mixed and matched, the CDR1, CDR2 and/or CDR3 sequence from a particular VL sequence should be replaced with a structurally similar CDR sequence (s). It will be readily apparent to the ordinarily skilled artisan that novel VH and VL sequences can be created by mutating one or more VH and/or VL CDR region sequences with structurally similar sequences from the CDR sequences shown herein for monoclonal antibodies of the present disclosure.

Accordingly, the present disclosure provides an anti-CD32b antibody molecule comprising a heavy chain variable region CDR1 comprising an amino acid sequence selected from any of SEQ ID NOs: 3, 11, 19, 27, 35, 43, 51, 59, 67, 75, 83, 91, 99, 107, 115, 123, 131, 202, 210, 218, 226, 234, 242, 246, 254, 262, 270, 302, 314 and 327; a heavy chain variable region CDR2 comprising an amino acid sequence selected from any of SEQ ID NOs: 4, 12, 20, 28, 36, 44, 52, 60, 68, 76, 84, 92, 100, 108, 116, 124, 132, 203, 211, 219, 227, 235, 243, 247, 255, 263, 271, 303, 315 and 328; a heavy chain variable region CDR3 comprising an amino acid sequence selected from any of SEQ ID NOs: 5, 13, 21, 29, 37, 45, 53, 61, 69, 77, 85, 93, 101, 109, 117, 125, 133, 204, 212, 220, 228, 236, 244, 248, 256, 264, 272, 304, 316, and 329; a light chain variable region CDR1 comprising an amino acid sequence selected from any of SEQ ID NOs: 6, 14, 22, 30, 38, 46, 54, 62, 70, 78, 86, 94, 102, 110, 118, 126, 134, 206, 214, 222, 230, 238, 250, 258, 266, 274, 305, 318, and 324; a light chain variable region CDR2 comprising an amino acid sequence selected from any of SEQ ID NOs: 7, 15, 23, 31, 39, 47, 55, 63, 71, 79, 87, 95, 103, 111, 119, 127, 135, 207, 215, 223, 231, 239, 251, 259, 267, 275, 306, 307, 308, 319 and 325; and a light chain variable region CDR3 comprising an amino acid sequence selected from any of SEQ ID NOs: 8, 16, 24, 32, 40, 48, 56, 64, 72, 80, 88, 96, 104, 112, 120, 128, 136, 208, 216, 224, 232, 240, 252, 260, 268, 276, 309, 320 and 326; wherein the antibody specifically binds CD32b.

The present disclosure also provides an anti-CD32b antibody molecule comprising a heavy chain variable region comprising an amino acid sequence selected from any of SEQ ID NOs: 1, 9, 17, 25, 33, 41, 49, 57, 65, 73, 81, 89, 97, 105, 113, 121, 129, 201, 209, 217, 225, 233, 241, 245, 253, 261, 269, 300, 313, 317 and 323; and a light chain variable region comprising an amino acid sequence selected from any of SEQ ID NOs: 2, 10, 18, 26, 34, 42, 50, 58, 66, 74, 82, 90, 98, 106, 114, 122, 130, 205, 213, 221, 229, 237, 249, 257, 265, 273, 301, 310, 311, 312, 321, and 322.

In one embodiment, an antibody that specifically binds to CD32b is an antibody that is described in Tables 1, 2 or 3. In one embodiment, an antibody that specifically binds to CD32b is 6G11. In one embodiment, an antibody that specifically binds to CD32b is 5C04. In one embodiment, an antibody that specifically binds to CD32b is 5D07. In one embodiment, an antibody that specifically binds to CD32b is 5G08. In one embodiment, an antibody that specifically binds to CD32b is 5H06. In one embodiment, an antibody that specifically binds to CD32b is 6B01. In one embodiment, an antibody that specifically binds to CD32b is 6C11. In one embodiment, an antibody that specifically binds to CD32b is 6C12. In one embodiment, an antibody that specifically binds to CD32b is 6D01. In one embodiment, an antibody that specifically binds to CD32b is 6G03. In one embodiment, an antibody that specifically binds to CD32b is 7C07. In one embodiment, an antibody that specifically binds to CD32b is 4B02. In one embodiment, an antibody that specifically binds to CD32b is 6G08. In one embodiment, an antibody that specifically binds to CD32b is 5C05. In one embodiment, an antibody that specifically binds to CD32b is 5E12. In one embodiment, an antibody that specifically binds to CD32b is 6A09. In one embodiment, an antibody that specifically binds to CD32b is 6H08. In one embodiment, an antibody that specifically binds to CD32b is Antibody 016. In one embodiment, an antibody that specifically binds to CD32b is Antibody 020. In one embodiment, an antibody that specifically binds to CD32b is Antibody 022. In one embodiment, an antibody that specifically binds to CD32b is Antibody 024. In one embodiment, an antibody that specifically binds to CD32b is Antibody 026. In one embodiment, an antibody that specifically binds to CD32b is Antibody 028. In one embodiment, an antibody that specifically binds to CD32b is Antibody 034. In one embodiment, an antibody that specifically binds to CD32b is Antibody 038. In one embodiment, an antibody that specifically binds to CD32b is Antibody 053. In one embodiment, an antibody that specifically binds to CD32b is Antibody 063. In one embodiment, an antibody that specifically binds to CD32b is 2B6. In one embodiment, an antibody that specifically binds to CD32b is 3H7. In one embodiment, an antibody that specifically binds to CD32b is 8B5. In one embodiment, an antibody that specifically binds to CD32b is an antibody produced by hybridoma clone 1D5, having ATCC accession number PTA-5958. In one embodiment, an antibody that specifically binds to CD32b is an antibody produced by hybridoma clone 2E1 having ATCC accession number PTA-5961. In one embodiment, an antibody that specifically binds to CD32b is an antibody produced by hybridoma clone 2H9 having ATCC accession number PTA-5962. In one embodiment, an antibody that specifically binds to CD32b is an antibody produced by hybridoma clone 2D11 having ATCC accession number PTA-5960. In one embodiment, an antibody that specifically binds to CD32b is an antibody produced by hybridoma clone 1F2 having ATCC accession number PTA-5959.

In some embodiments of the CD32b-binding antibody molecules disclosed herein, the antibodies comprise a wild type (WT) Fc sequence. In some embodiments, the antibodies are afucosylated. In other embodiments, the antibodies comprise a modified Fc region comprising mutations which enhance ADCC (eADCC) activity of the antibodies. In yet other embodiments, the antibodies comprise a modified Fc region comprising mutations which silence the ADCC activity of the Fc region (Fc silent mutants).

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutations. However, in the VH or VL framework regions, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, a recombinant human antibody will display no more than 10 amino acid differences from the amino acid sequence encoded by the human germline immunoglobulin gene in the VH or VL framework regions. In certain cases, the human antibody may display no more than 5, or even no more than 4, 3, 2, or 1 amino acid difference from the amino acid sequence encoded by the germline immunoglobulin gene.

### Homologous Antibodies

In yet another embodiment, the present disclosure provides an anti-CD32b antibody molecule comprising amino acid sequences that are homologous to the sequences described in Tables 1, 2 or 3, and said antibody binds to CD32b, and retains the desired functional properties of those antibodies described in Tables 1, 2 or 3.

For example, the disclosure provides an anti-CD32b antibody molecule comprising a heavy chain variable region and a light chain variable region, wherein the heavy chain variable region comprises an amino acid sequence that is at least 80%, at least 90%, or at least 95% identical to an amino acid sequence selected from the group consisting of SEQ ID NOs: 1, 9, 17, 25, 33, 41, 49, 57, 65, 73, 81, 89, 97, 105, 113, 121, 129, 201, 209, 217, 225, 233, 241, 245, 253, 261, 269, 300, 313, 317 and 323; the light chain variable region comprises an amino acid sequence that is at least 80%, at least 90%, or at least 95% identical to an amino acid sequence selected from the group consisting of SEQ ID NOs: 2, 10, 18, 26, 34, 42, 50, 58, 66, 74, 82, 90, 98, 106, 114, 122, 130, 205, 213, 221, 229, 237, 249, 257, 265, 273, 301, 310, 311, 312, 321, and 322; wherein the antibody specifically binds to human CD32b protein.

In one embodiment, the VH and/or VL amino acid sequences may be 50%, 60%, 70%, 80%, 90%, 95%, 96%, 97%, 98% or 99% identical to the sequences set forth in Tables 1, 2 or 3. In one embodiment, the VH and/or VL amino acid sequences may be identical except an amino acid substitution in no more than 1, 2, 3, 4 or 5 amino acid positions.

As used herein, the percent identity between the two sequences is a function of the number of identical positions shared by the sequences (i.e., % identity equals number of identical positions/total number of positions X 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below. Additionally or alternatively, the protein sequences of the present disclosure can further be used as a “query sequence” to perform a search against public databases to, for example, identify related sequences. For example, such searches can be performed using the BLAST program (version 2.0) of Altschul, et al., 1990 J. Mol. Biol. 215:403-10.

Antibodies with Conservative Modifications

In one embodiment, an antibody of the disclosure has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the CD32b-binding antibody molecules of the disclosure. Accordingly, the disclosure provides an anti-CD32b antibody molecule, consisting of a heavy chain variable region comprising CDR1, CDR2, and CDR3 sequences and a light chain variable region comprising CDR1, CDR2, and CDR3 sequences, wherein: the heavy chain variable region CDR1 comprises an amino acid sequence selected from any of SEQ ID NOs: 3, 11, 19, 27, 35, 43, 51, 59, 67, 75, 83, 91, 99, 107, 115, 123, 131, 202, 210, 218, 226, 234, 242, 246, 254, 262, 270, 302, 314 and 327, or conservative variants thereof; the heavy chain variable region CDR2 comprises an amino acid sequence selected from any of SEQ ID NOs: 4, 12, 20, 28, 36, 44, 52, 60, 68, 76, 84, 92, 100, 108, 116, 124, 132, 203, 211, 219, 227, 235, 243, 247, 255, 263, 271, 303, 315 and 328, or conservative variants thereof; the heavy chain variable region CDR3 comprises an amino acid sequence selected from any of SEQ ID NOs: 5, 13, 21, 29, 37, 45, 53, 61, 69, 77, 85, 93, 101, 109, 117, 125, 133, 204, 212, 220, 228, 236, 244, 248, 256, 264, 272, 304, 316, and 329, or conservative variants thereof; the light chain variable region CDR1 comprises an amino acid sequence selected from any of SEQ ID NOs: 6, 14, 22, 30, 38, 46, 54, 62, 70, 78, 86, 94, 102, 110, 118, 126, 134, 206, 214, 222, 230, 238, 250, 258, 266, 274, 305, 318, and 324, or conservative variants thereof; the light chain variable region CDR2 comprises an amino acid sequence selected from any of SEQ ID NOs: 7, 15, 23, 31, 39, 47, 55, 63, 71, 79, 87, 95, 103, 111, 119, 127, 135, 207, 215, 223, 231, 239, 251, 259, 267, 275, 306, 307, 308, 319 and 325, or conservative variants thereof; and the light chain variable region CDR3 comprises an amino acid sequence selected from any of SEQ ID NOs: 8, 16, 24, 32, 40, 48, 56, 64, 72, 80, 88, 96, 104, 112, 120, 128, 136, 208, 216, 224, 232, 240, 252, 260, 268, 276, 309, 320 and 326, or conservative variants thereof; wherein the anti-CD32b antibody molecule specifically binds to CD32b and mediates both macrophage and NK cell killing of antibody bound, CD32b positive target cells.

In one embodiment, an antibody of the disclosure optimized for expression in a mammalian cell has a heavy chain variable region and a light chain variable region, wherein one or more of these sequences have specified amino acid sequences based on the antibodies described herein or conservative modifications thereof, and wherein the antibodies retain the desired functional properties of the CD32b-binding antibody molecules of the disclosure. Accordingly, the disclosure provides an isolated monoclonal antibody optimized for expression in a mammalian cell comprising a heavy chain variable region and a light chain variable region wherein: the heavy chain variable region comprises an amino acid sequence selected from any of SEQ ID NOs: 1, 9, 17, 25, 33, 41, 49, 57, 65, 73, 81, 89, 97, 105, 113, 121, 129, 201, 209, 217, 225, 233, 241, 245, 253, 261, 269, 300, 313, 317 and 323, and conservative modifications thereof; and the light chain variable region comprises an amino acid sequence selected from any of SEQ ID NOs: 2, 10, 18, 26, 34, 42, 50, 58, 66, 74, 82, 90, 98, 106, 114, 122, 130, 205, 213, 221, 229, 237, 249, 257, 265, 273, 301, 310, 311, 312, 321, and 322, and conservative modifications thereof; wherein the antibody specifically binds to CD32b and mediates both macrophage and NK cell killing of antibody bound, CD32b positive target cells.

Antibodies that Bind to the Same Epitope

The present disclosure provides antibodies that bind to the same epitope as do the CD32b-binding antibodies listed in Tables 1, 2 or 3. Additional antibodies can therefore be identified based on their ability to cross-compete (e.g., to competitively inhibit the binding of, in a statistically significant manner) with other antibodies and antigen-binding fragments thereof of the disclosure inCD32b binding assays. The ability of a test antibody to inhibit the binding of antibodies and antigen-binding fragments thereof of the present disclosure to CD32b protein demonstrates that the test antibody can compete with that antibody for binding to CD32b; such an antibody may, according to non-limiting theory, bind to the same or a related (e.g., a structurally similar or spatially proximal) epitope on CD32B as the antibody with which it competes. In a certain embodiment, the antibody that binds to the same epitope on CD32B as the antibodies and antigen-binding fragments thereof of the present disclosure is a human monoclonal antibody. Such human monoclonal antibodies can be prepared and isolated as described herein.

Once a desired epitope on an antigen is determined, it is possible to generate antibodies to that epitope, e.g., using the techniques described in the present disclosure. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. A high throughput process for “binning” antibodies based upon their cross-competition is described in International Patent Application No. WO 2003/48731. As will be appreciated by one of skill in the art, practically anything to which an antibody can specifically bind could be an epitope. An epitope can comprises those residues to which the antibody binds.

Generally, antibodies specific for a particular target antigen will preferentially recognize an epitope on the target antigen in a complex mixture of proteins and/or macromolecules. Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids CD32bsuch as by, e.g., hydrogen/deuterium exchange, x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

### Engineered and Modified Antibodies

An antibody of the disclosure further can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region (s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Such framework sequences can be obtained from public DNA databases or published references that include germ line antibody gene sequences or rearranged antibody sequences. For example, germ line DNA sequences for human heavy and light chain variable region genes can be found in the “VBase” human germline sequence database (available on the Internet at www.mrc-cpe.cam.ac.uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. fol. Biol. 227:776-798; and Cox, J. P. L. et al., 1994 Eur. J Immunol. 24:827-836; the contents of each of which are expressly incorporated herein by reference. For example, germline DNA sequences for human heavy and light chain variable region genes and rearranged antibody sequences can be found in “IMGT” database (available on the Internet at www.imgt.org; see Lefranc, M. P. et al., 1999 Nucleic Acids Res. 27:209-212; the contents of each of which are expressly incorporated herein by reference.)

An example of framework sequences for use in the antibodies and antigen-binding fragments thereof of the disclosure are those that are structurally similar to the framework sequences used by selected antibodies and antigen-binding fragments thereof of the disclosure, e.g., consensus sequences and/or framework sequences used by monoclonal antibodies of the disclosure. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation (s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

Grafting Antigen-Binding Domains into Alternative Frameworks or Scaffolds

A wide variety of antibody/immunoglobulin frameworks or scaffolds can be employed so long as the resulting polypeptide includes at least one binding region which specifically binds to CD32b. Such frameworks or scaffolds include the 5 main idiotypes of human immunoglobulins, antigen-binding fragments thereof, and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

In one aspect, the disclosure pertains to a method of generating non-immunoglobulin based antibodies using non-immunoglobulin scaffolds onto which CDRs of the disclosure can be grafted. Known or future non-immunoglobulin frameworks and scaffolds may be employed, as long as they comprise a binding region specific for the target CD32b protein. Known non-immunoglobulin frameworks or scaffolds include, but are not limited to, fibronectin (Compound Therapeutics, Inc., Waltham, Mass.), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (SciI Proteins GmbH, Halle, Germany).

The fibronectin scaffolds are based on fibronectin type III domain (e.g., the tenth module of the fibronectin type III (10 Fn3 domain)) The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprises the entire antigen recognition unit in camel and llama IgG. Because of this structure, the non-immunoglobulin antibody mimics antigen binding properties that are similar in nature and affinity for those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the disclosure using standard cloning techniques.

The ankyrin technology is based on using proteins with ankyrin derived repeat modules as scaffolds for bearing variable regions which can be used for binding to different targets. The ankyrin repeat module is a 33 amino acid polypeptide consisting of two anti-parallel alpha-helices and a beta-turn. Binding of the variable regions is mostly optimized by using ribosome display.

Avimers are derived from natural A-domain containing protein such as LRP-1. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

The human CD32B-binding antibodies can be generated using methods that are known in the art. For example, the humaneering technology used for converting non-human antibodies into engineered human antibodies. U.S. Patent Publication No. 20050008625 describes an in vivo method for replacing a nonhuman antibody variable region with a human variable region in an antibody while maintaining the same or providing better binding characteristics relative to that of the nonhuman antibody. The method relies on epitope guided replacement of variable regions of a non-human reference antibody with a fully human antibody. The resulting human antibody is generally unrelated structurally to the reference nonhuman antibody, but binds to the same epitope on the same antigen as the reference antibody. Briefly, the serial epitope-guided complementarity replacement approach is enabled by setting up a competition in cells between a “competitor” and a library of diverse hybrids of the reference antibody (“test antibodies”) for binding to limiting amounts of antigen in the presence of a reporter system which responds to the binding of test antibody to antigen. The competitor can be the reference antibody or derivative thereof such as a single-chain Fv fragment. The competitor can also be a natural or artificial ligand of the antigen which binds to the same epitope as the reference antibody. The only requirements of the competitor are that it binds to the same epitope as the reference antibody, and that it competes with the reference antibody for antigen binding. The test antibodies have one antigen-binding V-region in common from the nonhuman reference antibody, and the other V-region selected at random from a diverse source such as a repertoire library of human antibodies. The common V-region from the reference antibody serves as a guide, positioning the test antibodies on the same epitope on the antigen, and in the same orientation, so that selection is biased toward the highest antigen-binding fidelity to the reference antibody.

Many types of reporter system can be used to detect desired interactions between test antibodies and antigen. For example, complementing reporter fragments may be linked to antigen and test antibody, respectively, so that reporter activation by fragment complementation only occurs when the test antibody binds to the antigen. When the test antibody- and antigen-reporter fragment fusions are co-expressed with a competitor, reporter activation becomes dependent on the ability of the test antibody to compete with the competitor, which is proportional to the affinity of the test antibody for the antigen. Other reporter systems that can be used include the reactivator of an auto-inhibited reporter reactivation system (RAIR) as disclosed in U.S. patent application Ser. No. 10/208,730 (Publication No. 20030198971), or competitive activation system disclosed in U.S. patent application Ser. No. 10/076,845 (Publication No. 20030157579).

With the serial epitope-guided complementarity replacement system, selection is made to identify cells expresses a single test antibody along with the competitor, antigen, and reporter components. In these cells, each test antibody competes one-on-one with the competitor for binding to a limiting amount of antigen. Activity of the reporter is proportional to the amount of antigen bound to the test antibody, which in turn is proportional to the affinity of the test antibody for the antigen and the stability of the test antibody. Test antibodies are initially selected on the basis of their activity relative to that of the reference antibody when expressed as the test antibody. The result of the first round of selection is a set of “hybrid” antibodies, each of which is comprised of the same non-human V-region from the reference antibody and a human V-region from the library, and each of which binds to the same epitope on the antigen as the reference antibody. One of more of the hybrid antibodies selected in the first round will have an affinity for the antigen comparable to or higher than that of the reference antibody.

In the second V-region replacement step, the human V-regions selected in the first step are used as guide for the selection of human replacements for the remaining non-human reference antibody V-region with a diverse library of cognate human V-regions. The hybrid antibodies selected in the first round may also be used as competitors for the second round of selection. The result of the second round of selection is a set of fully human antibodies which differ structurally from the reference antibody, but which compete with the reference antibody for binding to the same antigen. Some of the selected human antibodies bind to the same epitope on the same antigen as the reference antibody. Among these selected human antibodies, one or more binds to the same epitope with an affinity which is comparable to or higher than that of the reference antibody.

In addition, human CD32b-binding antibodies can also be commercially obtained from companies which customarily produce human antibodies, e.g., KaloBios, Inc. (Mountain View, Calif.).

### Camelid Antibodies

Antibody proteins obtained from members of the camel and dromedary (Camelus bactrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals See PCT/EP93/02214 (WO 94/04678 published 3 Mar. 1994).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520. Engineered libraries of camelid antibodies and antibody fragments are commercially available, for example, from Ablynx, Ghent, Belgium. As with other antibodies and antigen-binding fragments thereof of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. Further, these molecules can be fully expressed in prokaryotic cells such as E. coli and are expressed as fusion proteins with bacteriophage and are functional.

Accordingly, a feature of the present disclosure is a camelid antibody or nanobody having high affinity for CD32b. In one embodiment herein, the camelid antibody or nanobody is naturally produced in the camelid animal, i.e., is produced by the camelid following immunization with CD32b or a peptide fragment thereof, using techniques described herein for other antibodies. Alternatively, the CD32b-binding camelid nanobody is engineered, i.e., produced by selection for example from a library of phage displaying appropriately mutagenized camelid nanobody proteins using panning procedures with CD32b as a target as described in the examples herein. Engineered nanobodies can further be customized by genetic engineering to have a half-life in a recipient subject of from 45 minutes to two weeks. In a specific embodiment, the camelid antibody or nanobody is obtained by grafting the CDRs sequences of the heavy or light chain of the human antibodies of the disclosure into nanobody or single domain antibody framework sequences, as described for example in PCT/EP93/02214.

### Bispecific Molecules and Multivalent Antibodies

In another aspect, the present disclosure features bispecific or multispecific molecules comprising a CD32b-binding antibody, or a fragment thereof, of the disclosure. An antibody of the disclosure, or antigen-binding regions thereof, can be derivatized or linked to another functional molecule, e.g., another peptide or protein (e.g., another antibody or ligand for a receptor) to generate a bispecific molecule that binds to at least two different binding sites or target molecules. The antibody of the disclosure may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the disclosure, an antibody of the disclosure can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present disclosure includes bispecific molecules comprising at least one first binding specificity for CD32b and a second binding specificity for a second target epitope. For example, the second target epitope is another epitope of CD32b different from the first target epitope. Additionally, for the disclosure in which the bispecific molecule is multi-specific, the molecule can further include a third binding specificity, in addition to the first and second target epitope. In one embodiment, the bispecific molecules of the disclosure comprise as a binding specificity at least one antibody, or an antibody fragment thereof, including, e.g., an Fab, Fab′, F (ab′)2, Fv, or a single chain Fv. The antibody may also be a light chain or heavy chain dimer, or any minimal fragment thereof such as a Fv or a single chain construct as described in Ladner et al. U.S. Pat. No. 4,946,778.

Diabodies are bivalent, bispecific molecules in which VH and VL domains are expressed on a single polypeptide chain, connected by a linker that is too short to allow for pairing between the two domains on the same chain. The VH and VL domains pair with complementary domains of another chain, thereby creating two antigen binding sites (see e.g., Holliger et al., 1993 Proc. Natl. Acad. Sci. USA 90:6444-6448; Poijak et al., 1994 Structure 2:1121-1123). Diabodies can be produced by expressing two polypeptide chains with either the structure VHA-VLB and VHB-VLA (VH-VL configuration), or VLA-VHB and VLB-VHA (VL-VH configuration) within the same cell. Most of them can be expressed in soluble form in bacteria. Single chain diabodies (scDb) are produced by connecting the two diabody-forming polypeptide chains with linker of approximately 15 amino acid residues (see Holliger and Winter, 1997 Cancer Immunol. Immunother., 45 (3-4):128-30; Wu et al., 1996 Immunotechnology, 2 (1):21-36). scDb can be expressed in bacteria in soluble, active monomeric form (see Holliger and Winter, 1997 Cancer Immunol. Immunother., 45 (34): 128-30; Wu et al., 1996 Immunotechnology, 2 (1):21-36; Pluckthun and Pack, 1997 Immunotechnology, 3 (2): 83-105; Ridgway et al., 1996 Protein Eng., 9 (7):617-21). A diabody can be fused to Fc to generate a “di-diabody” (see Lu et al., 2004 J. Biol. Chem., 279 (4):2856-65).

Other antibodies which can be employed in the bispecific molecules of the disclosure are murine, chimeric and humanized monoclonal antibodies.

The bispecific molecules of the present disclosure can be prepared by conjugating the constituent binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis (2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, III.).

When the binding specificities are antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb X mAb, mAb X Fab, Fab X F (ab′)2 or ligand X Fab fusion protein. A bispecific molecule of the disclosure can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In another aspect, the present disclosure provides multivalent compounds comprising at least two identical or different antigen-binding portions of the antibodies and antigen-binding fragments thereof of the disclosure binding to CD32b. The antigen-binding portions can be linked together via protein fusion or covalent or non-covalent linkage. Alternatively, methods of linkage have been described for the bispecific molecules. Tetravalent compounds can be obtained for example by cross-linking antibodies and antigen-binding fragments thereof of the disclosure with an antibody or antigen-binding fragment that binds to the constant regions of the antibodies and antigen-binding fragments thereof of the disclosure, for example the Fc or hinge region.

Trimerizing domain are described for example in Borean patent EP 1 012 280B1. Pentamerizing modules are described for example in PCT/EP97/05897.

Antibodies with Extended Half Life

The present disclosure provides for antibodies that specifically bind to CD32b which have an extended half-life in vivo.

Many factors may affect a protein's half-life in vivo. For examples, kidney filtration, metabolism in the liver, degradation by proteolytic enzymes (proteases), and immunogenic responses (e.g., protein neutralization by antibodies and uptake by macrophages and dentritic cells). A variety of strategies can be used to extend the half-life of the antibodies and antigen-binding fragments thereof of the present disclosure. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nanocarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, antigen-binding fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10)alkoxy- or aryloxy-polyethylene glycol or polyethylene glycol-maleimide. In one embodiment, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies and antigen-binding fragments thereof of the disclosure. See for example, EP 0 154 316 by Nishimura et al. and EP 0 401 384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a normative amino acid any place an amber codon is positioned, converting the amber from a stop codon to one that signals incorporation of the chemically specified amino acid.

Recombinant pegylation technology (rPEG) can also be used for serum halflife extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum halflife of the protein is greatly increased. In contrast to traditional PEGylation, which requires chemical conjugation and repurification, the manufacturing process is greatly simplified and the product is homogeneous. Polysialylation is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin in order to make the antibody or antibody fragment more stable in vivo or have a longer half-life in vivo. The techniques are well-known in the art, see, e.g., International Publication Nos. WO 93/15199, WO 93/15200, and WO 01/77137; and European Patent No. EP 413,622.

The strategies for increasing half-life is especially useful in nanobodies, fibronectin-based binders, and other antibodies or proteins for which increased in vivo half-life is desired.

### Antibody Conjugates

The present disclosure provides antibodies or antigen-binding fragments thereof that specifically bind to CD32b recombinantly fused or chemically conjugated (including both covalent and non-covalent conjugations) to a heterologous protein or polypeptide (or antigen-binding fragment thereof, preferably to a polypeptide of at least 10, at least 20, at least 30, at least 40, at least 50, at least 60, at least 70, at least 80, at least 90 or at least 100 amino acids) to generate fusion proteins. In particular, the disclosure provides fusion proteins comprising an antigen-binding fragment of an antibody described herein (e.g., a Fab fragment, Fd fragment, Fv fragment, F (ab)2 fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies and antigen-binding fragments thereof of the disclosure (e.g., antibodies and antigen-binding fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16 (2):76-82; Hansson, et al., 1999, J. Mol. Biol. 287:265-76; and Lorenzo and Blasco, 1998, Biotechniques 24 (2):308-313 (each of these patents and publications are hereby incorporated by reference in its entirety). Antibodies and antigen-binding fragments thereof, or the encoded antibodies and antigen-binding fragments thereof, may be altered by being subjected to random mutagenesis by error-prone PCR, random nucleotide insertion or other methods prior to recombination. A polynucleotide encoding an antibody antigen-binding fragment thereof that specifically binds to CD32b may be recombined with one or more components, motifs, sections, parts, domains, fragments, etc. of one or more heterologous molecules.

Moreover, the antibodies and antigen-binding fragments thereof can be fused to marker sequences, such as a peptide to facilitate purification. In one embodiment, the marker amino acid sequence is a hexa-histidine peptide (SEQ ID NO: 1648), such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine (SEQ ID NO: 1648) provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In one embodiment, CD32b binding antibodies and antigen-binding fragments thereof of the present disclosure may be conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149 Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and nonradioactive paramagnetic metal ions.

The present disclosure further encompasses uses of antibodies and antigen-binding fragments thereof conjugated to a therapeutic moiety. An antibody antigen-binding fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody antigen-binding fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, alpha-interferon, beta-interferon, nerve growth factor, platelet derived growth factor, tissue plasminogen activator, an apoptotic agent, an anti-angiogenic agent; or, a biological response modifier such as, for example, a lymphokine.

Moreover, an antibody can be conjugated to therapeutic moieties such as a radioactive metal ion, such as alpha-emitters such as 213Bi or macrocyclic chelators useful for conjugating radiometal ions, including but not limited to, 131In, 131LU, 131Y, 131Ho, 131Sm, to polypeptides. In one embodiment, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4 (10):2483-90; Peterson et al., 1999, Bioconjug. Chem. 10 (4):553-7; and Zimmerman et al., 1999, Nucl. Med. Biol. 26 (8):943-50, each incorporated by reference in their entireties.

Techniques for conjugating therapeutic moieties to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Methods of Producing Antibodies of the Disclosure

### Nucleic Acids Encoding the Antibodies

The disclosure provides substantially purified nucleic acid molecules which encode polypeptides comprising segments or domains of the CD32b-binding antibody chains described above. In some embodiments, the nucleic acid molecule comprises nucleotide sequences that encode heavy and light chain variable regions, and CDRs or hypervariable regions of the antibody molecules, as described herein. The nucleic acid molecule can comprise a nucleotide sequence as set forth herein, or a sequence substantially identical thereto (e.g., a sequence at least 85%, 90%, 95%, 99% or more identical thereto, or which differs by no more than 3, 6, 15, 30, or 45 nucleotides from the sequences shown in Table 4). In some embodiments, the nucleic acid molecule comprises a nucleotide sequence encoding any of the amino acids disclosed in Tables 1, 2 or 3, or a sequence substantially identical thereto (e.g., a sequence at least 85%, 90%, 95%, 99% or more identical thereto, or which differs by no more than 3, 6, 15, 30, or 45 nucleotides from a sequence encoding an amino acid disclosed in Tables 1, 2 or 3).

In some embodiments, the nucleic acid molecule comprises a nucleotide sequence that encodes a variable light chain disclosed in Tables 1, 2 or 3, or a nucleotide sequence at least 85%, 90%, 95%, 99% or more identical thereto. In some embodiments, the nucleic acid molecule comprises a nucleotide sequence that encodes a variable heavy chain disclosed in Tables 1, 2 or 3, or a nucleotide sequence at least 85%, 90%, 95%, 99% or more identical thereto. In some embodiments, the nucleic acid molecule comprises a nucleotide sequence that encodes a variable heavy chain and a variable light chain disclosed in Tables 1, 2, or 3, or a sequence at least 85%, 90%, 95%, 99% or more identical thereto.

In some embodiments, the nucleic acid molecule comprises a nucleotide sequence encoding a heavy chain variable region disclosed in Table 4, or a nucleotide sequence at least 85%, 90%, 95%, 99% or more identical thereto. In some embodiments, the nucleic acid molecule comprises a nucleotide sequence encoding a light chain variable region disclosed in Table 4, or a nucleotide sequence at least 85%, 90%, 95%, 99% or more identical thereto. In some embodiments, the nucleic acid molecule comprises a nucleotide sequence encoding a heavy chain variable region and a light chain variable region disclosed in Table 4, or a nucleotide sequence at least 85%, 90%, 95%, 99% or more identical thereto.

In some embodiments, the nucleic acid molecule comprises a nucleotide sequence encoding a heavy chain variable region shown in any of SEQ ID NOs: 333, 335, 336, or 339, or a sequence at least 85%, 90%, 95%, 99% or more identical thereto. In some embodiments, the nucleic acid molecule comprises a nucleotide sequence encoding a light chain variable region shown in any of SEQ ID NOs: 330, 331, 332, 334, 337, or 338, or a sequence at least 85%, 90%, 95%, 99% or more identical thereto. In some embodiments, the nucleic acid molecule comprises a nucleotide sequence encoding a heavy chain variable region shown in any of SEQ ID NOs: 333, 335, 336, or 339, or a sequence at least 85%, 90%, 95%, 99% or more identical thereto, and/or a nucleotide sequence encoding a light chain variable region shown in any of SEQ ID NOs: 330, 331, 332, 334, 337, or 338, or a sequence at least 85%, 90%, 95%, 99% or more identical thereto.

In an embodiment, the nucleic acid molecules are those identified in Table 4, or those that encode any of the amino acids disclosed in Tables 1, 2, or 3. Some other nucleic acid molecules of the disclosure comprise nucleotide sequences that are substantially identical (e.g., at least 80%, 95%, or 99%) to the nucleotide sequences disclosed in Table 4, or to nucleotide sequences that encode any of the amino acids disclosed in Tables 1, 2, or 3. When expressed from appropriate expression vectors, polypeptides encoded by these polynucleotides are capable of exhibiting CD32b antigen binding capacity.

In some embodiments, the nucleic acid molecule comprises a nucleotide sequence that encodes an amino acid sequence comprising at least one CDR region and usually all three CDR regions from a heavy or light chain of a CD32b-binding antibody set forth in Tables 1, 2 or 3. In some embodiments, the nucleic acid molecule comprises a nucleotide sequence that encodes at least one CDR region and usually all three CDR regions from a heavy or light chain of a CD32b-binding antibody as disclosed in Table 4.

Some other polynucleotides encode all or substantially all of the variable region sequence of the heavy chain and/or the light chain of the CD32b-binding antibody set forth in Tables 1, 2 or 3. In some embodiments, the nucleic acid molecule comprises a nucleotide sequence that encodes all or substantially all of the variable region sequence of a heavy chain and/or light chain of the CD32b-binding antibody set forth in Table 4. Because of the degeneracy of the code, a variety of nucleic acid sequences will encode each of the immunoglobulin amino acid sequences.

The nucleic acid molecules of the disclosure can encode both a variable region and a constant region of the antibody. In some embodiments, the nucleic acid molecule comprises a nucleotide sequence that encodes an amino acid of a mature heavy chain variable region sequence that is identical or substantially identical (e.g., at least 80%, 90%, or 99%) to the nucleotide sequence encoding the mature heavy chain variable region of any of the heavy chain variable regions disclosed in Tables 1, 2 or 3. In some embodiments, the nucleic acid molecule comprises a nucleotide sequence that encodes an amino acid of a mature light chain variable region sequence that is identical or substantially identical (e.g., at least 80%, 90%, or 99%) to the nucleotide sequence encoding the mature light chain variable region of any of the light chain variable regions disclosed in Tables 1, 2 or 3. In some embodiments, the nucleic acid molecule comprises a nucleotide sequence that encodes a mature heavy chain variable region sequence that is identical or substantially identical (e.g., at least 80%, 90%, or 99%) to the mature heavy chain variable region sequence of any of SEQ ID NOs: 333, 335, 336, or 339. In some embodiments, the nucleic acid molecule comprises a nucleotide sequence that encodes a mature light chain variable region sequence that is identical or substantially identical (e.g., at least 80%, 90%, or 99%) to the mature light chain variable region sequence of any of SEQ ID NOs: 330, 331, 332, 334, 337, or 338.

TABLE 4
Exemplary nucleotide sequences encoding anti-
CD32b antibody molecules
Clone: 2B6

| | | |
|---|---|---|
| 330 | Human- | GAAATTGTGCTGACTCAGTCTCCAGACTTTCAGTCTGTG |
| | ized | ACTCCAAAGGAGAAAGTCACCATCACCTGCAGGACCAG |
| | 2B6 | TCAGAGCATTGGCACAAACATACACTGGTACCAGCAGA |
| | VL-1 | AACCAGATCAGTCTCCAAAGCTCCTCATCAAGAATGTTT |
| | | CTGAGTCTATCTCTGGAGTCCCATCGAGGTTCAGTGGCA |
| | | GTGGATCTGGGACAGATTTCACCCTCACCATCAATAGCC |
| | | TGGAAGCTGAAGATGCTGCAACGTATTACTGTCAACAAA |
| | | GTAATACCTGGCCGTTCACGTTCGGCGGAGGGACCAAGG |
| | | TGGAGATCAAA |
| 331 | Human- | GAAATTGTGCTGACTCAGTCTCCAGACTTTCAGTCTGTG |
| | ized | ACTCCAAAGGAGAAAGTCACCATCACCTGCAGGACCAG |
| | 2B6 | TCAGAGCATTGGCACAAACATACACTGGTACCAGCAGA |
| | VL-2 | AACCAGATCAGTCTCCAAAGCTCCTCATCAAGTATGTTT |
| | | CTGAGTCTATCTCTGGAGTCCCATCGAGGTTCAGTGGCA |
| | | GTGGATCTGGGACAGATTTCACCCTCACCATCAATAGCC |
| | | TGGAAGCTGAAGATGCTGCAACGTATTACTGTCAACAAA |
| | | GTAATACCTGGCCGTTCACGTTCGGCGGAGGGACCAAGG |
| | | TGGAGATCAAA |
| 332 | Human- | GAAATTGTGCTGACTCAGTCTCCAGACTTTCAGTCTGTG |
| | ized | ACTCCAAAGGAGAAAGTCACCATCACCTGCAGGACCAG |
| | 2B6 | TCAGAGCATTGGCACAAACATACACTGGTACCAGCAGA |
| | VL-3 | AACCAGATCAGTCTCCAAAGCTCCTCATCAAGTATGCTT |
| | | CTGAGTCTATCTCTGGAGTCCCATCGAGGTTCAGTGGCA |
| | | GTGGATCTGGGACAGATTTCACCCTCACCATCAATAGCC |
| | | TGGAAGCTGAAGATGCTGCAACGTATTACTGTCAACAAA |
| | | GTAATACCTGGCCGTTCACGTTCGGCGGAGGGACCAAGG |
| | | TGGAGATCAAA |
| 333 | Human- | CAGGTTCAGCTGGTGCAGTCTGGAGCTGAGGTGAAGAA |
| | ized | GCCTGGGGCCTCAGTGAAGGTCTCCTGCAAGGCTTCTGG |
| | 2B6 | TTACACCTTTACCAACTACTGGATACACTGGGTGCGACA |
| | VH | GGCCCCTGGACAAGGGCTTGAGTGGATGGGAGTGATTG |
| | | ATCCTTCTGATACTTATCCAAATTACAATAAAAAGTTCA |
| | | AGGGCAGAGTCACCATGACCACAGACACATCCACGAGC |
| | | ACAGCCTACATGGAGCTGAGGAGCCTGAGATCTGACGA |
| | | CACGGCCGTGTATTACTGTGCGAGAAACGGTGATTCCGA |
| | | TTATTACTCTGGTATGGACTACTGGGGGCAAGGGACCAC |
| | | GGTCACCGTCTCCTCA |
| 334 | Mouse | GACATCTTGCTGACTCAGTCTCCAGCCATCCTGTCTGTGA |
| | VL | GTCCAGGAGAGAGAGTCAGTTTTTCCTGCAGGACCAGTC |
| | | AGAGCATTGGCACAAACATACACTGGTATCAGCAAAGA |
| | | ACAAATGGTTTTCCAAGGCTTCTCATAAAGAATGTTTCT |
| | | GAGTCTATCTCTGGGATCCCTTCCAGGTTTAGTGGCAGT |
| | | GGATCAGGGACAGATTTTATTCTTAGCATCAACAGTGTG |
| | | GAGTCTGAAGATATTGCAGATTATTATTGTCAACAAAGT |
| | | AATACCTGGCCGTTCACGTTCGGAGGGGGGACCAAGCTG |
| | | GAAATAAAA |
| 335 | Mouse | CAGGTCCAATTGCAGCAGCCTGTGACTGAGCTGGTGAGG |
| | VH | CCGGGGGCTTCAGTGATGTTGTCCTGCAAGGCTTCTGAC |
| | | TACCCCTTCACCAACTACTGGATACACTGGGTAAAGCAG |
| | | AGGCCTGGACAAGGCCTGGAGTGGATCGGAGTGATTGA |
| | | TCCTTCTGATACTTATCCAAATTACAATAAAAAGTTCAA |
| | | GGGCAAGGCCACATTGACTGTAGTCGTATCCTCCAGCAC |
| | | AGCCTACATGCAGCTCAGCAGCCTGACATCTGACGATTC |
| | | TGCGGTCTATTACTGTGCAAGAAACGGTGATTCCGATTA |
| | | TTACTCTGGTATGGACTACTGGGGTCAAGGAACCTCAGT |
| | | CACCGTCTCCTCA |

Clone: 3H7

| | | |
|---|---|---|
| 336 | VH | GAAGTGAAGTTTGAGGAGTCTGGAGGAGGCTTGGTGCA |
| | | ACCTGGAGGATCCATGAAACTCTCTTGTGCTGCCTCTGG |
| | | ATTCACTTTTAGTGACGCCTGGATGGACTGGGTCCGCCA |
| | | GGGTCCAGAGAAGGGGCTTGAGTGGGTTGCTGAAATTA |
| | | GAAACAAAGCTAATAATCTTGCAACATACTATGCTGAGT |
| | | CTGTGAAAGGGAGGTTCACCATCCCAAGAGATGATTCCA |
| | | AAAGTAGTCCCTTTGCTTACTGGGGCCAAGGGACTCTGG |
| | | TCACTGTCTCTGCA |
| 337 | VL | GACATCCAGATGACCCAGTCTCCATCCTCCTTATCTGCCT |
| | | CTCTGGGAGAAAGAGTCAGTCTCACTTGTCGGGCAAGTC |
| | | AGGAAATTAGTGGTTACTTAAGCTGGCTTCAGCAGAAAC |
| | | CAGATGGAACTATTAGACGCCTGATCTACGCCGCATCCA |
| | | CTTTAGATTCTGGTGTCCCAAAAAGGTTCAGTGGCAGTT |
| | | GGTCTGGGTCAGATTATTCTCTCACCATCAGCAGCCTTG |
| | | AGTCTGAAGATTTTGCAGACTATTACTGTCTACAATATG |
| | | TTAGTTATCCGTATACGTTCGGAGGGGGGACCAAGCTGG |
| | | AAATAAAA |

Clone: 8B5

| | | |
|---|---|---|
| 338 | VL | GACATTCAGATGACACAGTCTCCATCCTCCCTACTTGCG |
| | | GCGCTGGGAGAAAGAGTCAGTCTCACTTGTCGGGCAAGT |
| | | CAGGAAATTAGTGGTTACTTAAGCTGGCTTCAGCAGAAA |
| | | CCAGATGGAACTATTAAACGCCTGATCTACGCCGCATCC |
| | | ACTTTAGATTCTGGTGTCCCAAAAAGGTTCAGTGGCAGT |
| | | GAGTCTGGGTCAGATTATTCTCTCACCATCAGCAGTCTT |
| | | GAGTCTGAAGATTTTGCAGACTATTACTGTCTACAATAT |
| | | TTTAGTTATCCGCTCACGTTCGGTGCTGGGACCAAGCTG |
| | | GAGCTGAAA |
| 339 | VH | GAAGTGAAGCTTGAGGAGTCTGGAGGAGGCTTGGTGCA |
| | | ACCTGGAGGATCCATGAAACTCTCTTGTGAAGCCTCTGG |
| | | ATTCACTTTTAGTGACGCCTGGATGGACTGGGTCCGTCA |
| | | GTCTCCAGAGAAGGGGCTTGAGTGGGTTGCTGAAATTAG |
| | | AAACAAAGCTAAAAATCATGCAACATACTATGCTGAGTC |
| | | TGTGATAGGGAGGTTCACCATCTCAAGAGATGATTCCAA |
| | | AAGTAGTGTCTACCTGCAAATGAACAGCTTAAGAGCTGA |
| | | AGACACTGGCATTTATTACTGTGGGGCTCTGGGCCTTGA |
| | | CTACTGGGGCCAAGGCACCACTCTCACAGTCTCCTCG |

The polynucleotide sequences can be produced by de novo solid-phase DNA synthesis or by PCR mutagenesis of an existing sequence (e.g., sequences as described in the Examples below) encoding a CD32b-binding antibody or its binding fragment. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the disclosure are expression vectors and host cells for producing the CD32b-binding antibodies described above. Various expression vectors can be employed to express the polynucleotides encoding the CD32b-binding antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet. 15:345, 1997). For example, nonviral vectors useful for expression of the CD32b-binding polynucleotides and polypeptides in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a CD32b-binding antibody chain antigen-binding fragment. In one embodiment, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of a CD32b-binding antibody chain antigen-binding fragment. These elements typically include an ΔTG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted CD32b-binding antibody sequences. More often, the inserted CD32b-binding antibody sequences are linked to a signal sequences before inclusion in the vector. Vectors to be used to receive sequences encoding CD32b-binding antibody light and heavy chain variable domains sometimes also encode constant regions or parts thereof. Such vectors allow expression of the variable regions as fusion proteins with the constant regions thereby leading to production of intact antibodies and antigen-binding fragments thereof. Typically, such constant regions are human.

The host cells for harboring and expressing the CD32b-binding antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express CD32b-binding polypeptides of the disclosure. Insect cells in combination with baculovirus vectors can also be used.

In one embodiment, mammalian host cells are used to express and produce the CD32b-binding polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP poIIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express CD32b-binding antibody chains or binding fragments can be prepared using expression vectors of the disclosure which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Generation of Monoclonal Antibodies of the Disclosure

Monoclonal antibodies (mAbs) can be produced by a variety of techniques, including conventional monoclonal antibody methodology e.g., the standard somatic cell hybridization technique of Kohler and Milstein, 1975 Nature 256: 495. Many techniques for producing monoclonal antibody can be employed e.g., viral or oncogenic transformation of B lymphocytes.

An animal system for preparing hybridomas is the murine system. Hybridoma production in the mouse is a well established procedure Immunization protocols and techniques for isolation of immunized splenocytes for fusion are known in the art. Fusion partners (e.g., murine myeloma cells) and fusion procedures are also known.

In a certain embodiment, the antibodies of the disclosure are humanized monoclonal antibodies. Chimeric or humanized antibodies and antigen-binding fragments thereof of the present disclosure can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In a certain embodiment, the antibodies of the disclosure are human monoclonal antibodies. Such human monoclonal antibodies directed against CD32b can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (mu and gamma) and kappa light chain immunoglobulin sequences, together with targeted mutations that inactivate the endogenous mu and kappa chain loci (see e.g., Lonberg, et al., 1994 Nature 368 (6474): 856-859). Accordingly, the mice exhibit reduced expression of mouse IgM or K, and in response to immunization, the introduced human heavy and light chain transgenes undergo class switching and somatic mutation to generate high affinity human IgG-kappa monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N.Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et al., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/103918, WO 93/12227, WO 94/25585, WO 97/113852, WO 98/24884 and WO 99/45962, all to Lonberg and Kay; and PCT Publication No. WO 01/14424 to Korman et al.

In another embodiment, human antibodies of the disclosure can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise CD32b-binding antibodies and antigen-binding fragments thereof of the disclosure. For example, an alternative transgenic system referred to as the Xenomouse (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise CD32b-binding antibodies of the disclosure. For example, mice carrying both a human heavy chain transchromosome and a human light chain transchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise CD32b-binding antibodies of the disclosure.

Human monoclonal antibodies of the disclosure can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and U.S. Pat. No. 5,571,698 to Ladner et al; U.S. Pat. Nos. 5,427,908 and 5,580,717 to Dower et al; U.S. Pat. Nos. 5,969,108 and 6,172,197 to McCafferty et al; and U.S. Pat. Nos. 5,885,793; 6,521,404; 6,544,731; 6,555,313; 6,582,915 and 6,593,081 to Griffiths et al.

Human monoclonal antibodies of the disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Framework or Fc Engineering

Engineered antibodies and antigen-binding fragments thereof of the disclosure include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis. Such “backmutated” antibodies are also intended to be encompassed by the disclosure.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the disclosure may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the disclosure may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In one embodiment, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fc-gamma receptor by modifying one or more amino acids. This approach is described further, for example, in PCT Publication WO 00/42072 by Presta and by Lazar et al., 2006 PNAS 103(110): 4005-4010. Moreover, the binding sites on human IgG1 for Fc-gamma RI, Fc-gamma RII, Fc-gamma RIII and FcRn have been mapped and variants with improved binding have been described (see Shields, R. L. et al., 2001 J. Biol. Chen. 276:6591-6604).

In still another embodiment, the glycosylation of an antibody is modified. For example, an aglycoslated antibody can be made (i.e., the antibody lacks glycosylation). Glycosylation can be altered to, for example, increase the affinity of the antibody for “antigen”. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated or afucosylated antibody having reduced amounts of fucosyl residues, or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, LecI3 cells, with reduced ability to attach fucose to Asn (297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180). Von Horsten et al. in 2010 Glycobiology 20(12):1607-18 also describe a method of producing non-fucosylated antibodies by co-expression of antibodies with a heterologous GDP-6-deoxy-D-lyxo-4-hexulose reductase in CHO cells.

### Methods of Engineering Altered Antibodies

As discussed above, the CD32b-binding antibodies having VH and VL sequences or full length heavy and light chain sequences shown herein can be used to create new CD32b-binding antibodies by modifying full length heavy chain and/or light chain sequences, VH and/or VL sequences, or the constant region (s) attached thereto. Thus, in another aspect of the disclosure, the structural features of CD32b-binding antibody of the disclosure are used to create structurally related CD32b-binding antibodies that retain at least one functional property of the antibodies and antigen-binding fragments thereof of the disclosure, such as binding to human CD32b and also inhibiting one or more functional properties of CD32b.

For example, one or more CDR regions of the antibodies and antigen-binding fragments thereof of the present disclosure, or mutations thereof, can be combined recombinantly with known framework regions and/or other CDRs to create additional, recombinantly-engineered, CD32b-binding antibodies and antigen-binding fragments thereof of the disclosure, as discussed above. Other types of modifications include those described in the previous section. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence (s) is used as the starting material to create a “second generation” sequence (s) derived from the original sequence (s) and then the “second generation” sequence (s) is prepared and expressed as a protein.

The altered antibody sequence can also be prepared by screening antibody libraries having fixed CDR3 sequences or minimal essential binding determinants as described in US20050255552 and diversity on CDR1 and CDR2 sequences. The screening can be performed according to any screening technology appropriate for screening antibodies from antibody libraries, such as phage display technology.

Standard molecular biology techniques can be used to prepare and express the altered antibody sequence. The antibody encoded by the altered antibody sequence (s) is one that retains one, some or all of the functional properties of the CD32b-binding antibodies described herein, which functional properties include, but are not limited to, specifically binding to human CD32b protein and/or inhibiting one or more functional properties of CD32b.

The functional properties of the altered antibodies can be assessed using standard assays available in the art and/or described herein, such as those set forth in the Examples (e.g., ELISAs). In one embodiment of the methods of engineering antibodies and antigen-binding fragments thereof of the disclosure, mutations can be introduced randomly or selectively along all or part of a CD32b-binding antibody coding sequence and the resulting modified CD32b-binding antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Characterization of the Antibody Molecules

The antibody molecules can be characterized by various functional assays. For example, they can be characterized by their ability to inhibit CD32b.

The ability of an antibody to bind to CD32b can be detected by labelling the antibody of interest directly, or the antibody may be unlabeled and binding detected indirectly using various sandwich assay formats known in the art.

In one embodiment, the CD32b-binding antibody molecules block or compete with binding of a reference CD32b-binding antibody to CD32b polypeptide. These can be fully human or humanized CD32b-binding antibodies described above. They can also be other human, mouse, chimeric or humanized CD32b-binding antibodies which bind to the same epitope as the reference antibody. The capacity to block or compete with the reference antibody binding indicates that CD32b-binding antibody under test binds to the same or similar epitope as that defined by the reference antibody, or to an epitope which is sufficiently proximal to the epitope bound by the reference CD32b-binding antibody. Such antibodies are especially likely to share the advantageous properties identified for the reference antibody. The capacity to block or compete with the reference antibody may be determined by, e.g., a competition binding assay. With a competition binding assay, the antibody under test is examined for ability to inhibit specific binding of the reference antibody to a common antigen, such as CD32b polypeptide. A test antibody competes with the reference antibody for specific binding to the antigen if an excess of the test antibody substantially inhibits binding of the reference antibody. Substantial inhibition means that the test antibody reduces specific binding of the reference antibody usually by at least 10%, 25%, 50%, 75%, or 90%.

There are a number of known competition binding assays that can be used to assess competition of an antibody with a reference antibody for binding to a particular protein, in this case, CD32b. These include, e.g., solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol. 137:3614-3619, 1986); solid phase direct labeled assay, solid phase direct labeled sandwich assay (see Harlow & Lane, supra); solid phase direct label RIA using 1-125 label (see Morel et al., Molec. Immunol. 25:7-15, 1988); solid phase direct biotin-avidin EIA (Cheung et al., Virology 176:546-552, 1990); and direct labeled RIA (Moldenhauer et al., Scand. J. Immunol. 32:77-82, 1990). Typically, such an assay involves the use of purified antigen bound to a solid surface or cells bearing either of these, an unlabelled test CD32b-binding antibody and a labelled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

To determine if the selected CD32b-binding monoclonal antibodies bind to unique epitopes, each antibody can be biotinylated using commercially available reagents (e.g., reagents from Pierce, Rockford, Ill.). Competition studies using unlabeled monoclonal antibodies and biotinylated monoclonal antibodies can be performed using CD32b polypeptide coated-ELISA plates. Biotinylated MAb binding can be detected with a strep-avidin-alkaline phosphatase probe. To determine the isotype of a purified CD32b-binding antibody, isotype ELISAs can be performed. For example, wells of microtiter plates can be coated with 1 μg/ml of anti-human IgG overnight at 4 degrees C. After blocking with 1% BSA, the plates are reacted with 1 μg/ml or less of the monoclonal CD32b-binding antibody or purified isotype controls, at ambient temperature for one to two hours. The wells can then be reacted with either human IgG1 or human IgM-specific alkaline phosphatase-conjugated probes. Plates are then developed and analyzed so that the isotype of the purified antibody can be determined.

To demonstrate binding of monoclonal CD32b-binding antibodies to live cells expressing CD32b polypeptide, flow cytometry can be used. Briefly, cell lines expressing CD32b (grown under standard growth conditions) can be mixed with various concentrations of CD32b-binding antibody in PBS containing 0.1% BSA and 10% fetal calf serum, and incubated at 37 degrees C. for 1 hour. After washing, the cells are reacted with Fluorescein-labeled anti-human IgG antibody under the same conditions as the primary antibody staining. The samples can be analyzed by FACScan instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

CD32b-binding antibodies and antigen-binding fragments thereof of the disclosure can be further tested for reactivity with CD32b polypeptide or antigenic fragment by Western blotting. Briefly, purified CD32b polypeptides or fusion proteins, or cell extracts from cells expressing CD32b can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

### Anti-Cd32B Activities

Exemplary activities of CD32b protein which can be modulated by the anti-CD32b antibodies disclosed herein include one or more of the following.

The CD32b protein comprises intracellular ITIM domains and extracellular Fc-binding domains that can bind to immunoglobulin Fc molecules. The extracellular Fc-binding domains of CD32b can bind to, e.g., Fc domains on antibody molecules or Fc-fusion proteins. Binding of Fc molecules to the extracellular Fc-binding domains of CD32b results in the activation of CD32b and ITIM phosphorylation which in turn results in inhibition of activatory FcγR functions (Smith and Clatworthy, Nat. Rev. Immunol. 2010: (5) 328-343) or, when cross-linked to the B cell receptor, reduced B cell function (Horton et al., J. Immunol. 2011: 186(7):4223-4233).

Without wishing to be bound by theory, it is believed that CD32b may exert its inhibitory effects on immune cells, at least in part, by the mechanisms described herein. CD32b can control humoral immunity by regulating B cell activation and survival of plasma cells. CD32b can negatively regulate B cell activation by modulating, e.g., increasing, the B cell receptor (BCR) threshold and suppressing antigen presentation to T cells (Smith and Clatworthy, Nat. Rev. Immunol. 2010: (5) 328-343).

On macrophages, CD32b mediated immunosuppressive signaling can reduce macrophage activity. For example, crosslinking of CD32b on macrophages results in inhibition of Fc gamma receptor mediated phagocytosis, superoxide production, cytokine release (e.g., release of tumor necrosis factor, interleukin-6 and/or IL-1alpha), and Toll-like receptor mediated activation, e.g., TLR4 activation., CD32b ablation can also enhance macrophage ADCP and ADCC activity in vivo (as further disclosed in, e.g., Clynes et al., Nature Medicine 6, 443-446 (2000).

It has also been shown that CD32b ITIM immunosuppressive signaling reduces, e.g., inhibits, DC maturation. CD32b blockade or knockout can improve DC maturation and T cell priming, thus enhancing immunologic memory (Brochov et al., JCI 2005; Dhodapkar et al., PNSA 2005).

Without wishing to be bound by theory, it is believed that binding of the anti-CD32b antibodies or antigen-binding fragments thereof disclosed herein to CD32b, can prevent, e.g., inhibit, the inhibitory activites of CD32b described herein, thus blocking the immunosuppressive activity of CD32b. In some embodiments, the anti-CD32b antibodies or antigen-binding fragments thereof disclosed herein can result in one or more of:

decreased B cell inhibition,

increased B cell activation;

enhanced immune cell-mediated ADCC, e.g., macrophage- or NK cell-mediated ADCC;

enhanced macrophage-mediated ADCP; or

enhanced DC activity, e.g., DC maturation, antigen presentation and T cell priming.

### Prophylactic and Therapeutic Uses

The present disclosure provides methods of treating a disease or disorder, e.g., a hyperproliferative disease or disorder (e.g., a cancer), associated with increased CD32b activity or expression by administering to a subject in need thereof an effective amount of an anti-CD32b antibody or antigen-binding fragment thereof disclosed herein, alone or in combination with one or more of a second or additional therapeutic agent described herein, e.g., one or more second therapeutic agents, wherein the second therapeutic agent is chosen from one or more of:

(i) an antibody that binds a cell surface antigen on a cancer cell, tumor cell, or an immune cell;

(ii) an immunomodulatory compound; or

(iii) an anti-cancer therapy. In a specific embodiment, the present disclosure provides a method of treating indications including, but not limited to, B cell malignancies including Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma and follicular lymphoma as well as other diseases including systemic light chain amyloidosis.

In one embodiment, the present disclosure provides methods of treating a CD32b-related disease or disorder, e.g., a hyperproliferative disease or disorder (e.g., a cancer). Examples of known CD32b related diseases or disorders for which the disclosed CD32b binding antibodies, or antigen-binding fragments thereof, may be useful include but is not limited to: B cell malignancies including Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma and follicular lymphoma as well as other diseases including systemic light chain amyloidosis. In certain embodiments, the cancer is an epithelial, mesenchymal or hematologic malignancy.

In certain embodiments, the cancer treated is a solid tumor (e.g., carcinoid, carcinoma or sarcoma), a soft tissue tumor (e.g., a heme malignancy), and a metastatic lesion, e.g., a metastatic lesion of any of the cancers disclosed herein. In one embodiment, the cancer treated is a fibrotic or desmoplastic solid tumor, e.g., a tumor having one or more of: limited tumor perfusion, compressed blood vessels, fibrotic tumor interstitium, or increased interstitial fluid pressure. In one embodiment, the solid tumor is chosen from one or more of pancreatic (e.g., pancreatic adenocarcinoma or pancreatic ductal adenocarcinoma), breast, colon, colorectal, lung (e.g., small cell lung cancer (SCLC) or non-small cell lung cancer (NSCLC)), skin, ovarian, liver cancer, esophageal cancer, endometrial cancer, gastric cancer, head and neck cancer, kidney, or prostate cancer.

By “hyperproliferative disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.”

Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In addition, the antibodies or antigen-binding fragments thereof of the disclosure can be used, inter alia, in combination with another antibody that binds to a cell surface antigen co-expressed with CD32b, to increase efficacy of the other antibody. In some embodiments, CD32b and the cell surface antigen are co-expressed on B cells. In some embodiments, the cell surface antigen is selected from the group consisting of CD20, CD38, CD52, CS1/SLAMF7, KiR, CD56, CD138, CD19, CD40, Thy-1, Ly-6, CD49, Fas, Cd95, APO-1, EGFR, HER2, CXCR4, HLA molecules, GM1, CD22, CD23, CD80, CD74, DRD, CD33, ERBB2 (HER2/Neu), TSHR, CD171, CS-1, CLL-1, GD3, Tn Ag, FLT3, CD44v6, B7H3, KIT, IL-13Ra2, IL-11Ra, PSCA, PRSS21, VEGF, VEGFR2, LewisY, CD24, PDGFR-beta, SSEA-4, MUC1, NCAM, CAIX, LMP2, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, and IGLL1. In some embodiments, the cell surface antigen is CD38. In some embodiments, the other CD32b-binding antibodies, of the disclosure are used in combination with an antibody that binds to a cell surface antigen selected from the group consisting of rituximab, obinutuzumab, ofatumumab, daratuximab, elotuzumab, alemtuzumab, Ocrelizumab, Veltuzumab, GA101, Gemtuzumab ogozamicin, lintuzumab, cetuximab, panitumumab, zalutumumab, nimotuzumab, matuzumab, trastuzumab, pertuzumab, bevacizumab, trastuzumab, ibritumomab tiuxetan, or any other antibody that targets a cell surface antigen co-expressed with CD32b. In some embodiments, the antibody is daratumumab.

Without wishing to be bound by theory, an explanation for the observation that the anti-CD32b antibodies, or antigen-binding fragments thereof, of the disclosure can enhance the activity of other antibodies that bind to cell surface antigens co-expressed with CD32b is that the anti-CD32b antibodies bind to CD32b and can block CD32b from binding the Fc region of the cell surface antigen-binding antibody, which allows the cell surface antigen-binding antibody to engage immune effectors cells and mediate cell killing functions (e.g. via ADCC), and potentially prevents the cell surface antigen-binding antibody from being internalized into the cell and therefore not mediate cell killing (e.g. via ADCC).

Furthermore, the CD32b binding antibodies or antigen-binding fragments thereof of the disclosure can be used, inter alia, to treat, e.g., delay or reverse disease progression of patients who have become resistant or refractory to treatments using antibodies that bind to cell surface antigens, e.g., cell surface antigens that are co-expressed with CD32b. Without wishing to be bound by theory, it is believed that by blocking CD32b with the CD32b-binding antibodies, or antigen binding fragments thereof disclosed herein, the efficacy of the cell surface antigen binding antibodies may be enhanced and therefore resistance to such antibodies can be reversed, in full or in part.

### Combination Therapies

In one embodiment, the anti-CD32b antibody molecules described herein can be administered to a patient in need thereof in conjunction with a therapeutic method or procedure, such as described herein or known in the art. In addition, anti-CD32b antibody molecules, of the present disclosure, either alone or in combination with one or more antibodies may be further combined with another therapeutic agent as discussed below.

For example, the combination therapy can include a composition of the present disclosure co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the antibody molecules are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

Therapeutic antibodies achieve their effects through various mechanisms. The antibodies can have direct effects, e.g., inducing apoptosis on target cells, or indirect effects, e.g., recruiting immune effector cells that have cytotoxicity, e.g., monocytes, macrophages, or natural killer cells, resulting in elimination of the target cells via antibody-dependent cell mediated cytotoxicity (ADCC). The recruitment of immune effector cells to target cells by antibody molecules occurs via the Fc region of antibodies, e.g., binding of Fc region of antibodies to Fc gamma receptors, e.g., activating or inhibitory Fc gamma receptors, on immune effector cells. While binding of activating Fc gamma receptors by therapeutic antibodies can result in ADCC, binding of inhibitory Fc gamma receptors, e.g., CD32b, by therapeutic antibodies can result in any of the inhibitory effects of CD32b described herein, e.g., reduced ADCC or ADCP by macrophages, inhibition of DC maturation and antigen presentation, reduced T cell priming and inhibition of activating signals on B cells, inter alia.

It has been demonstrated, e.g., by Lim et al. (Blood 2011: 118(9) 2530-2540) and Vaughan et al. (Blood 2014: 123(5) 669-677) that CD32b binds the Fc of CD20 bound rituximab causing the tripartite complex to internalize and ultimately resulting in reduced CD20 bound rituximab coating the lymphoma cell surface. It has also been proposed that CD32b on lymphoma cells engage the Fc region of, for example, CD20 bound rituximab in cis effectively masking the rituximab Fc. The anticipated consequence of the rituximab Fc masking is a reduced opportunity to engage the activatory FcγR on effector cells in trans (Vaughan et al. Blood 2014: 123(5) 669-677).

Without wishing to be bound by theory, it is believed that, an anti-CD32b antibody described herein can prevent the ligation of CD32b by therapeutic antibodies by occupying the Fc binding site on CD32b thus allowing the Fc domain of therapeutic antibodies to engage activatory Fc gamma receptors. In some embodiments, this effect can be achieved by co-administering an anti-CD32b antibody with a second antibody, e.g., an antibody with an Fc domain. In some embodiments, the second antibody can be chosen from one or more of any antibody described herein.

Binding of anti-CD32b antibody molecules disclosed herein to CD32b, e.g., binding of anti-CD32b antibodies to extracellular Fc-binding domains on CD32b, can also inhibit, e.g., block, binding of other immunoglobulin Fc molecules, e.g., immunoglobulin Fc molecules of other antibodies, to CD32b. In this regard, anti-CD32b antibodies can act as inhibitors of inhibitors, e.g., preventing an inhibitory activity of ITIM signaling upon engagement of the extracellular CD32b Fc-binding domain with an Fc molecule, by blocking said extracellular Fc-binding sites on CD32b from being bound by other immunoglobulin Fc molecules by binding, e.g., occupying, said sites.

Without wishing to be bound by theory, it is believed that in some embodiments, co-administration of an anti-CD32b antibody molecule disclosed herein with a second antibody, e.g., an antibody comprising an Fc domain, prevents, e.g., inhibits, the inhibitory activities of CD32b on the second antibody, e.g., the non-CD32b binding antibody, thus blocking the immunosuppressive activity of CD32b.

In some embodiments, co-administration of an anti-CD32b antibody molecule disclosed herein with a second antibody, e.g., an antibody comprising an Fc domain, can result in one or more of:

decreased B cell inhibition,

increased B cell activation;

enhanced immune cell-mediated ADCC, e.g., macrophage- or NK cell-mediated ADCC;

enhanced macrophage-mediated ADCP; or enhanced DC activity, e.g., DC maturation, antigen presentation and T cell priming thereby increasing the efficacy of said second antibody.

The phrase “in combination with,” is not intended to imply that the therapy or the therapeutic agents must be administered at the same time and/or formulated for delivery together, although these methods of delivery are within the scope described herein. The anti-CD32b antibody molecules can be administered concurrently with, prior to, or subsequent to, one or more other additional therapies or therapeutic agents. The anti-CD32b antibody molecule and the other agent or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

Exemplary combinations of anti-CD32b antibody molecules include using such antibodies in combination with compounds that are standard of care agents for treating hematologic malignancies, including multiple myeloma, non-Hodgkins lymphoma, and chronic lymphocytic lymphoma, including but not limited to ofatumumab, ibrutinib, belinostat, romidepsin, brentuximab vedotin, obinutuzumab, pralatrexate, pentostatin, dexamethasone, idelalisib, ixazomib, liposomal doxyrubicin, pomalidomide, panobinostat, elotuzumab, daratumumab, alemtuzumab, thalidomide, and lenalidomide. In some embodiments, the agent is daratumumab.

In one embodiment, the anti-CD32b antibody molecule is administered in combination with one or more of a second or additional therapeutic agent chosen from: (i) an antibody that binds a cell surface antigen, e.g., a surface antigen on an immune cell or a cancer or a tumor cell; (ii) an immunomodulatory compound or an immune-based therapy, e.g., one or more of a cytokine, an activator of a costimulatory molecule, or an inhibitor of an inhibitory molecule (e.g., an inhibitor of a checkpoint inhibitor), as described herein; (iii) an anti-cancer therapy, e.g., one or more of a targeted anti-cancer therapy (e.g., an antibody molecule), or a cytotoxic agent (e.g., a chemotherapy, an oncolytic drug, or a small molecule inhibitor as described herein).

In one embodiment, the anti-CD32b antibody molecule is administered in combination with an immunomodulator, e.g., a cytokine. In embodiments, the cytokine is chosen from one or more of IL-2, IL-6, IL-7, IL-9, IL-12, IL-15, IL-18, IL-21, IL-23, or IL-27, including variant forms thereof (e.g., a cytokine derivative, a complex comprising the cytokine molecule with a polypeptide, e.g., a cytokine receptor complex, and other agonist forms thereof). In one embodiment, the cytokine molecule is an IL-15 including variant forms thereof.

In one embodiment, the anti-CD32b antibody molecule is administered in combination with an immunomodulator, e.g., an agonist, of a costimulatory molecule. In one embodiment, the agonist of the costimulatory molecule is chosen from an agonist (e.g., an agonistic antibody or antigen-binding fragment thereof, or a soluble fusion) of STING, OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In one embodiment, the anti-CD32b antibody molecule is administered in combination with an immunomodulatory, e.g., an inhibitor of an inhibitory molecule. In embodiments, the inhibitor is chosen from an inhibitor of PD-1, PD-L1, PD-L2, CTLA-4, TIM-3, LAG-3, CEACAM (e.g., CEACAM-1, CEACAM-3, and/or CEACAM-5), VISTA, BTLA, TIGIT, LAIR1, CD160, 2B4, TGF beta, and IDO (indoleamine-2,3 dioxygenase). Inhibition of an inhibitory molecule can be performed by inhibition at the DNA, RNA or protein level.

### Exemplary PD-1 Inhibitors

In certain embodiments, the anti-CD32b antibodies disclosed herein herein can be administered in combination with a PD-1 inhibitor. The PD-1 inhibitor may be an antibody, an antigen binding fragment thereof, an immunoadhesin, a fusion protein, or an oligopeptide. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

### Exemplary Anti-PD-1 Antibody Molecules

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of BAP049-Clone-E or BAP049-Clone-B disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In some embodiments, the CDRs are according to the combined CDR definitions of both Kabat and Chothia (e.g., as set out in Table 5). In one embodiment, the combination of Kabat and Chothia CDR of VH CDR1 comprises the amino acid sequence GYTFTTYWMH (SEQ ID NO: 1541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-PD-1 antibody molecule comprises a heavy chain variable region (VH) comprising a VHCDR1 amino acid sequence of SEQ ID NO: 1501, a VHCDR2 amino acid sequence of SEQ ID NO: 1502, and a VHCDR3 amino acid sequence of SEQ ID NO: 1503; and a light chain variable region (VL) comprising a VLCDR1 amino acid sequence of SEQ ID NO: 1510, a VLCDR2 amino acid sequence of SEQ ID NO: 1511, and a VLCDR3 amino acid sequence of SEQ ID NO: 1512, each disclosed in Table 5.

In one embodiment, the antibody molecule comprises a VH comprising a VHCDR1 encoded by the nucleotide sequence of SEQ ID NO: 1524, a VHCDR2 encoded by the nucleotide sequence of SEQ ID NO: 1525, and a VHCDR3 encoded by the nucleotide sequence of SEQ ID NO: 1526; and a VL comprising a VLCDR1 encoded by the nucleotide sequence of SEQ ID NO: 1529, a VLCDR2 encoded by the nucleotide sequence of SEQ ID NO: 1530, and a VLCDR3 encoded by the nucleotide sequence of SEQ ID NO: 1531, each disclosed in Table 5.

In one embodiment, the anti-PD-1 antibody molecule comprises a VH comprising the amino acid sequence of SEQ ID NO: 1506, or an amino acid sequence having at least about 85%, 90%, 95%, or 99% sequence identity or higher to SEQ ID NO: 1506. In one embodiment, the anti-PD-1 antibody molecule comprises a VL comprising the amino acid sequence of SEQ ID NO: 1520, or an amino acid sequence having at least about 85%, 90%, 95%, or 99% sequence identity or higher to SEQ ID NO: 1520. In one embodiment, the anti-PD-1 antibody molecule comprises a VL comprising the amino acid sequence of SEQ ID NO: 1516, or an amino acid sequence having at least about 85%, 90%, 95%, or 99% sequence identity or higher to SEQ ID NO: 1516. In one embodiment, the anti-PD-1 antibody molecule comprises a VH comprising the amino acid sequence of SEQ ID NO: 1506 and a VL comprising the amino acid sequence of SEQ ID NO: 1520. In one embodiment, the anti-PD-1 antibody molecule comprises a VH comprising the amino acid sequence of SEQ ID NO: 1506 and a VL comprising the amino acid sequence of SEQ ID NO: 1516.

In one embodiment, the antibody molecule comprises a VH encoded by the nucleotide sequence of SEQ ID NO: 1507, or a nucleotide sequence having at least about 85%, 90%, 95%, or 99% sequence identity or higher to SEQ ID NO: 1507. In one embodiment, the antibody molecule comprises a VL encoded by the nucleotide sequence of SEQ ID NO: 1521 or 1517, or a nucleotide sequence having at least about 85%, 90%, 95%, or 99% sequence identity or higher to SEQ ID NO: 1521 or 1517. In one embodiment, the antibody molecule comprises a VH encoded by the nucleotide sequence of SEQ ID NO: 1507 and a VL encoded by the nucleotide sequence of SEQ ID NO: 1521 or 1517.

In one embodiment, the anti-PD-1 antibody molecule comprises a heavy chain comprising the amino acid sequence of SEQ ID NO: 1508, or an amino acid sequence having at least about 85%, 90%, 95%, or 99% sequence identity or higher to SEQ ID NO: 1508. In one embodiment, the anti-PD-1 antibody molecule comprises a light chain comprising the amino acid sequence of SEQ ID NO: 1522, or an amino acid sequence having at least about 85%, 90%, 95%, or 99% sequence identity or higher to SEQ ID NO: 1522. In one embodiment, the anti-PD-1 antibody molecule comprises a light chain comprising the amino acid sequence of SEQ ID NO: 1518, or an amino acid sequence having at least about 85%, 90%, 95%, or 99% sequence identity or higher to SEQ ID NO: 1518. In one embodiment, the anti-PD-1 antibody molecule comprises a heavy chain comprising the amino acid sequence of SEQ ID NO: 1508 and a light chain comprising the amino acid sequence of SEQ ID NO: 1522. In one embodiment, the anti-PD-1 antibody molecule comprises a heavy chain comprising the amino acid sequence of SEQ ID NO: 1508 and a light chain comprising the amino acid sequence of SEQ ID NO: 1518.

In one embodiment, the antibody molecule comprises a heavy chain encoded by the nucleotide sequence of SEQ ID NO: 1509, or a nucleotide sequence having at least about 85%, 90%, 95%, or 99% sequence identity or higher to SEQ ID NO: 1509. In one embodiment, the antibody molecule comprises a light chain encoded by the nucleotide sequence of SEQ ID NO: 1523 or 1519, or a nucleotide sequence having at least about 85%, 90%, 95%, or 99% sequence identity or higher to SEQ ID NO: 1523 or 1519. In one embodiment, the antibody molecule comprises a heavy chain encoded by the nucleotide sequence of SEQ ID NO: 1509 and a light chain encoded by the nucleotide sequence of SEQ ID NO: 1523 or 1519.

The antibody molecules described herein can be made by vectors, host cells, and methods described in US 2015/0210769, incorporated by reference in its entirety.

TABLE 5
Amino acid and nucleotide sequences of exemplary
anti-PD-1 antibody molecules
BAP049-Clone-B HC

| | | |
|---|---|---|
| SEQ ID NO: 1501 | HCDR1 | TYWMH |
| (Kabat) | | |
| SEQ ID NO: 1502 | HCDR2 | NIYPGTGGSNFDEKFKN |
| (Kabat) | | |
| SEQ ID NO: 1503 | HCDR3 | WTTGTGAY |
| (Kabat) | | |
| SEQ ID NO: 1504 | HCDR1 | GYTFTTY |
| (Chothia) | | |
| SEQ ID NO: 1505 | HCDR2 | YPGTGG |
| (Chothia) | | |
| SEQ ID NO: 1503 | HCDR3 | WTTGTGAY |
| (Chothia) | | |
| SEQ ID NO: 1506 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 1507 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 1508 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 1509 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |

BAP049-Clone-B LC

| | | |
|---|---|---|
| SEQ ID NO: 1510 | LCDR1 | KSSQSLLDSGNQKNFLT |
| (Kabat) | | |
| SEQ ID NO: 1511 | LCDR2 | WASTRES |
| (Kabat) | | |
| SEQ ID NO: 1512 | LCDR3 | QNDYSYPYT |
| (Kabat) | | |
| SEQ ID NO: 1513 | LCDR1 | SQSLLDSGNQKNF |
| (Chothia) | | |
| SEQ ID NO: 1514 | LCDR2 | WAS |
| (Chothia) | | |
| SEQ ID NO: 1515 | LCDR3 | DYSYPY |
| (Chothia) | | |
| SEQ ID NO: 1516 | VL | EIVLTQSPATLSLSPGERATLSCKSSQSLLDSGNQKNFLTWY |
| | | QQKPGKAPKLLIYWASTRESGVPSRFSGSGSGTDFTFTISSLQ |
| | | PEDIATYYCQNDYSYPYTFGQGTKVEIK |
| SEQ ID NO: 1517 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAG |
| SEQ ID NO: 1518 | Light | EIVLTQSPATLSLSPGERATLSCKSSQSLLDSGNQKNFLTWY |
| | chain | QQKPGKAPKLLIYWASTRESGVPSRFSGSGSGTDFTFTISSLQ |
| | | PEDIATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 1519 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |

BAP049-Clone-E HC

| | | |
|---|---|---|
| SEQ ID NO: 1501 | HCDR1 | TYWMH |
| (Kabat) | | |
| SEQ ID NO: 1502 | HCDR2 | NIYPGTGGSNFDEKFKN |
| (Kabat) | | |
| SEQ ID NO: 1503 | HCDR3 | WTTGTGAY |
| (Kabat) | | |
| SEQ ID NO: 1504 | HCDR1 | GYTFTTY |
| (Chothia) | | |
| SEQ ID NO: 1505 | HCDR2 | YPGTGG |
| (Chothia) | | |
| SEQ ID NO: 1503 | HCDR3 | WTTGTGAY |
| (Chothia) | | |
| SEQ ID NO: 1506 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 1507 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 1508 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 1509 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |

BAP049-Clone-E LC

| | | |
|---|---|---|
| SEQ ID NO: 1510 | LCDR1 | KSSQSLLDSGNQKNFLT |
| (Kabat) | | |
| SEQ ID NO: 1511 | LCDR2 | WASTRES |
| (Kabat) | | |
| SEQ ID NO: 1512 | LCDR3 | QNDYSYPYT |
| (Kabat) | | |
| SEQ ID NO: 1513 | LCDR1 | SQSLLDSGNQKNF |
| (Chothia) | | |
| SEQ ID NO: 1514 | LCDR2 | WAS |
| (Chothia) | | |
| SEQ ID NO: 1515 | LCDR3 | DYSYPY |
| (Chothia) | | |
| SEQ ID NO: 1520 | VL | EIVLTQSPATLSLSPGERATLSCKSSQSLLDSGNQKNFLTWY |
| | | QQKPGQAPRLLIYWASTRESGVPSRFSGSGSGTDFTFTISSLE |
| | | AEDAATYYCQNDYSYPYTFGQGTKVEIK |
| SEQ ID NO: 1521 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAG |
| SEQ ID NO: 1522 | Light | EIVLTQSPATLSLSPGERATLSCKSSQSLLDSGNQKNFLTWY |
| | chain | QQKPGQAPRLLIYWASTRESGVPSRFSGSGSGTDFTFTISSLE |
| | | AEDAATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 1523 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |

BAP049-Clone-B HC

| | | |
|---|---|---|
| SEQ ID NO: 1524 | HCDR1 | ACCTACTGGATGCAC |
| (Kabat) | | |
| SEQ ID NO: 1525 | HCDR2 | AATATCTACCCCGGCACCGGCGGCTCTAACTTCGACGAG |
| (Kabat) | | AAGTTTAAGAAT |
| SEQ ID NO: 1526 | HCDR3 | TGGACTACCGGCACAGGCGCCTAC |
| (Kabat) | | |
| SEQ ID NO: 1527 | HCDR1 | GGCTACACCTTCACTACCTAC |
| (Chothia) | | |
| SEQ ID NO: 1528 | HCDR2 | TACCCCGGCACCGGCGGC |
| (Chothia) | | |
| SEQ ID NO: 1526 | HCDR3 | TGGACTACCGGCACAGGCGCCTAC |
| (Chothia) | | |

BAP049-Clone-B LC

| | | |
|---|---|---|
| SEQ ID NO: 1529 | LCDR1 | AAATCTAGTCAGTCACTGCTGGATAGCGGTAATCAGAAG |
| (Kabat) | | AACTTCCTGACC |
| SEQ ID NO: 1530 | LCDR2 | TGGGCCTCTACTAGAGAATCA |
| (Kabat) | | |
| SEQ ID NO: 1531 | LCDR3 | CAGAACGACTATAGCTACCCCTACACC |
| (Kabat) | | |
| SEQ ID NO: 1532 | LCDR1 | AGTCAGTCACTGCTGGATAGCGGTAATCAGAAGAACTTC |
| (Chothia) | | |
| SEQ ID NO: 1533 | LCDR2 | TGGGCCTCT |
| (Chothia) | | |
| SEQ ID NO: 1534 | LCDR3 | GACTATAGCTACCCCTAC |
| (Chothia) | | |

BAP049-Clone-E HC

| | | |
|---|---|---|
| SEQ ID NO: 1524 | HCDR1 | ACCTACTGGATGCAC |
| (Kabat) | | |
| SEQ ID NO: 1525 | HCDR2 | AATATCTACCCCGGCACCGGCGGCTCTAACTTCGACGAG |
| (Kabat) | | AAGTTTAAGAAT |
| SEQ ID NO: 1526 | HCDR3 | TGGACTACCGGCACAGGCGCCTAC |
| (Kabat) | | |
| SEQ ID NO: 1527 | HCDR1 | GGCTACACCTTCACTACCTAC |
| (Chothia) | | |
| SEQ ID NO: 1528 | HCDR2 | TACCCCGGCACCGGCGGC |
| (Chothia) | | |
| SEQ ID NO: 1526 | HCDR3 | TGGACTACCGGCACAGGCGCCTAC |
| (Chothia) | | |

BAP049-Clone-E LC

| | | |
|---|---|---|
| SEQ ID NO: 1529 | LCDR1 | AAATCTAGTCAGTCACTGCTGGATAGCGGTAATCAGAAG |
| (Kabat) | | AACTTCCTGACC |
| SEQ ID NO: 1530 | LCDR2 | TGGGCCTCTACTAGAGAATCA |
| (Kabat) | | |
| SEQ ID NO: 1531 | LCDR3 | CAGAACGACTATAGCTACCCCTACACC |
| (Kabat) | | |
| SEQ ID NO: 1532 | LCDR1 | AGTCAGTCACTGCTGGATAGCGGTAATCAGAAGAACTTC |
| (Chothia) | | |
| SEQ ID NO: 1533 | LCDR2 | TGGGCCTCT |
| (Chothia) | | |
| SEQ ID NO: 1534 | LCDR3 | GACTATAGCTACCCCTAC |
| (Chothia) | | |

### Other Exemplary PD-1 Inhibitors

In one embodiment, the anti-PD-1 antibody molecule is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 6.

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 6.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 6.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

TABLE 6
Amino acid sequences of other exemplary
anti-PD-1 antibody molecules
Nivolumab

| | | |
|---|---|---|
| SEQ | Heavy | QVQLVESGGGVVQPGRSLRLDCKASGITFSN |
| ID | chain | SGMHWVRQAPGKGLEWVAVIWYDGSKRYYAD |
| NO: | | SVKGRFTISRDNSKNTLFLQMNSLRAEDTAV |
| 1535 | | YYCATNDDYWGQGTLVTVSSASTKGPSVFPL |
| | | APCSRSTSESTAALGCLVKDYFPEPVTVSWN |
| | | SGALTSGVHTFPAVLQSSGLYSLSSVVTVPS |
| | | SSLGTKTYTCNVDHKPSNTKVDKRVESKYGP |
| | | PCPPCPAPEFLGGPSVFLFPPKPKDTLMISR |
| | | TPEVTCVVVDVSQEDPEVQFNWYVDGVEVHN |
| | | AKTKPREEQFNSTYRVVSVLTVLHQDWLNGK |
| | | EYKCKVSNKGLPSSIEKTISKAKGQPREPQV |
| | | YTLPPSQEEMTKNQVSLTCLVKGFYPSDIAV |
| | | EWESNGQPENNYKTTPPVLDSDGSFFLYSRL |
| | | TVDKSRWQEGNVFSCSVMHEALHNHYTQKSL |
| | | SLSLGK |
| SEQ | Light | EIVLTQSPATLSLSPGERATLSCRASQSVSS |
| ID | chain | YLAWYQQKPGQAPRLLIYDASNRATGIPARF |
| NO: | | SGSGSGTDFTLTISSLEPEDFAVYYCQQSSN |
| 1536 | | WPRTFGQGTKVEIKRTVAAPSVFIFPPSDEQ |
| | | LKSGTASVVCLLNNFYPREAKVQWKVDNALQ |
| | | SGNSQESVTEQDSKDSTYSLSSTLTLSKADY |
| | | EKHKVYACEVTHQGLSSPVTKSFNRGEC |

Pembrolizumab

| | | |
|---|---|---|
| SEQ | Heavy | QVQLVQSGVEVKKPGASVKVSCKASGYTFTN |
| ID | chain | YYMYWVRQAPGQGLEWMGGINPSNGGTNFNE |
| NO: | | KFKNRVTLTTDSSTTTAYMELKSLQFDDTAV |
| 1537 | | YYCARRDYRFDMGFDYWGQGTTVTVSSASTK |
| | | GPSVFPLAPCSRSTSESTAALGCLVKDYFPE |
| | | PVTVSWNSGALTSGVHTFPAVLQSSGLYSLS |
| | | SVVTVPSSSLGTKTYTCNVDHKPSNTKVDKR |
| | | VESKYGPPCPPCPAPEFLGGPSVFLFPPKPK |
| | | DTLMISRTPEVTCVVVDVSQEDPEVQFNWYV |
| | | DGVEVHNAKTKPREEQFNSTYRVVSVLTVLH |
| | | QDWLNGKEYKCKVSNKGLPSSIEKTISKAKG |
| | | QPREPQVYTLPPSQEEMTKNQVSLTCLVKGF |
| | | YPSDIAVEWESNGQPENNYKTTPPVLDSDGS |
| | | FFLYSRLTVDKSRWQEGNVFSCSVMHEALHN |
| | | HYTQKSLSLSLGK |
| SEQ | Light | EIVLTQSPATLSLSPGERATLSCRASKGVST |
| ID | chain | SGYSYLHWYQQKPGQAPRLLIYLASYLESGV |
| NO: | | PARFSGSGSGTDFTLTISSLEPEDFAVYYCQ |
| 1538 | | HSRDLPLTFGGGTKVEIKRTVAAPSVFIFPP |
| | | SDEQLKSGTASVVCLLNNFYPREAKVQWKVD |
| | | NALQSGNSQESVTEQDSKDSTYSLSSTLTLS |
| | | KADYEKHKVYACEVTHQGLSSPVTKSFNRGE |
| | | C |

Pidilizumab

| | | |
|---|---|---|
| SEQ | Heavy | QVQLVQSGSELKKPGASVKISCKASGYTFTN |
| ID | chain | YGMNWVRQAPGQGLQWMGWINTDSGESTYAE |
| NO: | | EFKGRFVFSLDTSVNTAYLQITSLTAEDTGM |
| 1539 | | YFCVRVGYDALDYWGQGTLVTVSSASTKGPS |
| | | VFPLAPSSKSTSGGTAALGCLVKDYFPEPVT |
| | | VSWNSGALTSGVHTFPAVLQSSGLYSLSSVV |
| | | TVPSSSLGTQTYICNVNHKPSNTKVDKRVEP |
| | | KSCDKTHTCPPCPAPELLGGPSVFLFPPKPK |
| | | DTLMISRTPEVTCVVVDVSHEDPEVKFNWYV |
| | | DGVEVHNAKTKPREEQYNSTYRVVSVLTVLH |
| | | QDWLNGKEYKCKVSNKALPAPIEKTISKAKG |
| | | QPREPQVYTLPPSREEMTKNQVSLTCLVKGF |
| | | YPSDIAVEWESNGQPENNYKTTPPVLDSDGS |
| | | FFLYSKLTVDKSRWQQGNVFSCSVMHEALHN |
| | | HYTQKSLSLSPGK |
| SEQ | Light | EIVLTQSPSSLSASVGDRVTITCSARSSVSY |
| ID | chain | MHWFQQKPGKAPKLWIYRTSNLASGVPSRFS |
| NO: | | GSGSGTSYCLTINSLQPEDFATYYCQQRSSF |
| 1540 | | PLTFGGGTKLEIKRTVAAPSVFIFPPSDEQL |
| | | KSGTASVVCLLNNFYPREAKVQWKVDNALQS |
| | | GNSQESVTEQDSKDSTYSLSSTLTLSKADYE |
| | | KHKVYACEVTHQGLSSPVTKSFNRGEC |

### Exemplary PD-L1 Inhibitors

In certain embodiments, the anti-CD32b antibodies disclosed herein can be administeredin combination with a PD-L1 inhibitor. The PD-L1 inhibitor may be an antibody, an antigen binding fragment thereof, an immunoadhesin, a fusion protein, or an oligopeptide. In some embodiments, the PD-L1 inhibitor is chosen from FAZ053 (Novartis), Atezolizumab (Genentech/Roche), Avelumab (Merck Serono and Pfizer), Durvalumab (Medlmmune/AstraZeneca), or BMS-936559 (Bristol-Myers Squibb).

### Exemplary Anti-PD-L1 Antibody Molecules

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of BAP058-Clone 0 or BAP058-Clone N disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In some embodiments, the CDRs are according to the combined CDR definitions of both Kabat and Chothia (e.g., as set out in Table 7). In one embodiment, the combination of Kabat and Chothia CDR of VH CDR1 comprises the amino acid sequence GYTFTSYWMY (SEQ ID NO: 1647). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-PD-L1 antibody molecule comprises a heavy chain variable region (VH) comprising a VHCDR1 amino acid sequence of SEQ ID NO: 1601, a VHCDR2 amino acid sequence of SEQ ID NO: 1602, and a VHCDR3 amino acid sequence of SEQ ID NO: 1603; and a light chain variable region (VL) comprising a VLCDR1 amino acid sequence of SEQ ID NO: 1609, a VLCDR2 amino acid sequence of SEQ ID NO: 1610, and a VLCDR3 amino acid sequence of SEQ ID NO: 1611, each disclosed in Table 7.

In one embodiment, the anti-PD-L1 antibody molecule comprises a VH comprising a VHCDR1 encoded by the nucleotide sequence of SEQ ID NO: 1628, a VHCDR2 encoded by the nucleotide sequence of SEQ ID NO: 1629, and a VHCDR3 encoded by the nucleotide sequence of SEQ ID NO: 1630; and a VL comprising a VLCDR1 encoded by the nucleotide sequence of SEQ ID NO: 1633, a VLCDR2 encoded by the nucleotide sequence of SEQ ID NO: 1634, and a VLCDR3 encoded by the nucleotide sequence of SEQ ID NO: 1635, each disclosed in Table 7.

In one embodiment, the anti-PD-L1 antibody molecule comprises a VH comprising the amino acid sequence of SEQ ID NO: 1606, or an amino acid sequence having at least about 85%, 90%, 95%, or 99% sequence identity to SEQ ID NO: 1606. In one embodiment, the anti-PD-L1 antibody molecule comprises a VL comprising the amino acid sequence of SEQ ID NO: 1616, or an amino acid sequence having at least about 85%, 90%, 95%, or 99% sequence identity or higher to SEQ ID NO: 1616. In one embodiment, the anti-PD-L1 antibody molecule comprises a VH comprising the amino acid sequence of SEQ ID NO: 1620, or an amino acid sequence having at least about 85%, 90%, 95%, or 99% sequence identity to SEQ ID NO: 1620. In one embodiment, the anti-PD-L1 antibody molecule comprises a VL comprising the amino acid sequence of SEQ ID NO: 1624, or an amino acid sequence having at least about 85%, 90%, 95%, or 99% sequence identity to SEQ ID NO: 1624. In one embodiment, the anti-PD-L1 antibody molecule comprises a VH comprising the amino acid sequence of SEQ ID NO: 1606 and a VL comprising the amino acid sequence of SEQ ID NO: 1616. In one embodiment, the anti-PD-L1 antibody molecule comprises a VH comprising the amino acid sequence of SEQ ID NO: 1620 and a VL comprising the amino acid sequence of SEQ ID NO: 1624.

In one embodiment, the antibody molecule comprises a VH encoded by the nucleotide sequence of SEQ ID NO: 1607, or a nucleotide sequence having at least about 85%, 90%, 95%, or 99% sequence identity to SEQ ID NO: 1607. In one embodiment, the antibody molecule comprises a VL encoded by the nucleotide sequence of SEQ ID NO: 1617, or a nucleotide sequence having at least about 85%, 90%, 95%, or 99% sequence identity to SEQ ID NO: 1617. In one embodiment, the antibody molecule comprises a VH encoded by the nucleotide sequence of SEQ ID NO: 1621, or a nucleotide sequence having at least about 85%, 90%, 95%, or 99% sequence identity to SEQ ID NO: 1621. In one embodiment, the antibody molecule comprises a VL encoded by the nucleotide sequence of SEQ ID NO: 1625, or a nucleotide sequence having at least about 85%, 90%, 95%, or 99% sequence identity to SEQ ID NO: 1625. In one embodiment, the antibody molecule comprises a VH encoded by the nucleotide sequence of SEQ ID NO: 1607 and a VL encoded by the nucleotide sequence of SEQ ID NO: 1617. In one embodiment, the antibody molecule comprises a VH encoded by the nucleotide sequence of SEQ ID NO: 1621 and a VL encoded by the nucleotide sequence of SEQ ID NO: 1625.

In one embodiment, the anti-PD-L1 antibody molecule comprises a heavy chain comprising the amino acid sequence of SEQ ID NO: 1608, or an amino acid sequence having at least about 85%, 90%, 95%, or 99% sequence identity to SEQ ID NO: 1608. In one embodiment, the anti-PD-L1 antibody molecule comprises a light chain comprising the amino acid sequence of SEQ ID NO: 1618, or an amino acid sequence having at least about 85%, 90%, 95%, or 99% sequence identity to SEQ ID NO: 1618. In one embodiment, the anti-PD-L1 antibody molecule comprises a heavy chain comprising the amino acid sequence of SEQ ID NO: 1622, or an amino acid sequence having at least about 85%, 90%, 95%, or 99% sequence identity to SEQ ID NO: 1622. In one embodiment, the anti-PD-L1 antibody molecule comprises a light chain comprising the amino acid sequence of SEQ ID NO: 1626, or an amino acid sequence having at least about 85%, 90%, 95%, or 99% sequence identity to SEQ ID NO: 1626. In one embodiment, the anti-PD-L1 antibody molecule comprises a heavy chain comprising the amino acid sequence of SEQ ID NO: 1608 and a light chain comprising the amino acid sequence of SEQ ID NO: 1618. In one embodiment, the anti-PD-L1 antibody molecule comprises a heavy chain comprising the amino acid sequence of SEQ ID NO: 1622 and a light chain comprising the amino acid sequence of SEQ ID NO: 1626.

In one embodiment, the antibody molecule comprises a heavy chain encoded by the nucleotide sequence of SEQ ID NO: 1615, or a nucleotide sequence having at least about 85%, 90%, 95%, or 99% sequence identity to SEQ ID NO: 1615. In one embodiment, the antibody molecule comprises a light chain encoded by the nucleotide sequence of SEQ ID NO: 1619, or a nucleotide sequence having at least about 85%, 90%, 95%, or 99% sequence identity to SEQ ID NO: 1619. In one embodiment, the antibody molecule comprises a heavy chain encoded by the nucleotide sequence of SEQ ID NO: 1623, or a nucleotide sequence having at least about 85%, 90%, 95%, or 99% sequence identity to SEQ ID NO: 1623. In one embodiment, the antibody molecule comprises a light chain encoded by the nucleotide sequence of SEQ ID NO: 1627, or a nucleotide sequence having at least about 85%, 90%, 95%, or 99% sequence identity to SEQ ID NO: 1627. In one embodiment, the antibody molecule comprises a heavy chain encoded by the nucleotide sequence of SEQ ID NO: 1615 and a light chain encoded by the nucleotide sequence of SEQ ID NO: 1619. In one embodiment, the antibody molecule comprises a heavy chain encoded by the nucleotide sequence of SEQ ID NO: 1623 and a light chain encoded by the nucleotide sequence of SEQ ID NO: 1627.

The antibody molecules described herein can be made by vectors, host cells, and methods described in US 2016/0108123, incorporated by reference in its entirety.

TABLE 7
Amino acid and nucleotide sequences of exemplary
anti-PD-L1 antibody molecules
BAP058-Clone O HC

| | | |
|---|---|---|
| SEQ ID NO: 1601 | HCDR1 | SYWMY |
| (Kabat) | | |
| SEQ ID NO: 1602 | HCDR2 | RIDPNSGSTKYNEKFKN |
| (Kabat) | | |
| SEQ ID NO: 1603 | HCDR3 | DYRKGLYAMDY |
| (Kabat) | | |
| SEQ ID NO: 1604 | HCDR1 | GYTFTSY |
| (Chothia) | | |
| SEQ ID NO: 1605 | HCDR2 | DPNSGS |
| (Chothia) | | |
| SEQ ID NO: 1603 | HCDR3 | DYRKGLYAMDY |
| (Chothia) | | |
| SEQ ID NO: 1606 | VH | EVQLVQSGAEVKKPGATVKISCKVSGYTFTSYWMYWVRQ |
| | | ARGQRLEWIGRIDPNSGSTKYNEKFKNRFTISRDNSKNTLY |
| | | LQMNSLRAEDTAVYYCARDYRKGLYAMDYWGQGTTVTV |
| | | SS |
| SEQ ID NO: 1607 | DNA | GAAGTGCAGCTGGTGCAGTCAGGCGCCGAAGTGAAGAA |
| | VH | ACCCGGCGCTACCGTGAAGATTAGCTGTAAAGTCTCAGG |
| | | CTACACCTTCACTAGCTACTGGATGTACTGGGTCCGACA |
| | | GGCTAGAGGGCAAAGACTGGAGTGGATCGGTAGAATCG |
| | | ACCCTAATAGCGGCTCTACTAAGTATAACGAGAAGTTTA |
| | | AGAATAGGTTCACTATTAGTAGGGATAACTCTAAGAACA |
| | | CCCTGTACCTGCAGATGAATAGCCTGAGAGCCGAGGAC |
| | | ACCGCCGTCTACTACTGCGCTAGAGACTATAGAAAGGGC |
| | | CTGTACGCTATGGACTACTGGGGTCAAGGCACTACCGTG |
| | | ACCGTGTCTTCA |
| SEQ ID NO: 1608 | Heavy | EVQLVQSGAEVKKPGATVKISCKVSGYTFTSYWMYWVRQ |
| | chain | ARGQRLEWIGRIDPNSGSTKYNEKFKNRFTISRDNSKNTLY |
| | | LQMNSLRAEDTAVYYCARDYRKGLYAMDYWGQGTTVTV |
| | | SSASTKGPSVFPLAPCSRSTSESTAALGCLVKDYFPEPVTVS |
| | | WNSGALTSGVHTFPAVLQSSGLYSLSSVVTVPSSSLGTKTY |
| | | TCNVDHKPSNTKVDKRVESKYGPPCPPCPAPEFLGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| SEQ ID NO: 1615 | DNA | GAAGTGCAGCTGGTGCAGTCAGGCGCCGAAGTGAAGAA |
| | heavy | ACCCGGCGCTACCGTGAAGATTAGCTGTAAAGTCTCAGG |
| | chain | CTACACCTTCACTAGCTACTGGATGTACTGGGTCCGACA |
| | | GGCTAGAGGGCAAAGACTGGAGTGGATCGGTAGAATCG |
| | | ACCCTAATAGCGGCTCTACTAAGTATAACGAGAAGTTTA |
| | | AGAATAGGTTCACTATTAGTAGGGATAACTCTAAGAACA |
| | | CCCTGTACCTGCAGATGAATAGCCTGAGAGCCGAGGAC |
| | | ACCGCCGTCTACTACTGCGCTAGAGACTATAGAAAGGGC |
| | | CTGTACGCTATGGACTACTGGGGTCAAGGCACTACCGTG |
| | | ACCGTGTCTTCAGCTAGCACTAAGGGCCCGTCCGTGTTC |
| | | CCCCTGGCACCTTGTAGCCGGAGCACTAGCGAATCCACC |
| | | GCTGCCCTCGGCTGCCTGGTCAAGGATTACTTCCCGGAG |
| | | CCCGTGACCGTGTCCTGGAACAGCGGAGCCCTGACCTCC |
| | | GGAGTGCACACCTTCCCCGCTGTGCTGCAGAGCTCCGGG |
| | | CTGTACTCGCTGTCGTCGGTGGTCACGGTGCCTTCATCTA |
| | | GCCTGGGTACCAAGACCTACACTTGCAACGTGGACCACA |
| | | AGCCTTCCAACACTAAGGTGGACAAGCGCGTCGAATCG |
| | | AAGTACGGCCCACCGTGCCCGCCTTGTCCCGCGCCGGAG |
| | | TTCCTCGGCGGTCCCTCGGTCTTTCTGTTCCCACCGAAGC |
| | | CCAAGGACACTTTGATGATTTCCCGCACCCCTGAAGTGA |
| | | CATGCGTGGTCGTGGACGTGTCACAGGAAGATCCGGAG |
| | | GTGCAGTTCAATTGGTACGTGGATGGCGTCGAGGTGCAC |
| | | AACGCCAAAACCAAGCCGAGGGAGGAGCAGTTCAACTC |
| | | CACTTACCGCGTCGTGTCCGTGCTGACGGTGCTGCATCA |
| | | GGACTGGCTGAACGGGAAGGAGTACAAGTGCAAAGTGT |
| | | CCAACAAGGGACTTCCTAGCTCAATCGAAAAGACCATCT |
| | | CGAAAGCCAAGGGACAGCCCCGGGAACCCCAAGTGTAT |
| | | ACCCTGCCACCGAGCCAGGAAGAAATGACTAAGAACCA |
| | | AGTCTCATTGACTTGCCTTGTGAAGGGCTTCTACCCATCG |
| | | GATATCGCCGTGGAATGGGAGTCCAACGGCCAGCCGGA |
| | | AAACAACTACAAGACCACCCCTCCGGTGCTGGACTCAGA |
| | | CGGATCCTTCTTCCTCTACTCGCGGCTGACCGTGGATAA |
| | | GAGCAGATGGCAGGAGGGAAATGTGTTCAGCTGTTCTGT |
| | | GATGCATGAAGCCCTGCACAACCACTACACTCAGAAGTC |
| | | CCTGTCCCTCTCCCTGGGA |

BAP058-Clone O LC

| | | |
|---|---|---|
| SEQ ID NO: 1609 | LCDR1 | KASQDVGTAVA |
| (Kabat) | | |
| SEQ ID NO: 1610 | LCDR2 | WASTRHT |
| (Kabat) | | |
| SEQ ID NO: 1611 | LCDR3 | QQYNSYPLT |
| (Kabat) | | |
| SEQ ID NO: 1612 | LCDR1 | SQDVGTA |
| (Chothia) | | |
| SEQ ID NO: 1613 | LCDR2 | WAS |
| (Chothia) | | |
| SEQ ID NO: 1614 | LCDR3 | YNSYPL |
| (Chothia) | | |
| SEQ ID NO: 1616 | VL | AIQLTQSPSSLSASVGDRVTITCKASQDVGTAVAWYLQKPG |
| | | QSPQLLIYWASTRHTGVPSRFSGSGSGTDFTFTISSLEAEDA |
| | | ATYYCQQYNSYPLTFGQGTKVEIK |
| SEQ ID NO: 1617 | DNA | GCTATTCAGCTGACTCAGTCACCTAGTAGCCTGAGCGCT |
| | VL | AGTGTGGGCGATAGAGTGACTATCACCTGTAAAGCCTCT |
| | | CAGGACGTGGGCACCGCCGTGGCCTGGTATCTGCAGAA |
| | | GCCTGGTCAATCACCTCAGCTGCTGATCTACTGGGCCTC |
| | | TACTAGACACACCGGCGTGCCCTCTAGGTTTAGCGGTAG |
| | | CGGTAGTGGCACCGACTTCACCTTCACTATCTCTTCACTG |
| | | GAAGCCGAGGACGCCGCTACCTACTACTGTCAGCAGTAT |
| | | AATAGCTACCCCCTGACCTTCGGTCAAGGCACTAAGGTC |
| | | GAGATTAAG |
| SEQ ID NO: 1618 | Light | AIQLTQSPSSLSASVGDRVTITCKASQDVGTAVAWYLQKPG |
| | chain | QSPQLLIYWASTRHTGVPSRFSGSGSGTDFTFTISSLEAEDA |
| | | ATYYCQQYNSYPLTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC |
| SEQ ID NO: 1619 | DNA | GCTATTCAGCTGACTCAGTCACCTAGTAGCCTGAGCGCT |
| | light | AGTGTGGGCGATAGAGTGACTATCACCTGTAAAGCCTCT |
| | chain | CAGGACGTGGGCACCGCCGTGGCCTGGTATCTGCAGAA |
| | | GCCTGGTCAATCACCTCAGCTGCTGATCTACTGGGCCTC |
| | | TACTAGACACACCGGCGTGCCCTCTAGGTTTAGCGGTAG |
| | | CGGTAGTGGCACCGACTTCACCTTCACTATCTCTTCACTG |
| | | GAAGCCGAGGACGCCGCTACCTACTACTGTCAGCAGTAT |
| | | AATAGCTACCCCCTGACCTTCGGTCAAGGCACTAAGGTC |
| | | GAGATTAAGCGTACGGTGGCCGCTCCCAGCGTGTTCATC |
| | | TTCCCCCCCAGCGACGAGCAGCTGAAGAGCGGCACCGC |
| | | CAGCGTGGTGTGCCTGCTGAACAACTTCTACCCCCGGGA |
| | | GGCCAAGGTGCAGTGGAAGGTGGACAACGCCCTGCAGA |
| | | GCGGCAACAGCCAGGAGAGCGTCACCGAGCAGGACAGC |
| | | AAGGACTCCACCTACAGCCTGAGCAGCACCCTGACCCTG |
| | | AGCAAGGCCGACTACGAGAAGCATAAGGTGTACGCCTG |
| | | CGAGGTGACCCACCAGGGCCTGTCCAGCCCCGTGACCA |
| | | AGAGCTTCAACAGGGGCGAGTGC |

BAP058-Clone N HC

| | | |
|---|---|---|
| SEQ ID NO: 1601 | HCDR1 | SYWMY |
| (Kabat) | | |
| SEQ ID NO: 1602 | HCDR2 | RIDPNSGSTKYNEKFKN |
| (Kabat) | | |
| SEQ ID NO: 1603 | HCDR3 | DYRKGLYAMDY |
| (Kabat) | | |
| SEQ ID NO: 1604 | HCDR1 | GYTFTSY |
| (Chothia) | | |
| SEQ ID NO: 1605 | HCDR2 | DPNSGS |
| (Chothia) | | |
| SEQ ID NO: 1603 | HCDR3 | DYRKGLYAMDY |
| (Chothia) | | |
| SEQ ID NO: 1620 | VH | EVQLVQSGAEVKKPGATVKISCKVSGYTFTSYWMYWVRQ |
| | | ATGQGLEWMGRIDPNSGSTKYNEKFKNRVTITADKSTSTA |
| | | YMELSSLRSEDTAVYYCARDYRKGLYAMDYWGQGTTVT |
| | | VSS |
| SEQ ID NO: 1621 | DNA | GAAGTGCAGCTGGTGCAGTCAGGCGCCGAAGTGAAGAA |
| | VH | ACCCGGCGCTACCGTGAAGATTAGCTGTAAAGTCTCAGG |
| | | CTACACCTTCACTAGCTACTGGATGTACTGGGTCCGACA |
| | | GGCTACCGGTCAAGGCCTGGAGTGGATGGGTAGAATCG |
| | | ACCCTAATAGCGGCTCTACTAAGTATAACGAGAAGTTTA |
| | | AGAATAGAGTGACTATCACCGCCGATAAGTCTACTAGCA |
| | | CCGCCTATATGGAACTGTCTAGCCTGAGATCAGAGGACA |
| | | CCGCCGTCTACTACTGCGCTAGAGACTATAGAAAGGGCC |
| | | TGTACGCTATGGACTACTGGGGTCAAGGCACTACCGTGA |
| | | CCGTGTCTTCA |
| SEQ ID NO: 1622 | Heavy | EVQLVQSGAEVKKPGATVKISCKVSGYTFTSYWMYWVRQ |
| | chain | ATGQGLEWMGRIDPNSGSTKYNEKFKNRVTITADKSTSTA |
| | | YMELSSLRSEDTAVYYCARDYRKGLYAMDYWGQGTTVT |
| | | VSSASTKGPSVFPLAPCSRSTSESTAALGCLVKDYFPEPVTV |
| | | SWNSGALTSGVHTFPAVLQSSGLYSLSSVVTVPSSSLGTKT |
| | | YTCNVDHKPSNTKVDKRVESKYGPPCPPCPAPEFLGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYK |
| | | CKVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| SEQ ID NO: 1623 | DNA | GAAGTGCAGCTGGTGCAGTCAGGCGCCGAAGTGAAGAA |
| | heavy | ACCCGGCGCTACCGTGAAGATTAGCTGTAAAGTCTCAGG |
| | chain | CTACACCTTCACTAGCTACTGGATGTACTGGGTCCGACA |
| | | GGCTACCGGTCAAGGCCTGGAGTGGATGGGTAGAATCG |
| | | ACCCTAATAGCGGCTCTACTAAGTATAACGAGAAGTTTA |
| | | AGAATAGAGTGACTATCACCGCCGATAAGTCTACTAGCA |
| | | CCGCCTATATGGAACTGTCTAGCCTGAGATCAGAGGACA |
| | | CCGCCGTCTACTACTGCGCTAGAGACTATAGAAAGGGCC |
| | | TGTACGCTATGGACTACTGGGGTCAAGGCACTACCGTGA |
| | | CCGTGTCTTCAGCTAGCACTAAGGGCCCGTCCGTGTTCC |
| | | CCCTGGCACCTTGTAGCCGGAGCACTAGCGAATCCACCG |
| | | CTGCCCTCGGCTGCCTGGTCAAGGATTACTTCCCGGAGC |
| | | CCGTGACCGTGTCCTGGAACAGCGGAGCCCTGACCTCCG |
| | | GAGTGCACACCTTCCCCGCTGTGCTGCAGAGCTCCGGGC |
| | | TGTACTCGCTGTCGTCGGTGGTCACGGTGCCTTCATCTAG |
| | | CCTGGGTACCAAGACCTACACTTGCAACGTGGACCACAA |
| | | GCCTTCCAACACTAAGGTGGACAAGCGCGTCGAATCGA |
| | | AGTACGGCCCACCGTGCCCGCCTTGTCCCGCGCCGGAGT |
| | | TCCTCGGCGGTCCCTCGGTCTTTCTGTTCCCACCGAAGCC |
| | | CAAGGACACTTTGATGATTTCCCGCACCCCTGAAGTGAC |
| | | ATGCGTGGTCGTGGACGTGTCACAGGAAGATCCGGAGG |
| | | TGCAGTTCAATTGGTACGTGGATGGCGTCGAGGTGCACA |
| | | ACGCCAAAACCAAGCCGAGGGAGGAGCAGTTCAACTCC |
| | | ACTTACCGCGTCGTGTCCGTGCTGACGGTGCTGCATCAG |
| | | GACTGGCTGAACGGGAAGGAGTACAAGTGCAAAGTGTC |
| | | CAACAAGGGACTTCCTAGCTCAATCGAAAAGACCATCTC |
| | | GAAAGCCAAGGGACAGCCCCGGGAACCCCAAGTGTATA |
| | | CCCTGCCACCGAGCCAGGAAGAAATGACTAAGAACCAA |
| | | GTCTCATTGACTTGCCTTGTGAAGGGCTTCTACCCATCGG |
| | | ATATCGCCGTGGAATGGGAGTCCAACGGCCAGCCGGAA |
| | | AACAACTACAAGACCACCCCTCCGGTGCTGGACTCAGAC |
| | | GGATCCTTCTTCCTCTACTCGCGGCTGACCGTGGATAAG |
| | | AGCAGATGGCAGGAGGGAAATGTGTTCAGCTGTTCTGTG |
| | | ATGCATGAAGCCCTGCACAACCACTACACTCAGAAGTCC |
| | | CTGTCCCTCTCCCTGGGA |

BAP058-Clone N LC

| | | |
|---|---|---|
| SEQ ID NO: 1609 | LCDR1 | KASQDVGTAVA |
| (Kabat) | | |
| SEQ ID NO: 1610 | LCDR2 | WASTRHT |
| (Kabat) | | |
| SEQ ID NO: 1611 | LCDR3 | QQYNSYPLT |
| (Kabat) | | |
| SEQ ID NO: 1612 | LCDR1 | SQDVGTA |
| (Chothia) | | |
| SEQ ID NO: 1613 | LCDR2 | WAS |
| (Chothia) | | |
| SEQ ID NO: 1614 | LCDR3 | YNSYPL |
| (Chothia) | | |
| SEQ ID NO: 1624 | VL | DVVMTQSPLSLPVTLGQPASISCKASQDVGTAVAWYQQKP |
| | | GQAPRLLIYWASTRHTGVPSRFSGSGSGTEFTLTISSLQPDD |
| | | FATYYCQQYNSYPLTFGQGTKVEIK |
| SEQ ID NO: 1625 | DNA | GACGTCGTGATGACTCAGTCACCCCTGAGCCTGCCCGTG |
| | VL | ACCCTGGGGCAGCCCGCCTCTATTAGCTGTAAAGCCTCT |
| | | CAGGACGTGGGCACCGCCGTGGCCTGGTATCAGCAGAA |
| | | GCCAGGGCAAGCCCCTAGACTGCTGATCTACTGGGCCTC |
| | | TACTAGACACACCGGCGTGCCCTCTAGGTTTAGCGGTAG |
| | | CGGTAGTGGCACCGAGTTCACCCTGACTATCTCTTCACT |
| | | GCAGCCCGACGACTTCGCTACCTACTACTGTCAGCAGTA |
| | | TAATAGCTACCCCCTGACCTTCGGTCAAGGCACTAAGGT |
| | | CGAGATTAAG |
| SEQ ID NO: 1626 | Light | DVVMTQSPLSLPVTLGQPASISCKASQDVGTAVAWYQQKP |
| | chain | GQAPRLLIYWASTRHTGVPSRFSGSGSGTEFTLTISSLQPDD |
| | | FATYYCQQYNSYPLTFGQGTKVEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| SEQ ID NO: 1627 | DNA | GACGTCGTGATGACTCAGTCACCCCTGAGCCTGCCCGTG |
| | light | ACCCTGGGGCAGCCCGCCTCTATTAGCTGTAAAGCCTCT |
| | chain | CAGGACGTGGGCACCGCCGTGGCCTGGTATCAGCAGAA |
| | | GCCAGGGCAAGCCCCTAGACTGCTGATCTACTGGGCCTC |
| | | TACTAGACACACCGGCGTGCCCTCTAGGTTTAGCGGTAG |
| | | CGGTAGTGGCACCGAGTTCACCCTGACTATCTCTTCACT |
| | | GCAGCCCGACGACTTCGCTACCTACTACTGTCAGCAGTA |
| | | TAATAGCTACCCCCTGACCTTCGGTCAAGGCACTAAGGT |
| | | CGAGATTAAGCGTACGGTGGCCGCTCCCAGCGTGTTCAT |
| | | CTTCCCCCCCAGCGACGAGCAGCTGAAGAGCGGCACCG |
| | | CCAGCGTGGTGTGCCTGCTGAACAACTTCTACCCCCGGG |
| | | AGGCCAAGGTGCAGTGGAAGGTGGACAACGCCCTGCAG |
| | | AGCGGCAACAGCCAGGAGAGCGTCACCGAGCAGGACAG |
| | | CAAGGACTCCACCTACAGCCTGAGCAGCACCCTGACCCT |
| | | GAGCAAGGCCGACTACGAGAAGCATAAGGTGTACGCCT |
| | | GCGAGGTGACCCACCAGGGCCTGTCCAGCCCCGTGACC |
| | | AAGAGCTTCAACAGGGGCGAGTGC |

BAP058-Clone O HC

| | | |
|---|---|---|
| SEQ ID NO: 1628 | HCDR1 | agctactggatgtac |
| (Kabat) | | |
| SEQ ID NO: 1629 | HCDR2 | agaatcgaccctaatagcggctctactaagtataac |
| (Kabat) | | gagaagtttaagaat |
| SEQ ID NO: 1630 | HCDR3 | gactatagaaagggcctgtac gctatggactac |
| (Kabat) | | |
| SEQ ID NO: 1631 | HCDR1 | ggctacaccttcactagctac |
| (Chothia) | | |
| SEQ ID NO: 1632 | HCDR2 | gaccctaatagcggctct |
| (Chothia) | | |
| SEQ ID NO: 1630 | HCDR3 | gactatagaaagggcctgtac gctatggactac |
| (Chothia) | | |

BAP058-Clone O LC

| | | |
|---|---|---|
| SEQ ID NO: 1633 | LCDR1 | aaagcctctcaggacgtgggcaccgccgtggcc |
| (Kabat) | | |
| SEQ ID NO: 1634 | LCDR2 | tgggcctctactagacacacc |
| (Kabat) | | |
| SEQ ID NO: 1635 | LCDR3 | cagcagtataatagctaccccctgacc |
| (Kabat) | | |
| SEQ ID NO: 1636 | LCDR1 | tctcaggacgtgggcaccgcc |
| (Chothia) | | |
| SEQ ID NO: 1637 | LCDR2 | tgggcctct |
| (Chothia) | | |
| SEQ ID NO: 1638 | LCDR3 | tataatagctaccccctg |
| (Chothia) | | |

BAP058-Clone N HC

| | | |
|---|---|---|
| SEQ ID NO: 1628 | HCDR1 | agctactggatgtac |
| (Kabat) | | |
| SEQ ID NO: 1629 | HCDR2 | agaatcgaccctaatagcggctctactaagtataac |
| (Kabat) | | gagaagtttaagaat |
| SEQ ID NO: 1630 | HCDR3 | gactatagaaagggcctgtacgctatggactac |
| (Kabat) | | |
| SEQ ID NO: 1631 | HCDR1 | ggctacaccttcactagctac |
| (Chothia) | | |
| SEQ ID NO: 1632 | HCDR2 | gaccctaatagcggctct |
| (Chothia) | | |
| SEQ ID NO: 1630 | HCDR3 | gactatagaaagggcctgtacgctatggactac |
| (Chothia) | | |

BAP058-Clone N LC

| | | |
|---|---|---|
| SEQ ID NO: 1633 | LCDR1 | aaagcctctcaggacgtgggcaccgccgtggcc |
| (Kabat) | | |
| SEQ ID NO: 1634 | LCDR2 | tgggcctctactagacacacc |
| (Kabat) | | |
| SEQ ID NO: 1635 | LCDR3 | cagcagtataatagctaccccctgacc |
| (Kabat) | | |
| SEQ ID NO: 1636 | LCDR1 | tctcaggacgtgggcaccgcc |
| (Chothia) | | |
| SEQ ID NO: 1637 | LCDR2 | tgggcctct |
| (Chothia) | | |
| SEQ ID NO: 1638 | LCDR3 | tataatagctaccccctg |
| (Chothia) | | |

### Other Exemplary PD-L1 Inhibitors

In one embodiment, the anti-PD-L1 antibody molecule is Atezolizumab (Genentech/Roche), also known as MPDL3280A, RG7446, R05541267, YW243.55.S70, or TECENTRIQ™. Atezolizumab and other anti-PD-L1 antibodies are disclosed in U.S. Pat. No. 8,217,149, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizuma, e.g., as disclosed in Table 8.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 8.

In one embodiment, the anti-PD-L1 antibody molecule is Durvalumab (Medlmmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 8.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 8.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 8
Amino acid sequences of other exemplary
anti-PD-L1 antibody molecules
Atezolizumab

| | | |
|---|---|---|
| SEQ | Heavy | EVQLVESGGGLVQPGGSLRLSCAASGFTFSDSWIHW |
| ID | chain | VRQAPGKGLEWVAWISPYGGSTYYADSVKGRFTISA |
| NO: | | DTSKNTAYLQMNSLRAEDTAVYYCARRHWPGGFDYW |
| 1639 | | GQGTLVTVSSASTKGPSVFPLAPSSKSTSGGTAALG |
| | | CLVKDYFPEPVTVSWNSGALTSGVHTFPAVLQSSGL |
| | | YSLSSVVTVPSSSLGTQTYICNVNHKPSNTKVDKKV |
| | | EPKSCDKTHTCPPCPAPELLGGPSVFLFPPKPKDTL |
| | | MISRTPEVTCVVVDVSHEDPEVKFNWYVDGVEVHNA |
| | | KTKPREEQYASTYRVVSVLTVLHQDWLNGKEYKCKV |
| | | SNKALPAPIEKTISKAKGQPREPQVYTLPPSREEMT |
| | | KNQVSLTCLVKGFYPSDIAVEWESNGQPENNYKTTP |
| | | PVLDSDGSFFLYSKLTVDKSRWQQGNVFSCSVMHEA |
| | | LHNHYTQKSLSLSPGK |
| SEQ | Light | DIQMTQSPSSLSASVGDRVTITCRASQDVSTAVAWY |
| ID | chain | QQKPGKAPKLLIYSASFLYSGVPSRFSGSGSGTDFT |
| NO: | | LTISSLQPEDFATYYCQQYLYHPATFGQGTKVEIKR |
| 1640 | | TVAAPSVFIFPPSDEQLKSGTASVVCLLNNFYPREA |
| | | KVQWKVDNALQSGNSQESVTEQDSKDSTYSLSSTLT |
| | | LSKADYEKHKVYACEVTHQGLSSPVTKSFNRGEC |

Avelumab

| | | |
|---|---|---|
| SEQ | Heavy | EVQLLESGGGLVQPGGSLRLSCAASGFTFSSYIMMW |
| ID | chain | VRQAPGKGLEWVSSIYPSGGITFYADTVKGRFTISR |
| NO: | | DNSKNTLYLQMNSLRAEDTAVYYCARIKLGTVTTVD |
| 1641 | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDK |
| | | KVEPKSCDKTHTCPPCPAPELLGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYKC |
| | | KVSNKALPAPIEKTISKAKGQPREPQVYTLPPSRDE |
| | | LTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| SEQ | Light | QSALTQPASVSGSPGQSITISCTGTSSDVGGYNYVS |
| ID | chain | WYQQHPGKAPKLMIYDVSNRPSGVSNRFSGSKSGNT |
| NO: | | ASLTISGLQAEDEADYYCSSYTSSSTRVFGTGTKVT |
| 1642 | | VLGQPKANPTVTLFPPSSEELQANKATLVCLISDFY |
| | | PGAVTVAWKADGSPVKAGVETTKPSKQSNNKYAASS |
| | | YLSLTPEQWKSHRSYSCQVTHEGSTVEKTVAPTECS |

Durvalumab

| | | |
|---|---|---|
| SEQ | Heavy | EVQLVESGGGLVQPGGSLRLSCAASGFTFSRYWMSW |
| ID | chain | VRQAPGKGLEWVANIKQDGSEKYYVDSVKGRFTISR |
| NO: | | DNAKNSLYLQMNSLRAEDTAVYYCAREGGWFGELAF |
| 1643 | | DYWGQGTLVTVSSASTKGPSVFPLAPSSKSTSGGTA |
| | | ALGCLVKDYFPEPVTVSWNSGALTSGVHTFPAVLQS |
| | | SGLYSLSSVVTVPSSSLGTQTYICNVNHKPSNTKVD |
| | | KRVEPKSCDKTHTCPPCPAPEFEGGPSVFLFPPKPK |
| | | DTLMISRTPEVTCVVVDVSHEDPEVKFNWYVDGVEV |
| | | HNAKTKPREEQYNSTYRVVSVLTVLHQDWLNGKEYK |
| | | CKVSNKALPASIEKTISKAKGQPREPQVYTLPPSRE |
| | | EMTKNQVSLTCLVKGFYPSDIAVEWESNGQPENNYK |
| | | TTPPVLDSDGSFFLYSKLTVDKSRWQQGNVFSCSVM |
| | | HEALHNHYTQKSLSLSPGK |
| SEQ | Light | EIVLTQSPGTLSLSPGERATLSCRASQRVSSSYLAW |
| ID | chain | YQQKPGQAPRLLIYDASSRATGIPDRFSGSGSGTDF |
| NO: | | TLTISRLEPEDFAVYYCQQYGSLPWTFGQGTKVEIK |
| 1644 | | RTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSFNRGEC |

BMS-936559

| | | |
|---|---|---|
| SEQ | VH | QVQLVQSGAEVKKPGSSVKVSCKTSGDTFSTYAISW |
| ID | | VRQAPGQGLEWMGGIIPIFGKAHYAQKFQGRVTITA |
| NO: | | DESTSTAYMELSSLRSEDTAVYFCARKFHFVSGSPF |
| 1645 | | GMDVWGQGTTVTVSS |
| SEQ | VL | EIVLTQSPATLSLSPGERATLSCRASQSVSSYLAWY |
| ID | | QQKPGQAPRLLIYDASNRATGIPARFSGSGSGTDFT |
| NO: | | LTISSLEPEDFAVYYCQQRSNWPTFGQGTKVEIK |
| 1646 | | |

### Exemplary LAG-3 Inhibitors

In certain embodiments, the anti-CD32b antibodies disclosed herein can be administered in combination with a LAG-3 inhibitor known in the art. The LAG-3 inhibitor may be an antibody, an antigen binding fragment thereof, an immunoadhesin, a fusion protein, or oligopeptide. In some embodiments, the LAG-3 inhibitor is chosen from LAG525 (Novartis), BMS-986016 (Bristol-Myers Squibb), TSR-033 (Tesaro), MK-4280 (Merck & Co), or REGN3767 (Regeneron).

Exemplary Anti-LAG-3 Antibody Molecules

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of BAP050-Clone I or BAP050-Clone J disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 5). In some embodiments, the CDRs are according to the combined CDR definitions of both Kabat and Chothia (e.g., as set out in Table 9). In one embodiment, the combination of Kabat and Chothia CDR of VH CDR1 comprises the amino acid sequence GFTLTNYGMN (SEQ ID NO: 766). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-LAG-3 antibody molecule comprises a heavy chain variable region (VH) comprising a VHCDR1 amino acid sequence of SEQ ID NO: 701, a VHCDR2 amino acid sequence of SEQ ID NO: 702, and a VHCDR3 amino acid sequence of SEQ ID NO: 703; and a light chain variable region (VL) comprising a VLCDR1 amino acid sequence of SEQ ID NO: 710, a VLCDR2 amino acid sequence of SEQ ID NO: 711, and a VLCDR3 amino acid sequence of SEQ ID NO: 712, each disclosed in Table 9.

In one embodiment, the anti-LAG-3 antibody molecule comprises a VH comprising a VHCDR1 encoded by the nucleotide sequence of SEQ ID NO: 736 or 737, a VHCDR2 encoded by the nucleotide sequence of SEQ ID NO: 738 or 739, and a VHCDR3 encoded by the nucleotide sequence of SEQ ID NO: 740 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 9. In one embodiment, the anti-LAG-3 antibody molecule comprises a VH comprising a VHCDR1 encoded by the nucleotide sequence of SEQ ID NO: 758 or 737, a VHCDR2 encoded by the nucleotide sequence of SEQ ID NO: 759 or 739, and a VHCDR3 encoded by the nucleotide sequence of SEQ ID NO: 760 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 9.

In one embodiment, the anti-LAG-3 antibody molecule comprises a VH comprising the amino acid sequence of SEQ ID NO: 706, or an amino acid sequence having at least about 85%, 90%, 95%, or 99% sequence identity to SEQ ID NO: 706. In one embodiment, the anti-LAG-3 antibody molecule comprises a VL comprising the amino acid sequence of SEQ ID NO: 718, or an amino acid sequence having at least about 85%, 90%, 95%, or 99% sequence identity to SEQ ID NO: 718. In one embodiment, the anti-LAG-3 antibody molecule comprises a VH comprising the amino acid sequence of SEQ ID NO: 724, or an amino acid sequence having at least about 85%, 90%, 95%, or 99% sequence identity to SEQ ID NO: 724. In one embodiment, the anti-LAG-3 antibody molecule comprises a VL comprising the amino acid sequence of SEQ ID NO: 730, or an amino acid sequence having at least about 85%, 90%, 95%, or 99% sequence identity to SEQ ID NO: 730. In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

In one embodiment, the antibody molecule comprises a VH encoded by the nucleotide sequence of SEQ ID NO: 707 or 708, or a nucleotide sequence having at least about 85%, 90%, 95%, or 99% sequence identity to SEQ ID NO: 707 or 708. In one embodiment, the antibody molecule comprises a VL encoded by the nucleotide sequence of SEQ ID NO: 719 or 720, or a nucleotide sequence having at least about 85%, 90%, 95%, or 99% sequence identity to SEQ ID NO: 719 or 720. In one embodiment, the antibody molecule comprises a VH encoded by the nucleotide sequence of SEQ ID NO: 725 or 726, or a nucleotide sequence having at least about 85%, 90%, 95%, or 99% sequence identity to SEQ ID NO: 725 or 726. In one embodiment, the antibody molecule comprises a VL encoded by the nucleotide sequence of SEQ ID NO: 731 or 732, or a nucleotide sequence having at least about 85%, 90%, 95%, or 99% sequence identity to SEQ ID NO: 731 or 732.

In one embodiment, the antibody molecule comprises a VH encoded by the nucleotide sequence of SEQ ID NO: 707 or 708 and a VL encoded by the nucleotide sequence of SEQ ID NO: 719 or 720. In one embodiment, the antibody molecule comprises a VH encoded by the nucleotide sequence of SEQ ID NO: 725 or 726 and a VL encoded by the nucleotide sequence of SEQ ID NO: 731 or 732. In one embodiment, the anti-LAG-3 antibody molecule comprises a heavy chain comprising the amino acid sequence of SEQ ID NO: 709, or an amino acid sequence having at least about 85%, 90%, 95%, or 99% sequence identity to SEQ ID NO: 709. In one embodiment, the anti-LAG-3 antibody molecule comprises a light chain comprising the amino acid sequence of SEQ ID NO: 721, or an amino acid sequence having at least about 85%, 90%, 95%, or 99% sequence identity to SEQ ID NO: 721. In one embodiment, the anti-LAG-3 antibody molecule comprises a heavy chain comprising the amino acid sequence of SEQ ID NO: 727, or an amino acid sequence having at least about 85%, 90%, 95%, or 99% sequence identity to SEQ ID NO: 727. In one embodiment, the anti-LAG-3 antibody molecule comprises a light chain comprising the amino acid sequence of SEQ ID NO: 733, or an amino acid sequence having at least about 85%, 90%, 95%, or 99% sequence identity to SEQ ID NO: 733. In one embodiment, the anti-LAG-3 antibody molecule comprises a heavy chain comprising the amino acid sequence of SEQ ID NO: 709 and a light chain comprising the amino acid sequence of SEQ ID NO: 721. In one embodiment, the anti-LAG-3 antibody molecule comprises a heavy chain comprising the amino acid sequence of SEQ ID NO: 727 and a light chain comprising the amino acid sequence of SEQ ID NO: 733.

In one embodiment, the antibody molecule comprises a heavy chain encoded by the nucleotide sequence of SEQ ID NO: 716 or 717, or a nucleotide sequence having at least about 85%, 90%, 95%, or 99% sequence identity to SEQ ID NO: 716 or 717. In one embodiment, the antibody molecule comprises a light chain encoded by the nucleotide sequence of SEQ ID NO: 722 or 723, or a nucleotide sequence having at least about 85%, 90%, 95%, or 99% sequence identity to SEQ ID NO: 722 or 723. In one embodiment, the antibody molecule comprises a heavy chain encoded by the nucleotide sequence of SEQ ID NO: 728 or 729, or a nucleotide sequence having at least about 85%, 90%, 95%, or 99% sequence identity to SEQ ID NO: 728 or 729. In one embodiment, the antibody molecule comprises a light chain encoded by the nucleotide sequence of SEQ ID NO: 734 or 735, or a nucleotide sequence having at least about 85%, 90%, 95%, or 99% sequence identity to SEQ ID NO: 734 or 735. In one embodiment, the antibody molecule comprises a heavy chain encoded by the nucleotide sequence of SEQ ID NO: 716 or 717 and a light chain encoded by the nucleotide sequence of SEQ ID NO: 722 or 723. In one embodiment, the antibody molecule comprises a heavy chain encoded by the nucleotide sequence of SEQ ID NO: 728 or 729 and a light chain encoded by the nucleotide sequence of SEQ ID NO: 734 or 735.

The antibody molecules described herein can be made by vectors, host cells, and methods described in US 2015/0259420, incorporated by reference in its entirety.

TABLE 9
Amino acid and nucleotide sequences of
exemplary anti-LAG-3 antibody molecules

| | | |
|---|---|---|
| BAP050-Clone I HC | | |
| SEQ ID NO: 701 | HCDR1 | NYGMN |
| (Kabat) | | |
| SEQ ID NO: 702 | HCDR2 | WINTDTGEPTYADDFKG |
| (Kabat) | | |
| SEQ ID NO: 703 | HCDR3 | NPPYYYGTNNAEAMDY |
| (Kabat) | | |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 706 | VH | QVQLVQSGAEVKKPGASVKVSCKASGFTLTNYGMNWVRQARGQR |
| | | LEWIGWINTDTGEPTYADDFKGRFVFSLDTSVSTAYLQISSLKA |
| | | EDTAVYYCARNPPYYYGTNNAEAMDYWGQGTTVTVSS |
| SEQ ID NO: 707 | DNA VH | CAAGTGCAGCTGGTGCAGTCGGGAGCCGAAGTGAAGAAGCCTGG |
| | | AGCCTCGGTGAAGGTGTCGTGCAAGGCATCCGGATTCACCCTCA |
| | | CCAATTACGGGATGAACTGGGTCAGACAGGCCCGGGGTCAACGG |
| | | CTGGAGTGGATCGGATGGATTAACACCGACACCGGGGAGCCTAC |
| | | CTACGCGGACGATTTCAAGGGACGGTTCGTGTTCTCCCTCGACA |
| | | CCTCCGTGTCCACCGCCTACCTCCAAATCTCCTCACTGAAAGCG |
| | | GAGGACACCGCCGTGTACTATTGCGCGAGGAACCCGCCCTACTA |
| | | CTACGGAACCAACAACGCCGAAGCCATGGACTACTGGGGCCAGG |
| | | GCACCACTGTGACTGTGTCCAGC |
| SEQ ID NO: 708 | DNA VH | CAGGTGCAGCTGGTGCAGTCTGGCGCCGAAGTGAAGAAACCTGG |
| | | CGCCTCCGTGAAGGTGTCCTGCAAGGCCTCTGGCTTCACCCTGA |
| | | CCAACTACGGCATGAACTGGGTGCGACAGGCCAGGGGCCAGCGG |
| | | CTGGAATGGATCGGCTGGATCAACACCGACACCGGCGAGCCTAC |
| | | CTACGCCGACGACTTCAAGGGCAGATTCGTGTTCTCCCTGGACA |
| | | CCTCCGTGTCCACCGCCTACCTGCAGATCTCCAGCCTGAAGGCC |
| | | GAGGATACCGCCGTGTACTACTGCGCCCGGAACCCCCCTTACTA |
| | | CTACGGCACCAACAACGCCGAGGCCATGGACTATTGGGGCCAGG |
| | | GCACCACCGTGACCGTGTCCTCT |
| SEQ ID NO: 709 | Heavy | QVQLVQSGAEVKKPGASVKVSCKASGFTLTNYGMNWVRQARGQR |
| | chain | LEWIGWINTDTGEPTYADDFKGRFVFSLDTSVSTAYLQISSLKA |
| | | EDTAVYYCARNPPYYYGTNNAEAMDYWGQGTTVTVSSASTKGPS |
| | | VFPLAPCSRSTSESTAALGCLVKDYFPEPVTVSWNSGALTSGVH |
| | | TFPAVLQSSGLYSLSSVVTVPSSSLGTKTYTCNVDHKPSNTKVD |
| | | KRVESKYGPPCPPCPAPEFLGGPSVFLFPPKPKDTLMISRTPEV |
| | | TCVVVDVSQEDPEVQFNWYVDGVEVHNAKTKPREEQFNSTYRVV |
| | | SVLTVLHQDWLNGKEYKCKVSNKGLPSSIEKTISKAKGQPREPQ |
| | | VYTLPPSQEEMTKNQVSLTCLVKGFYPSDIAVEWESNGQPENNY |
| | | KTTPPVLDSDGSFFLYSRLTVDKSRWQEGNVFSCSVMHEALHNH |
| | | YTQKSLSLSLG |
| SEQ ID NO: 716 | DNA | CAAGTGCAGCTGGTGCAGTCGGGAGCCGAAGTGAAGAAGCCTGG |
| | heavy | AGCCTCGGTGAAGGTGTCGTGCAAGGCATCCGGATTCACCCTCA |
| | chain | CCAATTACGGGATGAACTGGGTCAGACAGGCCCGGGGTCAACGG |
| | | CTGGAGTGGATCGGATGGATTAACACCGACACCGGGGAGCCTAC |
| | | CTACGCGGACGATTTCAAGGGACGGTTCGTGTTCTCCCTCGACA |
| | | CCTCCGTGTCCACCGCCTACCTCCAAATCTCCTCACTGAAAGCG |
| | | GAGGACACCGCCGTGTACTATTGCGCGAGGAACCCGCCCTACTA |
| | | CTACGGAACCAACAACGCCGAAGCCATGGACTACTGGGGCCAGG |
| | | GCACCACTGTGACTGTGTCCAGCGCGTCCACTAAGGGCCCGTCC |
| | | GTGTTCCCCCTGGCACCTTGTAGCCGGAGCACTAGCGAATCCAC |
| | | CGCTGCCCTCGGCTGCCTGGTCAAGGATTACTTCCCGGAGCCCG |
| | | TGACCGTGTCCTGGAACAGCGGAGCCCTGACCTCCGGAGTGCAC |
| | | ACCTTCCCCGCTGTGCTGCAGAGCTCCGGGCTGTACTCGCTGTC |
| | | GTCGGTGGTCACGGTGCCTTCATCTAGCCTGGGTACCAAGACCT |
| | | ACACTTGCAACGTGGACCACAAGCCTTCCAACACTAAGGTGGAC |
| | | AAGCGCGTCGAATCGAAGTACGGCCCACCGTGCCCGCCTTGTCC |
| | | CGCGCCGGAGTTCCTCGGCGGTCCCTCGGTCTTTCTGTTCCCAC |
| | | CGAAGCCCAAGGACACTTTGATGATTTCCCGCACCCCTGAAGTG |
| | | ACATGCGTGGTCGTGGACGTGTCACAGGAAGATCCGGAGGTGCA |
| | | GTTCAATTGGTACGTGGATGGCGTCGAGGTGCACAACGCCAAAA |
| | | CCAAGCCGAGGGAGGAGCAGTTCAACTCCACTTACCGCGTCGTG |
| | | TCCGTGCTGACGGTGCTGCATCAGGACTGGCTGAACGGGAAGGA |
| | | GTACAAGTGCAAAGTGTCCAACAAGGGACTTCCTAGCTCAATCG |
| | | AAAAGACCATCTCGAAAGCCAAGGGACAGCCCCGGGAACCCCAA |
| | | GTGTATACCCTGCCACCGAGCCAGGAAGAAATGACTAAGAACCA |
| | | AGTCTCATTGACTTGCCTTGTGAAGGGCTTCTACCCATCGGATA |
| | | TCGCCGTGGAATGGGAGTCCAACGGCCAGCCGGAAAACAACTAC |
| | | AAGACCACCCCTCCGGTGCTGGACTCAGACGGATCCTTCTTCCT |
| | | CTACTCGCGGCTGACCGTGGATAAGAGCAGATGGCAGGAGGGAA |
| | | ATGTGTTCAGCTGTTCTGTGATGCATGAAGCCCTGCACAACCAC |
| | | TACACTCAGAAGTCCCTGTCCCTCTCCCTGGGA |
| SEQ ID NO: 717 | DNA | CAGGTGCAGCTGGTGCAGTCTGGCGCCGAAGTGAAGAAACCTGG |
| | heavy | CGCCTCCGTGAAGGTGTCCTGCAAGGCCTCTGGCTTCACCCTGA |
| | chain | CCAACTACGGCATGAACTGGGTGCGACAGGCCAGGGGCCAGCGG |
| | | CTGGAATGGATCGGCTGGATCAACACCGACACCGGCGAGCCTAC |
| | | CTACGCCGACGACTTCAAGGGCAGATTCGTGTTCTCCCTGGACA |
| | | CCTCCGTGTCCACCGCCTACCTGCAGATCTCCAGCCTGAAGGCC |
| | | GAGGATACCGCCGTGTACTACTGCGCCCGGAACCCCCCTTACTA |
| | | CTACGGCACCAACAACGCCGAGGCCATGGACTATTGGGGCCAGG |
| | | GCACCACCGTGACCGTGTCCTCTGCTTCTACCAAGGGGCCCAGC |
| | | GTGTTCCCCCTGGCCCCCTGCTCCAGAAGCACCAGCGAGAGCAC |
| | | AGCCGCCCTGGGCTGCCTGGTGAAGGACTACTTCCCCGAGCCCG |
| | | TGACCGTGTCCTGGAACAGCGGAGCCCTGACCAGCGGCGTGCAC |
| | | ACCTTCCCCGCCGTGCTGCAGAGCAGCGGCCTGTACAGCCTGAG |
| | | CAGCGTGGTGACCGTGCCCAGCAGCAGCCTGGGCACCAAGACCT |
| | | ACACCTGTAACGTGGACCACAAGCCCAGCAACACCAAGGTGGAC |
| | | AAGAGGGTGGAGAGCAAGTACGGCCCACCCTGCCCCCCCTGCCC |
| | | AGCCCCCGAGTTCCTGGGCGGACCCAGCGTGTTCCTGTTCCCCC |
| | | CCAAGCCCAAGGACACCCTGATGATCAGCAGAACCCCCGAGGTG |
| | | ACCTGTGTGGTGGTGGACGTGTCCCAGGAGGACCCCGAGGTCCA |
| | | GTTCAACTGGTACGTGGACGGCGTGGAGGTGCACAACGCCAAGA |
| | | CCAAGCCCAGAGAGGAGCAGTTTAACAGCACCTACCGGGTGGTG |
| | | TCCGTGCTGACCGTGCTGCACCAGGACTGGCTGAACGGCAAAGA |
| | | GTACAAGTGTAAGGTCTCCAACAAGGGCCTGCCAAGCAGCATCG |
| | | AAAAGACCATCAGCAAGGCCAAGGGCCAGCCTAGAGAGCCCCAG |
| | | GTCTACACCCTGCCACCCAGCCAAGAGGAGATGACCAAGAACCA |
| | | GGTGTCCCTGACCTGTCTGGTGAAGGGCTTCTACCCAAGCGACA |
| | | TCGCCGTGGAGTGGGAGAGCAACGGCCAGCCCGAGAACAACTAC |
| | | AAGACCACCCCCCCAGTGCTGGACAGCGACGGCAGCTTCTTCCT |
| | | GTACAGCAGGCTGACCGTGGACAAGTCCAGATGGCAGGAGGGCA |
| | | ACGTCTTTAGCTGCTCCGTGATGCACGAGGCCCTGCACAACCAC |
| | | TACACCCAGAAGAGCCTGAGCCTGTCCCTGGGC |
| BAP050-Clone I LC | | |
| SEQ ID NO: 710 | LCDR1 | SSSQDISNYLN |
| (Kabat) | | |
| SEQ ID NO: 711 | LCDR2 | YTSTLHL |
| (Kabat) | | |
| SEQ ID NO: 712 | LCDR3 | QQYYNLPWT |
| (Kabat) | | |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 718 | VL | DIQMTQSPSSLSASVGDRVTITCSSSQDISNYLNWYLQKPGQSP |
| | | QLLIYYTSTLHLGVPSRFSGSGSGTEFTLTISSLQPDDFATYYC |
| | | QQYYNLPWTFGQGTKVEIK |
| SEQ ID NO: 719 | | GATATTCAGATGACTCAGTCACCTAGTAGCCTGAGCGCTAGTGT |
| | | GGGCGATAGAGTGACTATCACCTGTAGCTCTAGTCAGGATATCT |
| | | CTAACTACCTGAACTGGTATCTGCAGAAGCCCGGTCAATCACCT |
| | | CAGCTGCTGATCTACTACACTAGCACCCTGCACCTGGGCGTGCC |
| | | CTCTAGGTTTAGCGGTAGCGGTAGTGGCACCGAGTTCACCCTGA |
| | | CTATCTCTAGCCTGCAGCCCGACGACTTCGCTACCTACTACTGT |
| | | CAGCAGTACTATAACCTGCCCTGGACCTTCGGTCAAGGCACTAA |
| | | GGTCGAGATTAAG |
| SEQ ID NO: 720 | DNA VL | GACATCCAGATGACCCAGTCCCCCTCCAGCCTGTCTGCTTCCGT |
| | | GGGCGACAGAGTGACCATCACCTGTTCCTCCAGCCAGGACATCT |
| | | CCAACTACCTGAACTGGTATCTGCAGAAGCCCGGCCAGTCCCCT |
| | | CAGCTGCTGATCTACTACACCTCCACCCTGCACCTGGGCGTGCC |
| | | CTCCAGATTTTCCGGCTCTGGCTCTGGCACCGAGTTTACCCTGA |
| | | CCATCAGCTCCCTGCAGCCCGACGACTTCGCCACCTACTACTGC |
| | | CAGCAGTACTACAACCTGCCCTGGACCTTCGGCCAGGGCACCAA |
| | | GGTGGAAATCAAG |
| SEQ ID NO: 721 | Light | DIQMTQSPSSLSASVGDRVTITCSSSQDISNYLNWYLQKPGQ |
| | chain | SPQLLIYYTSTLHLGVPSRFSGSGSGTEFTLTISSLQPDDFATY |
| | | YCQQYYNLPWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| SEQ ID NO: 722 | DNA | GATATTCAGATGACTCAGTCACCTAGTAGCCTGAGCGCTAGTGT |
| | light | GGGCGATAGAGTGACTATCACCTGTAGCTCTAGTCAGGATATCT |
| | chain | CTAACTACCTGAACTGGTATCTGCAGAAGCCCGGTCAATCACCT |
| | | CAGCTGCTGATCTACTACACTAGCACCCTGCACCTGGGCGTGCC |
| | | CTCTAGGTTTAGCGGTAGCGGTAGTGGCACCGAGTTCACCCTGA |
| | | CTATCTCTAGCCTGCAGCCCGACGACTTCGCTACCTACTACTGT |
| | | CAGCAGTACTATAACCTGCCCTGGACCTTCGGTCAAGGCACTAA |
| | | GGTCGAGATTAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| SEQ ID NO: 723 | DNA | GACATCCAGATGACCCAGTCCCCCTCCAGCCTGTCTGCTTCCGT |
| | light | GGGCGACAGAGTGACCATCACCTGTTCCTCCAGCCAGGACATCT |
| | chain | CCAACTACCTGAACTGGTATCTGCAGAAGCCCGGCCAGTCCCCT |
| | | CAGCTGCTGATCTACTACACCTCCACCCTGCACCTGGGCGTGCC |
| | | CTCCAGATTTTCCGGCTCTGGCTCTGGCACCGAGTTTACCCTGA |
| | | CCATCAGCTCCCTGCAGCCCGACGACTTCGCCACCTACTACTGC |
| | | CAGCAGTACTACAACCTGCCCTGGACCTTCGGCCAGGGCACCAA |
| | | GGTGGAAATCAAGCGTACGGTGGCCGCTCCCAGCGTGTTCATCT |
| | | TCCCCCCAAGCGACGAGCAGCTGAAGAGCGGCACCGCCAGCGTG |
| | | GTGTGTCTGCTGAACAACTTCTACCCCAGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCACAAGGT |
| | | GTACGCCTGTGAGGTGACCCACCAGGGCCTGTCCAGCCCCGTGA |
| | | CCAAGAGCTTCAACAGGGGCGAGTGC |
| BAP050-Clone J HC | | |
| SEQ ID NO: 701 | HCDR1 | NYGMN |
| (Kabat) | | |
| SEQ ID NO: 702 | HCDR2 | WINTDTGEPTYADDFKG |
| (Kabat) | | |
| SEQ ID NO: 703 | HCDR3 | NPPYYYGTNNAEAMDY |
| (Kabat) | | |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 724 | VH | QVQLVQSGAEVKKPGASVKVSCKASGFTLTNYGMNWVRQAPGQG |
| | | LEWMGWINTDTGEPTYADDFKGRFVFSLDTSVSTAYLQISSLKA |
| | | EDTAVYYCARNPPYYYGTNNAEAMDYWGQGTTVTVSS |
| SEQ ID NO: 725 | DNA VH | CAGGTGCAGCTGGTGCAGTCAGGCGCCGAAGTGAAGAAACCCGG |
| | | CGCTAGTGTGAAAGTCAGCTGTAAAGCTAGTGGCTTCACCCTGA |
| | | CTAACTACGGGATGAACTGGGTCCGCCAGGCCCCAGGTCAAGGC |
| | | CTCGAGTGGATGGGCTGGATTAACACCGACACCGGCGAGCCTAC |
| | | CTACGCCGACGACTTTAAGGGCAGATTCGTGTTTAGCCTGGACA |
| | | CTAGTGTGTCTACCGCCTACCTGCAGATCTCTAGCCTGAAGGCC |
| | | GAGGACACCGCCGTCTACTACTGCGCTAGAAACCCCCCCTACTA |
| | | CTACGGCACTAACAACGCCGAGGCTATGGACTACTGGGGTCAAG |
| | | GCACTACCGTGACCGTGTCTAGC |
| SEQ ID NO: 726 | DNA VH | CAGGTGCAGCTGGTGCAGTCTGGCGCCGAAGTGAAGAAACCTGG |
| | | CGCCTCCGTGAAGGTGTCCTGCAAGGCCTCTGGCTTCACCCTGA |
| | | CCAACTACGGCATGAACTGGGTGCGACAGGCCCCTGGACAGGGC |
| | | CTGGAATGGATGGGCTGGATCAACACCGACACCGGCGAGCCTAC |
| | | CTACGCCGACGACTTCAAGGGCAGATTCGTGTTCTCCCTGGACA |
| | | CCTCCGTGTCCACCGCCTACCTGCAGATCTCCAGCCTGAAGGCC |
| | | GAGGATACCGCCGTGTACTACTGCGCCCGGAACCCCCCTTACTA |
| | | CTACGGCACCAACAACGCCGAGGCCATGGACTATTGGGGCCAGG |
| | | GCACCACCGTGACCGTGTCCTCT |
| SEQ ID NO: 727 | Heavy | QVQLVQSGAEVKKPGASVKVSCKASGFTLTNYGMNWVRQAPGQG |
| | chain | LEWMGWINTDTGEPTYADDFKGRFVFSLDTSVSTAYLQISSLKA |
| | | EDTAVYYCARNPPYYYGTNNAEAMDYWGQGTTVTVSSASTKGPS |
| | | VFPLAPCSRSTSESTAALGCLVKDYFPEPVTVSWNSGALTSGVH |
| | | TFPAVLQSSGLYSLSSVVTVPSSSLGTKTYTCNVDHKPSNTKVD |
| | | KRVESKYGPPCPPCPAPEFLGGPSVFLFPPKPKDTLMISRTPEV |
| | | TCVVVDVSQEDPEVQFNWYVDGVEVHNAKTKPREEQFNSTYRVV |
| | | SVLTVLHQDWLNGKEYKCKVSNKGLPSSIEKTISKAKGQPREPQ |
| | | VYTLPPSQEEMTKNQVSLTCLVKGFYPSDIAVEWESNGQPENNY |
| | | KTTPPVLDSDGSFFLYSRLTVDKSRWQEGNVFSCSVMHEALHNH |
| | | YTQKSLSLSLG |
| SEQ ID NO: 728 | DNA | CAGGTGCAGCTGGTGCAGTCAGGCGCCGAAGTGAAGAAACCCGG |
| | heavy | CGCTAGTGTGAAAGTCAGCTGTAAAGCTAGTGGCTTCACCCTGA |
| | chain | CTAACTACGGGATGAACTGGGTCCGCCAGGCCCCAGGTCAAGGC |
| | | CTCGAGTGGATGGGCTGGATTAACACCGACACCGGCGAGCCTAC |
| | | CTACGCCGACGACTTTAAGGGCAGATTCGTGTTTAGCCTGGACA |
| | | CTAGTGTGTCTACCGCCTACCTGCAGATCTCTAGCCTGAAGGCC |
| | | GAGGACACCGCCGTCTACTACTGCGCTAGAAACCCCCCCTACTA |
| | | CTACGGCACTAACAACGCCGAGGCTATGGACTACTGGGGTCAAG |
| | | GCACTACCGTGACCGTGTCTAGCGCTAGCACTAAGGGCCCGTCC |
| | | GTGTTCCCCCTGGCACCTTGTAGCCGGAGCACTAGCGAATCCAC |
| | | CGCTGCCCTCGGCTGCCTGGTCAAGGATTACTTCCCGGAGCCCG |
| | | TGACCGTGTCCTGGAACAGCGGAGCCCTGACCTCCGGAGTGCAC |
| | | ACCTTCCCCGCTGTGCTGCAGAGCTCCGGGCTGTACTCGCTGTC |
| | | GTCGGTGGTCACGGTGCCTTCATCTAGCCTGGGTACCAAGACCT |
| | | ACACTTGCAACGTGGACCACAAGCCTTCCAACACTAAGGTGGAC |
| | | AAGCGCGTCGAATCGAAGTACGGCCCACCGTGCCCGCCTTGTCC |
| | | CGCGCCGGAGTTCCTCGGCGGTCCCTCGGTCTTTCTGTTCCCAC |
| | | CGAAGCCCAAGGACACTTTGATGATTTCCCGCACCCCTGAAGTG |
| | | ACATGCGTGGTCGTGGACGTGTCACAGGAAGATCCGGAGGTGCA |
| | | GTTCAATTGGTACGTGGATGGCGTCGAGGTGCACAACGCCAAAA |
| | | CCAAGCCGAGGGAGGAGCAGTTCAACTCCACTTACCGCGTCGTG |
| | | TCCGTGCTGACGGTGCTGCATCAGGACTGGCTGAACGGGAAGGA |
| | | GTACAAGTGCAAAGTGTCCAACAAGGGACTTCCTAGCTCAATCG |
| | | AAAAGACCATCTCGAAAGCCAAGGGACAGCCCCGGGAACCCCAA |
| | | GTGTATACCCTGCCACCGAGCCAGGAAGAAATGACTAAGAACCA |
| | | AGTCTCATTGACTTGCCTTGTGAAGGGCTTCTACCCATCGGATA |
| | | TCGCCGTGGAATGGGAGTCCAACGGCCAGCCGGAAAACAACTAC |
| | | AAGACCACCCCTCCGGTGCTGGACTCAGACGGATCCTTCTTCCT |
| | | CTACTCGCGGCTGACCGTGGATAAGAGCAGATGGCAGGAGGGAA |
| | | ATGTGTTCAGCTGTTCTGTGATGCATGAAGCCCTGCACAACCAC |
| | | TACACTCAGAAGTCCCTGTCCCTCTCCCTGGGA |
| SEQ ID NO: 729 | DNA | CAGGTGCAGCTGGTGCAGTCTGGCGCCGAAGTGAAGAAACCTGG |
| | | CGCCTCCGTGAAGGTGTCCTGCAAGGCCTCTGGCTTCACCCTGA |
| | heavy | CCAACTACGGCATGAACTGGGTGCGACAGGCCCCTGGACAGGGC |
| | chain | CTGGAATGGATGGGCTGGATCAACACCGACACCGGCGAGCCTAC |
| | | CTACGCCGACGACTTCAAGGGCAGATTCGTGTTCTCCCTGGACA |
| | | CCTCCGTGTCCACCGCCTACCTGCAGATCTCCAGCCTGAAGGCC |
| | | GAGGATACCGCCGTGTACTACTGCGCCCGGAACCCCCCTTACTA |
| | | CTACGGCACCAACAACGCCGAGGCCATGGACTATTGGGGCCAGG |
| | | GCACCACCGTGACCGTGTCCTCTGCTTCTACCAAGGGGCCCAGC |
| | | GTGTTCCCCCTGGCCCCCTGCTCCAGAAGCACCAGCGAGAGCAC |
| | | AGCCGCCCTGGGCTGCCTGGTGAAGGACTACTTCCCCGAGCCCG |
| | | TGACCGTGTCCTGGAACAGCGGAGCCCTGACCAGCGGCGTGCAC |
| | | ACCTTCCCCGCCGTGCTGCAGAGCAGCGGCCTGTACAGCCTGAG |
| | | CAGCGTGGTGACCGTGCCCAGCAGCAGCCTGGGCACCAAGACCT |
| | | ACACCTGTAACGTGGACCACAAGCCCAGCAACACCAAGGTGGAC |
| | | AAGAGGGTGGAGAGCAAGTACGGCCCACCCTGCCCCCCCTGCCC |
| | | AGCCCCCGAGTTCCTGGGCGGACCCAGCGTGTTCCTGTTCCCCC |
| | | CCAAGCCCAAGGACACCCTGATGATCAGCAGAACCCCCGAGGTG |
| | | ACCTGTGTGGTGGTGGACGTGTCCCAGGAGGACCCCGAGGTCCA |
| | | GTTCAACTGGTACGTGGACGGCGTGGAGGTGCACAACGCCAAGA |
| | | CCAAGCCCAGAGAGGAGCAGTTTAACAGCACCTACCGGGTGGTG |
| | | TCCGTGCTGACCGTGCTGCACCAGGACTGGCTGAACGGCAAAGA |
| | | GTACAAGTGTAAGGTCTCCAACAAGGGCCTGCCAAGCAGCATCG |
| | | AAAAGACCATCAGCAAGGCCAAGGGCCAGCCTAGAGAGCCCCAG |
| | | GTCTACACCCTGCCACCCAGCCAAGAGGAGATGACCAAGAACCA |
| | | GGTGTCCCTGACCTGTCTGGTGAAGGGCTTCTACCCAAGCGACA |
| | | TCGCCGTGGAGTGGGAGAGCAACGGCCAGCCCGAGAACAACTAC |
| | | AAGACCACCCCCCCAGTGCTGGACAGCGACGGCAGCTTCTTCCT |
| | | GTACAGCAGGCTGACCGTGGACAAGTCCAGATGGCAGGAGGGCA |
| | | ACGTCTTTAGCTGCTCCGTGATGCACGAGGCCCTGCACAACCAC |
| | | TACACCCAGAAGAGCCTGAGCCTGTCCCTGGGC |
| BAP050-Clone J LC | | |
| SEQ ID NO: 710 | LCDR1 | SSSQDISNYLN |
| (Kabat) | | |
| SEQ ID NO: 711 | LCDR2 | YTSTLHL |
| (Kabat) | | |
| SEQ ID NO: 712 | LCDR3 | QQYYNLPWT |
| (Kabat) | | |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 730 | VL | DIQMTQSPSSLSASVGDRVTITCSSSQDISNYLNWYQQKPGKAP |
| | | KLLIYYTSTLHLGIPPRFSGSGYGTDFTLTINNIESEDAAYYFC |
| | | QQYYNLPWTFGQGTKVEIK |
| SEQ ID NO: 731 | DNA VL | GATATTCAGATGACTCAGTCACCTAGTAGCCTGAGCGCTAGTGT |
| | | GGGCGATAGAGTGACTATCACCTGTAGCTCTAGTCAGGATATCT |
| | | CTAACTACCTGAACTGGTATCAGCAGAAGCCCGGTAAAGCCCCT |
| | | AAGCTGCTGATCTACTACACTAGCACCCTGCACCTGGGAATCCC |
| | | CCCTAGGTTTAGCGGTAGCGGCTACGGCACCGACTTCACCCTGA |
| | | CTATTAACAATATCGAGTCAGAGGACGCCGCCTACTACTTCTGT |
| | | CAGCAGTACTATAACCTGCCCTGGACCTTCGGTCAAGGCACTAA |
| | | GGTCGAGATTAAG |
| SEQ ID NO: 732 | DNA VL | GACATCCAGATGACCCAGTCCCCCTCCAGCCTGTCTGCTTCCGT |
| | | GGGCGACAGAGTGACCATCACCTGTTCCTCCAGCCAGGACATCT |
| | | CCAACTACCTGAACTGGTATCAGCAGAAGCCCGGCAAGGCCCCC |
| | | AAGCTGCTGATCTACTACACCTCCACCCTGCACCTGGGCATCCC |
| | | CCCTAGATTCTCCGGCTCTGGCTACGGCACCGACTTCACCCTGA |
| | | CCATCAACAACATCGAGTCCGAGGACGCCGCCTACTACTTCTGC |
| | | CAGCAGTACTACAACCTGCCCTGGACCTTCGGCCAGGGCACCAA |
| | | GGTGGAAATCAAG |
| SEQ ID NO: 733 | Light | DIQMTQSPSSLSASVGDRVTITCSSSQDISNYLNWYQQKPGKAP |
| | chain | KLLIYYTSTLHLGIPPRFSGSGYGTDFTLTINNIESEDAAYYFC |
| | | QQYYNLPWTFGQGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 734 | DNA | GATATTCAGATGACTCAGTCACCTAGTAGCCTGAGCGCTAGTGT |
| | light | GGGCGATAGAGTGACTATCACCTGTAGCTCTAGTCAGGATATCT |
| | chain | CTAACTACCTGAACTGGTATCAGCAGAAGCCCGGTAAAGCCCCT |
| | | AAGCTGCTGATCTACTACACTAGCACCCTGCACCTGGGAATCCC |
| | | CCCTAGGTTTAGCGGTAGCGGCTACGGCACCGACTTCACCCTGA |
| | | CTATTAACAATATCGAGTCAGAGGACGCCGCCTACTACTTCTGT |
| | | CAGCAGTACTATAACCTGCCCTGGACCTTCGGTCAAGGCACTAA |
| | | GGTCGAGATTAAGCGTACGGTGGCCGCTCCCAGCGTGTTCATCT |
| | | TCCCCCCCAGCGACGAGCAGCTGAAGAGCGGCACCGCCAGCGTG |
| | | GTGTGCCTGCTGAACAACTTCTACCCCCGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCATAAGGT |
| | | GTACGCCTGCGAGGTGACCCACCAGGGCCTGTCCAGCCCCGTGA |
| | | CCAAGAGCTTCAACAGGGGCGAGTGC |
| SEQ ID NO: 735 | DNA | GACATCCAGATGACCCAGTCCCCCTCCAGCCTGTCTGCTTCCGT |
| | light | GGGCGACAGAGTGACCATCACCTGTTCCTCCAGCCAGGACATCT |
| | chain | CCAACTACCTGAACTGGTATCAGCAGAAGCCCGGCAAGGCCCCC |
| | | AAGCTGCTGATCTACTACACCTCCACCCTGCACCTGGGCATCCC |
| | | CCCTAGATTCTCCGGCTCTGGCTACGGCACCGACTTCACCCTGA |
| | | CCATCAACAACATCGAGTCCGAGGACGCCGCCTACTACTTCTGC |
| | | CAGCAGTACTACAACCTGCCCTGGACCTTCGGCCAGGGCACCAA |
| | | GGTGGAAATCAAGCGTACGGTGGCCGCTCCCAGCGTGTTCATCT |
| | | TCCCCCCAAGCGACGAGCAGCTGAAGAGCGGCACCGCCAGCGTG |
| | | GTGTGTCTGCTGAACAACTTCTACCCCAGGGAGGCCAAGGTGCA |
| | | GTGGAAGGTGGACAACGCCCTGCAGAGCGGCAACAGCCAGGAGA |
| | | GCGTCACCGAGCAGGACAGCAAGGACTCCACCTACAGCCTGAGC |
| | | AGCACCCTGACCCTGAGCAAGGCCGACTACGAGAAGCACAAGGT |
| | | GTACGCCTGTGAGGTGACCCACCAGGGCCTGTCCAGCCCCGTGA |
| | | CCAAGAGCTTCAACAGGGGCGAGTGC |
| BAP050-Clone I HC | | |
| SEQ ID NO: 736 | HCDR1 | AATTACGGGATGAAC |
| (Kabat) | | |
| SEQ ID NO: 737 | HCDR1 | AACTACGGCATGAAC |
| (Kabat) | | |
| SEQ ID NO: 738 | HCDR2 | TGGATTAACACCGACACCGGGGAGCCTACCTACGCGGACGATTT |
| (Kabat) | | CAAGGGA |
| SEQ ID NO: 739 | HCDR2 | TGGATCAACACCGACACCGGCGAGCCTACCTACGCCGACGACTT |
| | | CAAGGGC |
| SEQ ID NO: 740 | HCDR3 | AACCCGCCCTACTACTACGGAACCAACAACGCCGAAGCCATGGA |
| | | CTAC |
| SEQ ID NO: 741 | HCDR3 | AACCCCCCTTACTACTACGGCACCAACAACGCCGAGGCCATGGA |
| (Kabat) | | CTAT |
| SEQ ID NO: 742 | HCDR1 | GGATTCACCCTCACCAATTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 740 | HCDR3 | AACCCGCCCTACTACTACGGAACCAACAACGCCGAAGCCATGGA |
| (Chothia) | | CTAC |
| SEQ ID NO: 741 | HCDR3 | AACCCCCCTTACTACTACGGCACCAACAACGCCGAGGCCATGGA |
| (Chothia) | | CTAT |

BAP050-Clone I LC

| | | |
|---|---|---|
| SEQ ID NO: 746 | LCDR1 | AGCTCTAGTCAGGATATCTCTAACTACCTGAAC |
| (Kabat) | | |
| SEQ ID NO: 747 | LCDR1 | TCCTCCAGCCAGGACATCTCCAACTACCTGAAC |
| (Kabat) | | |
| SEQ ID NO: 748 | LCDR2 | TACACTAGCACCCTGCACCTG |
| (Kabat) | | |
| SEQ ID NO: 749 | LCDR2 | TACACCTCCACCCTGCACCTG |
| (Kabat) | | |
| SEQ ID NO: 750 | LCDR3 | CAGCAGTACTATAACCTGCCCTGGACC |
| (Kabat) | | |
| SEQ ID NO: 751 | LCDR3 | CAGCAGTACTACAACCTGCCCTGGACC |
| (Kabat) | | |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |
| BAP050-Clone J HC | | |
| SEQ ID NO: 758 | HCDR1 | AACTACGGGATGAAC |
| (Kabat) | | |
| SEQ ID NO: 737 | HCDR1 | AACTACGGCATGAAC |
| (Kabat) | | |
| SEQ ID NO: 759 | HCDR2 | TGGATTAACACCGACACCGGCGAGCCTACCTACGCCGACGACTT |
| (Kabat) | | TAAGGGC |
| SEQ ID NO: 739 | HCDR2 | TGGATCAACACCGACACCGGCGAGCCTACCTACGCCGACGACTT |
| (Kabat) | | CAAGGGC |
| SEQ ID NO: 760 | HCDR3 | AACCCCCCCTACTACTACGGCACTAACAACGCCGAGGCTATGGA |
| (Kabat) | | CTAC |
| SEQ ID NO: 741 | HCDR3 | AACCCCCCTTACTACTACGGCACCAACAACGCCGAGGCCATGGA |
| (Kabat) | | CTAT |
| SEQ ID NO: 761 | HCDR1 | GGCTTCACCCTGACTAACTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 760 | HCDR3 | AACCCCCCCTACTACTACGGCACTAACAACGCCGAGGCTATGGA |
| (Chothia) | | CTAC |
| SEQ ID NO: 741 | HCDR3 | AACCCCCCTTACTACTACGGCACCAACAACGCCGAGGCCATGGA |
| (Chothia) | | CTAT |
| BAP050-Clone J LC | | |
| SEQ ID NO: 746 | LCDR1 | AGCTCTAGTCAGGATATCTCTAACTACCTGAAC |
| (Kabat) | | |
| SEQ ID NO: 747 | LCDR1 | TCCTCCAGCCAGGACATCTCCAACTACCTGAAC |
| (Kabat) | | |
| SEQ ID NO: 748 | LCDR2 | TACACTAGCACCCTGCACCTG |
| (Kabat) | | |
| SEQ ID NO: 749 | LCDR2 | TACACCTCCACCCTGCACCTG |
| (Kabat) | | |
| SEQ ID NO: 750 | LCDR3 | CAGCAGTACTATAACCTGCCCTGGACC |
| (Kabat) | | |
| SEQ ID NO: 751 | LCDR3 | CAGCAGTACTACAACCTGCCCTGGACC |
| (Kabat) | | |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |

### Other Exemplary LAG-3 Inhibitors

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 10.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is MK-4280 (Merck & Co). In one embodiment, the anti-LAG-3 antibody molecule comprises one or more of the CDR sequences (or collectively all of the CDR sequences), the heavy chain or light chain variable region sequence, or the heavy chain or light chain sequence of MK-4280.

In one embodiment, the anti-LAG-3 antibody molecule is REGN3767 (Regeneron). In one embodiment, the anti-LAG-3 antibody molecule comprises one or more of the CDR sequences (or collectively all of the CDR sequences), the heavy chain or light chain variable region sequence, or the heavy chain or light chain sequence of REGN3767.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 10. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 10
Amino acid sequences of other exemplary
anti-LAG-3 antibody molecules

| | | |
|---|---|---|
| BMS-986016 | | |
| SEQ ID NO: 762 | Heavy chain | QVQLQQSGAGLLKPSETLSLTCAVYGGSFSDYYWNWIRQPPG |
| | | KGLEWIGEINHRGSTNSNPSLKSRVTLSLDTSKNQFSLKLRS |
| | | VTAADTAVYYCAFGYSDYEYNWFDPWGQGTLVTVSSASTKGP |
| | | SVFPLAPCSRSTSESTAALGCLVKDYFPEPVTVSWNSGALTS |
| | | GVHTFPAVLQSSLGYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPREE |
| | | QFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQEG |
| | | NVFSCSVMHEALHNHYTQKSLSLSLGK |
| SEQ ID NO: 763 | Light chain | EIVLTQSPATLSLSPGERATLSCRASQSISSYLAWYQQKPGQ |
| | | APRLLIYDASNRATGIPARFSGSGSGTDFTLTISSLEPEDFA |
| | | VYYCQQRSNWPLTFGQGTNLEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| IMP731 | | |
| SEQ ID NO: 764 | Heavy chain | QVQLKESGPGLVAPSQSLSITCTVSGFSLTAYGNVWVRQPPG |
| | | KGLEWLGMIWDDGSTDYNSALKSRLSISKDNSKQVFLKMNSL |
| | | QTDDTARYYCAREDGVAFDYWGQGTTLTVSSASTKGPSVFPL |
| | | APSSKSTSGGTAALGCLVKDYFPEPVTVSWNSGALTSGVHTF |
| | | PAVLQSSGLYSLSSVVTVPSSSLGTQTYICNVNHKPSNTKVD |
| | | KKVEPKSCDKTHTCPPCPAPELLGGPSVFLFPPKPKDTLMIS |
| | | RTPEVTCVVVDVSHEDPEVKFNWYVDGVEVHNAKTKPREEQY |
| | | NSTYRVVSVLTVLHQDWLNGKEYKCKVSNKALPAPIEKTISK |
| | | AKGQPREPQVYTLPPSRDELTKNQVSLTCLVKGFYPSDIAVE |
| | | WESNGQPENNYKTTPPVLDSDGSFFLYSKLTVDKSRWQQGNV |
| | | FSCSVMHEALHNHYTQKSLSLSPGK |
| SEQ ID NO: 765 | Light chain | DIVMTQSPSSLSAVSVGQKVTMSCKSSQSLLNGSNQKNYLAW |
| | | YQQKPGQSPKLLVYFASTRDSGVPDRFIGSGSGTDFTLTISS |
| | | VQAEDLADYFCLQHFGTPPTFGGGTKLEIKRTVAAPSVFIFP |
| | | PSDEQLKSGTASVVCLLNNFYPREAKVWQKVDNALQSGNSQE |
| | | SVTEQDSKDSTYSLSSTLTLSKADYEKHKVYACEVTHQGLSS |
| | | PVTKSFNRGEC |

### Exemplary TIM-3 Inhibitors

In certain embodiments, the anti-CD32b antibodies disclosed herein can be administered in combination with a TIM-3 inhibitor. The TIM-3 inhibitor may be an antibody, an antigen binding fragment thereof, an immunoadhesin, a fusion protein, or an oligopeptide. In some embodiments, the TIM-3 inhibitor is chosen from MGB453 (Novartis), TSR-022 (Tesaro), or LY3321367 (Eli Lilly).

### Exemplary Anti-TIM-3 Antibody Molecules

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 11 (e.g., from the heavy and light chain variable region sequences of ABTIM3-hum11 or ABTIM3-hum03 disclosed in Table 11), or encoded by a nucleotide sequence shown in Table 11. In some embodiments, the CDRs are according to the Kabat definition (e.g., as set out in Table 11). In some embodiments, the CDRs are according to the Chothia definition (e.g., as set out in Table 11). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 11, or encoded by a nucleotide sequence shown in Table 11.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 11. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 11.

In one embodiment, the anti-TIM-3 antibody molecule comprises a VH comprising the amino acid sequence of SEQ ID NO: 806, or an amino acid sequence having at least about 85%, 90%, 95%, or 99% sequence identity to SEQ ID NO: 806. In one embodiment, the anti-TIM-3 antibody molecule comprises a VL comprising the amino acid sequence of SEQ ID NO: 816, or an amino acid sequence having at least about 85%, 90%, 95%, or 99% sequence identity to SEQ ID NO: 816. In one embodiment, the anti-TIM-3 antibody molecule comprises a VH comprising the amino acid sequence of SEQ ID NO: 822, or an amino acid sequence having at least about 85%, 90%, 95%, or 99% sequence identity to SEQ ID NO: 822. In one embodiment, the anti-TIM-3 antibody molecule comprises a VL comprising the amino acid sequence of SEQ ID NO: 826, or an amino acid sequence having at least about 85%, 90%, 95%, or 99% sequence identity to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence having at least about 85%, 90%, 95%, or 99% sequence identity to SEQ ID NO: 807. In one embodiment, the antibody molecule comprises a VL encoded by the nucleotide sequence of SEQ ID NO: 817, or a nucleotide sequence having at least about 85%, 90%, 95%, or 99% sequence identity to SEQ ID NO: 817. In one embodiment, the antibody molecule comprises a VH encoded by the nucleotide sequence of SEQ ID NO: 823, or a nucleotide sequence having at least about 85%, 90%, 95%, or 99% sequence identity to SEQ ID NO: 823. In one embodiment, the antibody molecule comprises a VL encoded by the nucleotide sequence of SEQ ID NO: 827, or a nucleotide sequence having at least about 85%, 90%, 95%, or 99% sequence identity to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence having at least about 85%, 90%, 95%, or 99% sequence identity to SEQ ID NO: 808. In one embodiment, the anti-TIM-3 antibody molecule comprises a light chain comprising the amino acid sequence of SEQ ID NO: 818, or an amino acid sequence having at least about 85%, 90%, 95%, or 99% sequence identity to SEQ ID NO: 818. In one embodiment, the anti-TIM-3 antibody molecule comprises a heavy chain comprising the amino acid sequence of SEQ ID NO: 824, or an amino acid sequence having at least about 85%, 90%, 95%, or 99% sequence identity to SEQ ID NO: 824. In one embodiment, the anti-TIM-3 antibody molecule comprises a light chain comprising the amino acid sequence of SEQ ID NO: 828, or an amino acid sequence having at least about 85%, 90%, 95%, or 99% sequence identity to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence having at least about 85%, 90%, 95%, or 99% sequence identity to SEQ ID NO: 809. In one embodiment, the antibody molecule comprises a light chain encoded by the nucleotide sequence of SEQ ID NO: 819, or a nucleotide sequence having at least about 85%, 90%, 95%, or 99% sequence identity to SEQ ID NO: 819. In one embodiment, the antibody molecule comprises a heavy chain encoded by the nucleotide sequence of SEQ ID NO: 825, or a nucleotide sequence having at least about 85%, 90%, 95%, or 99% sequence identity to SEQ ID NO: 825. In one embodiment, the antibody molecule comprises a light chain encoded by the nucleotide sequence of SEQ ID NO: 829, or a nucleotide sequence having at least about 85%, 90%, 95%, or 99% sequence identity to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 11
Amino acid and nucleotide sequences of exemplary
anti-TIM-3 antibody molecules

| | | |
|---|---|---|
| ABTIM3-hum11 | | |
| SEQ ID NO: 801 | HCDR1 | SYNMH |
| (Kabat) | | |
| SEQ ID NO: 802 | HCDR2 | DIYPGNGDTSYNQKFKG |
| (Kabat) | | |
| SEQ ID NO: 803 | HCDR3 | VGGAFPMDY |
| (Kabat) | | |
| SEQ ID NO: 804 | HCDR1 | GYTFTSY |
| (Chothia) | | |
| SEQ ID NO: 805 | HCDR2 | YPGNGD |
| (Chothia) | | |
| SEQ ID NO: 803 | HCDR3 | VGGAFPMDY |
| (Chothia) | | |
| SEQ ID NO: 806 | VH | QVQLVQSGAEVKKPGSSVKVSCKASGYTFTSYNMHWVRQAPG |
| | | QGLEWMGDIYPGNGDTSYNQKFKGRVTITADKSTSTVYMELS |
| | | SLRSEDTAVYYCARVGGAFPMDYWGQGTTVTVSS |
| SEQ ID NO: 807 | DNA VH | CAGGTGCAGCTGGTGCAGTCAGGCGCCGAAGTGAAGAAACCC |
| | | GGCTCTAGCGTGAAAGTTTCTTGTAAAGCTAGTGGCTACACC |
| | | TTCACTAGCTATAATATGCACTGGGTTCGCCAGGCCCCAGGG |
| | | CAAGGCCTCGAGTGGATGGGCGATATCTACCCCGGGAACGGC |
| | | GACACTAGTTATAATCAGAAGTTTAAGGGTAGAGTCACTATC |
| | | ACCGCCGATAAGTCTACTAGCACCGTCTATATGGAACTGAGT |
| | | TCCCTGAGGTCTGAGGACACCGCCGTCTACTACTGCGCTAGA |
| | | GTGGGCGGAGCCTTCCCTATGGACTACTGGGGTCAAGGCACT |
| | | ACCGTGACCGTGTCTAGC |
| SEQ ID NO: 808 | DNA VH | QVQLVQSGAEVKKPGSSVKVSCKASGYTFTSYNMHWVRQAPG |
| | | QGLEWMGDIYPGNGDTSYNQKFKGRVTITADKSTSTVYMELS |
| | | SLRSEDTAVYYCARVGGAFPMDYWGQGTTVTVSSASTKGPSV |
| | | FPLAPCSRSTSESTAALGCLVKDYFPEPVTVSWNSGALTSGV |
| | | HTFPAVLQSSGLYSLSSVVTVPSSSLGTKTYTCNVDHKPSNT |
| | | KVDKRVESKYGPPCPPCPAPEFLGGPSVFLFPPKPKDTLMIS |
| | | RTPEVTCVVVDVSQEDPEVQFNWYVDGVEVHNAKTKPREEQF |
| | | NSTYRVVSVLTVLHQDWLNGKEYKCKVSNKGLPSSIEKTISK |
| | | AKGQPREPQVYTLPPSQEEMTKNQVSLTCLVKGFYPSDIAVE |
| | | WESNGQPENNYKTTPPVLDSDGSFFLYSRLTVDKSRWQEGNV |
| | | FSCSVMHEALHNHYTQKSLSLSLG |
| SEQ ID NO: 809 | DNA | CAGGTGCAGCTGGTGCAGTCAGGCGCCGAAGTGAAGAAACCC |
| | heavy chain | GGCTCTAGCGTGAAAGTTTCTTGTAAAGCTAGTGGCTACACC |
| | | TTCACTAGCTATAATATGCACTGGGTTCGCCAGGCCCCAGGG |
| | | CAAGGCCTCGAGTGGATGGGCGATATCTACCCCGGGAACGGC |
| | | GACACTAGTTATAATCAGAAGTTTAAGGGTAGAGTCACTATC |
| | | ACCGCCGATAAGTCTACTAGCACCGTCTATATGGAACTGAGT |
| | | TCCCTGAGGTCTGAGGACACCGCCGTCTACTACTGCGCTAGA |
| | | GTGGGCGGAGCCTTCCCTATGGACTACTGGGGTCAAGGCACT |
| | | ACCGTGACCGTGTCTAGCGCTAGCACTAAGGGCCCGTCCGTG |
| | | TTCCCCCTGGCACCTTGTAGCCGGAGCACTAGCGAATCCACC |
| | | GCTGCCCTCGGCTGCCTGGTCAAGGATTACTTCCCGGAGCCC |
| | | GTGACCGTGTCCTGGAACAGCGGAGCCCTGACCTCCGGAGTG |
| | | CACACCTTCCCCGCTGTGCTGCAGAGCTCCGGGCTGTACTCG |
| | | CTGTCGTCGGTGGTCACGGTGCCTTCATCTAGCCTGGGTACC |
| | | AAGACCTACACTTGCAACGTGGACCACAAGCCTTCCAACACT |
| | | AAGGTGGACAAGCGCGTCGAATCGAAGTACGGCCCACCGTGC |
| | | CCGCCTTGTCCCGCGCCGGAGTTCCTCGGCGGTCCCTCGGTC |
| | | TTTCTGTTCCCACCGAAGCCCAAGGACACTTTGATGATTTCC |
| | | CGCACCCCTGAAGTGACATGCGTGGTCGTGGACGTGTCACAG |
| | | GAAGATCCGGAGGTGCAGTTCAATTGGTACGTGGATGGCGTC |
| | | GAGGTGCACAACGCCAAAACCAAGCCGAGGGAGGAGCAGTTC |
| | | AACTCCACTTACCGCGTCGTGTCCGTGCTGACGGTGCTGCAT |
| | | CAGGACTGGCTGAACGGGAAGGAGTACAAGTGCAAAGTGTCC |
| | | AACAAGGGACTTCCTAGCTCAATCGAAAAGACCATCTCGAAA |
| | | GCCAAGGGACAGCCCCGGGAACCCCAAGTGTATACCCTGCCA |
| | | CCGAGCCAGGAAGAAATGACTAAGAACCAAGTCTCATTGACT |
| | | TGCCTTGTGAAGGGCTTCTACCCATCGGATATCGCCGTGGAA |
| | | TGGGAGTCCAACGGCCAGCCGGAAAACAACTACAAGACCACC |
| | | CCTCCGGTGCTGGACTCAGACGGATCCTTCTTCCTCTACTCG |
| | | CGGCTGACCGTGGATAAGAGCAGATGGCAGGAGGGAAATGTG |
| | | TTCAGCTGTTCTGTGATGCATGAAGCCCTGCACAACCACTAC |
| | | ACTCAGAAGTCCCTGTCCCTCTCCCTGGGA |
| SEQ ID NO: 810 | LCDR1 | RASESVEYYGTSLMQ |
| (Kabat) | | |
| SEQ ID NO: 811 | LCDR2 | AASNVES |
| (Kabat) | | |
| SEQ ID NO: 812 | LCDR3 | QQSRKDPST |
| (Kabat) | | |
| SEQ ID NO: 813 | LCDR1 | SESVEYYGTSL |
| (Chothia) | | |
| SEQ ID NO: 814 | LCDR2 | AAS |
| (Chothia) | | |
| SEQ ID NO: 815 | LCDR3 | SRKDPS |
| (Chothia) | | |
| SEQ ID NO: 816 | VL | AIQLTQSPSSLSASVGDRVTITCRASESVEYYGTSLMQWYQQ |
| | | KPGKAPKLLIYAASNVESGVPSRFSGSGSGTDFTLTISSLQP |
| | | EDFATYFCQQSRKDPSTFGGGTKVEIK |
| SEQ ID NO: 817 | DNA VL | GCTATTCAGCTGACTCAGTCACCTAGTAGCCTGAGCGCTAGT |
| | | GTGGGCGATAGAGTGACTATCACCTGTAGAGCTAGTGAATCA |
| | | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAG |
| SEQ ID NO: 818 | Light chain | AIQLTQSPSSLSASVGDRVTITCRASESVEYYGTSLMQWYQQ |
| | | KPGKAPKLLIYAASNVESGVPSRFSGSGSGTDFTLTISSLQP |
| | | EDFATYFCQQSRKDPSTFGGGTKVEIKRTVAAPSVFIFPPSD |
| | | EQLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 819 | DNA | GCTATTCAGCTGACTCAGTCACCTAGTAGCCTGAGCGCTAGT |
| | light chain | GTGGGCGATAGAGTGACTATCACCTGTAGAGCTAGTGAATCA |
| | | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAGCGTGGTGTGCCTGCTG |
| | | AACAACTTCTACCCCCGGGAGGCCAAGGTGCAGTGGAAGGTG |
| | | GACAACGCCCTGCAGAGCGGCAACAGCCAGGAGAGCGTCACC |
| | | GAGCAGGACAGCAAGGACTCCACCTACAGCCTGAGCAGCACC |
| | | CTGACCCTGAGCAAGGCCGACTACGAGAAGCATAAGGTGTAC |
| | | GCCTGCGAGGTGACCCACCAGGGCCTGTCCAGCCCCGTGACC |
| | | AAGAGCTTCAACAGGGGCGAGTGC |
| ABTIM3-hum03 | | |
| SEQ ID NO: 801 | HCDR1 | SYNMH |
| (Kabat) | | |
| SEQ ID NO: 820 | HCDR2 | DIYPGQGDTSYNQKFKG |
| (Kabat) | | |
| SEQ ID NO: 803 | HCDR3 | VGGAFPMDY |
| (Kabat) | | |
| SEQ ID NO: 804 | HCDR1 | GYTFTSY |
| (Chothia) | | |
| SEQ ID NO: 821 | HCDR2 | YPGQGD |
| (Chothia) | | |
| SEQ ID NO: 803 | HCDR3 | VGGAFPMDY |
| (Chothia) | | |
| SEQ ID NO: 822 | VH | QVQLVQSGAEVKKPGASVKVSCKASGYTFTSYNMHWVRQAPG |
| | | QGLEWIGDIYPGQGDTSYNQKFKGRATMTADKSTSTVYMELS |
| | | SLRSEDTAVYYCARVGGAFPMDYWGQGTLVTVSS |
| SEQ ID NO: 823 | DNA VH | CAGGTGCAGCTGGTGCAGTCAGGCGCCGAAGTGAAGAAACCC |
| | | GGCGCTAGTGTGAAAGTTAGCTGTAAAGCTAGTGGCTATACT |
| | | TTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAAGTTTAAGGGTAGAGCTACTATG |
| | | ACCGCCGATAAGTCTACTTCTACCGTCTATATGGAACTGAGT |
| | | TCCCTGAGGTCTGAGGACACCGCCGTCTACTACTGCGCTAGA |
| | | GTGGGCGGAGCCTTCCCAATGGACTACTGGGGTCAAGGCACC |
| | | CTGGTCACCGTGTCTAGC |
| SEQ ID NO: 824 | Heavy chain | QVQLVQSGAEVKKPGASVKVSCKASGYTFTSYNMHWVRQAPG |
| | | QGLEWIGDIYPGQGDTSYNQKFKGRATMTADKSTSTVYMELS |
| | | SLRSEDTAVYYCARVGGAFPMDYWGQGTLVTVSSASTKGPSV |
| | | FPLAPCSRSTSESTAALGCLVKDYFPEPVTVSWNSGALTSGV |
| | | HTFPAVLQSSGLYSLSSVVTVPSSSLGTKTYTCNVDHKPSNT |
| | | KVDKRVESKYGPPCPPCPAPEFLGGPSVFLFPPKPKDTLMIS |
| | | RTPEVTCVVVDVSQEDPEVQFNWYVDGVEVHNAKTKPREEQF |
| | | NSTYRVVSVLTVLHQDWLNGKEYKCKVSNKGLPSSIEKTISK |
| | | AKGQPREPQVYTLPPSQEEMTKNQVSLTCLVKGFYPSDIAVE |
| | | WESNGQPENNYKTTPPVLDSDGSFFLYSRLTVDKSRWQEGNV |
| | | FSCSVMHEALHNHYTQKSLSLSLG |
| SEQ ID NO: 825 | DNA | CAGGTGCAGCTGGTGCAGTCAGGCGCCGAAGTGAAGAAACCC |
| | heavy chain | GGCGCTAGTGTGAAAGTTAGCTGTAAAGCTAGTGGCTATACT |
| | | TTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAAGTTTAAGGGTAGAGCTACTATG |
| | | ACCGCCGATAAGTCTACTTCTACCGTCTATATGGAACTGAGT |
| | | TCCCTGAGGTCTGAGGACACCGCCGTCTACTACTGCGCTAGA |
| | | GTGGGCGGAGCCTTCCCAATGGACTACTGGGGTCAAGGCACC |
| | | CTGGTCACCGTGTCTAGCGCTAGCACTAAGGGCCCGTCCGTG |
| | | TTCCCCCTGGCACCTTGTAGCCGGAGCACTAGCGAATCCACC |
| | | GCTGCCCTCGGCTGCCTGGTCAAGGATTACTTCCCGGAGCCC |
| | | GTGACCGTGTCCTGGAACAGCGGAGCCCTGACCTCCGGAGTG |
| | | CACACCTTCCCCGCTGTGCTGCAGAGCTCCGGGCTGTACTCG |
| | | CTGTCGTCGGTGGTCACGGTGCCTTCATCTAGCCTGGGTACC |
| | | AAGACCTACACTTGCAACGTGGACCACAAGCCTTCCAACACT |
| | | AAGGTGGACAAGCGCGTCGAATCGAAGTACGGCCCACCGTGC |
| | | CCGCCTTGTCCCGCGCCGGAGTTCCTCGGCGGTCCCTCGGTC |
| | | TTTCTGTTCCCACCGAAGCCCAAGGACACTTTGATGATTTCC |
| | | CGCACCCCTGAAGTGACATGCGTGGTCGTGGACGTGTCACAG |
| | | GAAGATCCGGAGGTGCAGTTCAATTGGTACGTGGATGGCGTC |
| | | GAGGTGCACAACGCCAAAACCAAGCCGAGGGAGGAGCAGTTC |
| | | AACTCCACTTACCGCGTCGTGTCCGTGCTGACGGTGCTGCAT |
| | | CAGGACTGGCTGAACGGGAAGGAGTACAAGTGCAAAGTGTCC |
| | | AACAAGGGACTTCCTAGCTCAATCGAAAAGACCATCTCGAAA |
| | | GCCAAGGGACAGCCCCGGGAACCCCAAGTGTATACCCTGCCA |
| | | CCGAGCCAGGAAGAAATGACTAAGAACCAAGTCTCATTGACT |
| | | TGCCTTGTGAAGGGCTTCTACCCATCGGATATCGCCGTGGAA |
| | | TGGGAGTCCAACGGCCAGCCGGAAAACAACTACAAGACCACC |
| | | CCTCCGGTGCTGGACTCAGACGGATCCTTCTTCCTCTACTCG |
| | | CGGCTGACCGTGGATAAGAGCAGATGGCAGGAGGGAAATGTG |
| | | TTCAGCTGTTCTGTGATGCATGAAGCCCTGCACAACCACTAC |
| | | ACTCAGAAGTCCCTGTCCCTCTCCCTGGGA |
| SEQ ID NO: 810 | LCDR1 | RASESVEYYGTSLMQ |
| (Kabat) | | |
| SEQ ID NO: 811 | LCDR2 | AASNVES |
| (Kabat) | | |
| SEQ ID NO: 812 | LCDR3 | QQSRKDPST |
| (Kabat) | | |
| SEQ ID NO: 813 | LCDR1 | SESVEYYGTSL |
| (Chothia) | | |
| SEQ ID NO: 814 | LCDR2 | AAS |
| (Chothia) | | |
| SEQ ID NO: 815 | LCDR3 | SRKDPS |
| (Chothia) | | |
| SEQ ID NO: 826 | VL | DIVLTQSPDSLAVSLGERATINCRASESVEYYGTSLMQWYQQ |
| | | KPGQPPKLLIYAASNVESGVPDRFSGSGSGTDFTLTISSLQA |
| | | EDVAVYYCQQSRKDPSTFGGGTKVEIK |
| SEQ ID NO: 827 | DNA VL | GATATCGTCCTGACTCAGTCACCCGATAGCCTGGCCGTCAGC |
| | | CTGGGCGAGCGGGCTACTATTAACTGTAGAGCTAGTGAATCA |
| | | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAG |
| SEQ ID NO: 828 | Light chain | DIVLTQSPDSLAVSLGERATINCRASESVEYYGTSLMQWYQQ |
| | | KPGQPPKLLIYAASNVESGVPDRFSGSGSGTDFTLTISSLQA |
| | | EDVAVYYCQQSRKDPSTFGGGTKVEIKRTVAAPSVFIFPPSD |
| | | EQLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 829 | DNA | GATATCGTCCTGACTCAGTCACCCGATAGCCTGGCCGTCAGC |
| | light chain | CTGGGCGAGCGGGCTACTATTAACTGTAGAGCTAGTGAATCA |
| | | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAGCGTGGTGTGCCTGCTG |
| | | AACAACTTCTACCCCCGGGAGGCCAAGGTGCAGTGGAAGGTG |
| | | GACAACGCCCTGCAGAGCGGCAACAGCCAGGAGAGCGTCACC |
| | | GAGCAGGACAGCAAGGACTCCACCTACAGCCTGAGCAGCACC |
| | | CTGACCCTGAGCAAGGCCGACTACGAGAAGCATAAGGTGTAC |
| | | GCCTGCGAGGTGACCCACCAGGGCCTGTCCAGCCCCGTGACC |
| | | AAGAGCTTCAACAGGGGCGAGTGC |

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 12. APE5137, APE5121, and other anti-TIM-3 antibodies are disclosed in WO 2016/161270, incorporated by reference in its entirety.

In one embodiment, the anti-TIM-3 antibody molecule is LY3321367 (Eli Lilly). In one embodiment, the anti-TIM-3 antibody molecule comprises one or more of the CDR sequences (or collectively all of the CDR sequences), the heavy chain or light chain variable region sequence, or the heavy chain or light chain sequence of LY3321367.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 12
Amino acid sequences of other exemplary
anti-TIM-3 antibody molecules

| | | |
|---|---|---|
| APE5137 | | |
| SEQ ID NO: 830 | VH | EVQLLESGGGLVQPGGSLRLSCAAASGFTFSSYDMSWVRQAPGKGLDWV |
| | | STISGGGTYTYYQDSVKGRFTISRDNSKNTLYLQMNSLRAEDTAVYYCA |
| | | SMDYWGQGTTVTVSSA |
| SEQ ID NO: 831 | VL | DIQMTQSPSSLSASVGDRVTITCRASQSIRRYLNWYHQKPGKAPKLLIY |
| | | GASTLQSGVPSRFSGSGSGTDFTLTISSLQPEDFAVYYCQQSHSAPLTF |
| | | GGGTKVEIKR |
| APE5121 | | |
| SEQ ID NO: 832 | VH | EVQVLESGGGLVQPGGSLRLYCVASGFTFSGSYAMSWVRQAPGKGLEWV |
| | | SAISGSGGSTYYADSVKGRFTISRDNSKNTLYLQMNSLRAEDTAVYYCA |
| | | KKYYVGPADYWGQGTLVTVSSG |
| SEQ ID NO: 833 | VL | DIVMTQSPDSLAVSLGERATINCKSSQSVLYSSNNKNYLAWYQHKPGQP |
| | | PKLLIYWASTRESGVPDRFSGSGSGTDFTLTISSLQAEDVAVYYCQQYY |
| | | SSPLTFGGGTKIEVK |

### Exemplary CTLA-4 Inhibitors

In certain embodiments, the anti-CD32b antibodies disclosed herein can be administered in combination with a CTLA-4 inhibitor. The CTLA-4 inhibitor may be an antibody, an antigen binding fragment thereof, an immunoadhesin, a fusion protein, or an oligopeptide. In some embodiments, the CTLA-4 inhibitor is Ipilimumab (Yervoy®, Bristol-Myers Squibb) or Tremelimumab (Pfizer). The antibody Ipilimumab and other anti-CTLA-4 antibodies are disclosed in U.S. Pat. No. 6,984,720, herein incorporated by reference. The antibody Tremelimumab and other anti-CTLA-4 antibodies are disclosed in U.S. Pat. No. 7,411,057, herein incorporated by reference.

### Exemplary GITR Agonists

In certain embodiments, the anti-CD32b antibodies disclosed herein can be administered in combination with a GITR agonist. The GITR agonist may be an antibody, an antigen binding fragment thereof, an immunoadhesin, a fusion protein, or an oligopeptide. In some embodiments, the GITR agonist is GWN323 (Novartis), BMS-986156 (BMS), MK-4166 or MK-1248 (Merck), TRX518 (Leap Therapeutics), INCAGN1876 (Incyte/Agenus), AMG 228 (Amgen), or INBRX-110 (Inhibrx).

### Exemplary Anti-GITR Antibody Molecules

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 13 (e.g., from the heavy and light chain variable region sequences of MAB7 disclosed in Table 13), or encoded by a nucleotide sequence shown in Table 13. In some embodiments, the CDRs are according to the Kabat definition (e.g., as set out in Table 13). In some embodiments, the CDRs are according to the Chothia definition (e.g., as set out in Table 13). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 13, or encoded by a nucleotide sequence shown in Table 13.

In one embodiment, the anti-GITR antibody molecule comprises a heavy chain variable region (VH) comprising a VHCDR1 amino acid sequence of SEQ ID NO: 909, a VHCDR2 amino acid sequence of SEQ ID NO: 911, and a VHCDR3 amino acid sequence of SEQ ID NO: 913; and a light chain variable region (VL) comprising a VLCDR1 amino acid sequence of SEQ ID NO: 914, a VLCDR2 amino acid sequence of SEQ ID NO: 916, and a VLCDR3 amino acid sequence of SEQ ID NO: 918, each disclosed in Table 13.

In one embodiment, the anti-GITR antibody molecule comprises a VH comprising the amino acid sequence of SEQ ID NO: 901, or an amino acid sequence at least 85%, 90%, 95%, or 99% identical or higher to SEQ ID NO: 901. In one embodiment, the anti-GITR antibody molecule comprises a VL comprising the amino acid sequence of SEQ ID NO: 902, or an amino acid sequence at least 85%, 90%, 95%, or 99% identical or higher to SEQ ID NO: 902. In one embodiment, the anti-GITR antibody molecule comprises a VH comprising the amino acid sequence of SEQ ID NO: 901 and a VL comprising the amino acid sequence of SEQ ID NO: 902.

In one embodiment, the antibody molecule comprises a VH encoded by the nucleotide sequence of SEQ ID NO: 905, or a nucleotide sequence at least 85%, 90%, 95%, or 99% identical or higher to SEQ ID NO: 905. In one embodiment, the antibody molecule comprises a VL encoded by the nucleotide sequence of SEQ ID NO: 906, or a nucleotide sequence at least 85%, 90%, 95%, or 99% identical or higher to SEQ ID NO: 906. In one embodiment, the antibody molecule comprises a VH encoded by the nucleotide sequence of SEQ ID NO: 905 and a VL encoded by the nucleotide sequence of SEQ ID NO: 906.

In one embodiment, the anti-GITR antibody molecule comprises a heavy chain comprising the amino acid sequence of SEQ ID NO: 903, or an amino acid sequence at least 85%, 90%, 95%, or 99% identical or higher to SEQ ID NO: 903. In one embodiment, the anti-GITR antibody molecule comprises a light chain comprising the amino acid sequence of SEQ ID NO: 904, or an amino acid sequence at least 85%, 90%, 95%, or 99% identical or higher to SEQ ID NO: 904.

In one embodiment, the anti-GITR antibody molecule comprises a heavy chain comprising the amino acid sequence of SEQ ID NO: 903 and a light chain comprising the amino acid sequence of SEQ ID NO: 904.

In one embodiment, the antibody molecule comprises a heavy chain encoded by the nucleotide sequence of SEQ ID NO: 907, or a nucleotide sequence at least 85%, 90%, 95%, or 99% identical or higher to SEQ ID NO: 907. In one embodiment, the antibody molecule comprises a light chain encoded by the nucleotide sequence of SEQ ID NO: 908, or a nucleotide sequence at least 85%, 90%, 95%, or 99% identical or higher to SEQ ID NO: 908. In one embodiment, the antibody molecule comprises a heavy chain encoded by the nucleotide sequence of SEQ ID NO: 907 and a light chain encoded by the nucleotide sequence of SEQ ID NO: 908.

The antibody molecules described herein can be made by vectors, host cells, and methods described in WO 2016/057846, incorporated by reference in its entirety.

TABLE 13
Amino acid and nucleotide sequences of
exemplary anti-GITR antibody molecule

| | | |
|---|---|---|
| MAB7 | | |
| SEQ ID NO: 901 | VH | EVQLVESGGGLVQSGGSLRLSCAASGFSLSSYGVDWVRQAPGKGL |
| | | EWVGVIWGGGGTYYASSLMGRFTISRDNSKNTLYLQMNSLRAEDT |
| | | AVYYCARHAYGHDGGFAMDYWGQGTLVTVSS |
| SEQ ID NO: 902 | VL | EIVMTQSPATLSVSPGERATLSCRASESVSSNVAWYQQRPGQAPR |
| | | LLIYGASNRATGIPARFSGSGSGTDFTLTISRLEPEDFAVYYCGQ |
| | | SYSYPFTFGQGTKLEIK |
| SEQ ID NO: 903 | Heavy chain | EVQLVESGGGLVQSGGSLRLSCAASGFSLSSYGVDWVRQAPGKGL |
| | | EWVGVIWGGGGTYYASSLMGRFTISRDNSKNTLYLQMNSLRAEDT |
| | | AVYYCARHAYGHDGGFAMDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRE |
| | | EMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| | | K |
| SEQ ID NO: 904 | Light chain | EIVMTQSPATLSVSPGERATLSCRASESVSSNVAWYQQRPGQAPR |
| | | LLIYGASNRATGIPARFSGSGSGTDFTLTISRLEPEDFAVYYCGQ |
| | | SYSYPFTFGQGTKLEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSFNRGEC |
| SEQ ID NO: 905 | DNA VH | GAGGTGCAGCTGGTGGAATCTGGCGGCGGACTGGTGCAGTCCGGC |
| | | GGCTCTCTGAGACTGTCTTGCGCTGCCTCCGGCTTCTCCCTGTCC |
| | | TCTTACGGCGTGGACTGGGTGCGACAGGCCCCTGGCAAGGGCCTG |
| | | GAATGGGTGGGAGTGATCTGGGGCGGAGGCGGCACCTACTACGCC |
| | | TCTTCCCTGATGGGCCGGTTCACCATCTCCCGGGACAACTCCAAG |
| | | AACACCCTGTACCTGCAGATGAACTCCCTGCGGGCCGAGGACACC |
| | | GCCGTGTACTACTGCGCCAGACACGCCTACGGCCACGACGGCGGC |
| | | TTCGCCATGGATTATTGGGGCCAGGGCACCCTGGTGACAGTGTCC |
| | | TCC |
| SEQ ID NO: 906 | DNA VL | GAGATCGTGATGACCCAGTCCCCCGCCACCCTGTCTGTGTCTCCC |
| | | GGCGAGAGAGCCACCCTGAGCTGCAGAGCCTCCGAGTCCGTGTCC |
| | | TCCAACGTGGCCTGGTATCAGCAGAGACCTGGTCAGGCCCCTCGG |
| | | CTGCTGATCTACGGCGCCTCTAACCGGGCCACCGGCATCCCTGCC |
| | | AGATTCTCCGGCTCCGGCAGCGGCACCGACTTCACCCTGACCATC |
| | | TCCCGGCTGGAACCCGAGGACTTCGCCGTGTACTACTGCGGCCAG |
| | | TCCTACTCATACCCCTTCACCTTCGGCCAGGGCACCAAGCTGGAA |
| | | ATCAAG |
| SEQ ID NO: 907 | DNA | GAGGTGCAGCTGGTGGAATCTGGCGGCGGACTGGTGCAGTCCGGC |
| | Heavy chain | GGCTCTCTGAGACTGTCTTGCGCTGCCTCCGGCTTCTCCCTGTCC |
| | | TCTTACGGCGTGGACTGGGTGCGACAGGCCCCTGGCAAGGGCCTG |
| | | GAATGGGTGGGAGTGATCTGGGGCGGAGGCGGCACCTACTACGCC |
| | | TCTTCCCTGATGGGCCGGTTCACCATCTCCCGGGACAACTCCAAG |
| | | AACACCCTGTACCTGCAGATGAACTCCCTGCGGGCCGAGGACACC |
| | | GCCGTGTACTACTGCGCCAGACACGCCTACGGCCACGACGGCGGC |
| | | TTCGCCATGGATTATTGGGGCCAGGGCACCCTGGTGACAGTGTCC |
| | | TCCGCTAGCACCAAGGGCCCAAGTGTGTTTCCCCTGGCCCCCAGC |
| | | AGCAAGTCTACTTCCGGCGGAACTGCTGCCCTGGGTTGCCTGGTG |
| | | AAGGACTACTTCCCCGAGCCCGTGACAGTGTCCTGGAACTCTGGG |
| | | GCTCTGACTTCCGGCGTGCACACCTTCCCCGCCGTGCTGCAGAGC |
| | | AGCGGCCTGTACAGCCTGAGCAGCGTGGTGACAGTGCCCTCCAGC |
| | | TCTCTGGGAACCCAGACCTATATCTGCAACGTGAACCACAAGCCC |
| | | AGCAACACCAAGGTGGACAAGAGAGTGGAGCCCAAGAGCTGCGAC |
| | | AAGACCCACACCTGCCCCCCCTGCCCAGCTCCAGAACTGCTGGGA |
| | | GGGCCTTCCGTGTTCCTGTTCCCCCCCAAGCCCAAGGACACCCTG |
| | | ATGATCAGCAGGACCCCCGAGGTGACCTGCGTGGTGGTGGACGTG |
| | | TCCCACGAGGACCCAGAGGTGAAGTTCAACTGGTACGTGGACGGC |
| | | GTGGAGGTGCACAACGCCAAGACCAAGCCCAGAGAGGAGCAGTAC |
| | | AACAGCACCTACAGGGTGGTGTCCGTGCTGACCGTGCTGCACCAG |
| | | GACTGGCTGAACGGCAAAGAATACAAGTGCAAAGTCTCCAACAAG |
| | | GCCCTGCCAGCCCCAATCGAAAAGACAATCAGCAAGGCCAAGGGC |
| | | CAGCCACGGGAGCCCCAGGTGTACACCCTGCCCCCCAGCCGGGAG |
| | | GAGATGACCAAGAACCAGGTGTCCCTGACCTGTCTGGTGAAGGGC |
| | | TTCTACCCCAGCGATATCGCCGTGGAGTGGGAGAGCAACGGCCAG |
| | | CCCGAGAACAACTACAAGACCACCCCCCCAGTGCTGGACAGCGAC |
| | | GGCAGCTTCTTCCTGTACAGCAAGCTGACCGTGGACAAGTCCAGG |
| | | TGGCAGCAGGGCAACGTGTTCAGCTGCAGCGTGATGCACGAGGCC |
| | | CTGCACAACCACTACACCCAGAAGTCCCTGAGCCTGAGCCCCGGC |
| | | AAG |
| SEQ ID NO: 908 | DNA | GAGATCGTGATGACCCAGTCCCCCGCCACCCTGTCTGTGTCTCCC |
| | Light chain | GGCGAGAGAGCCACCCTGAGCTGCAGAGCCTCCGAGTCCGTGTCC |
| | | TCCAACGTGGCCTGGTATCAGCAGAGACCTGGTCAGGCCCCTCGG |
| | | CTGCTGATCTACGGCGCCTCTAACCGGGCCACCGGCATCCCTGCC |
| | | AGATTCTCCGGCTCCGGCAGCGGCACCGACTTCACCCTGACCATC |
| | | TCCCGGCTGGAACCCGAGGACTTCGCCGTGTACTACTGCGGCCAG |
| | | TCCTACTCATACCCCTTCACCTTCGGCCAGGGCACCAAGCTGGAA |
| | | ATCAAGCGTACGGTGGCCGCTCCCAGCGTGTTCATCTTCCCCCCC |
| | | AGCGACGAGCAGCTGAAGAGCGGCACCGCCAGCGTGGTGTGCCTG |
| | | CTGAACAACTTCTACCCCCGGGAGGCCAAGGTGCAGTGGAAGGTG |
| | | GACAACGCCCTGCAGAGCGGCAACAGCCAGGAGAGCGTCACCGAG |
| | | CAGGACAGCAAGGACTCCACCTACAGCCTGAGCAGCACCCTGACC |
| | | CTGAGCAAGGCCGACTACGAGAAGCATAAGGTGTACGCCTGCGAG |
| | | GTGACCCACCAGGGCCTGTCCAGCCCCGTGACCAAGAGCTTCAAC |
| | | AGGGGCGAGTGC |
| SEQ ID NO: 909 (KABAT) | HCDR1 | SYGVD |
| SEQ ID NO: 910 | HCDR1 | GFSLSSY |
| (CHOTHIA) | | |
| SEQ ID NO: 911 (KABAT) | HCDR2 | VIWGGGGTYYASSLMG |
| SEQ ID NO: 912 | HCDR2 | WGGGG |
| (CHOTHIA) | | |
| SEQ ID NO: 913 (KABAT) | HCDR3 | HAYGHDGGFAMDY |
| SEQ ID NO: 913 HCDR3 | | HAYGHDGGFAMDY |
| (CHOTHIA) | | |
| SEQ ID NO: 914 (KABAT) | LCDR1 | RASESVSSNVA |
| SEQ ID NO: 915 LCDR1 | | SESVSSN |
| (CHOTHIA) | | |
| SEQ ID NO: 916 (KABAT) | LCDR2 | GASNRAT |
| SEQ ID NO: 917 LCDR2 | | GAS |
| (CHOTHIA) | | |
| SEQ ID NO: 918 (KABAT) | LCDR3 | GQSYSYPFT |
| SEQ ID NO: 919 | LCDR3 | SYSYPF |
| (CHOTHIA) | | |

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 14.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (Medlmmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 14
Amino acid sequence of other exemplary
anti-GITR antibody molecules

| | | |
|---|---|---|
| BMS-986156 | | |
| SEQ ID NO: 920 | VH | QVQLVESGGGVVQPGRSLRLSCAASGFTFSSY |
| | | GMHWVRQAPGKGLEWVAVIWYEGSNKYYADSV |
| | | KGRFTISRDNSKNTLYLQMNSLRAEDTAVYYC |
| | | ARGGSMVRGDYYYGMDVWGQGTTVTVSS |
| SEQ ID NO: 921 | VL | AIQLTQSPSSLSASVGDRVTITCRASQGISSA |
| | | LAWYQQKPGKAPKLLIYDASSLESGVPSRFSG |
| | | SGSGTDFTLTISSLQPEDFATYYCQQFNSYPY |
| | | TFGQGTKLEIK |

### Exemplary STING Agonists

In certain embodiments, the anti-CD32b antibodies disclosed herein can be administered in combination with a STING agonist. In some embodiments, the combination is used to treat a cancer, e.g., a cancer described herein e.g., a solid tumor (e.g., a breast cancer, a squamous cell carcinoma, a melanoma, an ovarian cancer, a fallopian tube carcinoma, a peritoneal carcinoma, a soft tissue sarcoma, an esophageal cancer, a head and neck cancer, an endometrial cancer, a cervical cancer, or a basal cell carcinoma), e.g., a hematologic malignancy (e.g., a leukemia (e.g., a chronic lymphocytic leukemia (CLL), or a lymphoma (e.g., a marginal zone B-cell lymphoma, a small lymphocytic lymphoma, a follicular lymphoma, Hodgkin lymphoma, non-Hodgkin lymphoma)). In some embodiments, the cancer is chosen from a head and neck cancer (e.g., a head and neck squamous cell carcinoma (HNSCC), a skin cancer (e.g., melanoma), a lung cancer (e.g., a non-small cell lung cancer (NSCLC)), or a breast cancer (e.g., a triple negative breast cancer (TNBC)).

In some embodiments, the STING agonist is cyclic dinucleotide, e.g., a cyclic dinucleotide comprising purine or pyrimidine nucleobases (e.g., adenosine, guanine, uracil, thymine, or cytosine nucleobases). In some embodiments, the nucleobases of the cyclic dinucleotide comprise the same nucleobase or different nucleobases.

In some embodiments, the STING agonist comprises an adenosine or a guanosine nucleobase. In some embodiments, the STING agonist comprises one adenosine nucleobase and one guanosine nucleobase. In some embodiments, the STING agonist comprises two adenosine nucleobases or two guanosine nucleobases.

In some embodiments, the STING agonist comprises a modified cyclic dinucleotide, e.g., comprising a modified nucleobase, a modified ribose, or a modified phosphate linkage. In some embodiments, the modified cyclic dinucleotide comprises a modified phosphate linkage, e.g., a thiophosphate.

In some embodiments, the STING agonist comprises a cyclic dinucleotide (e.g., a modified cyclic dinucleotide) with 2′,5′ or 3′,5′ phosphate linkages. In some embodiments, the STING agonist comprises a cyclic dinucleotide (e.g., a modified cyclic dinucleotide) with Rp or Sp stereochemistry around the phosphate linkages.

In some embodiments, the STING agonist is Rp,Rp dithio 2′,3′ c-di-AMP (e.g., Rp,Rp-dithio c-[A(2′,5′)pA(3′,5′)p], or a cyclic dinucleotide analog thereof. In some embodiments, the STING agonist is a compound depicted in U.S. Patent Publication No. US2015/0056224 (e.g., a compound in FIG. 2c, e.g., compound 21 or compound 22). In some embodiments, the STING agonist is c-[G(2′,5′)pG(3′,5′)p], a dithio ribose 0-substituted derivative thereof, or a compound depicted in FIG. 4 of PCT Publication Nos. WO 2014/189805 and WO 2014/189806. In some embodiments, the STING agonist is c-[A(2′,5′)pA(3′,5′)p] or a dithio ribose O-substituted derivative thereof, or is a compound depicted in FIG. 5 of PCT Publication Nos. WO 2014/189805 and WO 2014/189806. In some embodiments, the STING agonist is c-[G(2′,5′)pA(3′,5′)p], or a dithio ribose O-substituted derivative thereof, or is a compound depicted in FIG. 5 of PCT Publication Nos. WO 2014/189805 and WO 2014/189806. In some embodiments, the STING agonist is 2′-O-propargyl-cyclic-[A(2′,5′)pA(3′,5′)p] (2′-O-propargyl-ML-CDA) or a compound depicted in FIG. 7 of PCT Publication No. WO 2014/189806. Other exemplary STING agonists are disclosed, e.g., in PCT Publication Nos. WO 2014/189805 and WO 2014/189806, and U.S. Publication No. 2015/0056225.

In one embodiment, the inhibitor of CEACAM (e.g., CEACAM-1, -3 and/or -5) is an anti-CEACAM antibody molecule. Carcinoembryonic antigen cell adhesion molecules (CEACAM), such as CEACAM-1 and CEACAM-5, are believed to mediate, at least in part, inhibition of an anti-tumor immune response (see e.g., Markel et al. J Immunol. 2002 Mar. 15; 168(6):2803-10; Markel et al. J Immunol. 2006 Nov. 1; 177(9):6062-71; Markel et al. Immunology. 2009 February; 126(2):186-200; Markel et al. Cancer Immunol Immunother. 2010 February; 59(2):215-30; Ortenberg et al. Mol Cancer Ther. 2012 June; 11(6):1300-10; Stern et al. J Immunol. 2005 Jun. 1; 174(11):6692-701; Zheng et al. PLoS One. 2010 Sep. 2; 5(9). pii: e12529). For example, CEACAM-1 has been described as a heterophilic ligand for TIM-3 and as playing a role in TIM-3-mediated T cell tolerance and exhaustion (see e.g., WO 2014/022332; Huang, et al. (2014) Nature doi:10.1038/nature13848). In embodiments, co-blockade of CEACAM-1 and TIM-3 has been shown to enhance an anti-tumor immune response in xenograft colorectal cancer models (see e.g., WO 2014/022332; Huang, et al. (2014), supra). In other embodiments, co-blockade of CEACAM-1 and PD-1 reduce T cell tolerance as described, e.g., in WO 2014/059251. Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or crossreacts with CEACAM-1 and CEACAM-5 as described in, e.g., WO 2013/054331 and US 2014/0271618, herein incorporated by reference in their entirety.

In one embodiment, the anti-CD32b antibody molecule is administered in combination with an antibody that binds a cell surface antigen, e.g., a surface antigen on an immune cell or a cancer or a tumor cell. In some embodiments, the cell surface antigen is co-expressed or not co-expressed, with CD32b. In other embodiments, the combination further comprises another therapeutic agent. In some embodiments, CD32b and the cell surface antigen are co-expressed on B cells. In some embodiments, the cell surface antigen is selected from the group consisting of CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-Al); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In other embodiments, the cell surface antigen is selected from the group consisting of CD20, CD38, CD52, CS1/SLAMF7, KiR, CD56, CD138, CD19, CD40, Thy-1, Ly-6, CD49, Fas, Cd95, APO-1, EGFR, HER2, CXCR4, HLA molecules, GM1, CD22, CD23, CD80, CD74, DRD, CD33, ERBB2 (HER2/Neu), TSHR, CD171, CS-1, CLL-1, GD3, Tn Ag, FLT3, CD44v6, B7H3, KIT, IL-13Ra2, IL-11Ra, PSCA, PRSS21, VEGF, VEGFR2, LewisY, CD24, PDGFR-beta, SSEA-4, MUC1, NCAM, CAIX, LMP2, EphA2, Fucosyl GM1, sLe, GM3, TGS5, HMWMAA, o-acetyl-GD2, Folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, and IGLL1. In some embodiments, the cell surface antigen is CD38.

In some embodiments, the CD32b-binding antibody molecules disclosed herein are administered in combination with one or more additional antibodies selected from the group consisting of: rituximab, obinutuzumab, ofatumumab, daratumumab, elotuzumab, alemtuzumab, Ocrelizumab, Veltuzumab, GA101, Gemtuzumab ogozamicin, lintuzumab, cetuximab, panitumumab, zalutumumab, nimotuzumab, matuzumab, trastuzumab, pertuzumab, bevacizumab, trastuzumab, or ibritumomab tiuxetan. In some embodiments, the additional antibody is daratumumab.

In one embodiment, an anti-CD32b antibody molecule is administered in combination with a second or additional therapeutic agent, e.g., an anti-cancer therapy, e.g., one or more of a targeted anti-cancer therapy (e.g., an antibody molecule), or a cytotoxic agent (e.g., a chemotherapy, an oncolytic drug, or a small molecule inhibitor) described herein. In one embodiment, the second therapeutic agent comprises any of the agents described herein.

Exemplary therapeutic agents that can comprise a second or additional therapeutic agent administered in combination with an anti-CD32b antibody molecule are disclosed below:

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 is an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against CLL-1 is an antigen binding portion, e.g., CDRs, of an antibody available from R&D, ebiosciences, Abcam, for example, PE-CLL1-hu Cat #353604 (BioLegend); and PE-CLL1 (CLEC12A) Cat #562566 (BD).

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6), Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012:683065 (2012), and Pizzitola et al., Leukemia doi:10.1038/Lue.2014.62 (2014).

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen binding portion, e.g., CDRs, of an antibody described in, e.g., WO2012163805, WO200112812, and WO2003062401. In one embodiment, an antigen binding domain against Tn antigen is an antigen binding portion, e.g., CDRs, of an antibody described in, e.g., U.S. Pat. No. 8,440,798, Brooks et al., PNAS 107(22):10056-10061 (2010), and Stone et al., OncoImmunology 1(6):863-873(2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam). In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013). In one embodiment, an antigen binding domain against CEA is an antigen binding portion, e.g., CDRs, of an antibody described in, e.g., Chmielewski et al., Gastoenterology 143(4):1095-1107 (2012).

In one embodiment, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-11Ra is an antigen binding portion, e.g., CDRs, of an antibody available from Abcam (cat # ab55262) or Novus Biologicals (cat # EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013(2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab.

In one embodiment, an antigen binding domain against NCAM is an antigen binding portion, e.g., CDRs, of the antibody clone 2-2B: MAB5324 (EMD Millipore)

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007

In one embodiment, an antigen binding domain against tyrosinase is an antigen binding portion, e.g., CDRs, of an antibody described in, e.g., U.S. Pat. No. 5,843,674; or U.S. Ser. No. 19/950,504048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., US20100297138; or WO2007/067992.

In one embodiment, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693.

In one embodiment, an antigen binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J.15(3):243-9 (1998), Lou et al., Proc Natl Acad Sci USA 111(7):2482-2487 (2014); MBr 1: Bremer E-G et al. J Biol Chem 259:14773-14777 (1984).

In one embodiment, an antigen binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100 (Lifespan Biosciences)

In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody Lifespan Biosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748-Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56th ASH Annual Meeting and Exposition, San Francisco, Calif. December 6-9 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from Pro Spec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog#10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences. In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1xCD3 BiTE Antibody” 53rd ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32. In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In some embodiments, the anti-CD32 antibody or antigen binding fragment thereof disclosed herein is administered in combination with any one or more of: HERCEPTIN® (trastuzumab, Genentech Inc.,); FASLODEX® (fulvestrant, AstraZeneca Pharmaceuticals); ARIMIDEX® (anastrozole, AstraZeneca Pharmaceuticals, LP); Aromasin® (exemestane, Pfizer Inc.,); FEMARA® (letrozole, Novartis Pharmaceuticals); and NOLVADEX® (tamoxifen, AstraZeneca Pharmaceuticals, LP) to treat breast cancer.

In some embodiments, the anti-CD32 antibody or antigen binding fragment thereof disclosed herein is administered in combination with any one or more of: AVASTIN®; ERBITUX® (cetuximab, ImClone Systems Inc.); GLEEVEC® (imatinib mesylate; a protein kinase inhibitor); and ERGAMISOL® (levamisole hydrochloride, Janssen Pharmaceutica Products, LP) to treat colorectal cancer.

In some embodiments, the anti-CD32 antibody or antigen binding fragment thereof disclosed herein is administed in combination with TARCEVA® (erlotinib HCL, OSI Pharmaceuticals Inc.) to treat lung cancer.

In some embodiments, the anti-CD32 antibody or antigen binding fragment thereof disclosed herein is administeredin combination with VELCADE® (bortezomib, Millennium Pharmaceuticals, Cambridge Mass.; a proteasome inhibitor), THALIDOMID® (Celgene Corporation) to treat multiple myeloma.

Additional exemplary therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

Exemplary combinations of anti-CD32b antibody molecules (alone or in combination with other stimulatory agents) and standard of care for cancer, include at least the following. Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” refers to radiation therapy delivered by a spatially confined radioactive material inserted into the body at or near a tumor or other proliferative tissue disease site. The term is intended without limitation to include exposure to radioactive isotopes (e.g., At-211, 1-131, 1-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as 1-125, 1-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

The anti-CD32b antibody molecules, alone or in combination with an antibody that binds a cell surface antigen, and/or in combination with an immunomodulatory compound (e.g., an anti-PD1, an anti-LAG3, anti-PD-L1 or anti-TIM-3 antibody molecule), may be used in combination with one or more of the existing modalities for treating cancers, including, but not limited to: surgery; radiation therapy (e.g., external-beam therapy which involves three dimensional, conformal radiation therapy where the field of radiation is designed, local radiation (e.g., radition directed to a preselected target or organ), or focused radiation). Focused radiation can be selected from the group consisting of stereotactic radiosurgery, fractionated stereotactic radiosurgery, and intensity-modulated radiation therapy. The focused radiation can have a radiation source selected from the group consisting of a particle beam (proton), cobalt-60 (photon), and a linear accelerator (x-ray), e.g., as decribed in WO 2012/177624.

As will be appreciated by the skilled artisan, the combination therapies involving the anti-CD32b antibody molecules, including those described in Tables 1, 2 or 3, may include combination therapies involving multiple classes of the agents described above. When the therapeutic agents of the present disclosure are administered together with another agent or agents, the two (or more) can be administered sequentially in any order or simultaneously. In some aspects, an antibody of the present disclosure is administered to a subject who is also receiving therapy with one or more other agents or methods. In other aspects, the binding molecule is administered in conjunction with surgical treatments. A combination therapy regimen may be additive, or it may produce synergistic results.

### Diagnostic Uses

In one aspect, the disclosure encompasses diagnostic assays for determining CD32b and/or nucleic acid expression as well as CD32b function, in the context of a biological sample (e.g., blood, serum, cells, tissue) or from an individual who is afflicted with a disease or disorder.

Diagnostic assays, such as competitive assays rely on the ability of a labelled analogue (the “tracer”) to compete with the test sample analyte for a limited number of binding sites on a common binding partner. The binding partner generally is insolubilized before or after the competition and then the tracer and analyte bound to the binding partner are separated from the unbound tracer and analyte. This separation is accomplished by decanting (where the binding partner was preinsolubilized) or by centrifuging (where the binding partner was precipitated after the competitive reaction). The amount of test sample analyte is inversely proportional to the amount of bound tracer as measured by the amount of marker substance. Dose-response curves with known amounts of analyte are prepared and compared with the test results in order to quantitatively determine the amount of analyte present in the test sample. These assays are called ELISA systems when enzymes are used as the detectable markers. In an assay of this form, competitive binding between antibodies and CD32b-binding antibodies results in the bound CD32b, preferably the CD32b epitopes of the disclosure, being a measure of antibodies in the serum sample, including neutralising antibodies in the serum sample.

A significant advantage of the assay is that measurement is made of neutralising antibodies directly (i.e., those which interfere with binding of CD32b, specifically, epitopes). Such an assay, particularly in the form of an ELISA test has considerable applications in the clinical environment and in routine blood screening.

In the clinical diagnosis or monitoring of patients with disorders associated with CD32b, the detection of elevated levels of CD32b protein or mRNA, in comparison to the levels in a corresponding biological sample from a normal subject is indicative of a patient with disorders associated with CD32b.

In vivo diagnostic or imaging is described in US2006/0067935. Briefly, these methods generally comprise administering or introducing to a patient a diagnostically effective amount of CD32b binding molecule that is operatively attached to a marker or label that is detectable by non-invasive methods. The antibody-marker conjugate is allowed sufficient time to localize and bind to CD32b. The patient is then exposed to a detection device to identify the detectable marker, thus forming an image of the location of the CD32b binding molecules in the tissue of a patient. The presence of CD32b binding antibody or an antigen-binding fragment thereof is detected by determining whether an antibody-marker binds to a component of the tissue. Detection of an increased level in CD32b proteins or a combination of protein in comparison to a normal individual may be indicative of a predisposition for and/or on set of disorders associated with CD32b. These aspects of the disclosure are also for use in tissue imaging methods and combined diagnostic and treatment methods.

The disclosure also pertains to the field of predictive medicine in which diagnostic assays, prognostic assays, pharmacogenomics, and monitoring clinical trials are used for prognostic (predictive) purposes to thereby treat an individual prophylactically.

The disclosure also provides for prognostic (or predictive) assays for determining whether an individual is at risk of developing a disorder associated with dysregulation of CD32b. For example, mutations in CD32b gene can be assayed in a biological sample. Such assays can be used for prognostic or predictive purpose to thereby prophylactically treat an individual prior to the onset of a disorder characterized by or associated with CD32b, nucleic acid expression or activity.

Another aspect of the disclosure provides methods for determining CD32b nucleic acid expression or CD32b activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the disclosure provides a method of monitoring the influence of agents (e.g., drugs) on the expression or activity of CD32b in clinical trials.

### Pharmaceutical Compositions

The disclosure provides pharmaceutical compositions comprising the CD32b-binding antibody or binding fragment thereof formulated together with a pharmaceutically acceptable carrier. The compositions can additionally contain one or more other therapeutic agents that are suitable for treating a CD32b-associated disease (e.g., B cell malignancies including including Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma and follicular lymphoma as well as other diseases including systemic light chain amyloidosis). Pharmaceutically acceptable carriers enhance or stabilize the composition, or facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present disclosure can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the disclosure can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the CD32b-binding antibody is employed in the pharmaceutical compositions of the disclosure. The CD32b-binding antibodies are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the antibodies and antigen-binding fragments thereof of the disclosure employed in the pharmaceutical composition at levels lower than that required to achieve the desired therapeutic effect and gradually increase the dosage until the desired effect is achieved. In general, effective doses of the compositions of the present disclosure, for the treatment of an allergic inflammatory disorder described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy. For systemic administration with an antibody, the dosage ranges from about 0.0001 to 100 mg/kg, and more usually 0.01 to 15 mg/kg, of the host body weight. An exemplary treatment regime entails systemic administration once per every two weeks or once a month or once every 3 to 6 months.

Antibody is usually administered on multiple occasions. Intervals between single dosages can be weekly, monthly or yearly. Intervals can also be irregular as indicated by measuring blood levels of CD32b-binding antibody in the patient. In some methods of systemic administration, dosage is adjusted to achieve a plasma antibody concentration of 1-1000 μg/ml and in some methods 25-500 μg/ml. Alternatively, antibody can be administered as a sustained release formulation, in which case less frequent administration is required. Dosage and frequency vary depending on the half-life of the antibody in the patient. In general, humanized antibodies show longer half-life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### EXAMPLES

The following examples are provided to further illustrate the disclosure but not to limit its scope. Other variants of the disclosure will be readily apparent to one of ordinary skill in the art and are encompassed by the appended claims.

### Example 1: Blockade of CD32b by Anti-CD32b Antibodies Promotes Maturation of Human Monocyte-Derived Dendritic Cells (moDC)

Human monocyte-derived dendritic cells (moDC) can express both activatory (CD32a, CD16a) and inhibitory (CD32b) Fcγ receptors (FcγRs). Blockade of inhibitory CD32b receptor with simultaneous engagement of activating CD32a receptor by IgG ligands from human serum can promote maturation of moDC (Dhodapkar et al., PNAS 102:2910-2915 (2005)).

Generation of Human moDC

Peripheral blood mononuclear cells (PBMCs) can be isolated from Leukopaks (HemaCare, Cat # PB001F-3) from donors using Ficol density gradient centrifugation. CD14+ monocytes are then isolated from the PBMCs by negative selection using the Monocyte Isolation Kit II, human (Miltenyi Biotec, Catalog#130-091-153) following the manufacturer's protocol. Isolated monocytes are differentiated into dendritic cells by culturing for 5 days in Dendritic Cell Medium (DCM) (CellGro DC Medium (CellGenix, Cat #20801-0500) supplemented with 10% FBS, 2 mM L-Glutamine (Thermo Fisher Scientific, Cat #25030-081), 200 IU/ml animal-free recombinant human IL-4 (Peprotech, Cat # AF-200-04) and 800 IU/ml animal-free recombinant human GM-CSF (Peprotech, Cat # AF-300-03)) supplemented with 10% FBS (Seradigm, Cat #1500-500). Media is replenished on day 3 post start of culture.

Human moDC can Simultaneously Express CD32a and CD32b

The expression of CD32a and CD32b receptors on human moDC can be determined. On day 5, moDC are harvested, resuspended in DCM supplemented with heat inactivated human AB serum (Valley Biomedical, Cat # HP1022HI) and cultured overnight in a 37° C., 5% CO2 incubator at a density of 100,000 cells per well of a 96-well flat-bottom microtiter plate (Corning, Catalog #3596). The following day, cells are resuspended in 50 ul of autoMACS® Running buffer (FACS Buffer) (Miltenyi Biotec, Cat #130-091-221) and incubated with either FITC conjugated anti-CD32a Ab (StemCell, Cat #60012FI) or FITC conjugated mouse IgG2b Isotype control Ab for 30 mins at 4° C. To determine the CD32b expression, the cells can be incubated with 0.5 ug/ml of either AF647 labelled 2B6 (Fc silenced anti-CD32b) Ab or AF647 labelled non-targeting isotype control IgG Fc silenced Ab. After 2 washes with FACS Buffer, cells are resuspended in 100 ul of FACS Buffer and acquired on a BD LSR Fortessa. Staining with either CD32a or CD32b Abs can be tested.

Determination of moDC Maturation after Treatment with Anti-CD32b Ab

On day 5, moDC can be harvested, resuspended in DCM and 100,000 cells plated per well of a 96-well flat-bottom microtiter plate (Corning, Catalog #3596). The cells are then treated with 10 ug/ml of either Fc wildtype, Fc silenced or Fc enhanced versions of an anti CD32b Ab for 4 hours in a 37° C., 5% CO2 incubator. Following the incubation, equal volume of DCM supplemented with heat inactivated human AB serum (Valley Biomedical, Cat # HP1022HI] is added and the cells are cultured overnight in a 37° C., 5% CO2 incubator. The following day, cells are resuspended in 25 ul of FACS Buffer containing Human TruStain FcX (Fc Block) [Biolegend, Cat #422302] and stained with BV421 conjugated anti-human CD14 Ab [Biolegend, Cat #301830].

These studies can demonstrate that maturation of CD32a positive moDC may be mediated by Fc-FcγR interaction, and that an anti-CD32b antibody can enhance this maturation by blocking the inhibitory CD32b while simultaneously engaging the activating CD32a via its Fc.

### Example 2: Anti-CD32b Antibody-Mediated ADCP Activates AKT and ERK in Macrophages

The first step of ADCP (antibody-dependent cellular phagocytosis) is initiated by crosslinking of FcγRs on the macrophages via FcγR-Fc binding between macrophages and opsonized target cells. The FcγR activation initiates multiple signaling pathways that promote phagocytosis and macrophage activation resulting in cytokine production. Activation of signaling pathways can be examined in macrophages by an anti-CD32b antibody-mediated phagocytosis using different versions of an anti-CD32b antibody: Fc-enhanced Ab, WT, or N297A (Fc-silenced). PBMCs can be isolated from a Leukopak (HemaCare, catalog# PB001F-3) using Ficoll gradient centrifugation. Monocytes can then be negatively selected using Miltenyi beads (catalog#130-091-153). Isolated monocytes are cultured for 7 d in complete macrophage medium [(X-VIVO15 (Lonza, catalog#04-744Q)+10% FBS)] supplemented with 10 ng/ml M-CSF (PeproTech, catalog#300-25). After 7 days of differentiation, macrophages are starved in serum free media overnight. On the following day, CFSE (0.05 μM in PBS) labelled Daudi cells are cocultured with macrophages in the presence of an anti-CD32b antibody or non-targeting, isotype control IgG Ab for 30 min. As controls, LPS (source: Sigma-Aldrich, Cat # L4391)/rhGM-CSF (Peprotech, Cat #300-03) or rhIL4 (Peprotech, Cat #200-04)/rhIL13 (Peprotech, Cat #200-13) can be added to polarize macrophages into M1 or M2 phenotype. After 30 min of co-culture, macrophages are immediately fixed with 2% PFA/PBS and detached from the wells using cold PBS with 5 mM EDTA to stain for anti-phospho-ERK1/2 (BD, #612566) and anti-phopho-AKT (BD, #561670) antibodies after permeabilization. After two successive washes with staining buffer, cells are suspended in 100 μl FACS buffer and acquired on a FACS Fortessa. Stained macrophages can first be gated on CFSE negative population and the percentage of ERK or AKT positive population can then be observed over untreated macrophages. This experiment can demonstrate that an anti-CD23b antibody-mediated ADCP can trigger rapid phosphorylation of both ERK and AKT in the macrophages, suggesting that both the MAPK and PI3K-AKT pathways can be activated by an anti-CD23b antibody.

### Example 3: Anti-CD32b Antibodies Induce Upregulation of the CD206 Marker and Cytokine Production in Macrophages

CD32b, also known as FcγR2b, is an inhibitory FcγR expressed on immune cells. Surface marker expression and cytokine release from macrophages can be assessed to determine whether blocking FcγR2b by an anti-CD32b antibody can result in modulation of macrophage activation. The experiments can be performed in the presence or absence of target Daudi cells to determine whether the modulation requires ADCP Human monocyte-derived macrophages can be differentiated as described in Example 1 above. After 7 days of differentiation, macrophages are either treated with an anti-CD32b antibody or isotype control IgG Ab in the presence or absence of Daudi cells for 24 h. As positive controls, LPS (source: Sigma-Aldrich, Cat # L4391)/rhGM-CSF (Peprotech, Cat #300-03) or rhIL4 (Peprotech, Cat #200-04)/rhIL13 Peprotech, Cat #200-13) can be added to polarize macrophages into M1 or M2 phenotype. After 24 h of treatment with or without Daudi cells, culture supernatants are harvested for analysis of cytokine levels. Macrophages are then detached from the wells using cold PBS with 5 mM EDTA to profile the levels of CD206 (BD, cat #:321106) expression.

Following an anti-CD32b antibody-mediated phagocytosis of Daudi cells, macrophages can be found to upregulate CD206 and this upregulation may not require functional Fc. The levels of inflammatory cytokines, IL6 and TNFα can be elevated in the co-culture media of Daudi and macrophages with an anti-CD32b antibody compared to IgG control. When macrophages are incubated with an anti-CD32b antibody in the absence of target cells, similar results can be observed. Results of such an exemplary experiment can show that, when FcγR2b is blocked by an anti-CD32b antibody, macrophages secrete higher levels of inflammatory cytokines IL6 and TNFα, and increase CD206 expression, indicating an activated state. This activation in macrophages may not require a functional Fc of an anti-CD32b antibody.

### Example 4: Blockade of CD32b by Anti-CD32b Antibodies Promotes Maturation of Human Monocyte Derived Dendritic Cells (moDC) in Response to Immune Complexes

Generation of Human moDCs

Peripheral blood mononuclear cells (PBMCs) can be isolated from Leukopaks (HemaCare, Cat # PB001F-3) from 10 donors using Ficol density gradient centrifugation. CD14+ monocytes are then isolated from the PBMCs by negative selection using the Monocyte Isolation Kit II, human (Miltenyi Biotec, Catalog#130-091-153) following the manufacturer's protocol. Isolated monocytes are differentiated into dendritic cells by culturing for 6 days in Roswell Park Memorial Institute (RPMI) 1640 Medium (Thermo Fisher Scientific, Cat #11875) supplemented with 10% FBS (Seradigm, Cat #1500-500), 2 mM L-Glutamine (Thermo Fisher Scientific, Cat #25030-081), 200 IU/ml recombinant human IL-4 (Peprotech, Cat #200-04) and 400 IU/ml recombinant human GM-CSF (Peprotech, Cat #300-03). Media is replenished on day 3 of culture.

Human moDC Simultaneously Express CD32a and CD32b

The expression of CD32a and CD32b receptors on human moDC is determined. On day 6, moDC are harvested, resuspended in AIM-V Media [Thermo Fisher Scientific, Cat #12055083] supplemented with 200 IU/mL recombinant human IL-4 and 400 IU/mL recombinant human GM-CSF, and cultured overnight in a 37° C., 5% CO2 incubator at a density of 100,000 cells per well of a 96-well round-bottom microtiter plate (Corning, Catalog #3799). The following day, cells are resuspended in 50 ul of autoMACS® Running buffer (FACS Buffer) (Miltenyi Biotec, Cat #130-091-221) and incubated with either FITC conjugated anti-CD32a Ab (StemCell, Cat #60012FI) or FITC conjugated mouse IgG2b Isotype control Ab for 30 mins at 4° C. To determine the CD32b expression, the cells are incubated with 0.5 μm/ml of either AF647 labelled 2B6 N297A (Fc silenced anti-CD32b) Ab or AF647 labelled Fc silenced Isotype control Ab. After 2 washes with FACS Buffer, cells are resuspended in 150 μl of FACS Buffer and acquired on a BD LSR Fortessa. Staining with either CD32a or CD32b Abs is used to detect CD32a or CD32b expression.

Determination of moDC Maturation after Treatment with Anti-CD32b Ab Followed by Treatment with Immune Complexes

On day 6, moDC are harvested, resuspended in AIM-V Media supplemented with 200 IU/mL recombinant human IL-4 and 400 IU/mL recombinant human GM-CSF, and 100,000 cells are plated per well of a 96-well round-bottom microtiter plate. Cells are then treated with 10 μg/ml of either Fc silenced (N297A) or Fc enhanced (afucosylated) versions of the anti CD32b Ab or Isotype control for 30 minutes on ice. Following this incubation, 10 μL of immune complexes (ICs; polyclonal human serum IgG [Millipore Sigma, Cat #14506-100MG] is prepared at 1000 μg/mL in PBS, heat-aggregated at 63° C. for 30 minutes) are added and cells are cultured overnight in a 37° C., 5% CO2 incubator. The following day, cells are washed twice with FACS buffer, resuspended in 30 μl of FACS Buffer containing Human TruStain FcX (Fc Block) (Biolegend, Cat #422302), and then stained with PE conjugated anti-human CD14 Ab (Biolegend, Cat #367104) and BV650 conjugated anti-human CD86 (Biolegend, Cat #305428) in a final volume of 50 μl . After 2 washes with FACS Buffer, cells are resuspended in 150 μl of FACS Buffer and acquired on a BD LSR Fortessa.

ICs can induce moDC maturation in a manner that may partially depend on CD32a expression, and anti-CD32b antibodies can enhance this maturation by blocking the inhibitory CD32b receptor, allowing ICs to bind exclusively to activating FcγRs and to thereby induce signaling pathways favoring maturation without engaging the negative feedback pathways induced downstream of CD32b activation. Fc enhanced anti-CD32b antibodies can also directly induce low levels of moDC maturation, potentially by binding in cis to simultaneously block CD32b signaling while engaging CD32a signaling.

### Example 5: Anti-CD32b Antibodies Induce Decrease of Surface Expression of CD163 on Macrophages

Surface marker expression of macrophages can be used to determine whether blocking CD32b with anti-CD32b antibodies results in modulation of macrophage activation. Peripheral blood mononuclear cells (PBMCs) are isolated from peripheral blood of five donors using Ficol density gradient centrifugation. CD14+ monocytes are then isolated from the PBMCs by negative selection using the Monocyte Isolation Kit II, human (Miltenyi Biotec, Catalog#130-091-153) following the manufacturer's protocol. Isolated monocytes are then differentiated into macrophages by culturing for 7 days in X-VIVO15 Medium (Thermo Fisher Scientific, Cat #11875) supplemented with 10% FBS (Seradigm, Cat #1500-500) and 20 ng/mL recombinant human M-CSF (Peprotech, Cat #300-25). Media is replenished on day 3 of culture. After 7 days of differentiation, macrophages are either treated with anti-CD32b antibodies or isotype control IgG Ab for 24 h. After 24 h of treatment, macrophages are detached from the wells using cold autoMACS® Running buffer (FACS Buffer) (Miltenyi Biotec, Cat #130-091-221). Cells are stained for surface CD163 expression with an APC conjugated anti-CD163 antibody (Biolegend, Cat #333610) for 20 minutes on ice, washed twice with FACS buffer, resuspended in 200 μL FACS buffer, and acquired on a BD LSR Fortessa.

This example demonstrates that surface expression of CD163 is decreased on macrophages after treatment with anti-CD32b antibodies. This decrease in CD163 surface expression is dependent on the presence of a functional Fc domain on CD32b antibodies, as Fc silenced anti-CD32b antibodies do not mediate any decrease in CD163 surface expression upon treatment.

### Example 6: Blockade of CD32b by Anti-CD32b Antibodies Disrupts Binding of Immune Complexes to CD32b-Expressing Cells

Human cells can express both activating (CD32a, CD16a) and inhibitory (CD32b) Fcγ receptors (FcγRs) Immune complexes are multimeric antibody-antigen constructs that can simultaneously engage both inhibitory and activating FcγRs through Fc:FcγR interactions.

### Generation of Human Immune Complexes

Fluorescently labeled human immune complexes (ICs) are generated using human polyclonal serum IgG (1000 μg/mL in PBS; Millipore Sigma, Cat #14506-100MG) heat-aggregated at 63° C. for 30 minutes. These ICs are fluorescently labeled using a 1:100 dilution of Goat F(ab′)2 Anti-Human IgG-(Fab′)2 DyLight® 550 pre-adsorbed (abcam, Cat # ab98601) on ice for 20 minutes, protected from light. Labeled ICs are used immediately for treatment of cells, as described below.

### IC Treatment of CHO Cells Expressing Human CD32 Variants

Stable CHO cell lines described above expressing huCD32a, huCD32b, or no human FcγRs (Parental) are plated in a 96-well round-bottom microtiter plate (Corning, Catalog #3799) at a density of 100,000 cells/well in Dulbecco's Modified Eagle Medium (DMEM; ThermoFisher Scientific, Cat #11320-033) supplemented with 10% FBS (Seradigm, Cat #1500-500). These cell lines are known to express levels of CD32a or CD32b that are markedly higher than observed on DCs. Cells are then treated with anti-CD32b antibodies or isotype control antibodies for 4 hr at 37° C. In one experiment, Parental, huCD32a, and huCD32b cells are treated with 0, 0.01, 0.1, 1, or 10 μg/mL of Fc silenced versions of anti-CD32b Ab or Isotype control. In another experiment, Parental and huCD32b cells are treated with 10 μg/mL of Fc Enhanced (Afucosylated) anti-CD32b Ab or Isotype control. After antibody treatment, 10 μg fluorescently labeled ICs are added to each well. Cells are then incubated on ice for 60 minutes, washed twice with autoMACS® Running buffer (FACS Buffer) (Miltenyi Biotec, Cat #130-091-221), and resuspended in a final volume of 200 μL FACS Buffer. Fluorescence is then acquired on a BD LSR Fortessa.

Pretreatment with Fc enhanced anti-CD32b Abs completely blocks IC binding to huCD32b. The preservation of IC binding to activating but not inhibitory FcγRs following anti-CD32b treatment offers a potential approach to selectively enhance cellular activation in response to ICs and other Fc-dependent stimuli.

What is claimed is:

1. A method of treating a cancer in a subject, comprising administering to the subject an anti-CD32b antibody molecule, in combination with one or more second therapeutic agents, wherein the second therapeutic agent is chosen from one or more of:
(i) an antibody that binds a cell surface antigen on a cancer cell, tumor cell, or an immune cell;
(ii) an immunomodulatory compound; or
(iii) an anti-cancer therapy,
wherein the anti-CD32b antibody molecule is chosen from an antibody disclosed in Table 1, 2, or 3; thereby treating the cancer.

2. Use of an anti-CD32b antibody molecule in combination with one or more second therapeutic agents to treat cancer in a subject, wherein the second therapeutic agent is chosen from one or more of:
(i) an antibody that binds a cell surface antigen on a cancer cell, tumor cell, or an immune cell;
(ii) an immunomodulatory compound; or
(iii) an anti-cancer therapy,
wherein the anti-CD32b antibody molecule is chosen from an antibody disclosed in Table 1, 2, or 3; thereby treating the cancer.

3. A composition comprising an anti-CD32b antibody molecule in combination with one or more second therapeutic agents, for use in treating a cancer in a subject, wherein the second agent is chosen from one or more of:
(i) an antibody that binds a cell surface antigen on a cancer cell, tumor cell, or an immune cell;
(ii) an immunomodulatory compound; or
(iii) an anti-cancer therapy,
wherein the anti-CD32b antibody molecule is chosen from an antibody disclosed in Table 1, 2, or 3.

4. Use of anti-CD32b antibody molecule in the manufacture of a medicament for use in combination with
(i) an antibody that binds a cell surface antigen on a cancer cell, tumor cell, or an immune cell;
(ii) an immunomodulatory compound; or
(iii) an anti-cancer therapy,
to treat cancer in a subject, wherein the anti-CD32b antibody molecule is chosen from an antibody disclosed in Table 1, 2, or 3.

5. The method, use, or composition of any one of the preceding claims, wherein the anti-CD32b antibody molecule comprises a heavy chain variable region comprising the amino acid sequence of SEQ ID NO: 1, or an amino acid sequence at least 95% identical thereto, and a light chain variable region comprising the amino acid sequence of SEQ ID NO: 2, or an amino acid sequence at least 95% identical thereto.

6. The method, use, or composition of any one of the preceding claims, wherein the anti-CD32b antibody molecule comprises a heavy chain variable region comprising the amino acid sequence of SEQ ID NO: 9, or an amino acid sequence at least 95% identical thereto, and a light chain variable region comprising the amino acid sequence of SEQ ID NO: 10, or an amino acid sequence at least 95% identical thereto.

7. The method, use, or composition of any one of the preceding claims, wherein the anti-CD32b antibody molecule comprises a heavy chain variable region comprising the amino acid sequence of SEQ ID NO: 17, or an amino acid sequence at least 95% identical thereto, and a light chain variable region comprising the amino acid sequence of SEQ ID NO: 18, or an amino acid sequence at least 95% identical thereto.

8. The method, use, or composition of any one of the preceding claims, wherein the anti-CD32b antibody molecule comprises a heavy chain variable region comprising the amino acid sequence of SEQ ID NO: 25, or an amino acid sequence at least 95% identical thereto, and a light chain variable region comprising the amino acid sequence of SEQ ID NO: 26, or an amino acid sequence at least 95% identical thereto.

9. The method, use, or composition of any one of the preceding claims, wherein the anti-CD32b antibody molecule comprises a heavy chain variable region comprising the amino acid sequence of SEQ ID NO: 33, or an amino acid sequence at least 95% identical thereto, and a light chain variable region comprising the amino acid sequence of SEQ ID NO: 34, or an amino acid sequence at least 95% identical thereto.

10. The method, use, or composition of any one of the preceding claims, wherein the anti-CD32b antibody molecule comprises a heavy chain variable region comprising the amino acid sequence of SEQ ID NO: 41, or an amino acid sequence at least 95% identical thereto, and a light chain variable region comprising the amino acid sequence of SEQ ID NO: 42, or an amino acid sequence at least 95% identical thereto.

11. The method, use, or composition of any one of the preceding claims, wherein the anti-CD32b antibody molecule comprises a heavy chain variable region comprising the amino acid sequence of SEQ ID NO: 49, or an amino acid sequence at least 95% identical thereto, and a light chain variable region comprising the amino acid sequence of SEQ ID NO: 50, or an amino acid sequence at least 95% identical thereto.

12. The method, use, or composition of any one of the preceding claims, wherein the anti-CD32b antibody molecule comprises a heavy chain variable region comprising the amino acid sequence of SEQ ID NO: 57, or an amino acid sequence at least 95% identical thereto, and a light chain variable region comprising the amino acid sequence of SEQ ID NO: 58, or an amino acid sequence at least 95% identical thereto.

13. The method, use, or composition of any one of the preceding claims, wherein the anti-CD32b antibody molecule comprises a heavy chain variable region comprising the amino acid sequence of SEQ ID NO: 65, or an amino acid sequence at least 95% identical thereto, and a light chain variable region comprising the amino acid sequence of SEQ ID NO: 66, or an amino acid sequence at least 95% identical thereto.

14. The method, use, or composition of any one of the preceding claims, wherein the anti-CD32b antibody molecule comprises a heavy chain variable region comprising the amino acid sequence of SEQ ID NO: 73, or an amino acid sequence at least 95% identical thereto, and a light chain variable region comprising the amino acid sequence of SEQ ID NO: 74, or an amino acid sequence at least 95% identical thereto.

15. The method, use, or composition of any one of the preceding claims, wherein the anti-CD32b antibody molecule comprises a heavy chain variable region comprising the amino acid sequence of SEQ ID NO: 81, or an amino acid sequence at least 95% identical thereto, and a light chain variable region comprising the amino acid sequence of SEQ ID NO: 82, or an amino acid sequence at least 95% identical thereto.

16. The method, use, or composition of any one of the preceding claims, wherein the anti-CD32b antibody molecule comprises a heavy chain variable region comprising the amino acid sequence of SEQ ID NO: 89, or an amino acid sequence at least 95% identical thereto, and a light chain variable region comprising the amino acid sequence of SEQ ID NO: 90, or an amino acid sequence at least 95% identical thereto.

17. The method, use, or composition of any one of the preceding claims, wherein the anti-CD32b antibody molecule comprises a heavy chain variable region comprising the amino acid sequence of SEQ ID NO: 97, or an amino acid sequence at least 95% identical thereto, and a light chain variable region comprising the amino acid sequence of SEQ ID NO: 98, or an amino acid sequence at least 95% identical thereto.

18. The method, use, or composition of any one of the preceding claims, wherein the anti-CD32b antibody molecule comprises a heavy chain variable region comprising the amino acid sequence of SEQ ID NO: 105, or an amino acid sequence at least 95% identical thereto, and a light chain variable region comprising the amino acid sequence of SEQ ID NO: 106, or an amino acid sequence at least 95% identical thereto.

19. The method, use, or composition of any one of the preceding claims, wherein the anti-CD32b antibody molecule comprises a heavy chain variable region comprising the amino acid sequence of SEQ ID NO: 113, or an amino acid sequence at least 95% identical thereto, and a light chain variable region comprising the amino acid sequence of SEQ ID NO: 114, or an amino acid sequence at least 95% identical thereto.

20. The method, use, or composition of any one of the preceding claims, wherein the anti-CD32b antibody molecule comprises a heavy chain variable region comprising the amino acid sequence of SEQ ID NO: 121, or an amino acid sequence at least 95% identical thereto, and a light chain variable region comprising the amino acid sequence of SEQ ID NO: 122, or an amino acid sequence at least 95% identical thereto.

21. The method, use, or composition of any one of the preceding claims, wherein the anti-CD32b antibody molecule comprises a heavy chain variable region comprising the amino acid sequence of SEQ ID NO: 129, or an amino acid sequence at least 95% identical thereto, and a light chain variable region comprising the amino acid sequence of SEQ ID NO: 130, or an amino acid sequence at least 95% identical thereto.

22. The method, use, or composition of any one of the preceding claims, wherein the anti-CD32b antibody molecule comprises a heavy chain variable region comprising the amino acid sequence of SEQ ID NO: 201, or an amino acid sequence at least 95% identical thereto, and a light chain variable region comprising the amino acid sequence of SEQ ID NO: 205, or an amino acid sequence at least 95% identical thereto.

23. The method, use, or composition of any one of the preceding claims, wherein the anti-CD32b antibody molecule comprises a heavy chain variable region comprising the amino acid sequence of SEQ ID NO: 209, or an amino acid sequence at least 95% identical thereto, and a light chain variable region comprising the amino acid sequence of SEQ ID NO: 213, or an amino acid sequence at least 95% identical thereto.

24. The method, use, or composition of any one of the preceding claims, wherein the anti-CD32b antibody molecule comprises a heavy chain variable region comprising the amino acid sequence of SEQ ID NO: 217, or an amino acid sequence at least 95% identical thereto, and a light chain variable region comprising the amino acid sequence of SEQ ID NO: 221, or an amino acid sequence at least 95% identical thereto.

25. The method, use, or composition of any one of the preceding claims, wherein the anti-CD32b antibody molecule comprises a heavy chain variable region comprising the amino acid sequence of SEQ ID NO: 225, or an amino acid sequence at least 95% identical thereto, and a light chain variable region comprising the amino acid sequence of SEQ ID NO: 229, or an amino acid sequence at least 95% identical thereto.

26. The method, use, or composition of any one of the preceding claims, wherein the anti-CD32b antibody molecule comprises a heavy chain variable region comprising the amino acid sequence of SEQ ID NO: 233, or an amino acid sequence at least 95% identical thereto, and a light chain variable region comprising the amino acid sequence of SEQ ID NO: 237, or an amino acid sequence at least 95% identical thereto.

27. The method, use, or composition of any one of the preceding claims, wherein the anti-CD32b antibody molecule comprises a heavy chain variable region comprising the amino acid sequence of SEQ ID NO: 241, or an amino acid sequence at least 95% identical thereto, and a light chain variable region comprising the amino acid sequence of SEQ ID NO: 213, or an amino acid sequence at least 95% identical thereto.

28. The method, use, or composition of any one of the preceding claims, wherein the anti-CD32b antibody molecule comprises a heavy chain variable region comprising the amino acid sequence of SEQ ID NO: 245, or an amino acid sequence at least 95% identical thereto, and a light chain variable region comprising the amino acid sequence of SEQ ID NO: 249, or an amino acid sequence at least 95% identical thereto.

29. The method, use, or composition of any one of the preceding claims, wherein the anti-CD32b antibody molecule comprises a heavy chain variable region comprising the amino acid sequence of SEQ ID NO: 253, or an amino acid sequence at least 95% identical thereto, and a light chain variable region comprising the amino acid sequence of SEQ ID NO: 257, or an amino acid sequence at least 95% identical thereto.

30. The method, use, or composition of any one of the preceding claims, wherein the anti-CD32b antibody molecule comprises a heavy chain variable region comprising the amino acid sequence of SEQ ID NO: 261, or an amino acid sequence at least 95% identical thereto, and a light chain variable region comprising the amino acid sequence of SEQ ID NO: 265, or an amino acid sequence at least 95% identical thereto.

31. The method, use, or composition of any one of the preceding claims, wherein the anti-CD32b antibody molecule comprises a heavy chain variable region comprising the amino acid sequence of SEQ ID NO: 269, or an amino acid sequence at least 95% identical thereto, and a light chain variable region comprising the amino acid sequence of SEQ ID NO: 273, or an amino acid sequence at least 95% identical thereto.

32. The method, use, or composition of any one of the preceding claims, wherein the anti-CD32b antibody molecule comprises a heavy chain variable region comprising the amino acid sequence of SEQ ID NO: 300, or an amino acid sequence at least 95% identical thereto, and a light chain variable region comprising the amino acid sequence of SEQ ID NO: 301, or an amino acid sequence at least 95% identical thereto.

33. The method, use, or composition of any one of the preceding claims, wherein the anti-CD32b antibody molecule comprises a heavy chain variable region comprising the amino acid sequence of SEQ ID NO: 313, or an amino acid sequence at least 95% identical thereto, and a light chain variable region comprising the amino acid sequence of SEQ ID NO: 310, or an amino acid sequence at least 95% identical thereto.

34. The method, use, or composition of any one of the preceding claims, wherein the anti-CD32b antibody molecule comprises a heavy chain variable region comprising the amino acid sequence of SEQ ID NO: 313, or an amino acid sequence at least 95% identical thereto, and a light chain variable region comprising the amino acid sequence of SEQ ID NO: 311, or an amino acid sequence at least 95% identical thereto.

35. The method, use, or composition of any one of the preceding claims, wherein the anti-CD32b antibody molecule comprises a heavy chain variable region comprising the amino acid sequence of SEQ ID NO: 313, or an amino acid sequence at least 95% identical thereto, and a light chain variable region comprising the amino acid sequence of SEQ ID NO: 312, or an amino acid sequence at least 95% identical thereto.

36. The method, use, or composition of any one of the preceding claims, wherein the anti-CD32b antibody molecule comprises a heavy chain variable region comprising the amino acid sequence of SEQ ID NO: 317, or an amino acid sequence at least 95% identical thereto, and a light chain variable region comprising the amino acid sequence of SEQ ID NO: 321, or an amino acid sequence at least 95% identical thereto.

37. The method, use, or composition of any one of the preceding claims, wherein the anti-CD32b antibody molecule comprises a heavy chain variable region comprising the amino acid sequence of SEQ ID NO: 323, or an amino acid sequence at least 95% identical thereto, and a light chain variable region comprising the amino acid sequence of SEQ ID NO: 322, or an amino acid sequence at least 95% identical thereto.

38. The method, use, or composition of any one of the preceding claims, wherein the anti-CD32b antibody molecule comprises a heavy chain variable region of an antibody produced by hybridoma clone 1D5 having ATCC accession number PTA-5958, or an amino acid sequence at least 95% identical thereto, and a light chain variable region of an antibody produced by hybridoma clone 1D5 having ATCC accession number PTA-5958, or an amino acid sequence at least 95% identical thereto.

39. The method, use, or composition of any one of the preceding claims, wherein the anti-CD32b antibody molecule comprises a heavy chain variable region of an antibody produced by hybridoma clone 2E1 having ATCC accession number PTA-5961, or an amino acid sequence at least 95% identical thereto, and a light chain variable region of an antibody produced by hybridoma clone 2E1 having ATCC accession number PTA-5961, or an amino acid sequence at least 95% identical thereto.

40. The method, use, or composition of any one of the preceding claims, wherein the anti-CD32b antibody molecule comprises a heavy chain variable region of an antibody produced by hybridoma clone 2H9 having ATCC accession number PTA-5962, or an amino acid sequence at least 95% identical thereto, and a light chain variable region of an antibody produced by hybridoma clone 2H9 having ATCC accession number PTA-5962, or an amino acid sequence at least 95% identical thereto.

41. The method, use, or composition of any one of the preceding claims, wherein the anti-CD32b antibody molecule comprises a heavy chain variable region of an antibody produced by clone 2D11 having ATCC accession number PTA-5960, or an amino acid sequence at least 95% identical thereto, and a light chain variable region of an antibody produced by hybridoma clone 2D11 having ATCC accession number PTA-5960, or an amino acid sequence at least 95% identical thereto.

42. The method, use, or composition of any one of the preceding claims, wherein the anti-CD32b antibody molecule comprises a heavy chain variable region of an antibody produced by clone 1F2 having ATCC accession number PTA-5959, or an amino acid sequence at least 95% identical thereto, and a light chain variable region of an antibody produced by hybridoma clone 1F2 having ATCC accession number PTA-5959, or an amino acid sequence at least 95% identical thereto.

43. The method, use, or composition of any one of the preceding claims, wherein the antibody is afucosylated.

44. The method, use, or composition of any one of the preceding claims, wherein the Fc portion of the antibody is modified to enhance ADCC activity.

45. The method, use, or composition of any one of the preceding claims, wherein the antibody or antigen-binding fragment thereof selectively binds human CD32b over human CD32a.

46. The method, use, or composition of any one of the preceding claims, wherein the anti-CD32b antibody molecule is an IgG chosen from an IgG1, an IgG2, an IgG3, or an IgG4.

47. The method, use, or composition of any one of the preceding claims, wherein the anti-CD32b antibody molecule is chosen from: a monoclonal antibody, a chimeric antibody, a single chain antibody, a Fab, or a scFv.

48. The method, use, or composition of any one of the preceding claims, wherein anti-CD32b antibody molecule is chimeric, humanized or fully human.

49. The method, use, or composition of any one of the preceding claims, wherein anti-CD32b antibody molecule inhibits binding of human CD32b to immunoglobulin Fc domains.

50. The method, use, or composition of any one of the preceding claims, wherein the anti-CD32b antibody molecule is a component of an immunoconjugate.

51. The method, use, or composition of any one of the preceding claims, wherein the second therapeutic agent comprises one or more antibodies that bind a cell surface antigen.

52. The method, use, or composition of claim 51, wherein the cell surface antigen and CD32b are co-expressed on B cells.

53. The method, use, or composition of claim 52, wherein the cell surface antigen is chosen from: CD20, CD38, CD52, CS1/SLAMF7, CD56, CD138, KiR,CD19, CD40, Thy-1, Ly-6, CD49, Fas, Cd95, APO-1, EGFR, HER2, CXCR4, HLA molecules, GM1, CD22, CD23, CD80, CD74, or DRD.

54. The method, use, or composition of claim 55, wherein the cell surface antigen is chosen from: CD20, CD38, CS1/SLAMF7 or CD52.

55. The method, use, or composition of claim 56, wherein the cell surface antigen is CD38.

56. The method, use, or composition of any one of the preceding claims, wherein the antibody that binds to the cell surface antigen is chosen from: elotuzumab, ofatumumab, obinutuzumab, daratumumab, or alemtuzumab.

57. The method, use, or composition of any one of the preceding claims, wherein the immunomodulatory compound is selected from a cytokine, an agonist of a costimulatory molecule, or an inhibitor of an inhibitory compound.

58. The method, use, or composition of any one of the preceding claims, wherein the immunomodulatory compound is a cytokine chosen from one or more of IL-15, IL-2, IL-6, IL-7, IL-9, IL-12, IL-18, IL-21, IL-23, or IL-27.

59. The method, use, or composition of any one of the preceding claims, wherein the immunomodulatory compound is an agonist of a costimulatory molecule selected from OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, CD83 ligand, or STING.

60. The method, use, or composition of any one of the preceding claims, wherein the immunomodulatory compound is an inhibitor of an inhibitory molecule selected from PD-1, PD-L1, PD-L2, CTLA-4, TIM-3, LAG-3, CEACAM-1, CEACAM-3, CEACAM-5, VISTA, BTLA, TIGIT, LAIR1, CD160, 2B4, TGF beta, or IDO.

61. The method, use, or composition of any one of the preceding claims, wherein the anti-cancer therapy is selected from a targeted anti-cancer therapy, a cytotoxic agent, or a chemotherapeutic agent.

62. The method, use, or composition of any one of the preceding claims, wherein the anti-cancer therapy comprises a targeted anti-cancer therapy selected from ofatumumab, romidepsin, brentuximab, obinutuzumab, elotuzumab, daratumumab, or alemtuzumab.

63. The method, use, or composition of any one of the preceding claims, wherein the cytotoxic agent is chosen from ibrutinib, belinostat, romidepsin, brentuximab vedotin, pralatrexate, pentostatin, dexamethasone, idelalisib, ixazomib, liposomal doxyrubicin, pomalidomide, panobinostat, thalidomide, or lenalidomide.

64. The method, use, or composition of any one of the preceding claims, wherein the anti-CD32b antibody molecule, is administered in an amount sufficient to result in one or more of:
decreased B cell inhibition,
increased B cell activation;
enhanced immune cell-mediated ADCC, e.g., macrophage- or NK cell-mediated ADCC;
enhanced macrophage-mediated ADCP; or
enhanced DC activity, e.g., DC maturation, antigen presentation and T cell priming.

65. The method, use, or composition of any one of the preceding claims, wherein the antibody that binds a cell surface antigen on a cancer cell, tumor cell, or an immune cell comprises an immunoglobulin Fc domain and an antigen binding domain against a cell surface antigen.

66. The method, use, or composition of any one of the preceding claims, wherein the subject has a CD32b-related condition or disorder.

67. The method, use, or composition of any one of the preceding claims, wherein the subject has a condition or disorder that is chosen from: B cell malignancies, Hodgkins lymphoma, Non-Hodgkins lymphoma, multiple myeloma, diffuse large B cell lymphoma, acute lymphocytic leukemia, chronic lymphocytic leukemia, small lymphocytic lymphoma, diffuse small cleaved cell lymphoma, MALT lymphoma, mantel cell lymphoma, marginal zone lymphoma, follicular lymphoma, systemic light chain amyloidosis, acute myeloid leukemia (AML), myelodysplasia, myelodysplastic syndrome, myelofibrosis, myeloproliferative neoplasms, acute lymphoid leukemia (ALL), hairy cell leukemia, prolymphocytic leukemia, chronic myeloid leukemia (CML), or blastic plasmacytoid dendritic cell neoplasm.

68. The method, use, or composition of any one of the preceding claims, wherein the subject has a solid cancer chosen from one or more of: pancreatic (e.g., pancreatic adenocarcinoma or pancreatic ductal adenocarcinoma), breast, colorectal, colon, lung (e.g., small or non-small cell lung cancer), skin, ovarian, prostate, cervix, gastrointestinal (e.g., carcinoid or stromal), stomach, head and neck, kidney, or liver cancer, or a metastatic lesion thereof.

70. A method of treating a cancer in a subject, comprising administering to the subject an anti-CD32b antibody molecule, in combination with an antibody that binds a cell surface antigen on a cancer cell, tumor cell, or an immune cell; wherein the anti-CD32b antibody is selected from an antibody that comprises:
a) a heavy chain variable region comprising the amino acid sequence of SEQ ID NO: 1, or an amino acid sequence at least 95% identical thereto, and a light chain variable region comprising the amino acid sequence of SEQ ID NO: 2, or an amino acid sequence at least 95% identical thereto;
b) a heavy chain variable region comprising the amino acid sequence of SEQ ID NO: 9, or an amino acid sequence at least 95% identical thereto, and a light chain variable region comprising the amino acid sequence of SEQ ID NO: 10, or an amino acid sequence at least 95% identical thereto;
c) a heavy chain variable region comprising the amino acid sequence of SEQ ID NO: 17, or an amino acid sequence at least 95% identical thereto, and a light chain variable region comprising the amino acid sequence of SEQ ID NO: 18, or an amino acid sequence at least 95% identical thereto;
d) a heavy chain variable region comprising the amino acid sequence of SEQ ID NO: 25, or an amino acid sequence at least 95% identical thereto, and a light chain variable region comprising the amino acid sequence of SEQ ID NO: 26, or an amino acid sequence at least 95% identical thereto;
e) a heavy chain variable region comprising the amino acid sequence of SEQ ID NO: 33, or an amino acid sequence at least 95% identical thereto, and a light chain variable region comprising the amino acid sequence of SEQ ID NO: 34, or an amino acid sequence at least 95% identical thereto;
f) a heavy chain variable region comprising the amino acid sequence of SEQ ID NO: 41, or an amino acid sequence at least 95% identical thereto, and a light chain variable region comprising the amino acid sequence of SEQ ID NO: 42, or an amino acid sequence at least 95% identical thereto;
g) a heavy chain variable region comprising the amino acid sequence of SEQ ID NO: 49, or an amino acid sequence at least 95% identical thereto, and a light chain variable region comprising the amino acid sequence of SEQ ID NO: 50, or an amino acid sequence at least 95% identical thereto;
h) a heavy chain variable region comprising the amino acid sequence of SEQ ID NO: 57, or an amino acid sequence at least 95% identical thereto, and a light chain variable region comprising the amino acid sequence of SEQ ID NO: 58, or an amino acid sequence at least 95% identical thereto;
i) a heavy chain variable region comprising the amino acid sequence of SEQ ID NO: 65, or an amino acid sequence at least 95% identical thereto, and a light chain variable region comprising the amino acid sequence of SEQ ID NO: 66, or an amino acid sequence at least 95% identical thereto;
j) a heavy chain variable region comprising the amino acid sequence of SEQ ID NO: 73, or an amino acid sequence at least 95% identical thereto, and a light chain variable region comprising the amino acid sequence of SEQ ID NO: 74, or an amino acid sequence at least 95% identical thereto;
k) a heavy chain variable region comprising the amino acid sequence of SEQ ID NO: 81, or an amino acid sequence at least 95% identical thereto, and a light chain variable region comprising the amino acid sequence of SEQ ID NO: 82, or an amino acid sequence at least 95% identical thereto;
l) a heavy chain variable region comprising the amino acid sequence of SEQ ID NO: 89, or an amino acid sequence at least 95% identical thereto, and a light chain variable region comprising the amino acid sequence of SEQ ID NO: 90, or an amino acid sequence at least 95% identical thereto;
m) a heavy chain variable region comprising the amino acid sequence of SEQ ID NO: 97, or an amino acid sequence at least 95% identical thereto, and a light chain variable region comprising the amino acid sequence of SEQ ID NO: 98, or an amino acid sequence at least 95% identical thereto;
n) a heavy chain variable region comprising the amino acid sequence of SEQ ID NO: 105, or an amino acid sequence at least 95% identical thereto, and a light chain variable region comprising the amino acid sequence of SEQ ID NO: 106, or an amino acid sequence at least 95% identical thereto;
o) a heavy chain variable region comprising the amino acid sequence of SEQ ID NO: 113, or an amino acid sequence at least 95% identical thereto, and a light chain variable region comprising the amino acid sequence of SEQ ID NO: 114, or an amino acid sequence at least 95% identical thereto;
p) a heavy chain variable region comprising the amino acid sequence of SEQ ID NO: 121, or an amino acid sequence at least 95% identical thereto, and a light chain variable region comprising the amino acid sequence of SEQ ID NO: 122, or an amino acid sequence at least 95% identical thereto; or
q) a heavy chain variable region comprising the amino acid sequence of SEQ ID NO: 129, or an amino acid sequence at least 95% identical thereto, and a light chain variable region comprising the amino acid sequence of SEQ ID NO: 130, or an amino acid sequence at least 95% identical thereto; and wherein the antibody that binds a cell surface antigen on a cancer cell is selected from elotuzumab, ofatumumab, obinutuzumab, daratumumab, or alemtuzumab.


SEQUENCE LISTING (1648 sequences)

SEQ ID NO: 1 - protein, 117 aa, Artificial Sequence
  source: /note=“Description of Artificial Sequence: Synthetic polypeptide”
EVQLLESGGGLVQPGGSLRLSCAASGFTFSSYGMHWVRQAPGKGLEWMAVISYDGSNKYYADSVKGRFTISRDNSKNTLYLQMNSLRAEDTAVYYCARELYDAFDIWGQGTLVTVSS

SEQ ID NO: 2 - protein, 112 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYADDHRPSGVPDRFSGSKSGTSASLAISGLRSEDEADYYCASWDDSQRAVIFGGGTKLTVLG

SEQ ID NO: 3 - protein, 5 aa, Artificial Sequence
  source: /note=“Description of Artificial Sequence: Synthetic peptide”
SYGMH

SEQ ID NO: 4 - protein, 17 aa, Artificial Sequence
  source: /note=“Description of Artificial Sequence: Synthetic peptide”
VISYDGSNKYYADSVKG

SEQ ID NO: 5 - protein, 8 aa, Artificial Sequence
  source: /note=“Description of Artificial Sequence: Synthetic peptide”
ELYDAFDI

SEQ ID NO: 6 - protein, 14 aa, Artificial Sequence
  source: /note=“Description of Artificial Sequence: Synthetic peptide”
TGSSSNIGAGYDVH

SEQ ID NO: 7 - protein, 7 aa, Artificial Sequence
  source: /note=“Description of Artificial Sequence: Synthetic peptide”
ADDHRPS

SEQ ID NO: 8 - protein, 11 aa, Artificial Sequence
  source: /note=“Description of Artificial Sequence: Synthetic peptide”
ASWDDSQRAVI

SEQ ID NO: 9 - protein, 117 aa, Artificial Sequence
  source: /note=“Description of Artificial Sequence: Synthetic polypeptide”
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAREWRDAFDIWGQGTLVTVSS

SEQ ID NO: 10 - protein, 113 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYSDNQRPSGVPDRFSGSKSGTSASLAISGLRSEDEADYYCAAWDDSLSGSWVFGGGTKLTVLG

SEQ ID NO: 11 - protein, 5 aa, Artificial Sequence
  source: /note=“Description of Artificial Sequence: Synthetic peptide”
NYGMH

SEQ ID NO: 12 - protein, 17 aa, Artificial Sequence
  source: /note=“Description of Artificial Sequence: Synthetic peptide”
VISYDGSNKYYADSVKG

SEQ ID NO: 13 - protein, 7 aa, Artificial Sequence
  source: /note=“Description of Artificial Sequence: Synthetic peptide”
WRDAFDI

SEQ ID NO: 14 - protein, 14 aa, Artificial Sequence
  source: /note=“Description of Artificial Sequence: Synthetic peptide”
TGSSSNIGAGYDVH

SEQ ID NO: 15 - protein, 7 aa, Artificial Sequence
  source: /note=“Description of Artificial Sequence: Synthetic peptide”
SDNQRPS

SEQ ID NO: 16 - protein, 12 aa, Artificial Sequence
  source: /note=“Description of Artificial Sequence: Synthetic peptide”
AAWDDSLSGSWV

SEQ ID NO: 17 - protein, 117 aa, Artificial Sequence
  source: /note=“Description of Artificial Sequence: Synthetic polypeptide”
EVQLLESGGGLVQPGGSLRLSCAASGFTFSTYGMHWVRQAPGKGLEWVAVIAYDGSKKDYADSVKGRFTISRDNSKNTLYLQMNSLRAEDTAVYYCAREYRDAFDIWGQGTLVTVSS

SEQ ID NO: 18 - protein, 112 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYGNSNRPSGVPDRFSGSKSGTTASLAISGLRSEDEADYYCAAWDDSVSGWMFGGGTKLTVLG

SEQ ID NO: 19 - protein, 5 aa, Artificial Sequence
  source: /note=“Description of Artificial Sequence: Synthetic peptide”
TYGMH

SEQ ID NO: 20 - protein, 17 aa, Artificial Sequence
  source: /note=“Description of Artificial Sequence: Synthetic peptide”
VIAYDGSKKDYADSVKG

SEQ ID NO: 21 - protein, 8 aa, Artificial Sequence
  source: /note=“Description of Artificial Sequence: Synthetic peptide”
EYRDAFDI

SEQ ID NO: 22 - protein, 14 aa, Artificial Sequence
  source: /note=“Description of Artificial Sequence: Synthetic peptide”
TGSSSNIGAGYDVH

SEQ ID NO: 23 - protein, 7 aa, Artificial Sequence
  source: /note=“Description of Artificial Sequence: Synthetic peptide”
GNSNRPS

SEQ ID NO: 24 - protein, 11 aa, Artificial Sequence
  source: /note=“Description of Artificial Sequence: Synthetic peptide”
AAWDDSVSGWM

SEQ ID NO: 25 - protein, 117 aa, Artificial Sequence
  source: /note=“Description of Artificial Sequence: Synthetic polypeptide”
EVQLLESGGGLVQPGGSLRLSCAASGFTFNNYGMHWVRQAPGKGLEWVAVISYDGSNRYYADSVKGRFTMSRDNSKNTLYLQMNSLRAEDTAVYYCARDRWNGMDVWGQGTLVTVSS

SEQ ID NO: 26 - protein, 113 aa, Artificial Sequence
  source: /note=“Description of Artificial Sequence: Synthetic polypeptide”
QSVLTQPPSASGTPGQRVTISCSGSSSNIGAGYDVHWYQQLPGTAPKLLIYANNQRPSGVPDRFSGSKSGTSASLAISGLRSEDEADYYCAAWDDSLNGPWVFGGGTKLTVLG

SEQ ID NO: 27 - protein, 5 aa, Artificial Sequence
  source: /note=“Description of Artificial Sequence: Synthetic peptide”
NYGMH

SEQ ID NO: 28 - protein, 17 aa, Artificial Sequence
  source: /note=“Description of Artificial Sequence: Synthetic peptide”
VISYDGSNRYYADSVKG

SEQ ID NO: 29 - protein, 8 aa, Artificial Sequence
  source: /note=“Description of Artificial Sequence: Synthetic peptide”
DRWNGMDV

SEQ ID NO: 30 - protein, 14 aa, Artificial Sequence
  source: /note=“Description of Artificial Sequence: Synthetic peptide”
SGSSSNIGAGYDVH

SEQ ID NO: 31 - protein, 7 aa, Artificial Sequence
  source: /note=“Description of Artificial Sequence: Synthetic peptide”
ANNQRPS

SEQ ID NO: 32 - protein, 12 aa, Artificial Sequence
  source: /note=“Description of Artificial Sequence: Synthetic peptide”
AAWDDSLNGPWV

SEQ ID NO: 33 - protein, 119 aa, Artificial Sequence
  source: /note=“Description of Artificial Sequence: Synthetic polypeptide”
EVQLLESGGGLVQPGGSLRLSCAASGFTFSSYGMHWVRQAPGKGLEWVAVISYDGSDTAYADSVKGRFTISRDNSKNTLYLQMNSLRAEDTAVYYCARDHSVIGAFDIWGQGTLVTVSS

SEQ ID NO: 34 - protein, 110 aa, Artificial Sequence
  source: /note=“Description of Artificial Sequence: Synthetic polypeptide”
QSVLTQPPSASGTPGQRVTISCSGSSSNIGSNTVNWYQQLPGTAPKLLIYDNNKRPSGVPDRFSGSKSGTSASLAISGLRSEDEADYYCSSYAGSNNVVFGGGTKLTVLG

SEQ ID NO: 35 - protein, 5 aa, Artificial Sequence
  source: /note=“Description of Artificial Sequence: Synthetic peptide”
SYGMH

SEQ ID NO: 36 - protein, 17 aa, Artificial Sequence
  source: /note=“Description of Artificial Sequence: Synthetic peptide”
VISYDGSDTAYADSVKG

SEQ ID NO: 37 - protein, 10 aa, Artificial Sequence
  source: /note=“Description of Artificial Sequence: Synthetic peptide”
DHSVIGAFDI

SEQ ID NO: 38 - protein, 13 aa, Artificial Sequence
  source: /note=“Description of Artificial Sequence: Synthetic peptide”
SGSSSNIGSNTVN

SEQ ID NO: 39 - protein, 7 aa, Artificial Sequence
  source: /note=“Description of Artificial Sequence: Synthetic peptide”
DNNKRPS

SEQ ID NO: 40 - protein, 10 aa, Artificial Sequence
  source: /note=“Description of Artificial Sequence: Synthetic peptide”
SSYAGSNNVV

SEQ ID NO: 41 - protein, 118 aa, Artificial Sequence
  source: /note=“Description of Artificial Sequence: Synthetic polypeptide”
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCARDQLGEAFDIWGQGTLVTVSS

SEQ ID NO: 42 - protein, 112 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYDNNKRPSGVPDRFSGSKSGTSASLAISGLRSEDEADYYCATWDDSLSGPVFGGGTKLTVLG

SEQ ID NO: 43 - protein, 5 aa, Artificial Sequence
  source: /note=“Description of Artificial Sequence: Synthetic peptide”
NYGMH

SEQ ID NO: 44 - protein, 17 aa, Artificial Sequence
  source: /note=“Description of Artificial Sequence: Synthetic peptide”
VISYDGSNKYYADSVKG

SEQ ID NO: 45 - protein, 9 aa, Artificial Sequence
  source: /note=“Description of Artificial Sequence: Synthetic peptide”
DQLGEAFDI

SEQ ID NO: 46 - protein, 14 aa, Artificial Sequence
  source: /note=“Description of Artificial Sequence: Synthetic peptide”
TGSSSNIGAGYDVH

SEQ ID NO: 47 - protein, 7 aa, Artificial Sequence
  source: /note=“Description of Artificial Sequence: Synthetic peptide”
DNNKRPS

SEQ ID NO: 48 - protein, 11 aa, Artificial Sequence
  source: /note=“Description of Artificial Sequence: Synthetic peptide”
ATWDDSLSGPV

SEQ ID NO: 49 - protein, 117 aa, Artificial Sequence
  source: /note=“Description of Artificial Sequence: Synthetic polypeptide”
EVQLLESGGGLVQPGGSLRLSCAASGFTFDDYGMSWVRQAPGKGLEWVSAISGSGSSTYYADSVKGRFTISRDNSKNTLYLQMNSLRAEDTAVYYCAGGDIDYFDYWGQGTLVTVSS

SEQ ID NO: 50 - protein, 112 aa, Artificial Sequence
  source: /note=“Description of Artificial Sequence: Synthetic polypeptide”
QSVLTQPPSASGTPGQRVTISCTGSSSNFGAGYDVHWYQQLPGTAPKLLIYENNKRPSGVPDRFSGSKSGTSASLAISGLRSEDEADYYCAAWDDSLNGPVFGGGTKLTVLG

SEQ ID NO: 51 - protein, 5 aa, Artificial Sequence
  source: /note=“Description of Artificial Sequence: Synthetic peptide”
DYGMS

SEQ ID NO: 52 - protein, 17 aa, Artificial Sequence
  source: /note=“Description of Artificial Sequence: Synthetic peptide”
AISGSGSSTYYADSVKG

SEQ ID NO: 53 - protein, 8 aa, Artificial Sequence
  source: /note=“Description of Artificial Sequence: Synthetic peptide”
GDIDYFDY

SEQ ID NO: 54 - protein, 14 aa, Artificial Sequence
  source: /note=“Description of Artificial Sequence: Synthetic peptide”
TGSSSNFGAGYDVH

SEQ ID NO: 55 - protein, 7 aa, Artificial Sequence
  source: /note=“Description of Artificial Sequence: Synthetic peptide”
ENNKRPS

SEQ ID NO: 56 - protein, 11 aa, Artificial Sequence
  source: /note=“Description of Artificial Sequence: Synthetic peptide”
AAWDDSLNGPV

SEQ ID NO: 57 - protein, 117 aa, Artificial Sequence
  source: /note=“Description of Artificial Sequence: Synthetic polypeptide”
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARERRDAFDIWGQGTLVTVSS

SEQ ID NO: 58 - protein, 110 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYSDNQRPSGVPDRFSGSKSGTSASLAISGLRSEDEADYYCATWDSDTPVFGGGTKLTVLG

SEQ ID NO: 59 - protein, 5 aa, Artificial Sequence
  source: /note=“Description of Artificial Sequence: Synthetic peptide”
SYGMH

SEQ ID NO: 60 - protein, 17 aa, Artificial Sequence
  source: /note=“Description of Artificial Sequence: Synthetic peptide”
VISYDGSNKYYADSVKG

SEQ ID NO: 61 - protein, 8 aa, Artificial Sequence
  source: /note=“Description of Artificial Sequence: Synthetic peptide”
ERRDAFDI

SEQ ID NO: 62 - protein, 14 aa, Artificial Sequence
  source: /note=“Description of Artificial Sequence: Synthetic peptide”
TGSSSNIGAGYDVH

SEQ ID NO: 63 - protein, 7 aa, Artificial Sequence
  source: /note=“Description of Artificial Sequence: Synthetic peptide”
SDNQRPS

SEQ ID NO: 64 - protein, 9 aa, Artificial Sequence
  source: /note=“Description of Artificial Sequence: Synthetic peptide”
ATWDSDTPV

SEQ ID NO: 65 - protein, 119 aa, Artificial Sequence
  source: /note=“Description of Artificial Sequence: Synthetic polypeptide”
EVQLLESGGGLVQPGGSLRLSCAASGFTFSSYGMHWVRQAPGKGLEWVAVISYDGSNKYYADSVKGRFTISRDNSKNTLYLQMNSLRAEDTAMYYCARDHSAAGYFDYWGQGTLVTVSS

SEQ ID NO: 66 - protein, 111 aa, Artificial Sequence
  source: /note=“Description of Artificial Sequence: Synthetic polypeptide”
QSVLTQPPSASGTPGQRVTISCSGSSSNIGSNTVNWYQQLPGTAPKLLIYGNSIRPSGGPDRFSGSKSGTSASLAISGLRSEDEADYYCASWDDSLSSPVFGGGTKLTVLG

SEQ ID NO: 67 - protein, 5 aa, Artificial Sequence
  source: /note=“Description of Artificial Sequence: Synthetic peptide”
SYGMH

SEQ ID NO: 68 - protein, 17 aa, Artificial Sequence
  source: /note=“Description of Artificial Sequence: Synthetic peptide”
VISYDGSNKYYADSVKG

SEQ ID NO: 69 - protein, 10 aa, Artificial Sequence
  source: /note=“Description of Artificial Sequence: Synthetic peptide”
DHSAAGYFDY

SEQ ID NO: 70 - protein, 13 aa, Artificial Sequence
  source: /note=“Description of Artificial Sequence: Synthetic peptide”
SGSSSNIGSNTVN

SEQ ID NO: 71 - protein, 7 aa, Artificial Sequence
  source: /note=“Description of Artificial Sequence: Synthetic peptide”
GNSIRPS

SEQ ID NO: 72 - protein, 11 aa, Artificial Sequence
  source: /note=“Description of Artificial Sequence: Synthetic peptide”
ASWDDSLSSPV

SEQ ID NO: 73 - protein, 119 aa, Artificial Sequence
  source: /note=“Description of Artificial Sequence: Synthetic polypeptide”
EVQLLESGGGLVQPGGSLRLSCAASGFTFGSYGMHWVRQAPGKGLEWVSGISWDSAIIDYAGSVKGRFTISRDNSKNTLYLQMNSLRAEDTAVYYCAKDEAAAGAFDIWGQGTLVTVSS

SEQ ID NO: 74 - protein, 113 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYGNTDRPSGVPDRFSGSKSGTSASLAISGLRSEDEADYYCAAWDDSLSGPVVFGGGTKLTVLG

SEQ ID NO: 75 - protein, 5 aa, Artificial Sequence
  source: /note=“Description of Artificial Sequence: Synthetic peptide”
SYGMH

SEQ ID NO: 76 - protein, 17 aa, Artificial Sequence
  source: /note=“Description of Artificial Sequence: Synthetic peptide”
GISWDSAIIDYAGSVKG

SEQ ID NO: 77 - protein, 10 aa, Artificial Sequence
  source: /note=“Description of Artificial Sequence: Synthetic peptide”
DEAAAGAFDI

SEQ ID NO: 78 - protein, 14 aa, Artificial Sequence
  source: /note=“Description of Artificial Sequence: Synthetic peptide”
TGSSSNIGAGYDVH

SEQ ID NO: 79 - protein, 7 aa, Artificial Sequence
  source: /note=“Description of Artificial Sequence: Synthetic peptide”
GNTDRPS

SEQ ID NO: 80 - protein, 12 aa, Artificial Sequence
  source: /note=“Description of Artificial Sequence: Synthetic peptide”
AAWDDSLSGPVV

SEQ ID NO: 81 - protein, 118 aa, Artificial Sequence
  source: /note=“Description of Artificial Sequence: Synthetic polypeptide”
EVQLLESGGGLVQPGGSLRLSCAASGFTFSSYGMHWVRQAPGKGLEWVAVISYDGSNKYYADSVKGRFTISRDNSQNTLYLQMNSLRAEDTAVYYCAREFGYIILDYWGQGTLVTVSS

SEQ ID NO: 82 - protein, 111 aa, Artificial Sequence
  source: /note=“Description of Artificial Sequence: Synthetic polypeptide”
QSVLTQPPSASGTPGQRVTISCSGSSSNIGSNTVNWYQQLPGTAPKLLIYRDYERPSGVPDRFSGSKSGTSASLAISGLRSEDEADYYCMAWDDSLSGVVFGGGTKLTVLG

SEQ ID NO: 83 - protein, 5 aa, Artificial Sequence
  source: /note=“Description of Artificial Sequence: Synthetic peptide”
SYGMH

SEQ ID NO: 84 - protein, 17 aa, Artificial Sequence
  source: /note=“Description of Artificial Sequence: Synthetic peptide”
VISYDGSNKYYADSVKG

SEQ ID NO: 85 - protein, 9 aa, Artificial Sequence
  source: /note=“Description of Artificial Sequence: Synthetic peptide”
EFGYIILDY

SEQ ID NO: 86 - protein, 13 aa, Artificial Sequence
  source: /note=“Description of Artificial Sequence: Synthetic peptide”
SGSSSNIGSNTVN

SEQ ID NO: 87 - protein, 7 aa, Artificial Sequence
  source: /note=“Description of Artificial Sequence: Synthetic peptide”
RDYERPS

SEQ ID NO: 88 - protein, 11 aa, Artificial Sequence
  source: /note=“Description of Artificial Sequence: Synthetic peptide”
MAWDDSLSGVV

SEQ ID NO: 89 - protein, 117 aa, Artificial Sequence
  source: /note=“Description of Artificial Sequence: Synthetic polypeptide”
EVQLLESGGGLVQPGGSLRLSCAASGFTFSNHGMHWVRQAPGKGLEWVAVISYDGTNKYYADSVRGRFTISRDNSKNTLYLQMNSLRAEDTAVYYCARETWDAFDVWGQGTLVTVSS

SEQ ID NO: 90 - protein, 109 aa, Artificial Sequence
  source: /note=“Description of Artificial Sequence: Synthetic polypeptide”
QSVLTQPPSASGTPGQRVTISCSGSSSNIGSNNANWYQQLPGTAPKLLIYDNNKRPSGVPDRFSGSKSGTSASLAISGLRSEDEADYYCQAWDSSTVVFGGGTKLTVLG

SEQ ID NO: 91 - protein, 5 aa, Artificial Sequence
  source: /note=“Description of Artificial Sequence: Synthetic peptide”
NHGMH

SEQ ID NO: 92 - protein, 17 aa, Artificial Sequence
  source: /note=“Description of Artificial Sequence: Synthetic peptide”
VISYDGTNKYYADSVRG

SEQ ID NO: 93 - protein, 8 aa, Artificial Sequence
  source: /note=“Description of Artificial Sequence: Synthetic peptide”
ETWDAFDV

SEQ ID NO: 94 - protein, 13 aa, Artificial Sequence
  source: /note=“Description of Artificial Sequence: Synthetic peptide”
SGSSSNIGSNNAN

SEQ ID NO: 95 - protein, 7 aa, Artificial Sequence
  source: /note=“Description of Artificial Sequence: Synthetic peptide”
DNNKRPS

SEQ ID NO: 96 - protein, 9 aa, Artificial Sequence
  source: /note=“Description of Artificial Sequence: Synthetic peptide”
QAWDSSTVV

SEQ ID NO: 97 - protein, 121 aa, Artificial Sequence
  source: /note=“Description of Artificial Sequence: Synthetic polypeptide”
EVQLLESGGGLVQPGGSLRLSCAASGFTLSSYGISWVRQAPGKGLEWVSGISGSGGNTYYADSVKGRFTISRDNSKNTLYLQMNSLRAEDTAVYYCASSVGAYANDAFDIWGQGTLVTVSS

SEQ ID NO: 98 - protein, 112 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYGDTNRPSGVPDRFSGSKSGTSASLAISGLRSEDEADYYCAAWDDSLNGPVFGGGTKLTVLG

SEQ ID NO: 99 - protein, 5 aa, Artificial Sequence
  source: /note=“Description of Artificial Sequence: Synthetic peptide”
SYGIS

SEQ ID NO: 100 - protein, 17 aa, Artificial Sequence
  source: /note=“Description of Artificial Sequence: Synthetic peptide”
GISGSGGNTYYADSVKG

SEQ ID NO: 101 - protein, 12 aa, Artificial Sequence
  source: /note=“Description of Artificial Sequence: Synthetic peptide”
SVGAYANDAFDI

SEQ ID NO: 102 - protein, 14 aa, Artificial Sequence
  source: /note=“Description of Artificial Sequence: Synthetic peptide”
TGSSSNIGAGYDVH

SEQ ID NO: 103 - protein, 7 aa, Artificial Sequence
  source: /note=“Description of Artificial Sequence: Synthetic peptide”
GDTNRPS

SEQ ID NO: 104 - protein, 11 aa, Artificial Sequence
  source: /note=“Description of Artificial Sequence: Synthetic peptide”
AAWDDSLNGPV

SEQ ID NO: 105 - protein, 117 aa, Artificial Sequence
  source: /note=“Description of Artificial Sequence: Synthetic polypeptide”
EVQLLESGGGLVQPGGSLRLSCAASGFTFSTYGMHWVRQAPGKGLEWVAVISYDGSNKYYADSVKGRFTISRDNSKNTLYLQMNSLRAEDTAVYYCARENFDAFDVWGQGTLVTVSS

SEQ ID NO: 106 - protein, 113 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYSNSQRPSGVPDRFSGSKSGTSASLAISGLRSEDEADYYCAAWDDSLNGQVVFGGGTKLTVLG

SEQ ID NO: 107 - protein, 5 aa, Artificial Sequence
  source: /note=“Description of Artificial Sequence: Synthetic peptide”
TYGMH

SEQ ID NO: 108 - protein, 17 aa, Artificial Sequence
  source: /note=“Description of Artificial Sequence: Synthetic peptide”
VISYDGSNKYYADSVKG

SEQ ID NO: 109 - protein, 8 aa, Artificial Sequence
  source: /note=“Description of Artificial Sequence: Synthetic peptide”
ENFDAFDV

SEQ ID NO: 110 - protein, 14 aa, Artificial Sequence
  source: /note=“Description of Artificial Sequence: Synthetic peptide”
TGSSSNIGAGYDVH

SEQ ID NO: 111 - protein, 7 aa, Artificial Sequence
  source: /note=“Description of Artificial Sequence: Synthetic peptide”
SNSQRPS

SEQ ID NO: 112 - protein, 12 aa, Artificial Sequence
  source: /note=“Description of Artificial Sequence: Synthetic peptide”
AAWDDSLNGQVV

SEQ ID NO: 113 - protein, 117 aa, Artificial Sequence
  source: /note=“Description of Artificial Sequence: Synthetic polypeptide”
EVQLLESGGGLVQPGGSLRLSCAASGFTFSSYGMHWVRQAPGKGLEWVAVISYDGINKDYADSMKGRFTISRDNSKNTLYLQMNSLRAEDTAVYYCARERKDAFDIWGQGTLVTVSS

SEQ ID NO: 114 - protein, 112 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYSNNQRPSGVPDRFSGSKSGTSASLAISGLRSEDEADYYCATWDDSLNGLVFGGGTKLTVLG

SEQ ID NO: 115 - protein, 5 aa, Artificial Sequence
  source: /note=“Description of Artificial Sequence: Synthetic peptide”
SYGMH

SEQ ID NO: 116 - protein, 17 aa, Artificial Sequence
  source: /note=“Description of Artificial Sequence: Synthetic peptide”
VISYDGINKDYADSMKG

SEQ ID NO: 117 - protein, 8 aa, Artificial Sequence
  source: /note=“Description of Artificial Sequence: Synthetic peptide”
ERKDAFDI

SEQ ID NO: 118 - protein, 14 aa, Artificial Sequence
  source: /note=“Description of Artificial Sequence: Synthetic peptide”
TGSSSNIGAGYDVH

SEQ ID NO: 119 - protein, 7 aa, Artificial Sequence
  source: /note=“Description of Artificial Sequence: Synthetic peptide”
SNNQRPS

SEQ ID NO: 120 - protein, 11 aa, Artificial Sequence
  source: /note=“Description of Artificial Sequence: Synthetic peptide”
ATWDDSLNGLV

SEQ ID NO: 121 - protein, 119 aa, Artificial Sequence
  source: /note=“Description of Artificial Sequence: Synthetic polypeptide”
EVQLLESGGGLVQPGGSLRLSCAASGFTFSSYGMHWVRQAPGKGLEWVAVTSYDGNTKYYANSVKGRFTISRDNSKNTLYLQMNSLRAEDTAVYYCAREDCGGDCFDYWGQGTLVTVSS

SEQ ID NO: 122 - protein, 112 aa, Artificial Sequence
  source: /note=“Description of Artificial Sequence: Synthetic polypeptide”
QSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSLNEGVFGGGTKLTVLG

SEQ ID NO: 123 - protein, 5 aa, Artificial Sequence
  source: /note=“Description of Artificial Sequence: Synthetic peptide”
SYGMH

SEQ ID NO: 124 - protein, 17 aa, Artificial Sequence
  source: /note=“Description of Artificial Sequence: Synthetic peptide”
VTSYDGNTKYYANSVKG

SEQ ID NO: 125 - protein, 10 aa, Artificial Sequence
  source: /note=“Description of Artificial Sequence: Synthetic peptide”
EDCGGDCFDY

SEQ ID NO: 126 - protein, 14 aa, Artificial Sequence
  source: /note=“Description of Artificial Sequence: Synthetic peptide”
TGSSSNIGAGYDVH

SEQ ID NO: 127 - protein, 7 aa, Artificial Sequence
  source: /note=“Description of Artificial Sequence: Synthetic peptide”
GNSNRPS

SEQ ID NO: 128 - protein, 11 aa, Artificial Sequence
  source: /note=“Description of Artificial Sequence: Synthetic peptide”
AAWDDSLNEGV

SEQ ID NO: 129 - protein, 117 aa, Artificial Sequence
  source: /note=“Description of Artificial Sequence: Synthetic polypeptide”
EVQLLESGGGLVQPGGSLRLSCAASGFTFNNYGMHWVRQAPGKGLEWVAVISYDGSNKYYADSVKGRFTISKDNSKNTLYLQMNSLRAEDTAVYYCAREYKDAFDIWGQGTLVTVSS

SEQ ID NO: 130 - protein, 109 aa, Artificial Sequence
  source: /note=“Description of Artificial Sequence: Synthetic polypeptide”
QSVLTQPPSASGTPGQRVTISCTGSSSNIGSNTVNWYQQLPGTAPKLLIYDNNKRPSGVPDRFSGSKSGTSASLAISGLRSEDEADYYCQAWGTGIRVFGGGTKLTVLG

SEQ ID NO: 131 - protein, 5 aa, Artificial Sequence
  source: /note=“Description of Artificial Sequence: Synthetic peptide”
NYGMH

SEQ ID NO: 132 - protein, 17 aa, Artificial Sequence
  source: /note=“Description of Artificial Sequence: Synthetic peptide”
VISYDGSNKYYADSVKG

SEQ ID NO: 133 - protein, 8 aa, Artificial Sequence
  source: /note=“Description of Artificial Sequence: Synthetic peptide”
EYKDAFDI

SEQ ID NO: 134 - protein, 13 aa, Artificial Sequence
  source: /note=“Description of Artificial Sequence: Synthetic peptide”
TGSSSNIGSNTVN

SEQ ID NO: 135 - protein, 7 aa, Artificial Sequence
  source: /note=“Description of Artificial Sequence: Synthetic peptide”
DNNKRPS

SEQ ID NO: 136 - protein, 9 aa, Artificial Sequence
  source: /note=“Description of Artificial Sequence: Synthetic peptide”
QAWGTGIRV

SEQ ID NO: 137-200 - skipped (64 entries)

SEQ ID NO: 201 - protein, 118 aa, Artificial Sequence
  source: /note=“Description of Artificial Sequence: Synthetic polypeptide”
QVQLVESGGGVVQPGRSLRLSCAASGFTFSSYGIHWVRQAPGKGLEWVAVIGYDGSDKNYADSVKGRFTIFRDNSKNTLYLQMNSLRAEDTAVYYCARDQLGDAFDIWGQGTMVTVSS

SEQ ID NO: 202 - protein, 5 aa, Artificial Sequence
  source: /note=“Description of Artificial Sequence: Synthetic peptide”
SYGIH

SEQ ID NO: 203 - protein, 17 aa, Artificial Sequence
  source: /note=“Description of Artificial Sequence: Synthetic peptide”
VIGYDGSDKNYADSVKG

SEQ ID NO: 204 - protein, 9 aa, Artificial Sequence
  source: /note=“Description of Artificial Sequence: Synthetic peptide”
DQLGDAFDI

SEQ ID NO: 205 - protein, 109 aa, Artificial Sequence
  source: /note=“Description of Artificial Sequence: Synthetic polypeptide”
EIVLTQSPATLSLSPGERATLSCKASQSVSSSLAWYQQKPGQAPRLLIYDASNRATGIPARFSGSGSGTDFTLTISSLEPEDFAVYYCQQRSNWPPYTFGQGTKLEIKR

SEQ ID NO: 206 - protein, 11 aa, Artificial Sequence
  source: /note=“Description of Artificial Sequence: Synthetic peptide”
KASQSVSSSLA

SEQ ID NO: 207 - protein, 7 aa, Artificial Sequence
  source: /note=“Description of Artificial Sequence: Synthetic peptide”
DASNRAT

SEQ ID NO: 208 - protein, 10 aa, Artificial Sequence
  source: /note=“Description of Artificial Sequence: Synthetic peptide”
QQRSNWPPYT

SEQ ID NO: 209 - protein, 118 aa, Artificial Sequence
  source: /note=“Description of Artificial Sequence: Synthetic polypeptide”
QVQLVQSGGEVKKPGASVKVSCKTSGYTFTSYGISWVRQAPGQGLEWMGWISAYNGNTKYAQKLQGRLTMTTDTSTTTAYMELRSLRSDDTAVYYCARDSAAHGMDVWGQGTTVTVSS

SEQ ID NO: 210 - protein, 5 aa, Artificial Sequence
  source: /note=“Description of Artificial Sequence: Synthetic peptide”
SYGIS

SEQ ID NO: 211 - protein, 17 aa, Artificial Sequence
  source: /note=“Description of Artificial Sequence: Synthetic peptide”
WISAYNGNTKYAQKLQG

SEQ ID NO: 212 - protein, 9 aa, Artificial Sequence
  source: /note=“Description of Artificial Sequence: Synthetic peptide”
DSAAHGMDV

SEQ ID NO: 213 - protein, 108 aa, Artificial Sequence
  source: /note=“Description of Artificial Sequence: Synthetic polypeptide”
DIQMTQSPSSLSASVGDRVTITCRASQGISSWLAWYQQKPEKAPKSLIYAASSLQSGVPSRFRGSGSGTDFTLTISSLQPEDFATYYCQQYNSYPYTFGQGTKLEIKR

SEQ ID NO: 214 - protein, 11 aa, Artificial Sequence
  source: /note=“Description of Artificial Sequence: Synthetic peptide”
RASQGISSWLA

SEQ ID NO: 215 - protein, 7 aa, Artificial Sequence
  source: /note=“Description of Artificial Sequence: Synthetic peptide”
AASSLQS

SEQ ID NO: 216 - protein, 9 aa, Artificial Sequence
  source: /note=“Description of Artificial Sequence: Synthetic peptide”
QQYNSYPYT

SEQ ID NO: 217 - protein, 118 aa, Artificial Sequence
  source: /note=“Description of Artificial Sequence: Synthetic polypeptide”
QVQLVQSGAEVKKPGASVKVSCKASGYTFTSYGLSWVRQAPGQGLEWMGWISPYNGNTHYAQKLQGRVTMTTDTSTSTADMDLRSLRSDDTAVYYCARASAAHGMDVWGQGTTVTVSS

SEQ ID NO: 218 - protein, 5 aa, Artificial Sequence
  source: /note=“Description of Artificial Sequence: Synthetic peptide”
SYGLS

SEQ ID NO: 219 - protein, 17 aa, Artificial Sequence
  source: /note=“Description of Artificial Sequence: Synthetic peptide”
WISPYNGNTHYAQKLQG

SEQ ID NO: 220 - protein, 9 aa, Artificial Sequence
  source: /note=“Description of Artificial Sequence: Synthetic peptide”
ASAAHGMDV

SEQ ID NO: 221 - protein, 108 aa, Artificial Sequence
  source: /note=“Description of Artificial Sequence: Synthetic polypeptide”
DIQMTQSPSSLSASVGDRVTITCRASQGISSWLAWYQQKPEKAPKSLIYAASSLQSGVPSRFSGSRSGTDFTLTISSLQPEDFATYYCQQYNSYPYTFGQGTKLEIKR

SEQ ID NO: 222 - protein, 11 aa, Artificial Sequence
  source: /note=“Description of Artificial Sequence: Synthetic peptide”
RASQGISSWLA

SEQ ID NO: 223 - protein, 7 aa, Artificial Sequence
  source: /note=“Description of Artificial Sequence: Synthetic peptide”
AASSLQS

SEQ ID NO: 224 - protein, 9 aa, Artificial Sequence
  source: /note=“Description of Artificial Sequence: Synthetic peptide”
QQYNSYPYT

SEQ ID NO: 225 - protein, 118 aa, Artificial Sequence
  source: /note=“Description of Artificial Sequence: Synthetic polypeptide”
QVQLVQSGAEVKKPGASVKVSCKASGYTFTSYGLSWVRQAPGQGLEWMGWISPYNGNTHYAQKLQGRVTMTTDTSTSTAYMDLRSLRSDDTAVYYCARDSAAHGMDVWGQGTTVTVSS

SEQ ID NO: 226 - protein, 5 aa, Artificial Sequence
  source: /note=“Description of Artificial Sequence: Synthetic peptide”
SYGLS

SEQ ID NO: 227 - protein, 17 aa, Artificial Sequence
  source: /note=“Description of Artificial Sequence: Synthetic peptide”
WISPYNGNTHYAQKLQG

SEQ ID NO: 228 - protein, 9 aa, Artificial Sequence
  source: /note=“Description of Artificial Sequence: Synthetic peptide”
DSAAHGMDV

SEQ ID NO: 229 - protein, 108 aa, Artificial Sequence; identical to SEQ ID NO: 221
  source: /note=“Description of Artificial Sequence: Synthetic polypeptide”

SEQ ID NO: 230 - protein, 11 aa, Artificial Sequence
  source: /note=“Description of Artificial Sequence: Synthetic peptide”
RASQGISSWLA

SEQ ID NO: 231 - protein, 7 aa, Artificial Sequence
  source: /note=“Description of Artificial Sequence: Synthetic peptide”
AASSLQS

SEQ ID NO: 232 - protein, 9 aa, Artificial Sequence
  source: /note=“Description of Artificial Sequence: Synthetic peptide”
QQYNSYPYT

SEQ ID NO: 233 - protein, 118 aa, Artificial Sequence
  source: /note=“Description of Artificial Sequence: Synthetic polypeptide”
QVQLVQSGAEVKKPGASVKVSCKASGYTFTSYGLSWVRQAPGQGLEWMGWISAYNGNTNYAQKLQGRVTMTTDTSTSTAYMDLRSLRSDDTAVYYCARDSAAHGMDVWGQGTTVTVSS

SEQ ID NO: 234 - protein, 5 aa, Artificial Sequence
  source: /note=“Description of Artificial Sequence: Synthetic peptide”
SYGLS

SEQ ID NO: 235 - protein, 17 aa, Artificial Sequence
  source: /note=“Description of Artificial Sequence: Synthetic peptide”
WISAYNGNTNYAQKLQG

SEQ ID NO: 236 - protein, 9 aa, Artificial Sequence
  source: /note=“Description of Artificial Sequence: Synthetic peptide”
DSAAHGMDV

SEQ ID NO: 237 - protein, 108 aa, Artificial Sequence; identical to SEQ ID NO: 221
  source: /note=“Description of Artificial Sequence: Synthetic polypeptide”

SEQ ID NO: 238 - protein, 11 aa, Artificial Sequence
  source: /note=“Description of Artificial Sequence: Synthetic peptide”
RASQGISSWLA

SEQ ID NO: 239 - protein, 7 aa, Artificial Sequence
  source: /note=“Description of Artificial Sequence: Synthetic peptide”
AASSLQS

SEQ ID NO: 240 - protein, 9 aa, Artificial Sequence
  source: /note=“Description of Artificial Sequence: Synthetic peptide”
QQYNSYPYT

SEQ ID NO: 241 - protein, 118 aa, Artificial Sequence
  source: /note=“Description of Artificial Sequence: Synthetic polypeptide”
QVQVVQSGAEVKKPGASVKVSCKTSGYTFTSYGISWVRQAPGQGLEWMGWISAYNGNTKYAQKLQGRLTMTTDTSTTTAYMELRSLRSDDTAVYYCARDSAAHGMDVWGQGTTVSVSS

SEQ ID NO: 242 - protein, 5 aa, Artificial Sequence
  source: /note=“Description of Artificial Sequence: Synthetic peptide”
SYGIS

SEQ ID NO: 243 - protein, 17 aa, Artificial Sequence
  source: /note=“Description of Artificial Sequence: Synthetic peptide”
WISAYNGNTKYAQKLQG

SEQ ID NO: 244 - protein, 9 aa, Artificial Sequence
  source: /note=“Description of Artificial Sequence: Synthetic peptide”
DSAAHGMDV

SEQ ID NO: 245 - protein, 117 aa, Artificial Sequence
  source: /note=“Description of Artificial Sequence: Synthetic polypeptide”
EVQLLESGGGLVQPGGSLRLSCAASGFTFSNFVMSWVRQAPGKGLEWVSGISGSGGNTDHADSVKGRFTISRDNSKNTVYLQMNSLRAEDTAVYYCAKDSGGLFDYWGLGTLVTVSS

SEQ ID NO: 246 - protein, 5 aa, Artificial Sequence
  source: /note=“Description of Artificial Sequence: Synthetic peptide”
NFVMS

SEQ ID NO: 247 - protein, 17 aa, Artificial Sequence
  source: /note=“Description of Artificial Sequence: Synthetic peptide”
GISGSGGNTDHADSVKG

SEQ ID NO: 248 - protein, 8 aa, Artificial Sequence
  source: /note=“Description of Artificial Sequence: Synthetic peptide”
DSGGLFDY

SEQ ID NO: 249 - protein, 109 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSRTDFTLTISSLEPEDFAVYYCQQRSNWPHLTFGGGTKVEIKR

SEQ ID NO: 250 - protein, 11 aa, Artificial Sequence
  source: /note=“Description of Artificial Sequence: Synthetic peptide”
RASQSVSSYLA

SEQ ID NO: 251 - protein, 7 aa, Artificial Sequence
  source: /note=“Description of Artificial Sequence: Synthetic peptide”
DASNRAT

SEQ ID NO: 252 - protein, 10 aa, Artificial Sequence
  source: /note=“Description of Artificial Sequence: Synthetic peptide”
QQRSNWPHLT

SEQ ID NO: 253 - protein, 119 aa, Artificial Sequence
  source: /note=“Description of Artificial Sequence: Synthetic polypeptide”
QVQLVESGGGVVQPGRSLRLSCAASGFTFSTYGMHWVRQAPGKGLEWVAVISHDGSDKYYADSVKGRFTISRDNSKNTLYLQMNSLRAEDTAVYYCARDQSIIETFDYWGQGTLVTVSS

SEQ ID NO: 254 - protein, 5 aa, Artificial Sequence
  source: /note=“Description of Artificial Sequence: Synthetic peptide”
TYGMH

SEQ ID NO: 255 - protein, 17 aa, Artificial Sequence
  source: /note=“Description of Artificial Sequence: Synthetic peptide”
VISHDGSDKYYADSVKG

SEQ ID NO: 256 - protein, 10 aa, Artificial Sequence
  source: /note=“Description of Artificial Sequence: Synthetic peptide”
DQSIIETFDY

SEQ ID NO: 257 - protein, 108 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWGFTFGPGTKVDIKR

SEQ ID NO: 258 - protein, 11 aa, Artificial Sequence
  source: /note=“Description of Artificial Sequence: Synthetic peptide”
RASQSVSSYLA

SEQ ID NO: 259 - protein, 7 aa, Artificial Sequence
  source: /note=“Description of Artificial Sequence: Synthetic peptide”
DASNRAT

SEQ ID NO: 260 - protein, 9 aa, Artificial Sequence
  source: /note=“Description of Artificial Sequence: Synthetic peptide”
QQRSNWGFT

SEQ ID NO: 261 - protein, 118 aa, Artificial Sequence
  source: /note=“Description of Artificial Sequence: Synthetic polypeptide”
QVQLVESGGGVVQPGRSLRLSCAVSGFTFRSYGMHWVRQAPGKGLEWVAVIWYDGSIKYYADSVKGRFTISRDNSKNTLYLQMNSLRAEDTAVYFCAREGGRDAFDIWGQGTMVTVSS

SEQ ID NO: 262 - protein, 5 aa, Artificial Sequence
  source: /note=“Description of Artificial Sequence: Synthetic peptide”
SYGMH

SEQ ID NO: 263 - protein, 17 aa, Artificial Sequence
  source: /note=“Description of Artificial Sequence: Synthetic peptide”
VIWYDGSIKYYADSVKG

SEQ ID NO: 264 - protein, 9 aa, Artificial Sequence
  source: /note=“Description of Artificial Sequence: Synthetic peptide”
EGGRDAFDI

SEQ ID NO: 265 - protein, 108 aa, Artificial Sequence
  source: /note=“Description of Artificial Sequence: Synthetic polypeptide”
AVQLTQSPSSLSASVGDRVTITCRASQGISSALAWYQQKPGKAPKLLIYDASSLESGVPSRFSGSGSGTDFTLTISSLQPEDFATYCCQQFNSYPHTFGGGTKVEIKR

SEQ ID NO: 266 - protein, 11 aa, Artificial Sequence
  source: /note=“Description of Artificial Sequence: Synthetic peptide”
RASQGISSALA

SEQ ID NO: 267 - protein, 7 aa, Artificial Sequence
  source: /note=“Description of Artificial Sequence: Synthetic peptide”
DASSLES

SEQ ID NO: 268 - protein, 9 aa, Artificial Sequence
  source: /note=“Description of Artificial Sequence: Synthetic peptide”
QQFNSYPHT

SEQ ID NO: 269 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISDSGGSTYYADSVKGRFTISRDNSKNTLYLQMNSLRAEDTAAYYCAKEIAVALFDYWGQGTLVTVSS

SEQ ID NO: 270 - protein, 5 aa, Artificial Sequence
  source: /note=“Description of Artificial Sequence: Synthetic peptide”
SYAMS

SEQ ID NO: 271 - protein, 17 aa, Artificial Sequence
  source: /note=“Description of Artificial Sequence: Synthetic peptide”
AISDSGGSTYYADSVKG

SEQ ID NO: 272 - protein, 9 aa, Artificial Sequence
  source: /note=“Description of Artificial Sequence: Synthetic peptide”
EIAVALFDY

SEQ ID NO: 273 - protein, 109 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSSWPPYTFGQGTKLEIKR

SEQ ID NO: 274 - protein, 11 aa, Artificial Sequence
  source: /note=“Description of Artificial Sequence: Synthetic peptide”
RASQSVSSYLA

SEQ ID NO: 275 - protein, 7 aa, Artificial Sequence
  source: /note=“Description of Artificial Sequence: Synthetic peptide”
DASNRAT

SEQ ID NO: 276 - protein, 10 aa, Artificial Sequence
  source: /note=“Description of Artificial Sequence: Synthetic peptide”
QQRSSWPPYT

SEQ ID NO: 277-299 - skipped (23 entries)

SEQ ID NO: 300 - protein, 121 aa, Artificial Sequence
  source: /note=“Description of Artificial Sequence: Synthetic polypeptide”
QVQLQQPVTELVRPGASVMLSCKASDYPFTNYWIHWVKQRPGQGLEWIGVIDPSDTYPNYNKKFKGKATLTVVVSSSTAYMQLSSLTSDDSAVYYCARNGDSDYYSGMDYWGQGTSVTVSS

SEQ ID NO: 301 - protein, 107 aa, Artificial Sequence
  source: /note=“Description of Artificial Sequence: Synthetic polypeptide”
DILLTQSPAILSVSPGERVSFSCRTSQSIGTNIHWYQQRTNGFPRLLIKNVSESISGIPSRFSGSGSGTDFILSINSVESEDIADYYCQQSNTWPFTFGGGTKLEIK

SEQ ID NO: 302 - protein, 5 aa, Artificial Sequence
  source: /note=“Description of Artificial Sequence: Synthetic peptide”
NYWIH

SEQ ID NO: 303 - protein, 17 aa, Artificial Sequence
  source: /note=“Description of Artificial Sequence: Synthetic peptide”
VIDPSDTYPNYNKKFKG

SEQ ID NO: 304 - protein, 12 aa, Artificial Sequence
  source: /note=“Description of Artificial Sequence: Synthetic peptide”
NGDSDYYSGMDY

SEQ ID NO: 305 - protein, 11 aa, Artificial Sequence
  source: /note=“Description of Artificial Sequence: Synthetic peptide”
RTSQSIGTNIH

SEQ ID NO: 306 - protein, 7 aa, Artificial Sequence
  source: /note=“Description of Artificial Sequence: Synthetic peptide”
NVSESIS

SEQ ID NO: 307 - protein, 7 aa, Artificial Sequence
  source: /note=“Description of Artificial Sequence: Synthetic peptide”
YVSESIS

SEQ ID NO: 308 - protein, 7 aa, Artificial Sequence
  source: /note=“Description of Artificial Sequence: Synthetic peptide”
YASESIS

SEQ ID NO: 309 - protein, 9 aa, Artificial Sequence
  source: /note=“Description of Artificial Sequence: Synthetic peptide”
QQSNTWPFT

SEQ ID NO: 310 - protein, 107 aa, Artificial Sequence
  source: /note=“Description of Artificial Sequence: Synthetic polypeptide”
EIVLTQSPDFQSVTPKEKVTITCRTSQSIGTNIHWYQQKPDQSPKLLIKNVSESISGVPSRFSGSGSGTDFTLTINSLEAEDAATYYCQQSNTWPFTFGGGTKVEIK

SEQ ID NO: 311 - protein, 107 aa, Artificial Sequence
  source: /note=“Description of Artificial Sequence: Synthetic polypeptide”
EIVLTQSPDFQSVTPKEKVTITCRTSQSIGTNIHWYQQKPDQSPKLLIKYVSESISGVPSRFSGSGSGTDFTLTINSLEAEDAATYYCQQSNTWPFTFGGGTKVEIK

SEQ ID NO: 312 - protein, 106 aa, Artificial Sequence
  source: /note=“Description of Artificial Sequence: Synthetic polypeptide”
EIVLTQSPDFQSVTPKEKVTITCRTSQSIGTNIHWYQQKPDQSPKLLIKYASESISGVPSRFSGSGSGTDFTLTINSLEAEDAATYCQQSNTWPFTFGGGTKVEIK

SEQ ID NO: 313 - protein, 121 aa, Artificial Sequence
  source: /note=“Description of Artificial Sequence: Synthetic polypeptide”
QVQLVQSGAEVKKPGASVKVSCKASGYTFTNYWIHWVRQAPGQGLEWMGVIDPSDTYPNYNKKFKGRVTMTTDTSTSTAYMELRSLRSDDTAVYYCARNGDSDYYSGMDYWGQGTTVTVSS

SEQ ID NO: 314 - protein, 5 aa, Artificial Sequence
  source: /note=“Description of Artificial Sequence: Synthetic peptide”
DAWMD

SEQ ID NO: 315 - protein, 19 aa, Artificial Sequence
  source: /note=“Description of Artificial Sequence: Synthetic peptide”
EIRNKANNLATYYAESVKG

SEQ ID NO: 316 - protein, 6 aa, Artificial Sequence
  source: /note=“Description of Artificial Sequence: Synthetic peptide”
YSPFAY

SEQ ID NO: 317 - protein, 115 aa, Artificial Sequence
  source: /note=“Description of Artificial Sequence: Synthetic polypeptide”
EVKFEESGGGLVQPGGSMKLSCAASGFTFSDAWMDWVRQGPEKGLEWVAEIRNKANNLATYYAESVKGRFTIPRDDSKSSVYLHMNSLRAEDTGIYYCYSPFAYWGQGTLVTVSA

SEQ ID NO: 318 - protein, 11 aa, Artificial Sequence
  source: /note=“Description of Artificial Sequence: Synthetic peptide”
RASQEISGYLS

SEQ ID NO: 319 - protein, 7 aa, Artificial Sequence
  source: /note=“Description of Artificial Sequence: Synthetic peptide”
AASTLDS

SEQ ID NO: 320 - protein, 9 aa, Artificial Sequence
  source: /note=“Description of Artificial Sequence: Synthetic peptide”
LQYVSYPYT

SEQ ID NO: 321 - protein, 107 aa, Artificial Sequence
  source: /note=“Description of Artificial Sequence: Synthetic polypeptide”
DIQMTQSPSSLSASLGERVSLTCRASQEISGYLSWLQQKPDGTIRRLIYAASTLDSGVPKRFSGSWSGSDYSLTISSLESEDFADYYCLQYVSYPYTFGGGTKLEIK

SEQ ID NO: 322 - protein, 107 aa, Artificial Sequence
  source: /note=“Description of Artificial Sequence: Synthetic polypeptide”
DIQMTQSPSSLLAALGERVSLTCRASQEISGYLSWLQQKPDGTIKRLIYAASTLDSGVPKRFSGSESGSDYSLTISSLESEDFADYYCLQYFSYPLTFGAGTKLELK

SEQ ID NO: 323 - protein, 116 aa, Artificial Sequence
  source: /note=“Description of Artificial Sequence: Synthetic polypeptide”
EVKLEESGGGLVQPGGSMKLSCEASGFTFSDAWMDWVRQSPEKGLEWVAEIRNKAKNHATYYAESVIGRFTISRDDSKSSVYLQMNSLRAEDTGIYYCGALGLDYWGQGTTLTVSS

SEQ ID NO: 324 - protein, 11 aa, Artificial Sequence
  source: /note=“Description of Artificial Sequence: Synthetic peptide”
RASQEISGYLS

SEQ ID NO: 325 - protein, 7 aa, Artificial Sequence
  source: /note=“Description of Artificial Sequence: Synthetic peptide”
AASTLDS

SEQ ID NO: 326 - protein, 9 aa, Artificial Sequence
  source: /note=“Description of Artificial Sequence: Synthetic peptide”
LQYFSYPLT

SEQ ID NO: 327 - protein, 5 aa, Artificial Sequence
  source: /note=“Description of Artificial Sequence: Synthetic peptide”
DAWMD

SEQ ID NO: 328 - protein, 19 aa, Artificial Sequence
  source: /note=“Description of Artificial Sequence: Synthetic peptide”
EIRNKAKNHATYYAESVIG

SEQ ID NO: 329 - protein, 7 aa, Artificial Sequence
  source: /note=“Description of Artificial Sequence: Synthetic peptide”
GALGLDY

SEQ ID NO: 330 - DNA, 321 bp, Artificial Sequence
  source: /note=“Description of Artificial Sequence: Synthetic polynucleotide”
gaaattgtgctgactcagtctccagactttcagtctgtgactccaaaggagaaagtcaccatcacctgcaggaccagtcagagcattggcacaaacatacactggtaccagcagaaaccagatcagtctccaaagctcctcatcaagaatgtttctgagtctatctctggagtcccatcgaggttcagtggcagtggatctgggacagatttcaccctcaccatcaatagcctggaagctgaagatgctgcaacgtattactgtcaacaaagtaatacctggccgttcacgttcggcggagggaccaaggtggagatcaaa

SEQ ID NO: 331 - DNA, 321 bp, Artificial Sequence
  source: /note=“Description of Artificial Sequence: Synthetic polynucleotide”
gaaattgtgctgactcagtctccagactttcagtctgtgactccaaaggagaaagtcaccatcacctgcaggaccagtcagagcattggcacaaacatacactggtaccagcagaaaccagatcagtctccaaagctcctcatcaagtatgtttctgagtctatctctggagtcccatcgaggttcagtggcagtggatctgggacagatttcaccctcaccatcaatagcctggaagctgaagatgctgcaacgtattactgtcaacaaagtaatacctggccgttcacgttcggcggagggaccaaggtggagatcaaa

SEQ ID NO: 332 - DNA, 321 bp, Artificial Sequence
  source: /note=“Description of Artificial Sequence: Synthetic polynucleotide”
gaaattgtgctgactcagtctccagactttcagtctgtgactccaaaggagaaagtcaccatcacctgcaggaccagtcagagcattggcacaaacatacactggtaccagcagaaaccagatcagtctccaaagctcctcatcaagtatgcttctgagtctatctctggagtcccatcgaggttcagtggcagtggatctgggacagatttcaccctcaccatcaatagcctggaagctgaagatgctgcaacgtattactgtcaacaaagtaatacctggccgttcacgttcggcggagggaccaaggtggagatcaaa

SEQ ID NO: 333 - DNA, 363 bp, Artificial Sequence
  source: /note=“Description of Artificial Sequence: Synthetic polynucleotide”
caggttcagctggtgcagtctggagctgaggtgaagaagcctggggcctcagtgaaggtctcctgcaaggcttctggttacacctttaccaactactggatacactgggtgcgacaggcccctggacaagggcttgagtggatgggagtgattgatccttctgatacttatccaaattacaataaaaagttcaagggcagagtcaccatgaccacagacacatccacgagcacagcctacatggagctgaggagcctgagatctgacgacacggccgtgtattactgtgcgagaaacggtgattccgattattactctggtatggactactgggggcaagggaccacggtcaccgtctcctca

SEQ ID NO: 334 - DNA, 321 bp, Mus sp.
gacatcttgctgactcagtctccagccatcctgtctgtgagtccaggagagagagtcagtttttcctgcaggaccagtcagagcattggcacaaacatacactggtatcagcaaagaacaaatggttttccaaggcttctcataaagaatgtttctgagtctatctctgggatcccttccaggtttagtggcagtggatcagggacagattttattcttagcatcaacagtgtggagtctgaagatattgcagattattattgtcaacaaagtaatacctggccgttcacgttcggaggggggaccaagctggaaataaaa

SEQ ID NO: 335 - DNA, 363 bp, Mus sp.
caggtccaattgcagcagcctgtgactgagctggtgaggccgggggcttcagtgatgttgtcctgcaaggcttctgactaccccttcaccaactactggatacactgggtaaagcagaggcctggacaaggcctggagtggatcggagtgattgatccttctgatacttatccaaattacaataaaaagttcaagggcaaggccacattgactgtagtcgtatcctccagcacagcctacatgcagctcagcagcctgacatctgacgattctgcggtctattactgtgcaagaaacggtgattccgattattactctggtatggactactggggtcaaggaacctcagtcaccgtctcctca

SEQ ID NO: 336 - DNA, 285 bp, Artificial Sequence
  source: /note=“Description of Artificial Sequence: Synthetic polynucleotide”
gaagtgaagtttgaggagtctggaggaggcttggtgcaacctggaggatccatgaaactctcttgtgctgcctctggattcacttttagtgacgcctggatggactgggtccgccagggtccagagaaggggcttgagtgggttgctgaaattagaaacaaagctaataatcttgcaacatactatgctgagtctgtgaaagggaggttcaccatcccaagagatgattccaaaagtagtccctttgcttactggggccaagggactctggtcactgtctctgca

SEQ ID NO: 337 - DNA, 321 bp, Artificial Sequence
  source: /note=“Description of Artificial Sequence: Synthetic polynucleotide”
gacatccagatgacccagtctccatcctccttatctgcctctctgggagaaagagtcagtctcacttgtcgggcaagtcaggaaattagtggttacttaagctggcttcagcagaaaccagatggaactattagacgcctgatctacgccgcatccactttagattctggtgtcccaaaaaggttcagtggcagttggtctgggtcagattattctctcaccatcagcagccttgagtctgaagattttgcagactattactgtctacaatatgttagttatccgtatacgttcggaggggggaccaagctggaaataaaa

SEQ ID NO: 338 - DNA, 321 bp, Artificial Sequence
  source: /note=“Description of Artificial Sequence: Synthetic polynucleotide”
gacattcagatgacacagtctccatcctccctacttgcggcgctgggagaaagagtcagtctcacttgtcgggcaagtcaggaaattagtggttacttaagctggcttcagcagaaaccagatggaactattaaacgcctgatctacgccgcatccactttagattctggtgtcccaaaaaggttcagtggcagtgagtctgggtcagattattctctcaccatcagcagtcttgagtctgaagattttgcagactattactgtctacaatattttagttatccgctcacgttcggtgctgggaccaagctggagctgaaa

SEQ ID NO: 339 - DNA, 348 bp, Artificial Sequence
  source: /note=“Description of Artificial Sequence: Synthetic polynucleotide”
gaagtgaagcttgaggagtctggaggaggcttggtgcaacctggaggatccatgaaactctcttgtgaagcctctggattcacttttagtgacgcctggatggactgggtccgtcagtctccagagaaggggcttgagtgggttgctgaaattagaaacaaagctaaaaatcatgcaacatactatgctgagtctgtgatagggaggttcaccatctcaagagatgattccaaaagtagtgtctacctgcaaatgaacagcttaagagctgaagacactggcatttattactgtggggctctgggccttgactactggggccaaggcaccactctcacagtctcctcg

SEQ ID NO: 340-676 - skipped (337 entries)

SEQ ID NO: 677 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 678 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 679 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 680-700 - skipped (21 entries)

SEQ ID NO: 701 - protein, 5 aa, Artificial Sequence
  source: /note=“Description of Artificial Sequence: Synthetic peptide”
NYGMN

SEQ ID NO: 702 - protein, 17 aa, Artificial Sequence
  source: /note=“Description of Artificial Sequence: Synthetic peptide”
WINTDTGEPTYADDFKG

SEQ ID NO: 703 - protein, 16 aa, Artificial Sequence
  source: /note=“Description of Artificial Sequence: Synthetic peptide”
NPPYYYGTNNAEAMDY

SEQ ID NO: 704 - protein, 7 aa, Artificial Sequence
  source: /note=“Description of Artificial Sequence: Synthetic peptide”
GFTLTNY

SEQ ID NO: 705 - protein, 6 aa, Artificial Sequence
  source: /note=“Description of Artificial Sequence: Synthetic peptide”
NTDTGE

SEQ ID NO: 706 - protein, 125 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707 - DNA, 375 bp, Artificial Sequence
  source: /note=“Description of Artificial Sequence: Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 708 - DNA, 375 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 709 - protein, 451 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 710 - protein, 11 aa, Artificial Sequence
  source: /note=“Description of Artificial Sequence: Synthetic peptide”
SSSQDISNYLN

SEQ ID NO: 711 - protein, 7 aa, Artificial Sequence
  source: /note=“Description of Artificial Sequence: Synthetic peptide”
YTSTLHL

SEQ ID NO: 712 - protein, 9 aa, Artificial Sequence
  source: /note=“Description of Artificial Sequence: Synthetic peptide”
QQYYNLPWT

SEQ ID NO: 713 - protein, 7 aa, Artificial Sequence
  source: /note=“Description of Artificial Sequence: Synthetic peptide”
SQDISNY

SEQ ID NO: 714 - protein, 3 aa, Artificial Sequence
  source: /note=“Description of Artificial Sequence: Synthetic peptide”
YTS

SEQ ID NO: 715 - protein, 6 aa, Artificial Sequence
  source: /note=“Description of Artificial Sequence: Synthetic peptide”
YYNLPW

SEQ ID NO: 716 - DNA, 1353 bp, Artificial Sequence
  source: /note=“Description of Artificial Sequence: Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 717 - DNA, 1353 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 718 - protein, 107 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719 - DNA, 321 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 720 - DNA, 321 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 721 - protein, 214 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 722 - DNA, 642 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 723 - DNA, 642 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 724 - protein, 125 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725 - DNA, 375 bp, Artificial Sequence
  source: /note=“Description of Artificial Sequence: Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 726 - DNA, 375 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 727 - protein, 451 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 728 - DNA, 1353 bp, Artificial Sequence
  source: /note=“Description of Artificial Sequence: Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 729 - DNA, 1353 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 730 - protein, 107 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731 - DNA, 321 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 732 - DNA, 321 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 733 - protein, 214 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 734 - DNA, 642 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 735 - DNA, 642 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 736 - DNA, 15 bp, Artificial Sequence
  source: /note=“Description of Artificial Sequence: Synthetic oligonucleotide”
aattacgggatgaac

SEQ ID NO: 737 - DNA, 15 bp, Artificial Sequence
  source: /note=“Description of Artificial Sequence: Synthetic oligonucleotide”
aactacggcatgaac

SEQ ID NO: 738 - DNA, 51 bp, Artificial Sequence
  source: /note=“Description of Artificial Sequence: Synthetic oligonucleotide”
tggattaacaccgacaccggggagcctacctacgcggacgatttcaaggga

SEQ ID NO: 739 - DNA, 51 bp, Artificial Sequence
  source: /note=“Description of Artificial Sequence: Synthetic oligonucleotide”
tggatcaacaccgacaccggcgagcctacctacgccgacgacttcaagggc

SEQ ID NO: 740 - DNA, 48 bp, Artificial Sequence
  source: /note=“Description of Artificial Sequence: Synthetic oligonucleotide”
aacccgccctactactacggaaccaacaacgccgaagccatggactac

SEQ ID NO: 741 - DNA, 48 bp, Artificial Sequence
  source: /note=“Description of Artificial Sequence: Synthetic oligonucleotide”
aaccccccttactactacggcaccaacaacgccgaggccatggactat

SEQ ID NO: 742 - DNA, 21 bp, Artificial Sequence
  source: /note=“Description of Artificial Sequence: Synthetic oligonucleotide”
ggattcaccctcaccaattac

SEQ ID NO: 743 - DNA, 21 bp, Artificial Sequence
  source: /note=“Description of Artificial Sequence: Synthetic oligonucleotide”
ggcttcaccctgaccaactac

SEQ ID NO: 744 - DNA, 18 bp, Artificial Sequence
  source: /note=“Description of Artificial Sequence: Synthetic oligonucleotide”
aacaccgacaccggggag

SEQ ID NO: 745 - DNA, 18 bp, Artificial Sequence
  source: /note=“Description of Artificial Sequence: Synthetic oligonucleotide”
aacaccgacaccggcgag

SEQ ID NO: 746 - DNA, 33 bp, Artificial Sequence
  source: /note=“Description of Artificial Sequence: Synthetic oligonucleotide”
agctctagtcaggatatctctaactacctgaac

SEQ ID NO: 747 - DNA, 33 bp, Artificial Sequence
  source: /note=“Description of Artificial Sequence: Synthetic oligonucleotide”
tcctccagccaggacatctccaactacctgaac

SEQ ID NO: 748 - DNA, 21 bp, Artificial Sequence
  source: /note=“Description of Artificial Sequence: Synthetic oligonucleotide”
tacactagcaccctgcacctg

SEQ ID NO: 749 - DNA, 21 bp, Artificial Sequence
  source: /note=“Description of Artificial Sequence: Synthetic oligonucleotide”
tacacctccaccctgcacctg

SEQ ID NO: 750 - DNA, 27 bp, Artificial Sequence
  source: /note=“Description of Artificial Sequence: Synthetic oligonucleotide”
cagcagtactataacctgccctggacc

SEQ ID NO: 751 - DNA, 27 bp, Artificial Sequence
  source: /note=“Description of Artificial Sequence: Synthetic oligonucleotide”
cagcagtactacaacctgccctggacc

SEQ ID NO: 752 - DNA, 21 bp, Artificial Sequence
  source: /note=“Description of Artificial Sequence: Synthetic oligonucleotide”
agtcaggatatctctaactac

SEQ ID NO: 753 - DNA, 21 bp, Artificial Sequence
  source: /note=“Description of Artificial Sequence: Synthetic oligonucleotide”
agccaggacatctccaactac

SEQ ID NO: 754 - DNA, 9 bp, Artificial Sequence
  source: /note=“Description of Artificial Sequence: Synthetic oligonucleotide”
tacactagc

SEQ ID NO: 755 - DNA, 9 bp, Artificial Sequence
  source: /note=“Description of Artificial Sequence: Synthetic oligonucleotide”
tacacctcc

SEQ ID NO: 756 - DNA, 18 bp, Artificial Sequence
  source: /note=“Description of Artificial Sequence: Synthetic oligonucleotide”
tactataacctgccctgg

SEQ ID NO: 757 - DNA, 18 bp, Artificial Sequence
  source: /note=“Description of Artificial Sequence: Synthetic oligonucleotide”
tactacaacctgccctgg

SEQ ID NO: 758 - DNA, 15 bp, Artificial Sequence
  source: /note=“Description of Artificial Sequence: Synthetic oligonucleotide”
aactacgggatgaac

SEQ ID NO: 759 - DNA, 51 bp, Artificial Sequence
  source: /note=“Description of Artificial Sequence: Synthetic oligonucleotide”
tggattaacaccgacaccggcgagcctacctacgccgacgactttaagggc

SEQ ID NO: 760 - DNA, 48 bp, Artificial Sequence
  source: /note=“Description of Artificial Sequence: Synthetic oligonucleotide”
aaccccccctactactacggcactaacaacgccgaggctatggactac

SEQ ID NO: 761 - DNA, 21 bp, Artificial Sequence
  source: /note=“Description of Artificial Sequence: Synthetic oligonucleotide”
ggcttcaccctgactaactac

SEQ ID NO: 762 - protein, 447 aa, Artificial Sequence
  source: /note=“Description of Artificial Sequence: Synthetic polypeptide”
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, Artificial Sequence
  source: /note=“Description of Artificial Sequence: Synthetic polypeptide”
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, Artificial Sequence
  source: /note=“Description of Artificial Sequence: Synthetic polypeptide”
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, Artificial Sequence
  source: /note=“Description of Artificial Sequence: Synthetic polypeptide”
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766 - protein, 10 aa, Artificial Sequence
  source: /note=“Description of Artificial Sequence: Synthetic peptide”
GFTLTNYGMN

SEQ ID NO: 767-800 - skipped (34 entries)

SEQ ID NO: 801 - protein, 5 aa, Artificial Sequence
  source: /note=“Description of Artificial Sequence: Synthetic peptide”
SYNMH

SEQ ID NO: 802 - protein, 17 aa, Artificial Sequence
  source: /note=“Description of Artificial Sequence: Synthetic peptide”
DIYPGNGDTSYNQKFKG

SEQ ID NO: 803 - protein, 9 aa, Artificial Sequence
  source: /note=“Description of Artificial Sequence: Synthetic peptide”
VGGAFPMDY

SEQ ID NO: 804 - protein, 7 aa, Artificial Sequence
  source: /note=“Description of Artificial Sequence: Synthetic peptide”
GYTFTSY

SEQ ID NO: 805 - protein, 6 aa, Artificial Sequence
  source: /note=“Description of Artificial Sequence: Synthetic peptide”
YPGNGD

SEQ ID NO: 806 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807 - DNA, 354 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 808 - protein, 444 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 809 - DNA, 1332 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 810 - protein, 15 aa, Artificial Sequence
  source: /note=“Description of Artificial Sequence: Synthetic peptide”
RASESVEYYGTSLMQ

SEQ ID NO: 811 - protein, 7 aa, Artificial Sequence
  source: /note=“Description of Artificial Sequence: Synthetic peptide”
AASNVES

SEQ ID NO: 812 - protein, 9 aa, Artificial Sequence
  source: /note=“Description of Artificial Sequence: Synthetic peptide”
QQSRKDPST

SEQ ID NO: 813 - protein, 11 aa, Artificial Sequence
  source: /note=“Description of Artificial Sequence: Synthetic peptide”
SESVEYYGTSL

SEQ ID NO: 814 - protein, 3 aa, Artificial Sequence
  source: /note=“Description of Artificial Sequence: Synthetic peptide”
AAS

SEQ ID NO: 815 - protein, 6 aa, Artificial Sequence
  source: /note=“Description of Artificial Sequence: Synthetic peptide”
SRKDPS

SEQ ID NO: 816 - protein, 111 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817 - DNA, 333 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 818 - protein, 218 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - DNA, 654 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 820 - protein, 17 aa, Artificial Sequence
  source: /note=“Description of Artificial Sequence: Synthetic peptide”
DIYPGQGDTSYNQKFKG

SEQ ID NO: 821 - protein, 6 aa, Artificial Sequence
  source: /note=“Description of Artificial Sequence: Synthetic peptide”
YPGQGD

SEQ ID NO: 822 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 824 - protein, 444 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 826 - protein, 111 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827 - DNA, 333 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 828 - protein, 218 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - DNA, 654 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 830 - protein, 114 aa, Artificial Sequence
  source: /note=“Description of Artificial Sequence: Synthetic polypeptide”
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  source: /note=“Description of Artificial Sequence: Synthetic polypeptide”
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  source: /note=“Description of Artificial Sequence: Synthetic polypeptide”
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  source: /note=“Description of Artificial Sequence: Synthetic polypeptide”
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903 - protein, 451 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 904 - protein, 214 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 905 - DNA, 363 bp, Artificial Sequence
  source: /note=“Description of Artificial Sequence: Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 906 - DNA, 321 bp, Artificial Sequence
  source: /note=“Description of Artificial Sequence: Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 907 - DNA, 1353 bp, Artificial Sequence
  source: /note=“Description of Artificial Sequence: Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 908 - DNA, 642 bp, Artificial Sequence
  source: /note=“Description of Artificial Sequence: Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 909 - protein, 5 aa, Artificial Sequence
  source: /note=“Description of Artificial Sequence: Synthetic peptide”
SYGVD

SEQ ID NO: 910 - protein, 7 aa, Artificial Sequence
  source: /note=“Description of Artificial Sequence: Synthetic peptide”
GFSLSSY

SEQ ID NO: 911 - protein, 16 aa, Artificial Sequence
  source: /note=“Description of Artificial Sequence: Synthetic peptide”
VIWGGGGTYYASSLMG

SEQ ID NO: 912 - protein, 5 aa, Artificial Sequence
  source: /note=“Description of Artificial Sequence: Synthetic peptide”
WGGGG

SEQ ID NO: 913 - protein, 13 aa, Artificial Sequence
  source: /note=“Description of Artificial Sequence: Synthetic peptide”
HAYGHDGGFAMDY

SEQ ID NO: 914 - protein, 11 aa, Artificial Sequence
  source: /note=“Description of Artificial Sequence: Synthetic peptide”
RASESVSSNVA

SEQ ID NO: 915 - protein, 7 aa, Artificial Sequence
  source: /note=“Description of Artificial Sequence: Synthetic peptide”
SESVSSN

SEQ ID NO: 916 - protein, 7 aa, Artificial Sequence
  source: /note=“Description of Artificial Sequence: Synthetic peptide”
GASNRAT

SEQ ID NO: 917 - protein, 3 aa, Artificial Sequence
  source: /note=“Description of Artificial Sequence: Synthetic peptide”
GAS

SEQ ID NO: 918 - protein, 9 aa, Artificial Sequence
  source: /note=“Description of Artificial Sequence: Synthetic peptide”
GQSYSYPFT

SEQ ID NO: 919 - protein, 6 aa, Artificial Sequence
  source: /note=“Description of Artificial Sequence: Synthetic peptide”
SYSYPF

SEQ ID NO: 920 - protein, 124 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, Artificial Sequence
  source: /note=“Description of Artificial Sequence: Synthetic polypeptide”
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922-1500 - skipped (579 entries)

SEQ ID NO: 1501 - protein, 5 aa, Artificial Sequence
  source: /note=“Description of Artificial Sequence: Synthetic peptide”
TYWMH

SEQ ID NO: 1502 - protein, 17 aa, Artificial Sequence
  source: /note=“Description of Artificial Sequence: Synthetic peptide”
NIYPGTGGSNFDEKFKN

SEQ ID NO: 1503 - protein, 8 aa, Artificial Sequence
  source: /note=“Description of Artificial Sequence: Synthetic peptide”
WTTGTGAY

SEQ ID NO: 1504 - protein, 7 aa, Artificial Sequence
  source: /note=“Description of Artificial Sequence: Synthetic peptide”
GYTFTTY

SEQ ID NO: 1505 - protein, 6 aa, Artificial Sequence
  source: /note=“Description of Artificial Sequence: Synthetic peptide”
YPGTGG

SEQ ID NO: 1506 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 1507 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 1508 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 1509 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 1510 - protein, 17 aa, Artificial Sequence
  source: /note=“Description of Artificial Sequence: Synthetic peptide”
KSSQSLLDSGNQKNFLT

SEQ ID NO: 1511 - protein, 7 aa, Artificial Sequence
  source: /note=“Description of Artificial Sequence: Synthetic peptide”
WASTRES

SEQ ID NO: 1512 - protein, 9 aa, Artificial Sequence
  source: /note=“Description of Artificial Sequence: Synthetic peptide”
QNDYSYPYT

SEQ ID NO: 1513 - protein, 13 aa, Artificial Sequence
  source: /note=“Description of Artificial Sequence: Synthetic peptide”
SQSLLDSGNQKNF

SEQ ID NO: 1514 - protein, 3 aa, Artificial Sequence
  source: /note=“Description of Artificial Sequence: Synthetic peptide”
WAS

SEQ ID NO: 1515 - protein, 6 aa, Artificial Sequence
  source: /note=“Description of Artificial Sequence: Synthetic peptide”
DYSYPY

SEQ ID NO: 1516 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 1517 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 1518 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1519 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 1520 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 1521 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 1522 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1523 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 1524 - DNA, 15 bp, Artificial Sequence
  source: /note=“Description of Artificial Sequence: Synthetic oligonucleotide”
acctactggatgcac

SEQ ID NO: 1525 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 1526 - DNA, 24 bp, Artificial Sequence
  source: /note=“Description of Artificial Sequence: Synthetic oligonucleotide”
tggactaccggcacaggcgcctac

SEQ ID NO: 1527 - DNA, 21 bp, Artificial Sequence
  source: /note=“Description of Artificial Sequence: Synthetic oligonucleotide”
ggctacaccttcactacctac

SEQ ID NO: 1528 - DNA, 18 bp, Artificial Sequence
  source: /note=“Description of Artificial Sequence: Synthetic oligonucleotide”
taccccggcaccggcggc

SEQ ID NO: 1529 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 1530 - DNA, 21 bp, Artificial Sequence
  source: /note=“Description of Artificial Sequence: Synthetic oligonucleotide”
tgggcctctactagagaatca

SEQ ID NO: 1531 - DNA, 27 bp, Artificial Sequence
  source: /note=“Description of Artificial Sequence: Synthetic oligonucleotide”
cagaacgactatagctacccctacacc

SEQ ID NO: 1532 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 1533 - DNA, 9 bp, Artificial Sequence
  source: /note=“Description of Artificial Sequence: Synthetic oligonucleotide”
tgggcctct

SEQ ID NO: 1534 - DNA, 18 bp, Artificial Sequence
  source: /note=“Description of Artificial Sequence: Synthetic oligonucleotide”
gactatagctacccctac

SEQ ID NO: 1535 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1536 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1537 - protein, 447 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1538 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1539 - protein, 447 aa, Artificial Sequence
  source: /note=“Description of Artificial Sequence: Synthetic polypeptide”
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1540 - protein, 213 aa, Artificial Sequence
  source: /note=“Description of Artificial Sequence: Synthetic polypeptide”
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1541 - protein, 10 aa, Artificial Sequence
  source: /note=“Description of Artificial Sequence: Synthetic peptide”
GYTFTTYWMH

SEQ ID NO: 1542-1600 - skipped (59 entries)

SEQ ID NO: 1601 - protein, 5 aa, Artificial Sequence
  source: /note=“Description of Artificial Sequence: Synthetic peptide”
SYWMY

SEQ ID NO: 1602 - protein, 17 aa, Artificial Sequence
  source: /note=“Description of Artificial Sequence: Synthetic peptide”
RIDPNSGSTKYNEKFKN

SEQ ID NO: 1603 - protein, 11 aa, Artificial Sequence
  source: /note=“Description of Artificial Sequence: Synthetic peptide”
DYRKGLYAMDY

SEQ ID NO: 1604 - protein, 7 aa, Artificial Sequence
  source: /note=“Description of Artificial Sequence: Synthetic peptide”
GYTFTSY

SEQ ID NO: 1605 - protein, 6 aa, Artificial Sequence
  source: /note=“Description of Artificial Sequence: Synthetic peptide”
DPNSGS

SEQ ID NO: 1606 - protein, 120 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 1607 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 1608 - protein, 446 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 1609 - protein, 11 aa, Artificial Sequence
  source: /note=“Description of Artificial Sequence: Synthetic peptide”
KASQDVGTAVA

SEQ ID NO: 1610 - protein, 7 aa, Artificial Sequence
  source: /note=“Description of Artificial Sequence: Synthetic peptide”
WASTRHT

SEQ ID NO: 1611 - protein, 9 aa, Artificial Sequence
  source: /note=“Description of Artificial Sequence: Synthetic peptide”
QQYNSYPLT

SEQ ID NO: 1612 - protein, 7 aa, Artificial Sequence
  source: /note=“Description of Artificial Sequence: Synthetic peptide”
SQDVGTA

SEQ ID NO: 1613 - protein, 3 aa, Artificial Sequence
  source: /note=“Description of Artificial Sequence: Synthetic peptide”
WAS

SEQ ID NO: 1614 - protein, 6 aa, Artificial Sequence
  source: /note=“Description of Artificial Sequence: Synthetic peptide”
YNSYPL

SEQ ID NO: 1615 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 1616 - protein, 107 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 1617 - DNA, 321 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 1618 - protein, 214 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1619 - DNA, 642 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 1620 - protein, 120 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 1621 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 1622 - protein, 446 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 1623 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 1624 - protein, 107 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 1625 - DNA, 321 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 1626 - protein, 214 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1627 - DNA, 642 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 1628 - DNA, 15 bp, Artificial Sequence
  source: /note=“Description of Artificial Sequence: Synthetic oligonucleotide”
agctactggatgtac

SEQ ID NO: 1629 - DNA, 51 bp, Artificial Sequence
  source: /note=“Description of Artificial Sequence: Synthetic oligonucleotide”
agaatcgaccctaatagcggctctactaagtataacgagaagtttaagaat

SEQ ID NO: 1630 - DNA, 33 bp, Artificial Sequence
  source: /note=“Description of Artificial Sequence: Synthetic oligonucleotide”
gactatagaaagggcctgtacgctatggactac

SEQ ID NO: 1631 - DNA, 21 bp, Artificial Sequence
  source: /note=“Description of Artificial Sequence: Synthetic oligonucleotide”
ggctacaccttcactagctac

SEQ ID NO: 1632 - DNA, 18 bp, Artificial Sequence
  source: /note=“Description of Artificial Sequence: Synthetic oligonucleotide”
gaccctaatagcggctct

SEQ ID NO: 1633 - DNA, 33 bp, Artificial Sequence
  source: /note=“Description of Artificial Sequence: Synthetic oligonucleotide”
aaagcctctcaggacgtgggcaccgccgtggcc

SEQ ID NO: 1634 - DNA, 21 bp, Artificial Sequence
  source: /note=“Description of Artificial Sequence: Synthetic oligonucleotide”
tgggcctctactagacacacc

SEQ ID NO: 1635 - DNA, 27 bp, Artificial Sequence
  source: /note=“Description of Artificial Sequence: Synthetic oligonucleotide”
cagcagtataatagctaccccctgacc

SEQ ID NO: 1636 - DNA, 21 bp, Artificial Sequence
  source: /note=“Description of Artificial Sequence: Synthetic oligonucleotide”
tctcaggacgtgggcaccgcc

SEQ ID NO: 1637 - DNA, 9 bp, Artificial Sequence
  source: /note=“Description of Artificial Sequence: Synthetic oligonucleotide”
tgggcctct

SEQ ID NO: 1638 - DNA, 18 bp, Artificial Sequence
  source: /note=“Description of Artificial Sequence: Synthetic oligonucleotide”
tataatagctaccccctg

SEQ ID NO: 1639 - protein, 448 aa, Artificial Sequence
  source: /note=“Description of Artificial Sequence: Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1640 - protein, 214 aa, Artificial Sequence
  source: /note=“Description of Artificial Sequence: Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1641 - protein, 450 aa, Artificial Sequence
  source: /note=“Description of Artificial Sequence: Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1642 - protein, 216 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1643 - protein, 451 aa, Artificial Sequence
  source: /note=“Description of Artificial Sequence: Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1644 - protein, 215 aa, Artificial Sequence
  source: /note=“Description of Artificial Sequence: Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1645 - protein, 123 aa, Artificial Sequence
  source: /note=“Description of Artificial Sequence: Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 1646 - protein, 106 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 1647 - protein, 10 aa, Artificial Sequence
  source: /note=“Description of Artificial Sequence: Synthetic peptide”
GYTFTSYWMY

SEQ ID NO: 1648 - protein, 6 aa, Artificial Sequence
  source: /note=“Description of Artificial Sequence: Synthetic 6xHis tag”
HHHHHH